(19) United States
(12) Patent Application Publication
(10) Pub. No.: US 2011/0130397 A1
(43) Pub. Date: Jun. 2, 2011

(54) PYRROLINONE COMPOUNDS AS INHIBITORS OF BACTERIAL PEPTIDYL TRNA HYDROLASE AND USES THEREOF

(76) Inventors: Soongyu Choi, Skillman, NJ (US); Arthur Branstrom, East Windsor, NJ (US); Scott A. Gothe, Morris Plains, NJ (US); Richard Lipman, Monmouth Junction, NJ (US); Tamil Arasu, Edison, NJ (US); Richard G. Wilde, Somerville, NJ (US)

(21) Appl. No.: 12/441,932

(22) PCT Filed: Sep. 21, 2007
(86) PCT No.: PCT/US07/20462

§ 371 Date: Jan. 21, 2010

Related U.S. Application Data
(60) Provisional application No. 60/846,799, filed on Sep. 22, 2006.

(51) Int. Cl. A61K 31/428; C07D 417/04; C07D 417/14; A61K 31/433; A61K 31/427; A61K 31/4439; C07D 413/14; A61K 31/423; C07D 473/30; A61K 31/52; C07D 405/14; A61K 31/506; C07D 473/34; C07D 487/04; A61K 31/519; A61K 31/5377; A61P 31/04; A01N 43/90; A01N 43/84; A01N 43/824; A01N 43/78

(52) U.S. Cl. 514/234.2; 548/162; 514/367; 548/141; 514/363; 548/159; 548/195; 514/371; 546/270.1; 514/338; 548/222; 514/375; 544/276; 514/263.23; 544/328; 514/256; 544/277; 544/262; 514/262.1; 544/118

(57) ABSTRACT

Provided herein are compounds that modulate the activity of a bacterial peptidyl tRNA hydrolase, including compositions and dosage forms comprising the compounds. Further provided herein are methods for preventing or inhibiting bacterial proliferation as well as methods for preventing, treating, and/or managing a bacterial infection using such compounds and compositions.

This application claims the benefit of U.S. provisional application No. 60/846,799, filed Sep. 22, 2006, which is incorporated by reference herein in its entirety.

### 1. FIELD

Provided herein are compounds that modulate the activity of a bacterial peptidyl tRNA hydrolase, including compositions and dosage forms comprising the compounds. Further provided herein are methods for screening and identifying compounds that modulate the activity of a bacterial peptidyl tRNA hydrolase. In particular, provided herein are assays for the identification of compounds that inhibit or reduce the activity of a bacterial peptidyl tRNA hydrolase. Further provided herein are methods for preventing or inhibiting bacterial proliferation as well as methods for preventing, treating, and/or managing a bacterial infection using such compounds and compositions.

### 2. BACKGROUND

Therapeutic challenge to pathogens creates selective pressure to evolve or acquire resistance, and the emergence and spread of bacterial resistance to every antibiotic available has been documented. Antibiotic resistance will certainly remain as a major issue for treating bacterial infections. Additional factors, such as treatment of non-susceptible infections and poor compliance with recommended dosage regimens accelerate the frequency and spread of resistance. Further, environmental dissemination of pathogenic strains as an act of bioterrorism is an important concern. Several organisms, including Francisella tularensis, Yersinia pestis, Brucella spp., Coxiella burnetii, Bacillus anthracis, and Mycobacterium tuberculosis are of concern as potential agents for biowarfare and bioterrorism. New antibiotics are thus necessary for the treatment of bacterial infections. In particular, it is advantageous to develop antibacterials that inhibit a novel molecular target, one that is different from the targets of currently available antibacterial compounds, because such a compound would not encounter preexisting resistance.

Peptidyl tRNA hydrolase (“Pth”) recycles tRNA from peptidyl-tRNAs that prematurely dissociate from the ribosome during translation (Kramer, B. et al. 1999 Proteins 37:228-241; Menez, J., et al. 2002 Mol. Microbiol. 45:123-129; Menninger, J. R. 1979 J. Bacteriol. 137:694-696; Menninger, J. R., et al. 1973. Mol. Gen. Genet. 121:307-324). Protein synthesis involves the concerted effort of a number of factors, including the ribosome, mRNA, tRNA, and assorted protein factors. In bacteria, it is estimated that only 76% of the initiated protein chains will actually complete the synthesis of the polypeptide (Jorgensen, F. and C. G. Kurland 1990 J. Mol. Biol. 215:511-521). To process these prematurely dissociated products, Pth cleaves the ester bond between the peptide and the tRNA (Kossel, H. 1970 Biochim. Biophys. Acta. 204:191-202; Shiloach, J., et al. 1975 Nucleic Acids Res. 2:1941-1950) and restores the tRNA portion of the peptidyl-tRNA for aminoacylation. This dissociation is believed to be the result of a general editing function to prevent the expression of mutant proteins resulting from the incorporation of incorrect amino acids due to improper codon-anticodon pairing, where the rate of mis-incorporation of amino acids in translation is estimated to occur once per 90 peptide elongation steps (Menninger, J. R. 1976 J. Biol. Chem. 251:3392-3398). Importantly, accumulated peptidyl-tRNA is toxic to the cell and must be cleared by Pth activity. This was demonstrated when an E. coli strain containing a temperature-sensitive mutation within the Pth enzyme was isolated (Atherly, A. G. and J. R. Menninger 1972 Nat. New Biol. 240:245-246). When grown under non-permissive conditions, the mutant cells accumulate peptidyl-tRNA, which reduces the availability of acylatable tRNAs, thus inhibiting protein synthesis and leading to cell death (Menninger, J. R. 1979 J. Bacteriol. 137:694-696). In fact, the bacterial peptidyl-tRNA hydrolase enzyme has been shown in genetic studies to be essential in E. coli (Heurgue-Hamard, V., et al. 1996 EMBO J. 15:2826-2833; Menninger, J. R. 1979 J. Bacteriol. 137:694-696) and Bacillus subtilis (Menez, J., R. H. et al. 2002 Mol. Microbiol. 45:123-129).

### 3. SUMMARY

The present embodiments are based, in part, on the use of bacterial Pth as a novel target for the identification and development of new antibacterial compounds. Pth is an attractive target for several reasons. First, the Pth gene is highly conserved among bacteria so that inhibitors of Pth activity may be used as a broad-spectrum antibacterial agent. Second, Pth is not targeted by currently available antibacterial compounds. Thus, a Pth inhibitor would be useful against strains of bacteria that have demonstrated resistance to currently available antibiotics. Third, Pth is an essential enzyme in bacteria but is nonessential in eukaryotes. Thus, Pth inhibitors demonstrate bactericidal activity while maintaining low cytotoxicity in mammalian cells. Fourth, Pth inhibitors have an advantage over antibiotics which target the ribosome and inhibit protein synthesis. This is because of the relatively small number of Pth enzyme molecules present in a bacterial cell, compared, for example, to the number of ribosomes. The number of Pth molecules per cell has been estimated to be at least one or two orders of magnitude less than the number of ribosomes (Cruz-Vera, L. R., et al. 2000. J. Bacteriol. 182:1523-1528; Dutka, S., et al. 1993 Nucleic Acids Res. 21:4025-4030). This means that Pth inhibitors have a stoichiometric advantage over currently available protein synthesis inhibitors that target the ribosome because a Pth inhibitor has fewer potential targets with which to interact. This also means that Pth inhibitors should be effective at lower concentrations compared to conventional antibiotics that target the ribosome. Fifth, Pth inhibitors are likely to be highly selective for inhibition of the bacterial enzyme versus mammalian homologs, permitting the use of lower doses and leading to fewer side effects. This is because the primary structure of human Pth active site differs somewhat from that of bacterial Pth, therefore inhibitors of bacterial Pth should not inhibit the eukaryotic enzyme. Likewise, bacterial Pth inhibitors should not inhibit the mammalian phosphodiesterase-type enzyme which has demonstrated peptidyl-tRNA hydrolase activity. Finally, the availability of the crystal structure of the E. coli enzyme, which was solved at a resolution of 1.2 Å (Schmitt, E., et al. 1997 Proteins 28:135-136; Schmitt, E., et al. 1997 EMBO J. 16:4760-4769) and which includes mapping of key amino acid residues, makes possible a comprehensive approach to identify inhibitory compounds through modeling of additional bacterial and eukaryotic peptidyl tRNA hydrolase enzymes and through de novo structure-based drug design of small molecule peptidyl tRNA hydrolase inhibitors.

In certain embodiments, provided herein are compounds having the formula I:

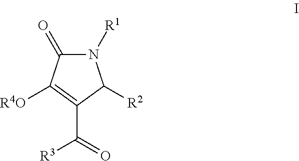

wherein R¹-R⁴ are as set forth below. In addition, compounds provided herein include the compounds set forth in Table 1.

In another embodiment, provided herein are pharmaceutical compositions comprising an effective amount of a compound provided herein and a pharmaceutically acceptable carrier, excipient or diluent.

In another embodiment, provided herein are methods of preventing or inhibiting replication of a bacterial organism, comprising contacting the bacterial organism with an effective amount of a compound provided herein.

In another embodiment, provided herein are methods of preventing, treating or managing a bacterial infection, comprising administering to a subject in need thereof (e.g., a subject having a bacterial infection) an effective amount of a compound provided herein.

### 4. BRIEF DESCRIPTION OF THE FIGURES

FIG. 1: Schematic showing the amino acid sequence of peptidyl tRNA hydrolase is highly conserved among bacteria.

FIG. 2: Schematic of a cell-free fluorescence polarization assay which can be utilized to assay a compound for the ability to enhance or inhibit the activity of a peptidyl tRNA hydrolase.

FIG. 3: Initial results of library screening for compounds having inhibitory activity against peptidyl tRNA hydrolase, showing hit compounds falling within a 95% confidence interval (at 2 Std. Dev.) which were selected for further analysis.

FIG. 4: Minimal Inhibitory Concentrations (MIC) for selected compounds identified using E. coli peptidyl tRNA hydrolase in an assay for antiproliferative activity, showing compounds having antibacterial activity combined with low cytotoxicity in mammalian cells (Huh7 cells).

FIG. 5: Minimal Inhibitory Concentrations (MIC) for selected compounds identified using E. coli. peptidyl tRNA hydrolase in an assay for antiproliferative activity, showing inhibition of vancomycin-resistant (VRE), methicillin-resistant (MRSA), or multi-drug resistant (MDR) bacteria.

FIG. 6: Bactericidal Curves (in vitro), showing a 3 log reduction in bacterial load at 18 hours for the tested peptidyl tRNA hydrolase inhibitors (series II and series I). S. epidermidis 12228 was used as the prototype bacteria in this assay.

FIG. 7. Sequence alignment of the loop region of peptidyl tRNA hydrolase from various bacteria.

FIGS. 8A-8C: Non-limiting list of bacteria that cause infections which can be reduced, inhibited, prevented, treated or managed in accordance with the invention.

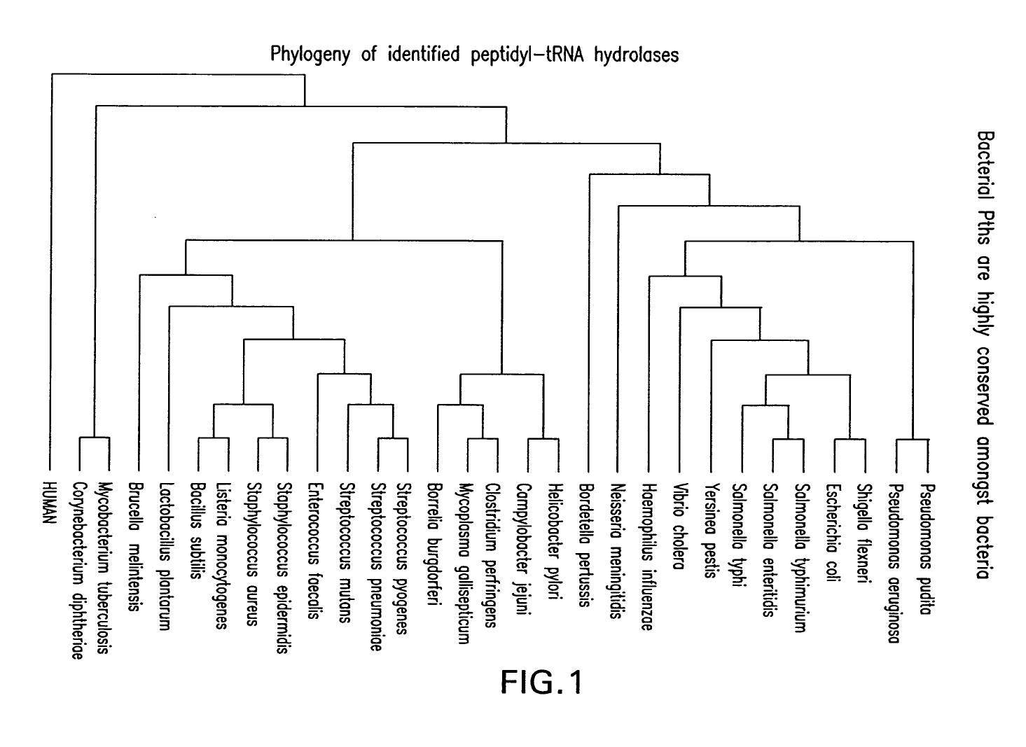

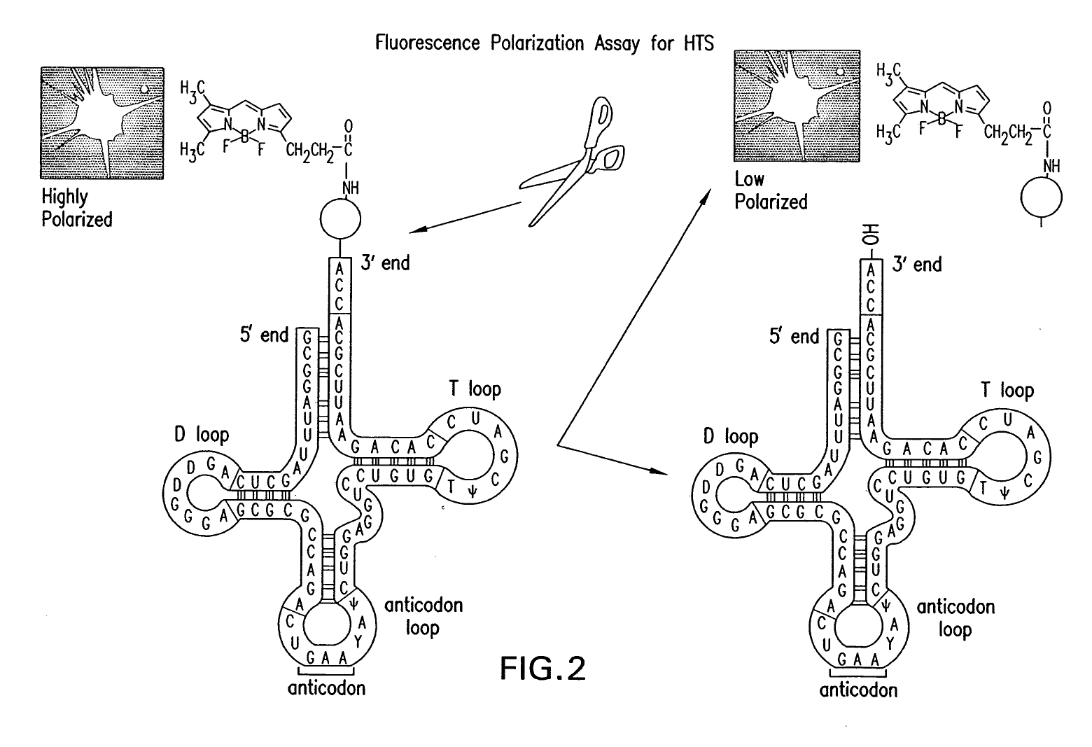

### 5. DETAILED DESCRIPTION

### 5.1 Terminology

As used herein, the terms “about” or “approximately” in the context of a numerical value refers to a number within 10% of the numerical value recited.

As used herein, the terms “compound” and “compounds provided herein” refer to any agent that is being tested for its ability to inhibit the activity of a peptidyl tRNA hydrolase or has been identified as inhibiting the activity of a peptidyl tRNA hydrolase, including the compounds provided herein, such as in Section 5.2 and Table 1, and pharmaceutically acceptable salts, solvates, hydrates, prodrugs and stereoisomers thereof.

As used herein, the term “effective amount” refers to the amount (e.g., of a compound, identified in accordance with the methods provided herein, including the compounds described in Section 5.2 and Table 1, infra) which is sufficient to (1) reduce, ameliorate, or prevent the progression of a bacterial infection; (2) reduce or inhibit bacterial replication and/or bacterial viability; (3) reduce or inhibit a bacterial infection; (4) reduce or inhibit the spread of a bacterial infection; (5) reduce or ameliorate the severity and/or duration of a bacterial infection or one or more symptoms thereof; (6) prevent the recurrence, development or onset of a bacterial infection or one or more symptoms thereof; (7) reduce or inhibit protein synthesis; and/or (8) enhance or improve the prophylactic and/or therapeutic effect(s) of another therapy.

As used herein, the term “in combination” refers to the use of more than one therapy (e.g., prophylactic and/or therapeutic agents). The use of the term “in combination” does not restrict the order in which therapies (e.g., prophylactic and/or therapeutic agents) are administered to a subject with a bacterial infection. A first therapy (e.g., a prophylactic or therapeutic agent, such as a compound identified in accordance with the methods provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a subject with a bacterial infection.

As used herein, the term “infection” means the invasion by and presence of a bacterial cell in a subject. In one embodiment, an infection is an “active” infection, i.e., one in which the bacteria are proliferating in the subject. Such an infection is characterized by the spread of bacteria to other cells, tissues, and organs, from the cells, tissues, or organs initially infected by the bacteria. An infection may also be a latent infection, i.e., one in which the bacteria are not proliferating. In one embodiment, an infection refers to the pathological state resulting from the presence of the bacteria in the body or by the invasion of the body by bacteria.

As used herein, the terms “manage,” “managing” and “management” in the context of the administration of a therapy to a subject, refer to the beneficial effects that a subject derives from a therapy (e.g., a prophylactic or therapeutic agent), which does not result in the eradication of the infection. In certain embodiments, a subject is administered one or more therapies to manage an infection so as to prevent the progression or worsening of the infection.

As used herein, the terms “non-responsive” and refractory” describe patients treated with a currently available therapy (e.g., a prophylactic or therapeutic agent) for a bacterial infection, which is not clinically adequate to eradicate such infection, and/or relieve one or more symptoms thereof. Typically, such patients suffer from severe, persistently active bacterial infection and require additional therapy to ameliorate the symptoms associated with the infection.

As used herein, the terms “prevent,” “preventing,” and “prevention” in the context of the administration of a therapy to a subject, refer to the prevention of the development, recurrence or onset of a bacterial infection or one or more symptoms thereof, resulting from the administration of one or more compounds identified in accordance the methods provided herein, including the compounds described in Section 5.2 and Table 1, or the administration of a combination of such a compound and another therapy for a bacterial infection.

As used herein, the terms “prophylactic agent” and “prophylactic agents” refer to any agent(s) which can be used in the prevention of a bacterial infection. In certain embodiments, the term “prophylactic agent” refers to a compound identified in the screening assays described herein, or a compound described in Section 5.2 and Table 1. In certain other embodiments, the term “prophylactic agent” refers to an agent other than a compound identified in the screening assays described herein, or a compound described in Section 5.2 and Table 1, which is known to be useful for, or has been or is currently being used to prevent or impede the onset, development, progression, replication, spread, and/or severity of a bacterial infection or one or more symptoms thereof.

As used herein, the phrase “prophylactically effective amount” refers to the amount of a therapy (e.g., a prophylactic agent) which is sufficient to result in the prevention of the development, recurrence or onset of a bacterial infection or one or more symptoms thereof.

As used herein, the terms “subject” and “patient” are used interchangeably herein. The terms “subject” and “subjects” refer to an animal, such as a mammal, including non-primates (e.g., cow, pig, horse, cat, dog, rat or mouse) and primates (e.g., monkey or human), and in one embodiment a human. In one embodiment, the subject is a farm animal (e.g., horse, cow, pig) or a pet (e.g., dog or cat). In one embodiment, the subject is a human. In certain embodiments, the subject is refractory or non-responsive to current therapies for a bacterial infection. In one embodiment, the subject is a premature human infant. In one embodiment, the subject is a human infant. In one embodiment, the subject is a human adult. In one embodiment, the subject is a human child. In one embodiment, the subject is an elderly human. In one embodiment, the subject is immunosuppressed or immunocompromised.

As used herein, the term “synergistic” refers to a combination of a compound identified using one of the methods described herein or a compound described herein, and another therapy (e.g., a prophylactic or therapeutic agent), which is more effective than the additive effects of the agents. In a specific embodiment, a synergistic effect of a combination of therapies permits the use of lower dosages of one or more of the therapies and/or less frequent administration of the therapies to a subject with a bacterial infection. The ability to utilize lower dosages of a therapy and/or to administer the therapy less frequently reduces the toxicity associated with the administration of the therapy to a subject without reducing the efficacy of the therapy in the prevention, treatment, management or amelioration of the bacterial infection or one or more symptoms thereof. In another embodiment, a synergistic effect results in improved efficacy of therapies in the prevention, treatment, and/or management of a bacterial infection or one or more symptoms thereof. In another embodiment, a synergistic effect of a combination of therapies may avoid or reduce adverse or unwanted side effects associated with the use of either therapy alone.

As used herein, the terms “therapeutic agent” and “therapeutic agents” refer to any agent(s) which can be used in the prevention, treatment, and/or management of a bacterial infection or one or more symptoms thereof. In certain embodiments, the term “therapeutic agent” refers to a compound provided herein. In other embodiments, the term “therapeutic agent” refers to an agent other than a compound provided herein (e.g., a compound described in Section 5.2 and Table 1). In a specific embodiment, such a therapeutic agent is known to be useful for, or has been or is currently being used for the prevention, treatment, and/or management of a bacterial infection or one or more symptoms thereof.

As used herein, the term “therapeutically effective amount” refers to that amount of the therapy (e.g., a therapeutic agent) sufficient to (1) reduce or inhibit bacterial cell proliferation; (2) reduce or inhibit the viability of bacteria; (3) reduce or inhibit the spread of bacteria from one tissue or organ to another tissue or organ, and/or from one subject to another subject; (4) reduce the severity of a bacterial infection; (5) reduce the duration of a bacterial infection; (6) ameliorate one or more symptoms of a bacterial infection; (7) prevent advancement of a bacterial infection; and/or (8) enhance or improve the therapeutic effect(s) of another therapy. In a specific embodiment, a therapeutically effective amount refers to the amount of a therapy (e.g., therapeutic agent) that inhibits or reduces the replication and/or viability of bacterial cells, inhibits or reduces the onset, development or progression of a bacterial infection or one or more symptoms thereof, or inhibits or reduces the spread of a bacterial infection from one tissue, organ or cell to another tissue, organ or cell. In another specific embodiment, a therapeutically effective amount of a therapy (e.g., a therapeutic agent) reduces the replication of bacterial cells by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, relative to a negative control, such as PBS.

As used herein, the terms “therapy” and “therapies” refer to any protocol(s), method(s) and/or agent(s) that can be used in the prevention, treatment, management or amelioration of a bacterial infection or one or more symptoms thereof. In certain embodiments, the terms “therapy” and “therapies” refer to antibacterial therapy, supportive therapy and/or other therapies useful in the prevention, treatment, management or amelioration of a bacterial infection or one or more symptoms thereof known to skilled medical personnel.

As used herein, the terms “treat,” “treatment,” and “treating” in the context of the administration of a therapy to a subject, refer to (1) the reduction or inhibition of bacterial cell proliferation; (2) the reduction or inhibition of bacterial viability; (3) the reduction or inhibition of a bacterial infection; (4) the reduction or amelioration of the progression, severity and/or duration of a bacterial infection or one or more symptoms thereof, (5) the amelioration of a symptom of a bacterial infection; and/or (6) the reduction or inhibition of the spread of the bacteria from one organ, tissue or cell to another organ, tissue or cell, resulting from the administration of one or more therapies (e.g., one or more compounds provided herein), or a combination of therapies. In specific embodiments, such terms refer to the inhibition or reduction in the replication and/or viability of bacterial cells.

### ABBREVIATION

Pth Peptidyl tRNA hydrolase
HTS High-throughput Screen
FP Fluorescence Polarization
FRET Fluorescence Resonance Energy Transfer
HPLC High-Performance Liquid Chromatography
FPLC Fast Performance Liquid Chromatography

A “(C₁₋₈)alkyl” group is a saturated straight chain or branched non-cyclic hydrocarbon having from 1 to 8 carbon atoms. Representative (C₁₋₈)alkyls include -methyl, -ethyl, -n-propyl, -n-butyl, -n-pentyl, -n-hexyl, -n-heptyl and -n-octyl; while saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. A (C₁₋₈)alkyl group can be substituted or unsubstituted.

A “(C₁₋₈)alkoxy” group is an —O—(C₁₋₈)alkyl group, wherein (C₁₋₈)alkyl is as defined above. Representative (C₁₋₈)alkoxy groups include —O-methyl, —O-ethyl, —O-n-propyl, —O-n-butyl, —O-n-pentyl, —O-n-hexyl, —O-n-heptyl and —O-n-octyl, —O-isopropyl, —O-sec-butyl, —O-isobutyl, —O-tert-butyl, —O-isopentyl, —O-2-methylpentyl, —O-3-methylpentyl, —O-4-methylpentyl, —O-2,3-dimethylbutyl and the like. A (C₁₋₈)alkoxy group can be substituted or unsubstituted.

The terms “halogen” and “halo” mean fluorine, chlorine, bromine and iodine.

An “aryl” group is an unsaturated aromatic carbocyclic group of from 6 to 14 carbon atoms having a single ring (e.g., phenyl) or multiple condensed rings (e.g., naphthyl or anthryl). Particular aryls include phenyl, biphenyl, naphthyl and the like. An aryl group can be substituted or unsubstituted.

A “heteroaryl” group is an aryl ring system having one to four heteroatoms (e.g., O, S or N) as ring atoms in a heteroaromatic ring system, wherein the remainder of the atoms are carbon atoms. Suitable heteroatoms include oxygen, sulfur and nitrogen. In certain embodiments, the heterocyclic ring system is monocyclic or bicyclic. Non-limiting examples include aromatic groups selected from the following:

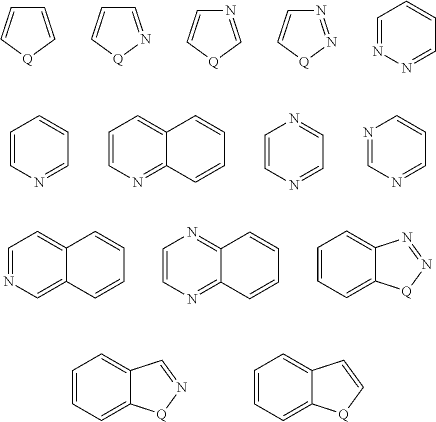

wherein Q is CH₂, CH═CH, O, S or NH. Further representative examples of heteroaryl groups include, but are not limited to, benzofuranyl, benzothienyl, indolyl, benzopyrazolyl, coumarinyl, furanyl, isothiazolyl, imidazolyl, isoxazolyl, thiazolyl, triazolyl, tetrazolyl, thiophenyl, pyrimidinyl, isoquinolinyl, quinolinyl, pyridinyl, pyrrolyl, pyrazolyl, 1H-indolyl, 1H-indazolyl, benzo[d]thiazolyl and pyrazinyl. Further representative examples of heteroaryl groups include those of the compounds disclosed herein. Heteroaryls can be bonded at any ring atom (i.e., at any carbon atom or heteroatom of the heteroaryl ring). A heteroaryl group can be substituted or unsubstituted. In one embodiment, the heteroaryl group is a C₃₋₁₀heteroaryl group.

A “cycloalkyl” group is a saturated or unsaturated non-aromatic carbocyclic ring. Representative cycloalkyl groups include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. Further representative examples of cycloalkyl groups included those of the compounds disclosed herein. A cycloalkyl group can be substituted or unsubstituted. In one embodiment, the cycloalkyl group is a C₃₋₈cycloalkyl group.

A “heterocycloalkyl” group is a non-aromatic cycloalkyl in which one to four of the ring carbon atoms are independently replaced with a heteroatom from the group consisting of O, S and N. Representative examples of a heterocycloalkyl group include, but are not limited to, morpholinyl, pyrrolyl, pyrrolidinyl, thienyl, furanyl, thiazolyl, imidazolyl, pyrazolyl, triazolyl, piperizinyl, isothiazolyl, isoxazolyl, (1,4)-dioxane, (1,3)-dioxolane, 4,5-dihydro-1H-imidazolyl and tetrazolyl. Further representative examples of heterocycloalkyl groups included those of the compounds disclosed herein. Heterocycloalkyls can also be bonded at any ring atom (i.e., at any carbon atom or heteroatom of the Heteroaryl ring). A heterocycloalkyl group can be substituted or unsubstituted. In one embodiment, the heterocycloalkyl is a 3-7 membered heterocycloalkyl.

When the groups described herein are said to be “substituted or unsubstituted,” when substituted, they may be substituted with one or more of any substituent. Examples of substituents are those found in the exemplary compounds and embodiments disclosed herein, as well as halo (e.g., chloro, iodo, bromo, or fluoro); C₁₋₈ alkyl; C₂₋₈ alkenyl; C₂₋₈ alkynyl; hydroxyl; C₁₋₈ alkoxyl; amino; nitro; thiol; thioether; imine; cyano; amido; phosphonato; phosphine; carboxyl; carbamoyl; carbamate; acetal; urea; thiocarbonyl; sulfonyl; sulfonamide; ketone; aldehyde; ester; acetyl; acetoxy; oxygen (═O); haloalkyl (e.g., trifluoromethyl); substituted aminoacyl and aminoalkyl; carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, furanyl, or thiazinyl); carbocyclic or heterocyclic, monocyclic or fused or non-fused polycyclic aryl (e.g., phenyl, naphthyl, pyrrolyl, indolyl, furanyl, thienyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothienyl, or benzofuranyl); amino (primary, secondary, or tertiary); —O-lower alkyl; —O-aryl; aryl; aryl-lower alkyl; CO₂CH₃; CONH₂; OCH₂CONH₂; NH₂; N(C₁₋₄alkyl)₂; NHC(O)C₁₋₄alkyl; SO₂NH₂; SO₂C₁₋₄alkyl; OCHF₂; CF₃; OCF₃; and such moieties may also be optionally substituted by a fused-ring structure or bridge, for example —OCH₂O— or —O-lower alkylene-O—. These substituents may optionally be further substituted with a substituent selected from such groups.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “solvate” means a compound, or a salt thereof, that further includes a stoichiometric or non-stoichiometric amount of a solvent bound by non-covalent intermolecular forces.

As used herein and unless otherwise indicated, the term “prodrug” means a compound derivative that can hydrolyze, oxidize, or otherwise react under biological conditions (in vitro or in vivo) to provide an active compound, particularly a compound provided herein. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

Various compounds contain one or more chiral centers, and can exist as racemic mixtures of enantiomers, mixtures of diastereomers or enantiomerically or optically pure compounds. The use of stereomerically pure forms of such compound, as well as the use of mixtures of those forms are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 5.2 Compounds

In one embodiment, provided herein are compounds of formula I:

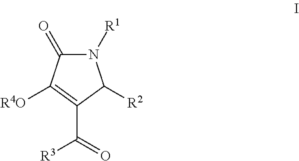

and pharmaceutically acceptable salts thereof, wherein:

R¹ is H, substituted or unsubstituted (C₁₋₈)alkyl, substituted or unsubstituted aryl, substituted or unsubstituted heteroaryl, substituted or unsubstituted aryl(C₁₋₈)alkyl, substituted or unsubstituted heteroaryl(C₁₋₈)alkyl, or substituted or unsubstituted cycloalkyl;

R² is H, substituted or unsubstituted (C₁₋₈)alkyl, substituted or unsubstituted aryl, substituted or unsubstituted heteroaryl, substituted or unsubstituted aryl(C₁₋₈)alkyl, substituted or unsubstituted cycloalkyl, or substituted or unsubstituted heterocycloalkyl;

R³ is substituted or unsubstituted (C₁₋₈)alkyl, (C₁₋₈)alkoxy, substituted or unsubstituted aryl, substituted or unsubstituted heteroaryl, substituted or unsubstituted cycloalkyl, substituted or unsubstituted heterocycloalkyl, a 7-membered saturated or partially unsaturated bridged cyclic hydrocarbon (e.g. norbornenyl), (C₁₋₈)alkyl-O—(C₁₋₈)alkyl, aryl-O—(C₁₋₈)alkyl wherein the aryl ring can be substituted or unsubstituted, or R⁵OOC—(C₁₋₈)alkyl;

R⁴ is H, C₁₋₈alkyl, (C₁₋₈)alkyl(CO), substituted or unsubstituted aryl(C₁₋₈)alkyl, substituted or unsubstituted heteroaryl(C₁₋₈)alkyl, substituted or unsubstituted cycloalkyl; and

R⁵ is H or substituted or unsubstituted C₁₋₈alkyl.

In one embodiment, the compounds of formula I are those wherein R¹ is H, substituted or unsubstituted aryl, substituted or unsubstituted heteroaryl, substituted or unsubstituted aryl(C₁₋₈)alkyl, substituted or unsubstituted heteroaryl(C₁₋₈)alkyl, substituted or unsubstituted three to six membered cycloalkyl.

In one embodiment, the compounds of formula I are those wherein R² is C₁₋₈alkyl, substituted or unsubstituted aryl, substituted or unsubstituted heteroaryl, substituted or unsubstituted aryl(C₁₋₈)alkyl, substituted or unsubstituted three to six membered cycloalkyl wherein one CH₂ group can be replaced by oxygen.

In one embodiment, the compounds of formula I do not include compounds wherein R³ is C₁₋₈alkoxy. In one embodiment, the compounds of formula I do not include compounds wherein R⁴ is H and R³ is ethoxy.

In one embodiment, the compounds of formula I do not include compounds wherein R³ is C₁₋₈alkoxy. In one embodiment, the compounds of formula I do not include compounds wherein R⁴ is methyl and R³ is ethoxy.

In one embodiment, the compounds of formula I do not include compounds wherein R⁴ is H and R¹ is —CH₂CH₂N(CH₂CH₃)₂.

In one embodiment, the compounds of formula I do not include compounds wherein R⁴ is H and R¹ is alkyl, in one embodiment ethyl, substituted with oxopiperizine.

In one embodiment, the compounds of formula I do not include compounds wherein R⁴ is H and R¹ is CH₂COOH.

In one embodiment, the compounds of formula I do not include compounds wherein R⁴ is H and R¹ is —(CH₂)₃NH₂ or —CH₂C(O)NH₂.

In one embodiment, the compounds of formula I do not include compounds wherein R⁴ is H and R¹ is —(CH₂)₂NH₂, —(CH₂)₂OH or alkyl substituted with pyrrolinone.

In one embodiment, the compounds of formula I do not include compounds wherein R⁴ is H and R¹ is methyl, —(CH₂)₂N(alkyl)₂, -alkyl-COOH, —CH₂C(O)NH₂, substituted or unsubstituted phenyl, or substituted or unsubstituted heterocycloalkyl or heteroaryl.

In one embodiment, the compounds of formula I do not include compounds wherein R¹ is pyridine.

In one embodiment, the compounds of formula I do not include one or more of:
4-(benzofuran-2-carbonyl)-5-(3-ethoxy-4-hydroxyphenyl)-3-hydroxy-1-(5-(naphthalen-1-ylmethylthio)-1,3,4-thiadiazol-2-yl)-1H-pyrrol-2(5H)-one;
4-(benzofuran-2-carbonyl)-1-(5-(benzylthio)-1,3,4-thiadiazol-2-yl)-5-(3-ethoxy-4-hydroxyphenyl)-3-hydroxy-1H-pyrrol-2(5H)-one;
4-(benzofuran-2-carbonyl)-3-hydroxy-5-(3-hydroxyphenyl)-1-(6-methylbenzo[d]thiazol-2-yl)-1H-pyrrol-2(5H)-one;
4-(benzofuran-2-carbonyl)-5-(4-(benzyloxy)-3-methoxyphenyl)-3-hydroxy-1-(5-methyl-1,3,4-thiadiazol-2-yl)-1H-pyrrol-2(5H)-one;
1-(5-acetyl-4-methylthiazol-2-yl)-4-(benzofuran-2-carbonyl)-5-(4-(benzyloxy)phenyl)-3-hydroxy-1H-pyrrol-2(5H)-one;
1-(5-acetyl-4-methylthiazol-2-yl)-4-(benzofuran-2-carbonyl)-5-(4-(benzyloxy)-3-methoxyphenyl)-3-hydroxy-1H-pyrrol-2(5H)-one;
4-(benzofuran-2-carbonyl)-1-(5-(4-chlorobenzylthio)-1,3,4-thiadiazol-2-yl)-3-hydroxy-5-(3-hydroxyphenyl)-1H-pyrrol-2(5H)-one;
4-benzoyl-3-hydroxy-5-phenyl-1-(pyridin-2-yl)-1H-pyrrol-2(5H)-one;
4-acetyl-3-hydroxy-5-phenyl-1-(pyridin-2-yl)-1H-pyrrol-2(5H)-one; or
4-benzoyl-3-hydroxy-5-phenyl-1-(thiazol-2-yl)-1H-pyrrol-2(5H)-one.

In another embodiment, provided herein are compounds of formula IA:

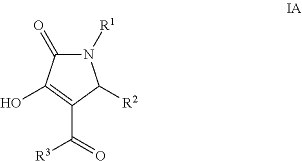

and pharmaceutically acceptable salts thereof, wherein:

R¹ is H, substituted or unsubstituted (C₁₋₈)alkyl, substituted or unsubstituted aryl, substituted or unsubstituted heteroaryl, substituted or unsubstituted aryl(C₁₋₈)alkyl, substituted or unsubstituted heteroaryl(C₁₋₈)alkyl, or substituted or unsubstituted three to six membered cycloalkyl;

R² is H, substituted or unsubstituted (C₁₋₈)alkyl, substituted or unsubstituted aryl, substituted or unsubstituted heteroaryl, substituted or unsubstituted aryl(C₁₋₈)alkyl, substituted or unsubstituted three to six membered cycloalkyl wherein one CH₂ group can be replaced by oxygen; and

R³ is substituted or unsubstituted C₁₋₈alkyl, substituted or unsubstituted (C₁₋₈)cycloalkyl, or substituted or unsubstituted (C₁₋₈)alkyl-O—(C₁₋₈)alkyl.

In a particular embodiment, R³ is —CH₂OMe, substituted or unsubstituted cyclopropyl or methyl.

In a further embodiment of the compounds having formula IA, R¹ is substituted or unsubstituted aryl, or substituted or unsubstituted heteroaryl.

In a further embodiment of the compounds having formula IA, R¹ is substituted or unsubstituted heteroaryl.

In a further embodiment of the compounds having formula IA, R¹ is substituted or unsubstituted 5-membered heteroaryl. In one embodiment, R¹ is substituted with a substituted or unsubstituted aryl. In a particular embodiment, R′ is substituted or unsubstituted 4-phenyl-thiazole-2-yl.

In a further embodiment of the compounds having formula IA, R¹ is substituted or unsubstituted 8-10-membered fused heterobiaryl having one to six heteroatoms independently chosen from N, O and S. In one embodiment, R¹ is substituted or unsubstituted 2-benzothiazolyl. In one embodiment, R¹ is substituted with halo, nitro, substituted or unsubstituted (C₁₋₈)alkyl, substituted or unsubstituted (C₁₋₈)alkoxy, or (C₁₋₈)alkylsulfone.

In a further embodiment of the compounds having formula IA, R¹ is H, substituted or unsubstituted (C₁₋₈)alkyl, substituted or unsubstituted aryl, substituted or unsubstituted heteroaryl, substituted or unsubstituted aryl(C₁₋₈)alkyl, substituted or unsubstituted heteroaryl(C₁₋₈)alkyl, or substituted or unsubstituted three to six membered cycloalkyl; R² is H, (C₁₋₈)alkyl, substituted or unsubstituted aryl, substituted or unsubstituted heteroaryl, substituted or unsubstituted aryl(C₁₋₈)alkyl, or substituted or unsubstituted three to six membered cycloalkyl, wherein one CH₂ group can be replaced by oxygen; and R³ is substituted or unsubstituted aryl or substituted or unsubstituted heteroaryl.

Representative compounds are set forth in Table 1.

TABLE 1

| | | E. coli | S. aureus | CC₅₀ (μM) |
|---|---|---|---|---|
| No. | Compound | BAS849 | 29213 | Huh7 cells |
| 1 | 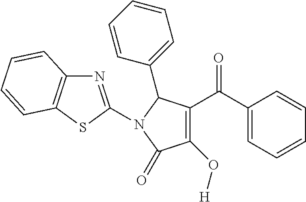 | * | * | |
| 2 | 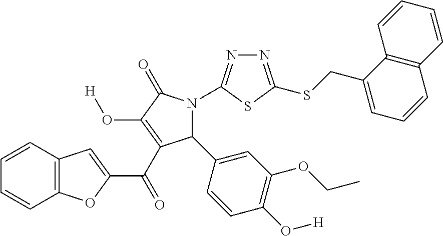 | * | * | >79 |
| 3 | 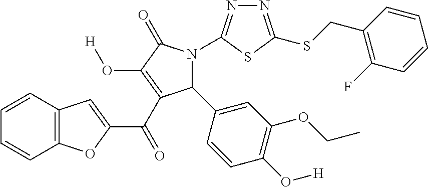 | * | * | >83 |
| 4 | 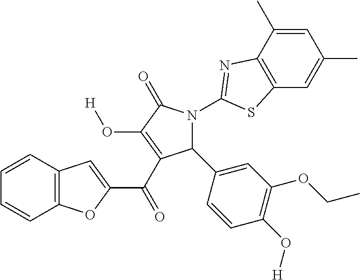 | ***/**** | **** | 53.35 |
| 5 | 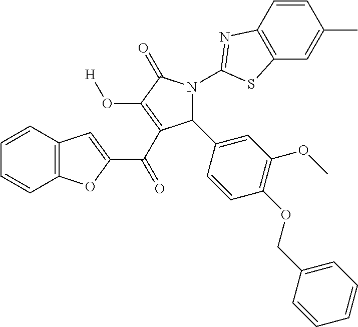 | * | * | |
| 6 | 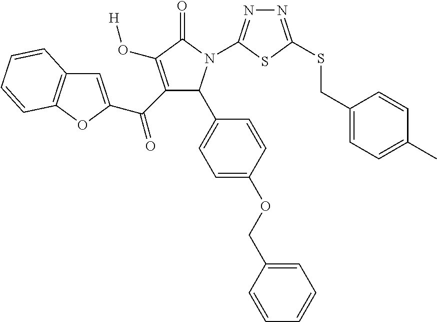 | * | * | |
| 7 | 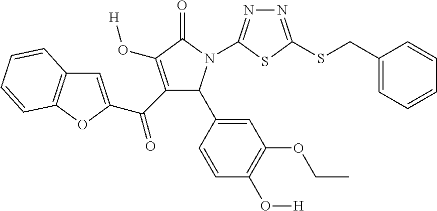 | * | **** | >85 |
| 8 | 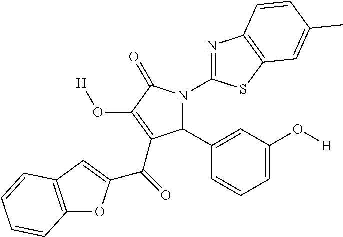 | *** | **** | 80.09 |
| 9 | 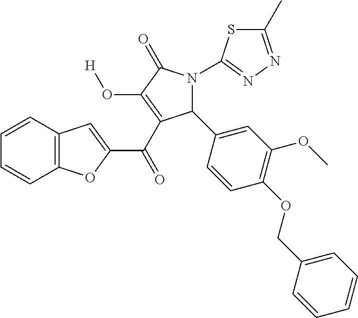 | * | *** | >90 |
| 10 | 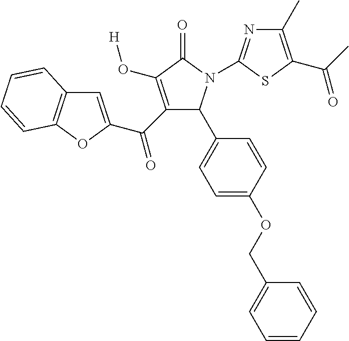 | * | * | >89 |
| 11 | 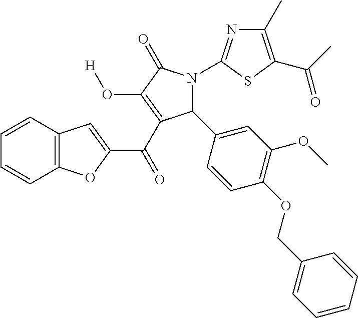 | * | **** | >84 |
| 12 | 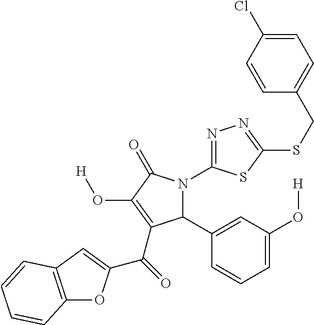 | * | * | >87 |
| 13 | 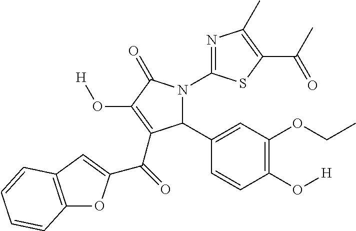 | * | ** | 59.84 |
| 14 | 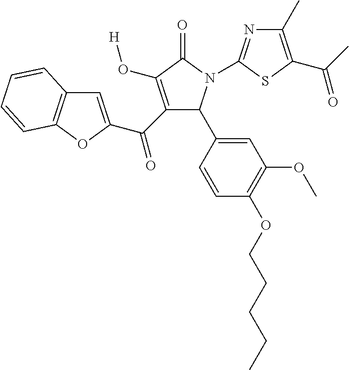 | * | * | 74.17 |
| 15 | 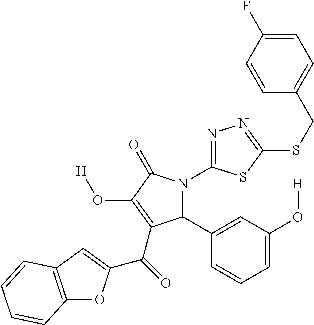 | * | * | >89 |
| 16 | 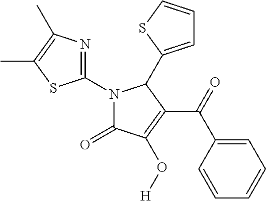 | * | * | |
| 17 | 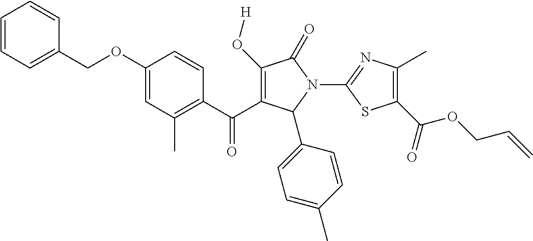 | * | * | |
| 18 | 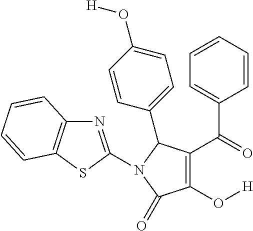 | * | * | |
| 19 | 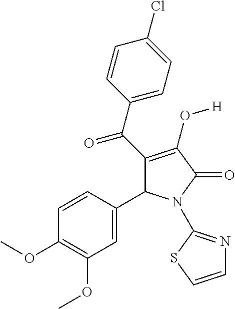 | * | * | |
| 20 | 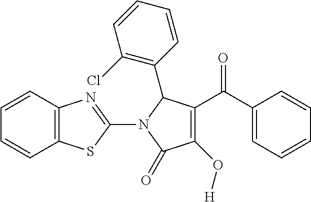 | ** | ** | |
| 21 | 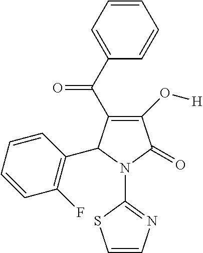 | * | * | |
| 22 | 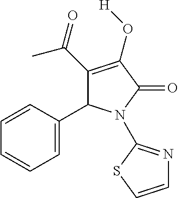 | * | * | 29.65 |
| 23 | 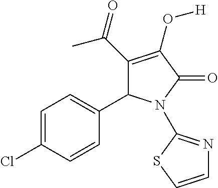 | * | * | |
| 24 | 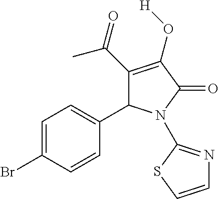 | * | * | |
| 25 | 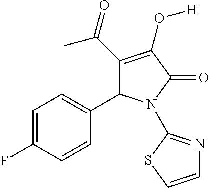 | * | * | |
| 26 | 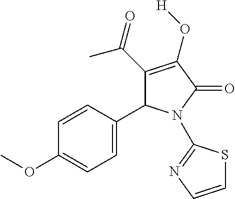 | * | * | |
| 27 | 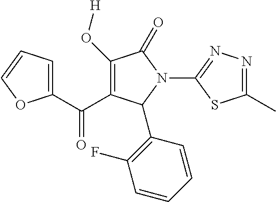 | * | * | |
| 28 | 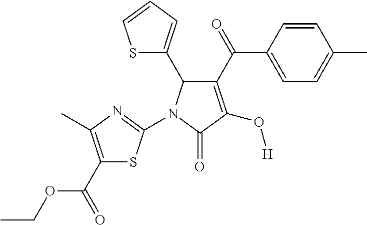 | *** | ** | 89.06 |
| 29 | 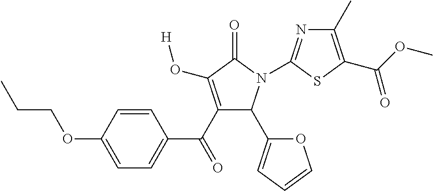 | ** | * | |
| 30 | 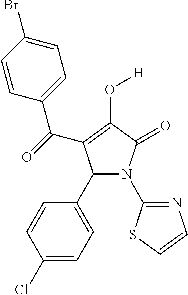 | ** | * | |
| 31 | 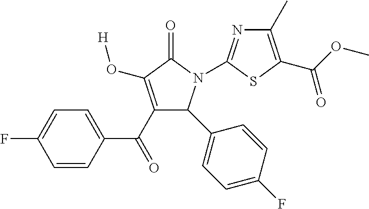 | * | * | 46.46 |
| 32 | 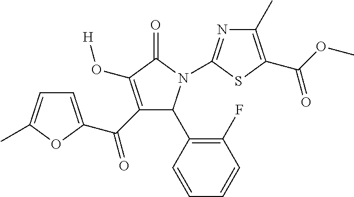 | ** | ** | |
| 33 | 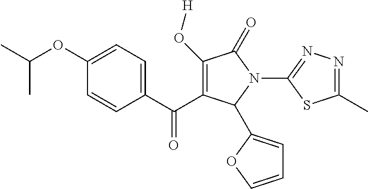 | * | * | >110 |
| 34 | 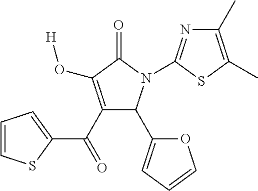 | * | * | |
| 35 | 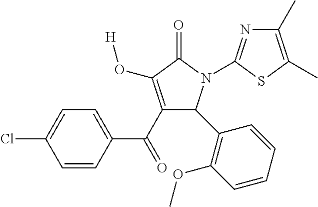 | ** | * | |
| 36 | 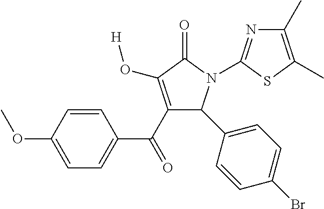 | *** | ** | |
| 37 | 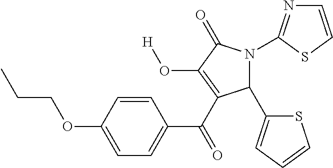 | * | * | |
| 38 | 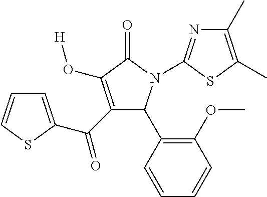 | * | * | |
| 39 | 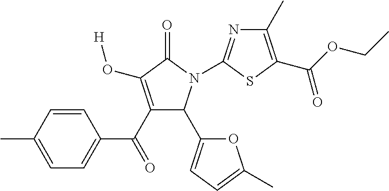 | ** | * | |
| 40 | 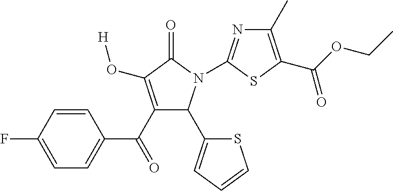 | * | * | 62.86 |
| 41 | 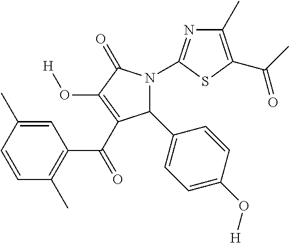 | * | * | |
| 42 | 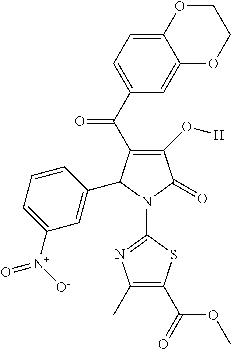 | ** | * | |
| 43 | 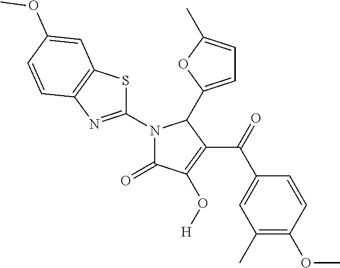 | ** | *** | |
| 44 | 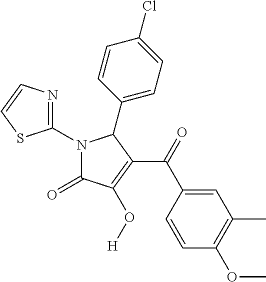 | ** | * | 51.54 |
| 45 | 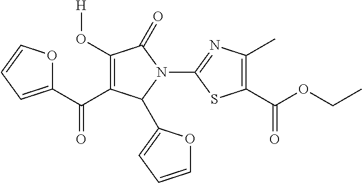 | * | * | 96.16 |
| 46 | 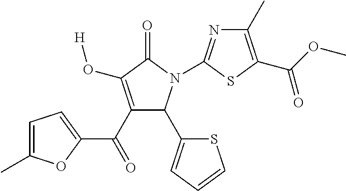 | * | * | |
| 47 | 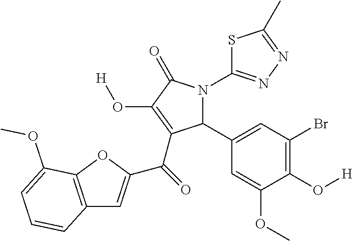 | * | * | |
| 48 | 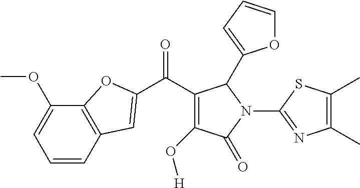 | ** | ** | |
| 49 | 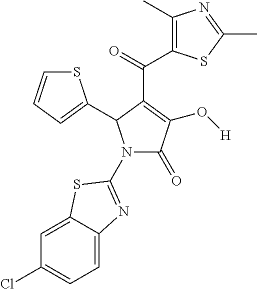 | ** | * | >111 |
| 50 | 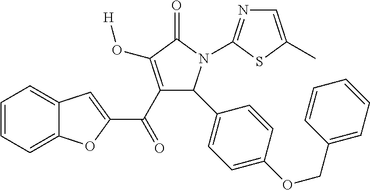 | * | | 82.05 |
| 51 | 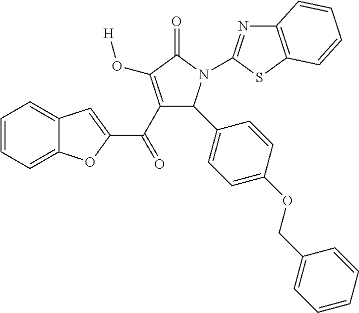 | * | | 82.31 |
| 52 | 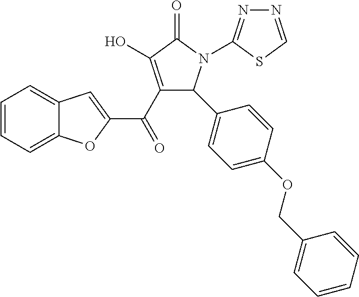 | * | | >90 |
| 53 | 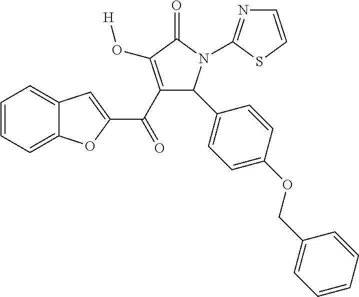 | * | | >98 |
| 54 | 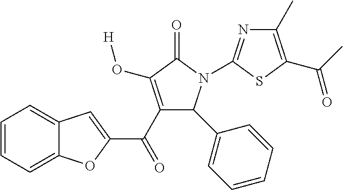 | *** | | 78.52 |
| 55 | 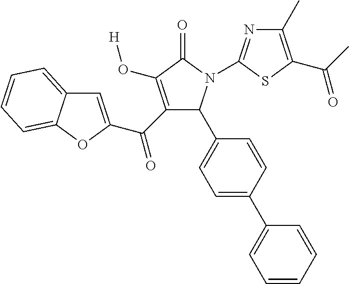 | * | | 42.89 |
| 56 | 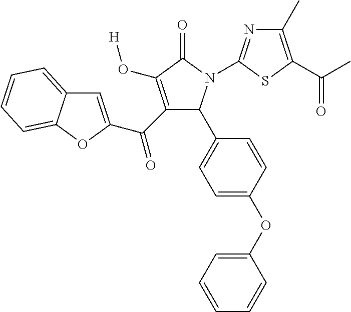 | * | | >984 |
| 57 | 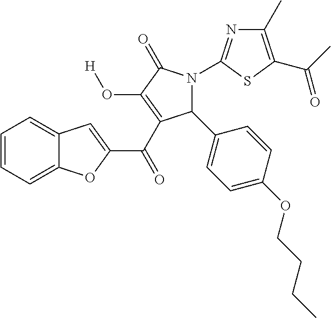 | * | | >91 |
| 58 | 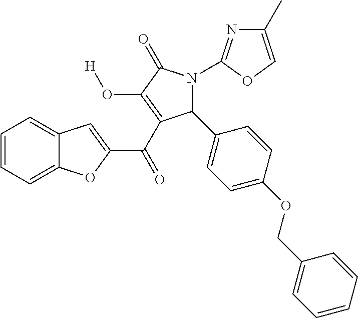 | * | | >94 |
| 59 | 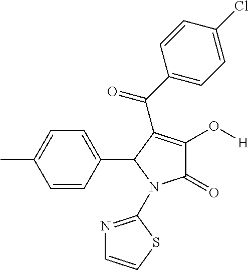 | * | * | 60.49 |
| 60 | 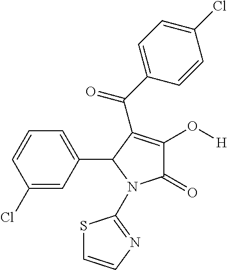 | * | * | |
| 61 | 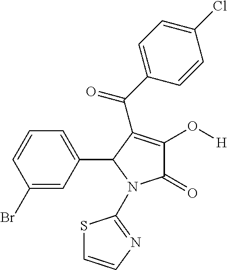 | ** | * | |
| 62 | 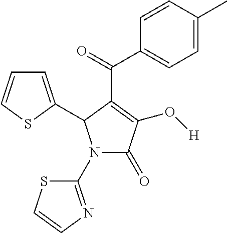 | * | * | |
| 63 | 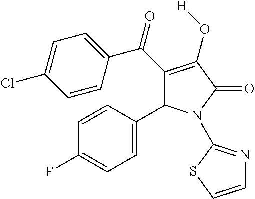 | * | * | |
| 64 | 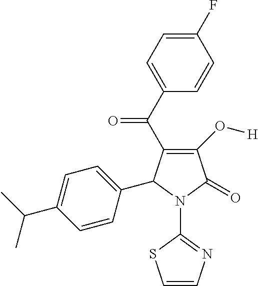 | *** | * | |
| 65 | 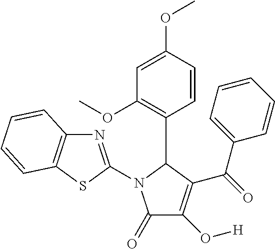 | ** | * | |
| 66 | 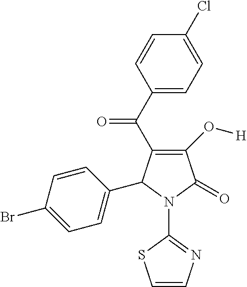 | *** | * | |
| 67 | 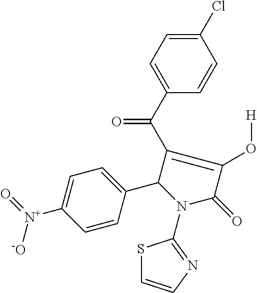 | * | * | |
| 68 | 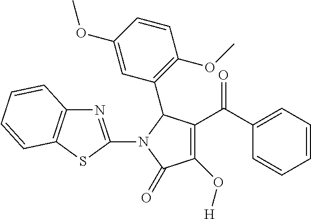 | * | * | |
| 69 | 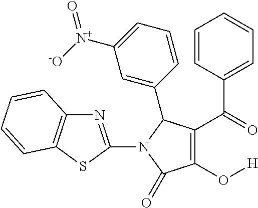 | ** | * | |
| 70 | 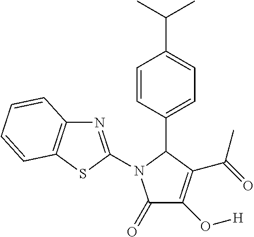 | ** | *** | |
| 71 | 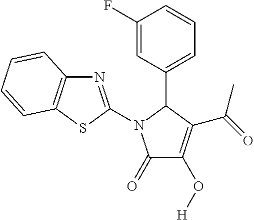 | * | * | |
| 72 | 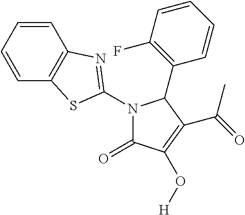 | * | * | |
| 73 | 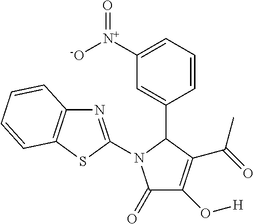 | * | * | |
| 74 | 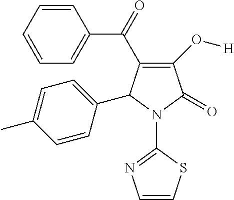 | * | * | |
| 75 | 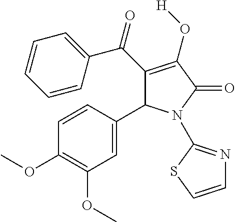 | ** | * | |
| 76 | 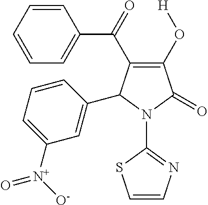 | * | * | |
| 77 | 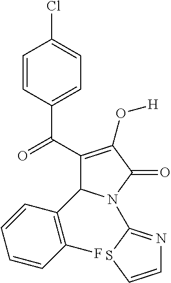 | * | * | |
| 78 | 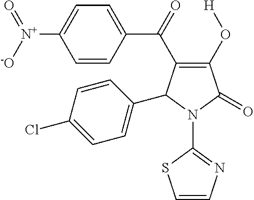 | * | * | |
| 79 | 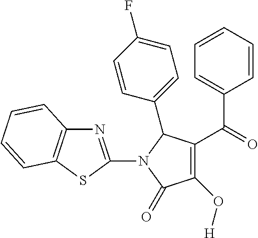 | ** | ** | |
| 80 | 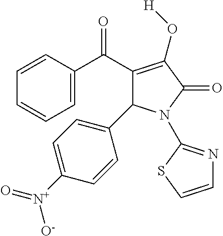 | * | * | |
| 81 | 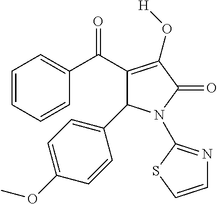 | ** | * | |
| 82 | 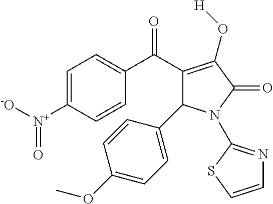 | * | * | |
| 83 | 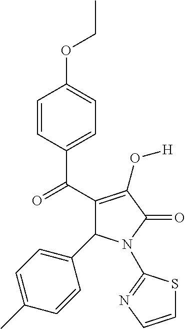 | ** | * | |
| 84 | 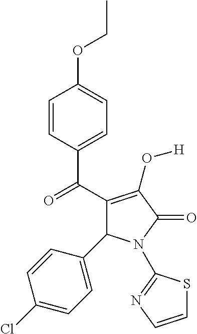 | ** | * | |
| 85 | 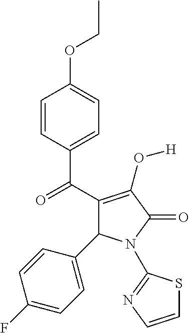 | * | * | |
| 86 | 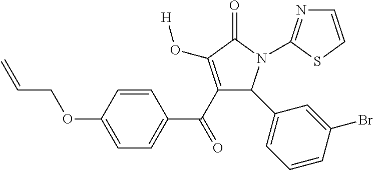 | *** | ** | |
| 87 | 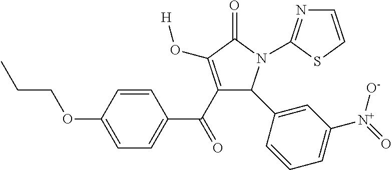 | * | * | |
| 88 | 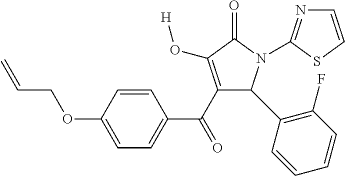 | * | * | |
| 89 | 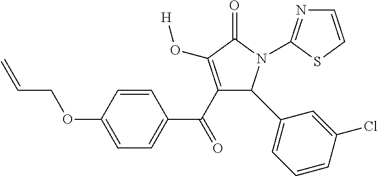 | ** | * | |
| 90 | 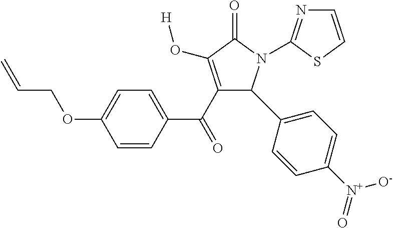 | * | * | |
| 91 | 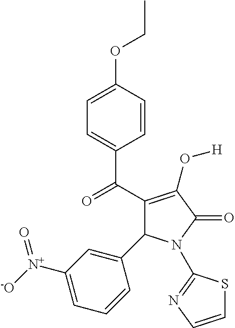 | * | * | |
| 92 | 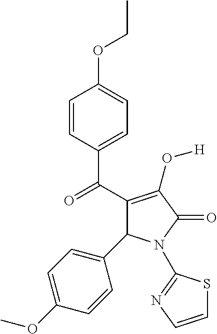 | * | * | |
| 93 | 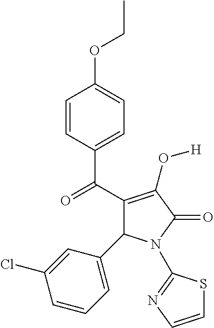 | * | * | |
| 94 | 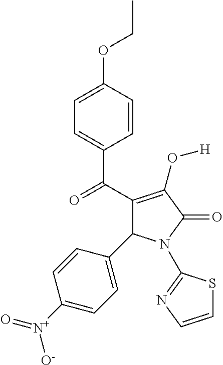 | * | * | |
| 95 | 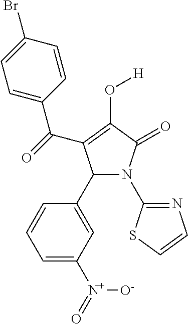 | * | * | |
| 96 | 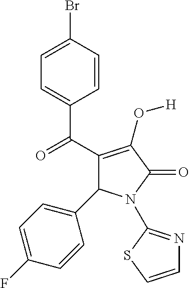 | * | * | |
| 97 | 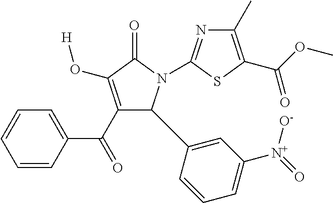 | * | * | |
| 98 | 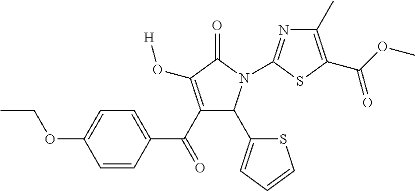 | ** | ** | |
| 99 | 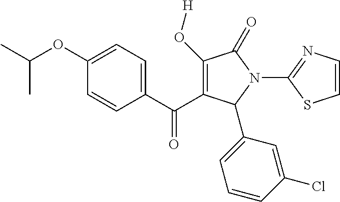 | ** | * | |
| 100 | 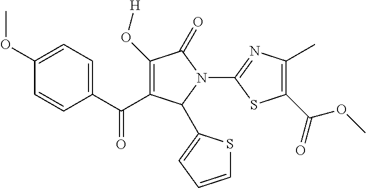 | * | * | |
| 101 | 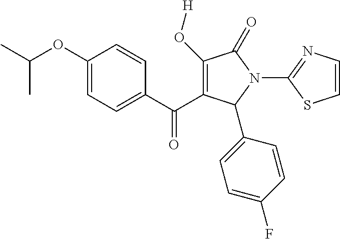 | * | * | |
| 102 | 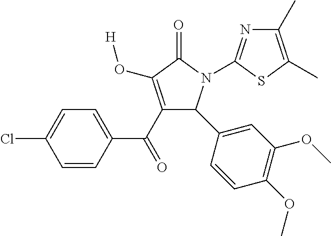 | * | * | |
| 103 | 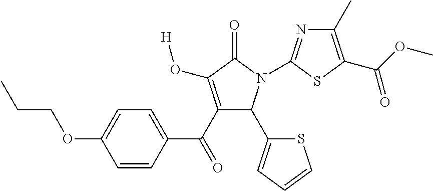 | *** | *** | |
| 104 | 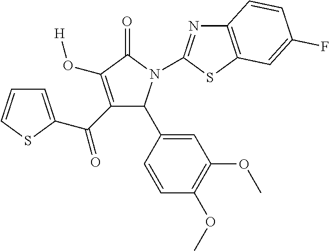 | * | * | |
| 105 | 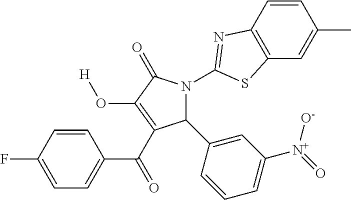 | *** | *** | |
| 106 | 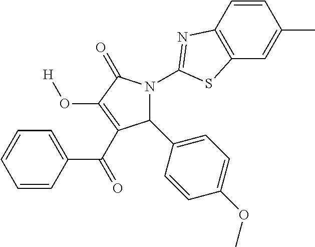 | ** | *** | |
| 107 | 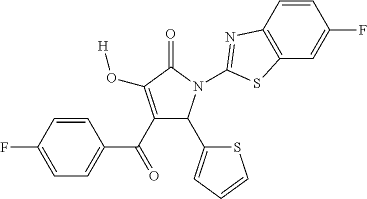 | *** | ** | |
| 108 | 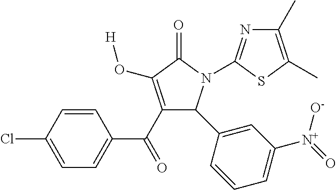 | ** | * | |
| 109 | 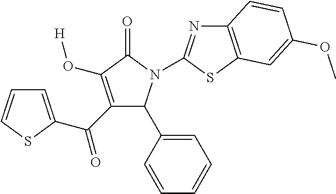 | * | ** | |
| 110 | 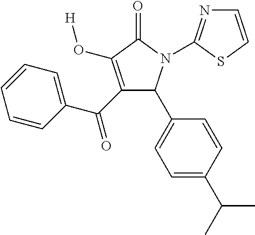 | *** | * | |
| 111 | 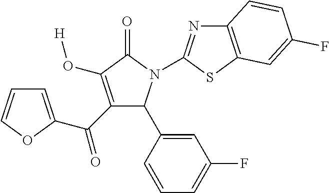 | *** | *** | |
| 112 | 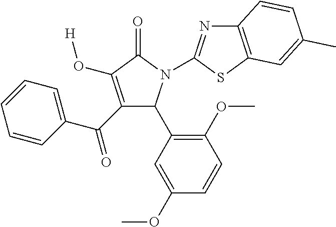 | * | ** | |
| 113 | 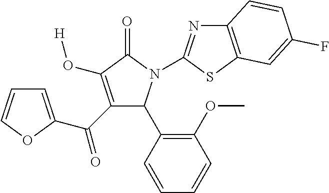 | ** | *** | |
| 114 | 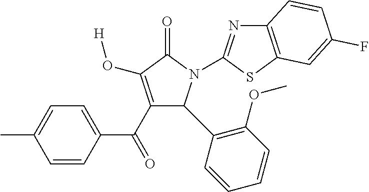 | *** | *** | |
| 115 | 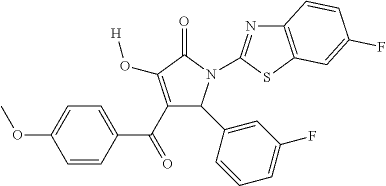 | *** | *** | |
| 116 | 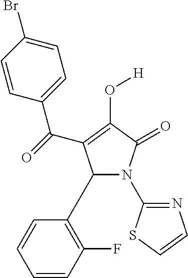 | * | * | |
| 117 | 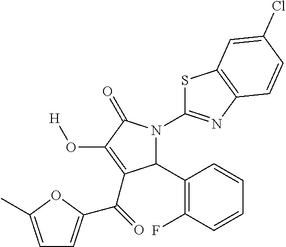 | **** | **** | |
| 118 | 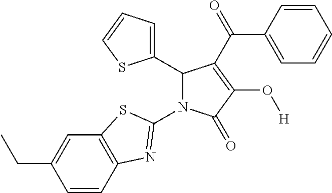 | *** | *** | 10.79 |
| 119 | 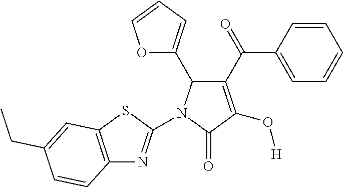 | ** | ** | 10.79 |
| 120 | 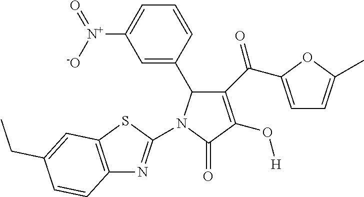 | **** | **** | |
| 121 | 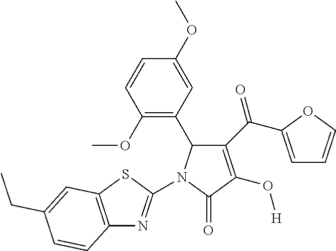 | ** | **** | |
| 122 | 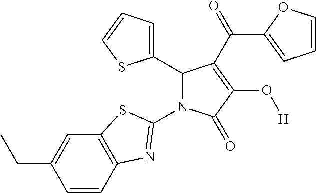 | **** | **** | 58.2 |
| 123 | 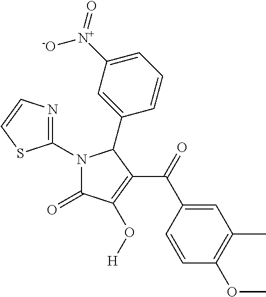 | * | * | |
| 124 | 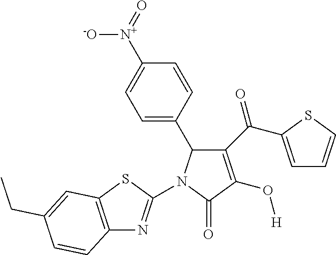 | **** | *** | |
| 125 | 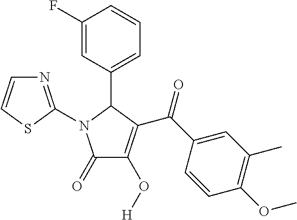 | * | * | |
| 126 | 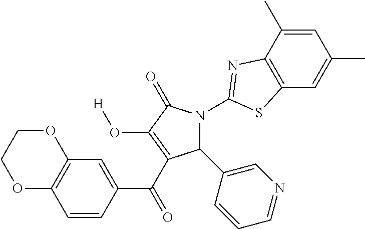 | ** | * | 27.43 |
| 127 | 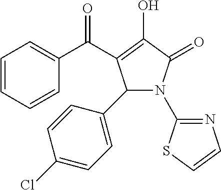 | * | * | |
| 128 | 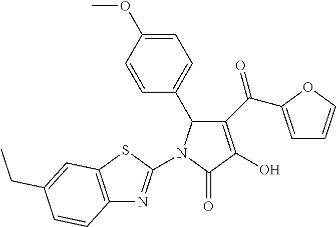 | **** | **** | |
| 129 | 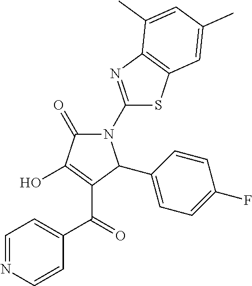 | * | * | |
| 130 | 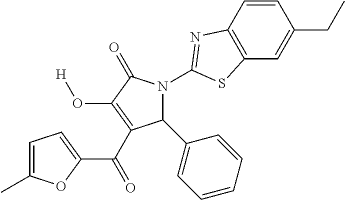 | *** | **** | |
| 131 | 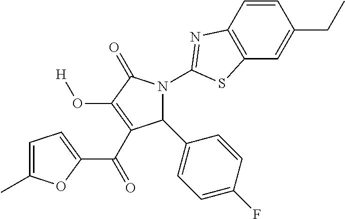 | *** | **** | |
| 132 | 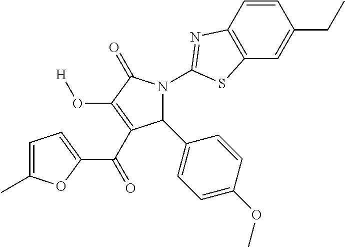 | **** | **** | |
| 133 | 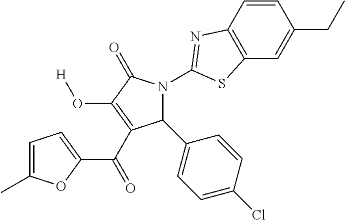 | **** | **** | |
| 134 | 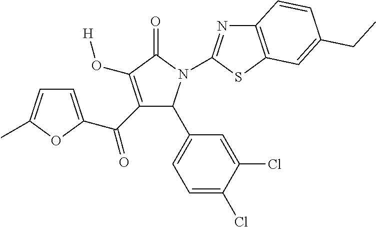 | * | * | |
| 135 | 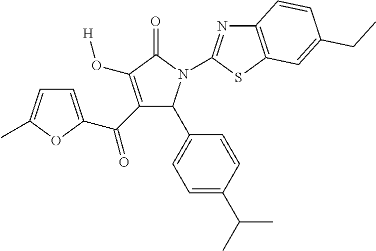 | **** | * | |
| 136 | 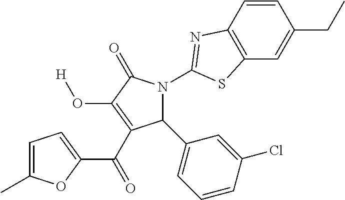 | *** | *** | |
| 137 | 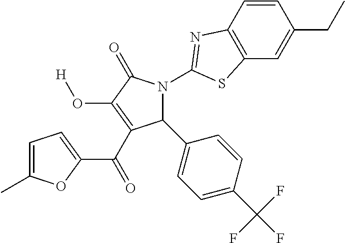 | **** | * | |
| 138 | 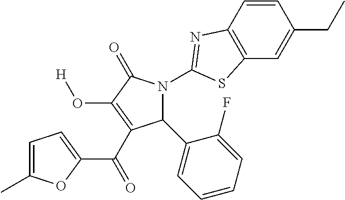 | *** | **** | |
| 139 | 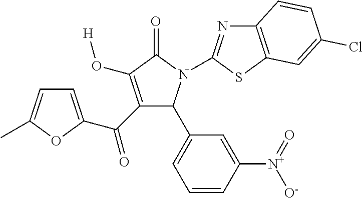 | **** | **** | |
| 140 | 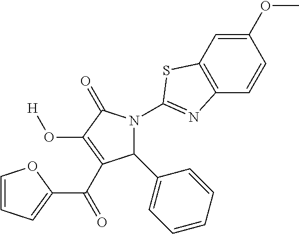 | * | ** | |
| 141 | 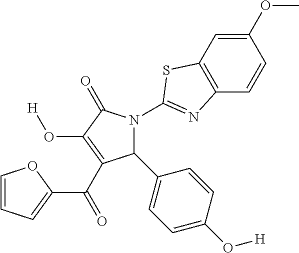 | * | * | |
| 142 | 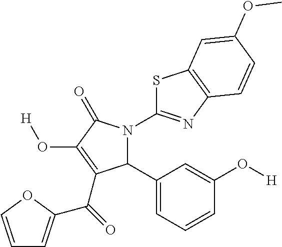 | * | * | |
| 143 | 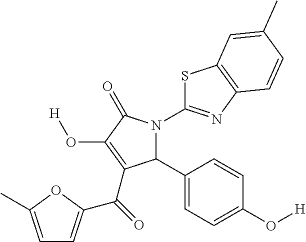 | ** | *** | |
| 144 | 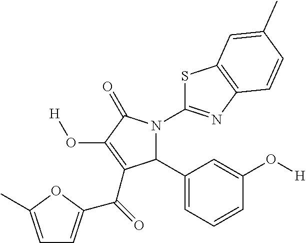 | * | ** | |
| 145 | 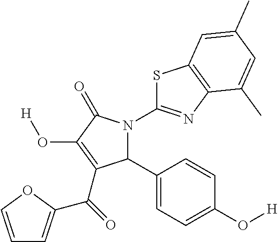 | **** | *** | |
| 146 | 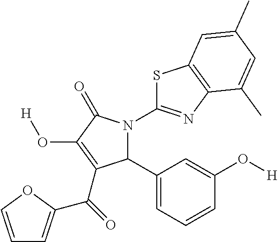 | *** | *** | |
| 147 | 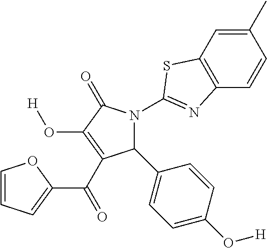 | * | * | |
| 148 | 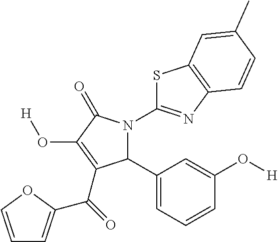 | * | * | |
| 149 | 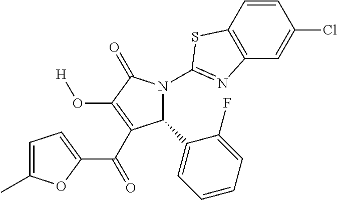 | ** | **** | |
| 150 | 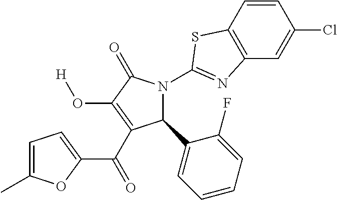 | **** | **** | |
| 151 | 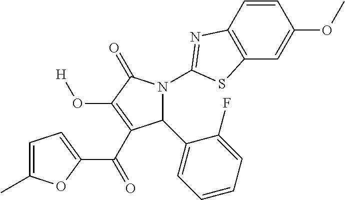 | *** | **** | |
| 152 | 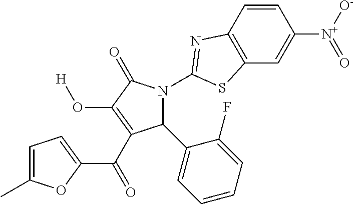 | **** | **** | |
| 153 | 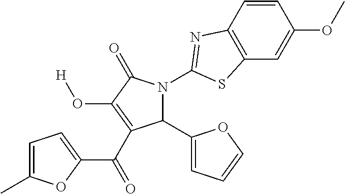 | * | ** | |
| 154 | 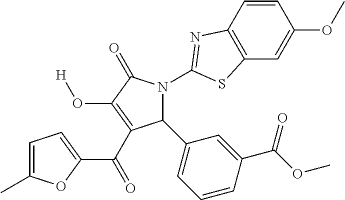 | * | **** | |
| 155 | 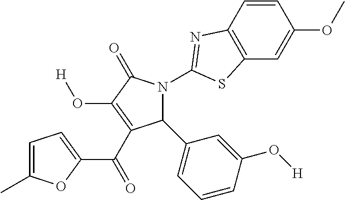 | * | * | |
| 156 | 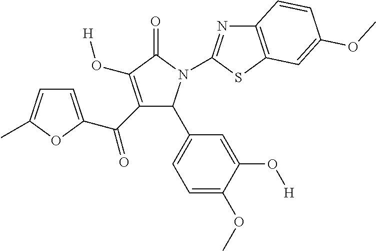 | ** | *** | |
| 157 | 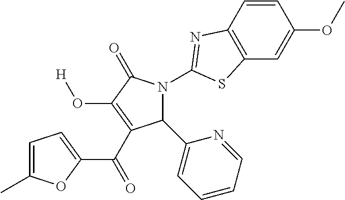 | * | * | |
| 158 | 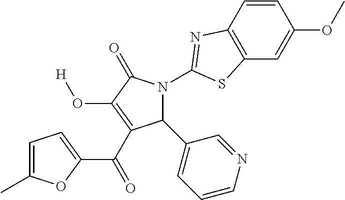 | * | * | |
| 159 | 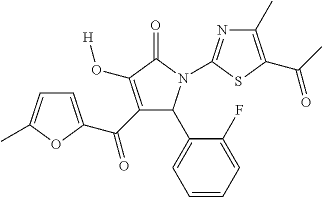 | * | * | |
| 160 | 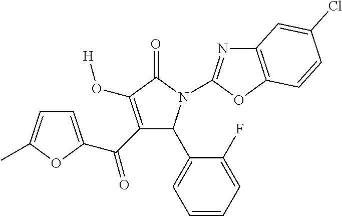 | *** | *** | |
| 161 | 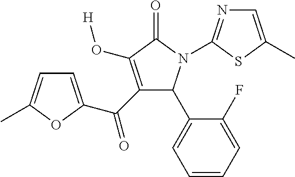 | * | * | |
| 162 | 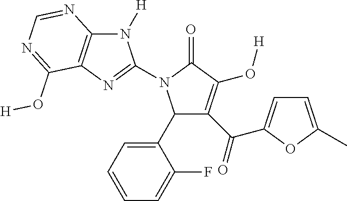 | * | * | |
| 163 | 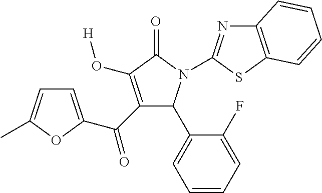 | **** | **** | |
| 164 | 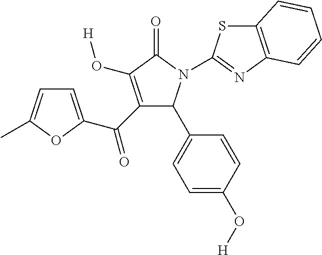 | * | * | |
| 165 | 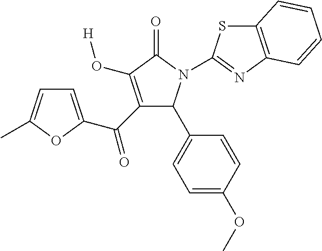 | **** | **** | |
| 166 | 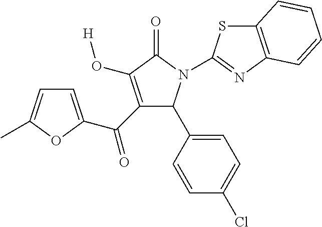 | **** | **** | |
| 167 | 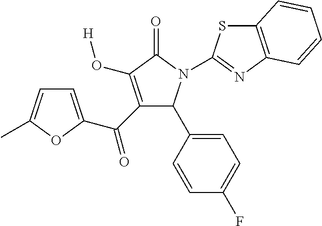 | **** | **** | |
| 168 | 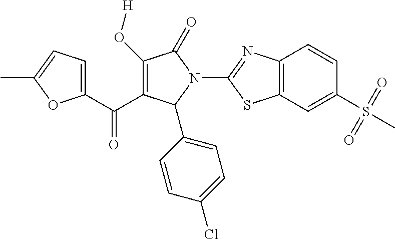 | * | * | |
| 169 | 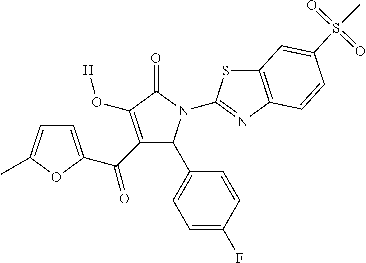 | * | * | |
| 170 | 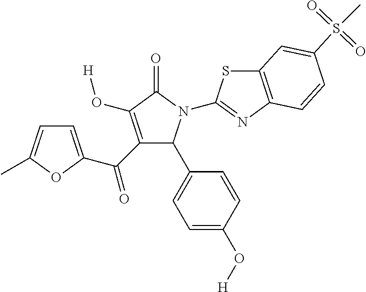 | * | * | |
| 171 | 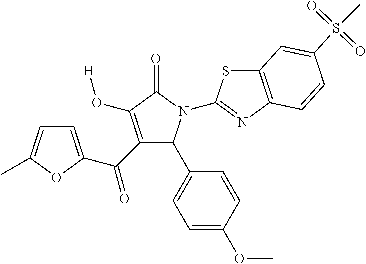 | * | ** | |
| 172 | 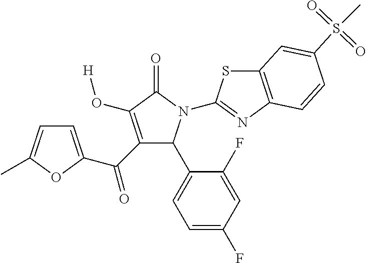 | * | * | |
| 173 | 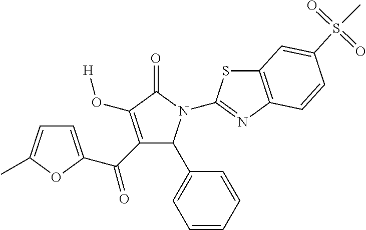 | * | * | |
| 174 | 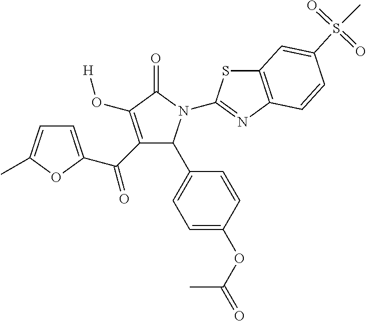 | * | * | |
| 175 | 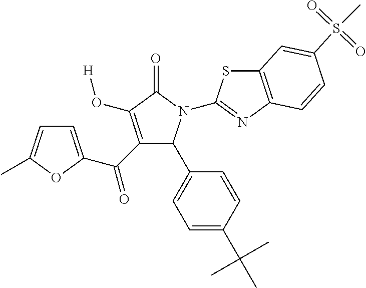 | **** | **** | |
| 176 | 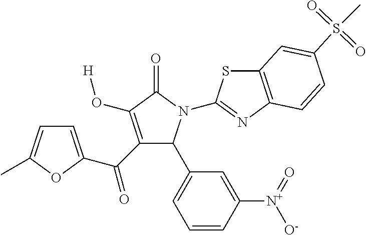 | * | * | |
| 177 | 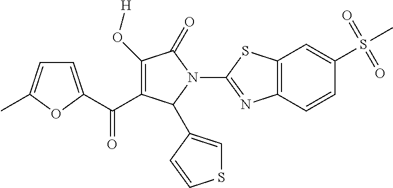 | * | * | |
| 178 | 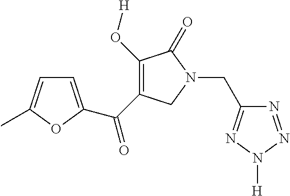 | * | * | |
| 179 | 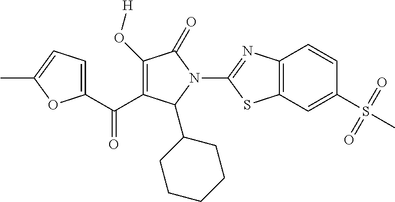 | ** | ** | |
| 180 | 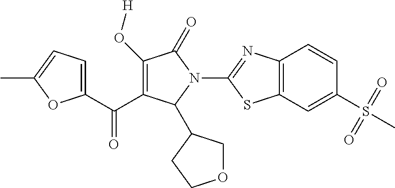 | * | * | |
| 181 | 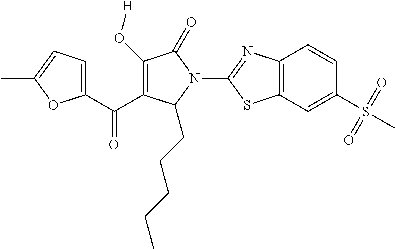 | * | * | |
| 182 | 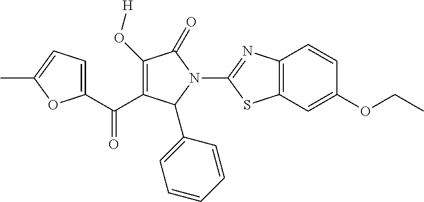 | **** | **** | |
| 183 | 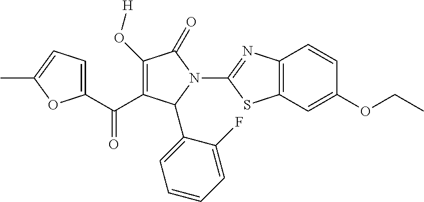 | **** | **** | |
| 184 | 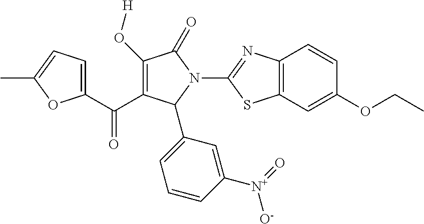 | **** | **** | |
| 185 | 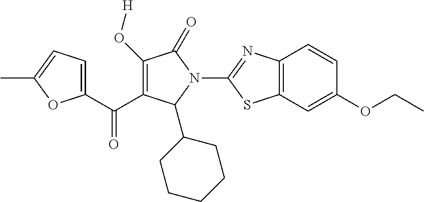 | * | **** | |
| 186 | 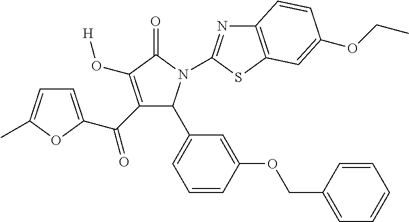 | * | * | |
| 187 | 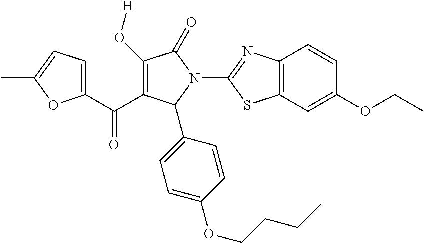 | * | * | |
| 188 | 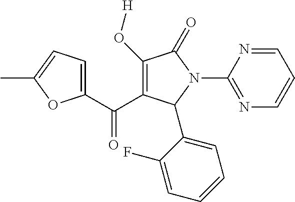 | * | * | |
| 189 | 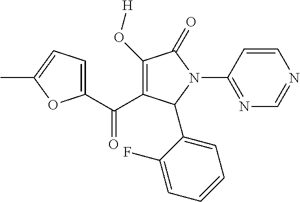 | * | * | |
| 190 | 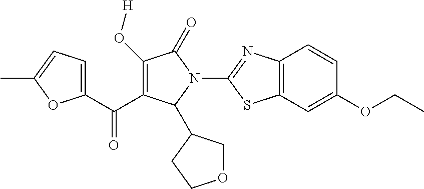 | * | * | |
| 191 | 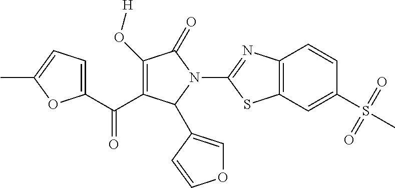 | * | * | |
| 192 | 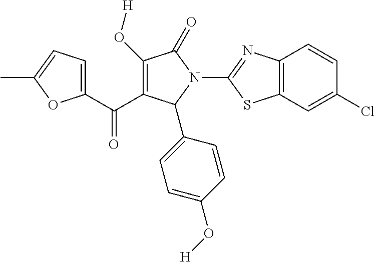 | *** | *** | |
| 193 | 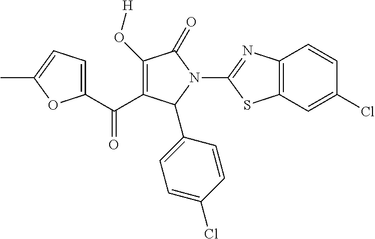 | **** | **** | |
| 194 | 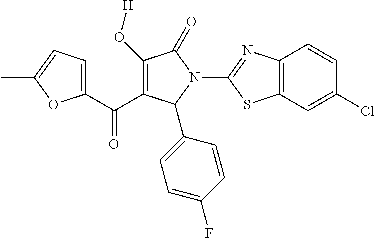 | **** | **** | |
| 195 | 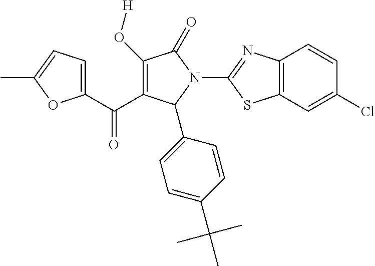 | **** | * | |
| 196 | 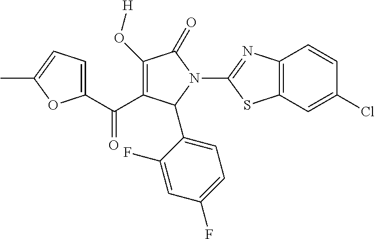 | **** | **** | |
| 197 | 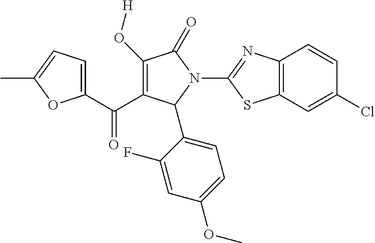 | **** | **** | |
| 198 | 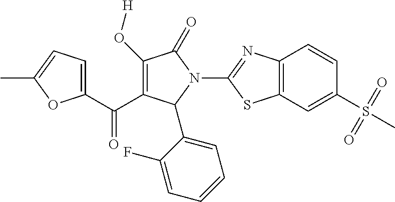 | * | * | |
| 199 | 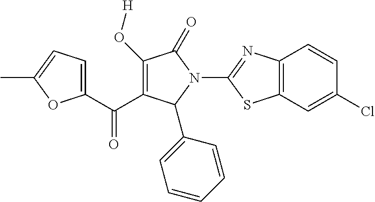 | **** | **** | |
| 200 | 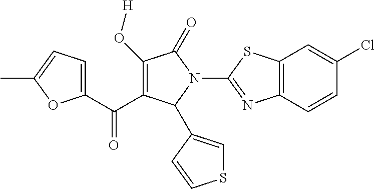 | **** | **** | |
| 201 | 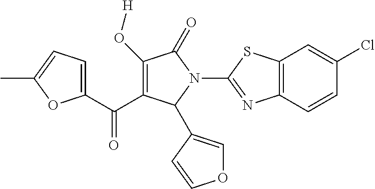 | *** | **** | |
| 202 | 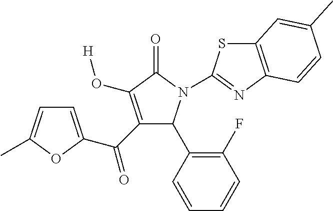 | **** | **** | |
| 203 | 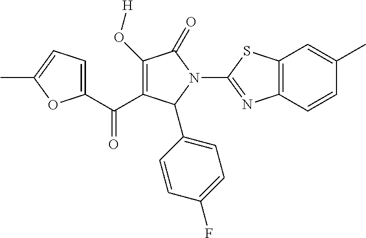 | **** | **** | |
| 204 | 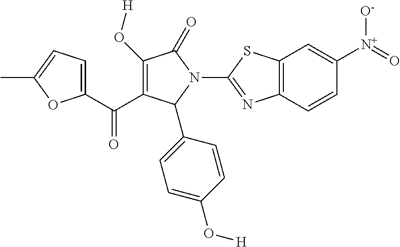 | * | * | |
| 205 | 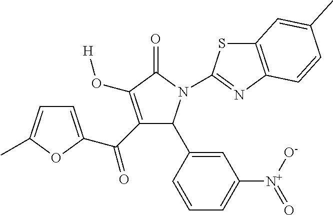 | **** | **** | |
| 206 | 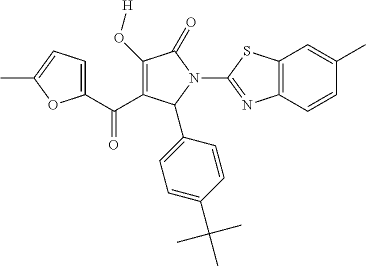 | **** | *** | |
| 207 | 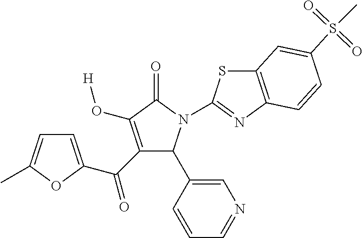 | * | * | |
| 208 | 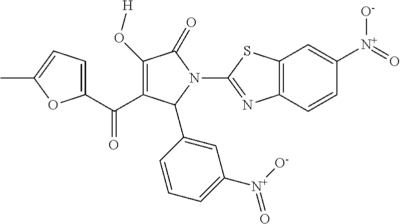 | * | **** | |
| 209 | 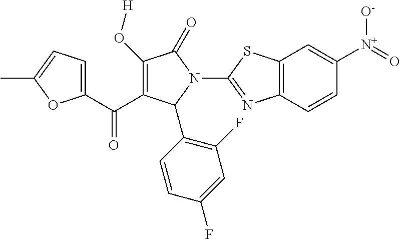 | **** | **** | |
| 210 | 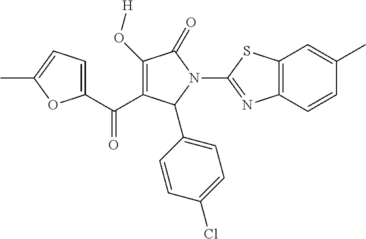 | **** | **** | |
| 211 | 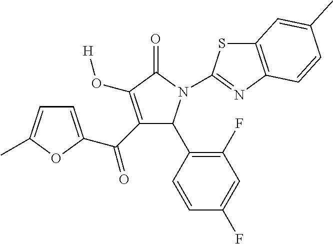 | **** | **** | |
| 212 | 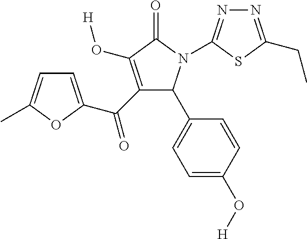 | * | * | |
| 213 | 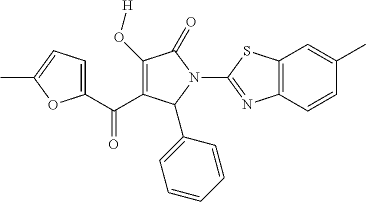 | *** | **** | |
| 214 | 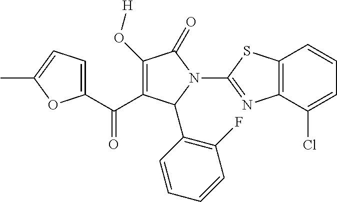 | **** | **** | |
| 215 | 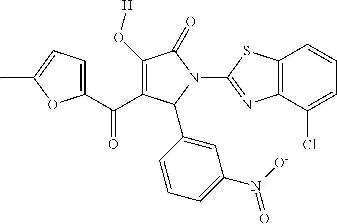 | **** | *8*** | |
| 216 | 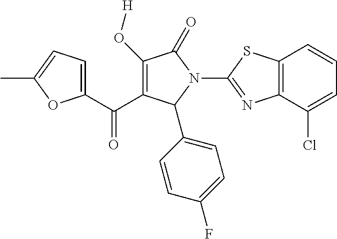 | **** | **** | |
| 217 | 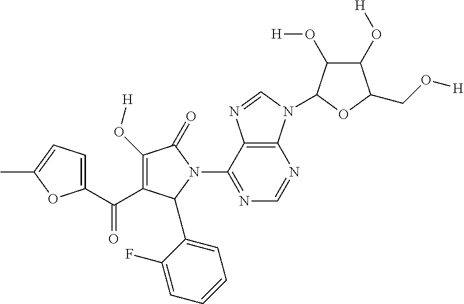 | * | * | |
| 218 | 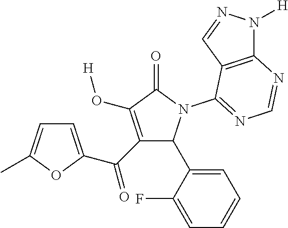 | * | * | |
| 219 | 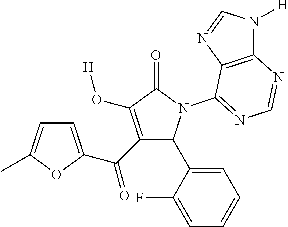 | * | * | |
| 220 | 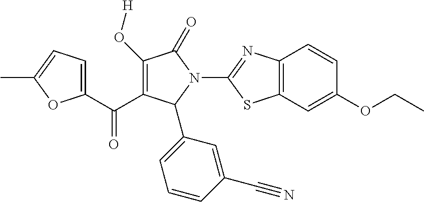 | **** | **** | |
| 221 | 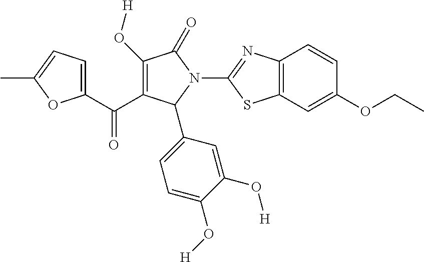 | * | * | |
| 222 | 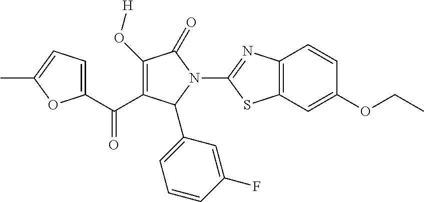 | **** | **** | |
| 223 | 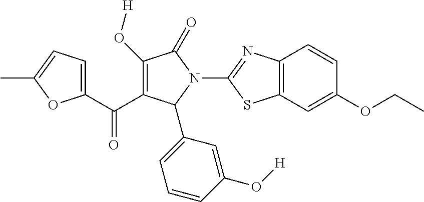 | * | ** | |
| 224 | 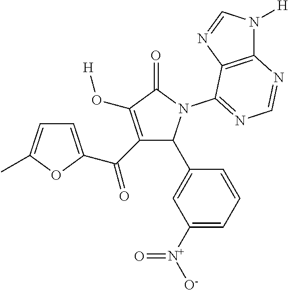 | * | * | |
| 225 | 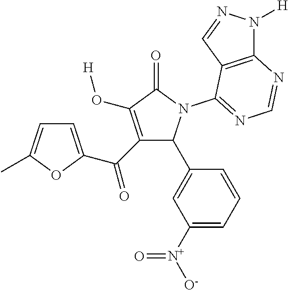 | * | * | |
| 226 | 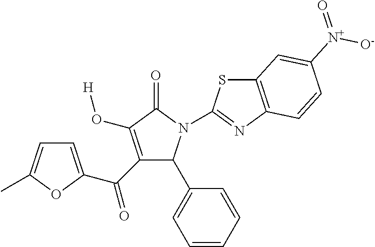 | **** | **** | |
| 227 | 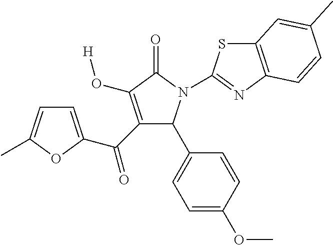 | **** | **** | |
| 228 | 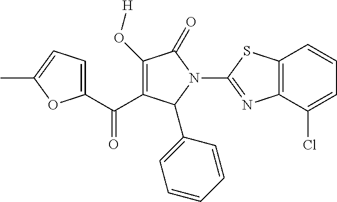 | **** | **** | |
| 229 | 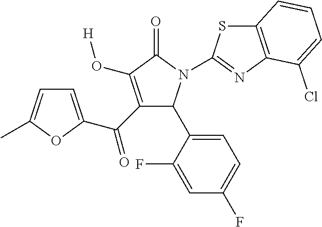 | **** | **** | |
| 230 | 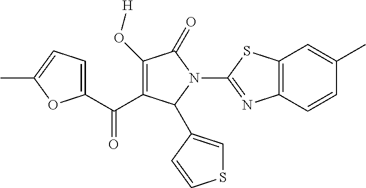 | **** | **** | |
| 231 | 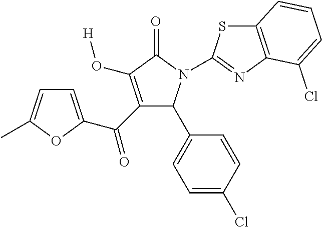 | **** | **** | |
| 232 | 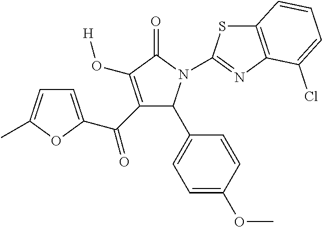 | **** | **8** | |
| 233 | 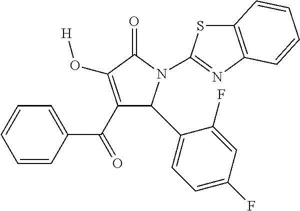 | *** | ** | |
| 234 | 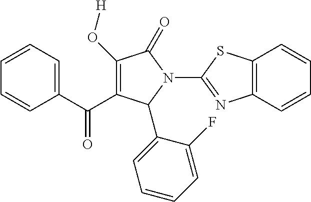 | ** | * | |
| 235 | 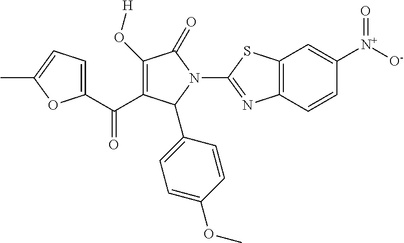 | **** | **** | |
| 236 | 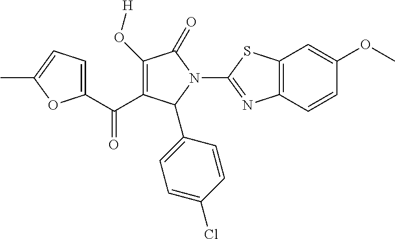 | **** | **** | |
| 237 | 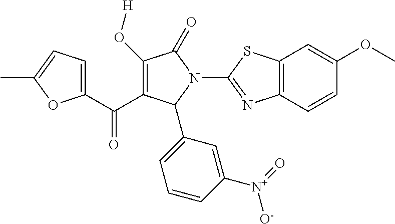 | **** | **** | |
| 238 | 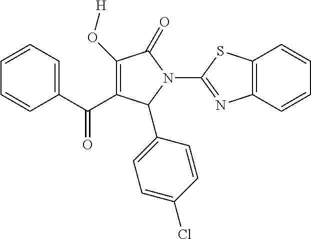 | **** | *** | |
| 239 | 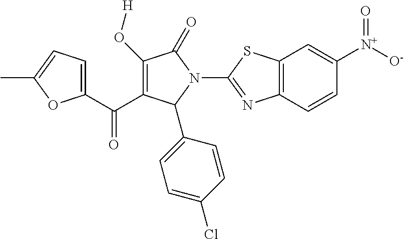 | **** | ***** | |
| 240 | 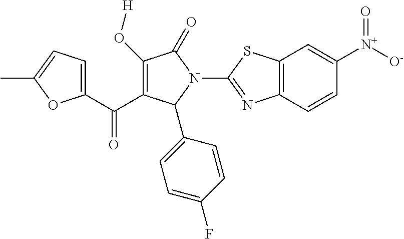 | **** | **** | |
| 241 | 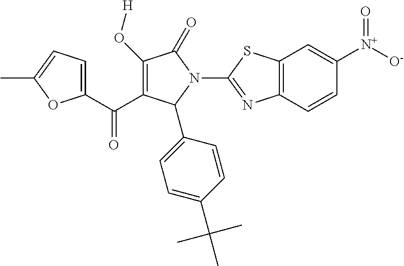 | ***** | **** | |
| 242 | 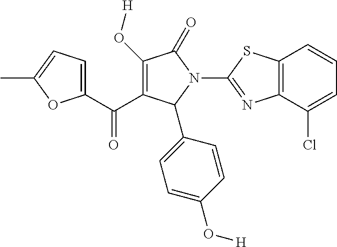 | *** | **** | |
| 243 | 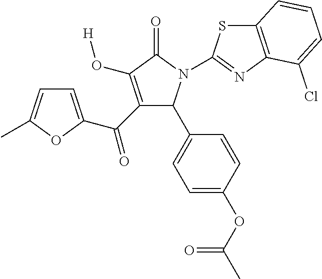 | *** | **** | |
| 244 | 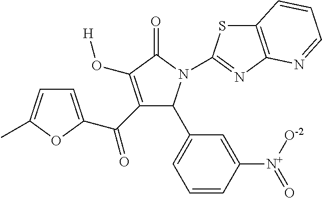 | * | * | |
| 245 | 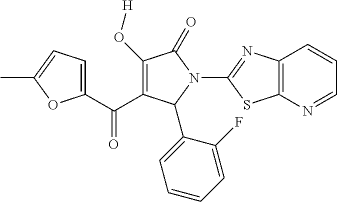 | * | * | |
| 246 | 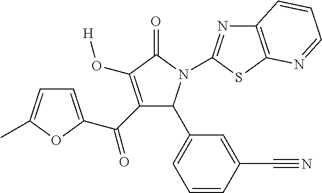 | * | * | |
| 247 | 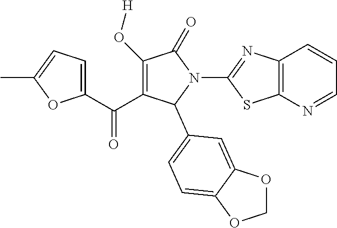 | * | * | |
| 248 | 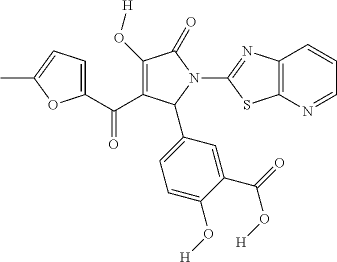 | * | * | |
| 249 | 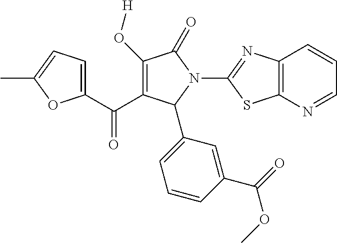 | * | * | |
| 250 | 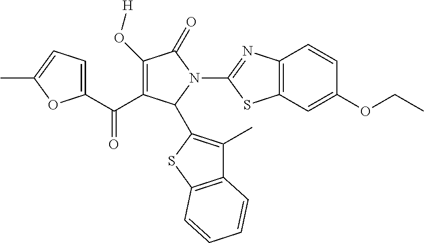 | * | **** | |
| 251 | 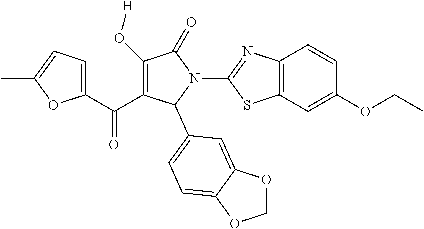 | **** | **** | |
| 252 | 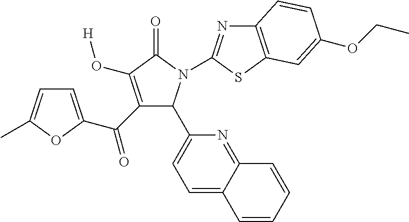 | * | **** | |
| 253 | 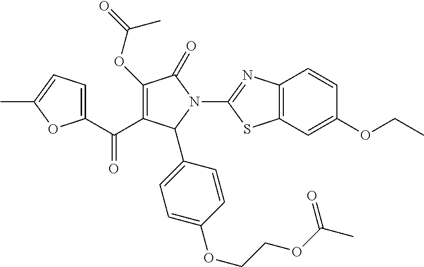 | * | **** | |
| 254 | 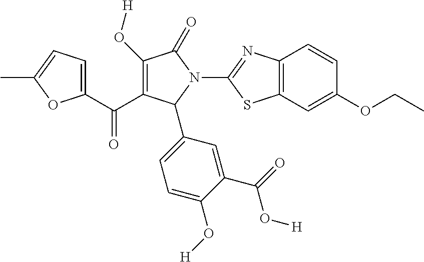 | * | * | |
| 255 | 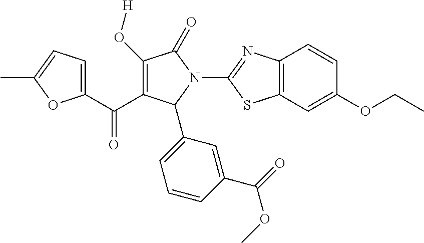 | * | **** | |
| 256 | 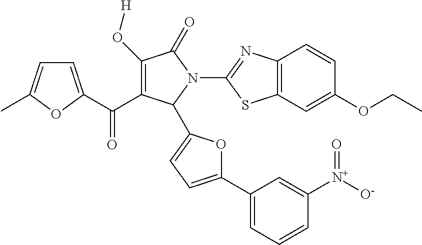 | * | **** | |
| 257 | 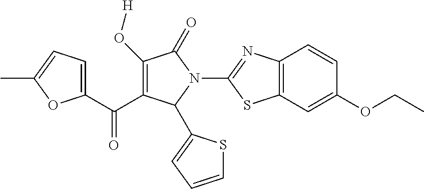 | **** | **** | |
| 258 | 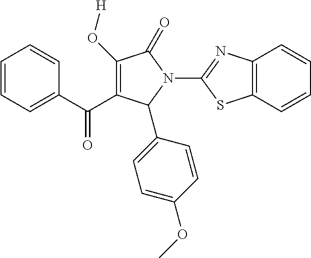 | * | ** | |
| 259 | 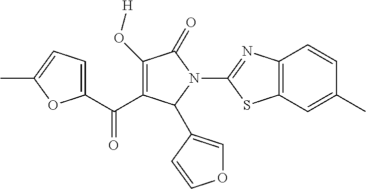 | ** | **** | |
| 260 | 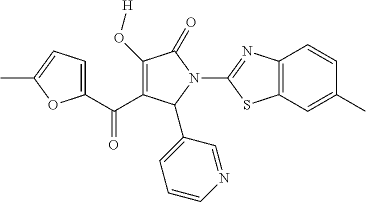 | * | * | |
| 261 | 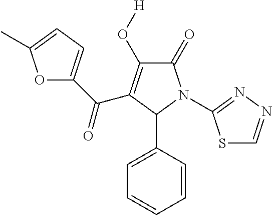 | * | * | |
| 262 | 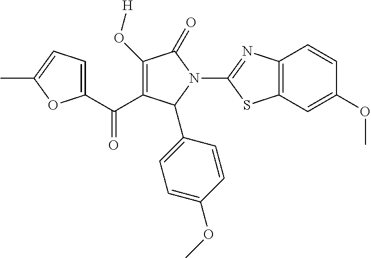 | **** | **** | |
| 263 | 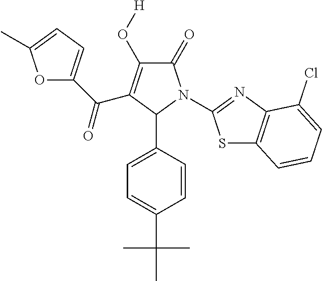 | ** | ** | |
| 264 | 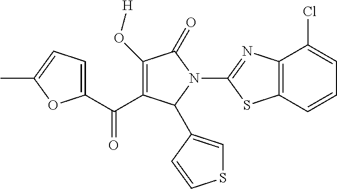 | **** | **** | |
| 265 | 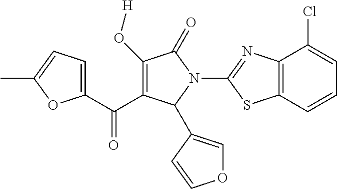 | **** | **** | |
| 266 | 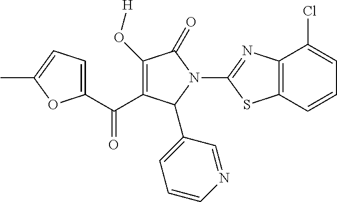 | * | * | |
| 267 | 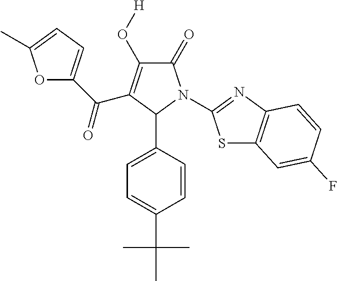 | **** | **** | |
| 268 | 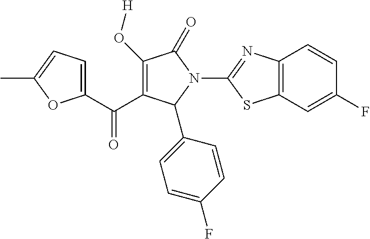 | **** | **** | |
| 269 | 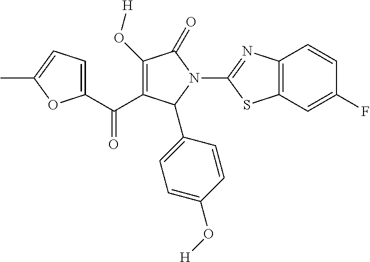 | * | ** | |
| 270 | 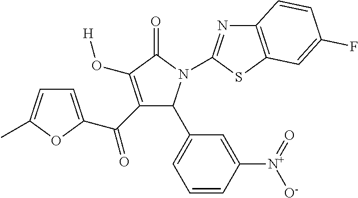 | **** | **** | |
| 271 | 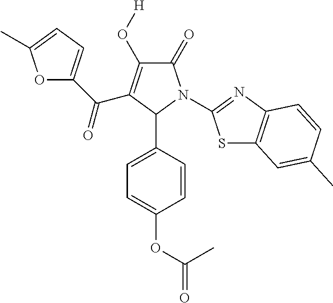 | ** | *** | |
| 272 | 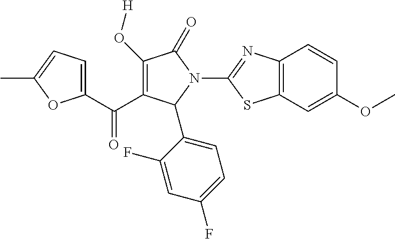 | *** | **** | |
| 273 | 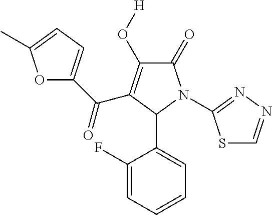 | * | * | |
| 274 | 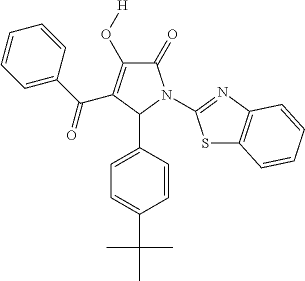 | *** | **** | |
| 275 | 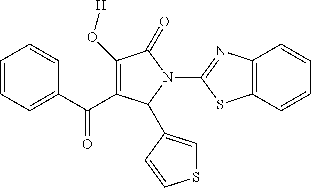 | * | * | |
| 276 | 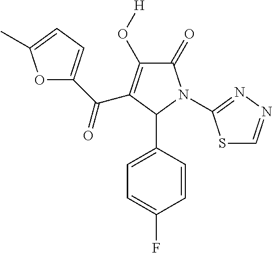 | * | * | |
| 277 | 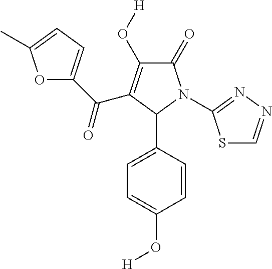 | * | * | |
| 278 | 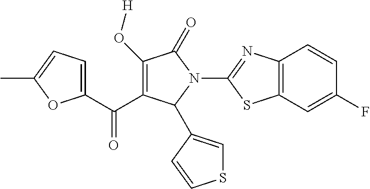 | **** | **** | |
| 279 | 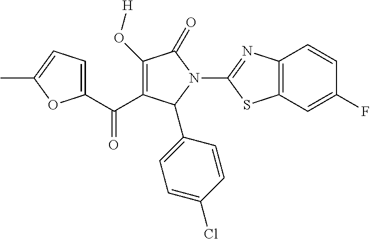 | **** | **** | |
| 280 | 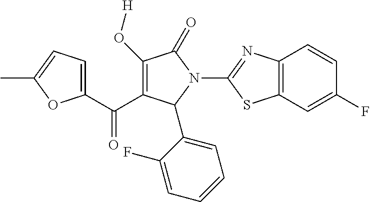 | **** | **** | |
| 281 | 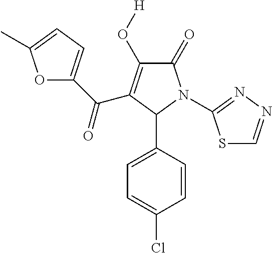 | * | * | |
| 282 | 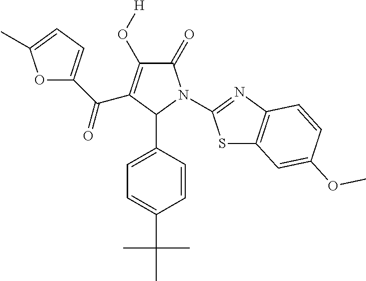 | * | ***** | |
| 283 | 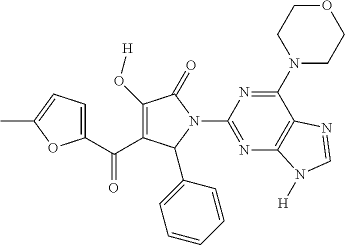 | * | * | |
| 284 | 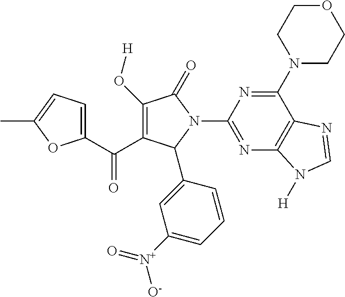 | * | * | |
| 285 | 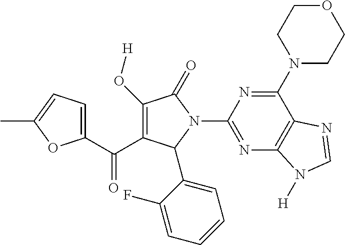 | * | * | |
| 286 | 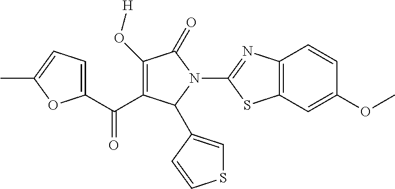 | * | *** | |
| 287 | 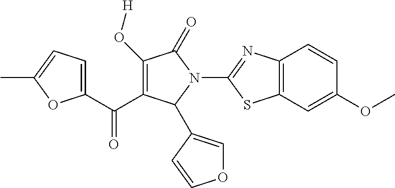 | * | ** | |
| 288 | 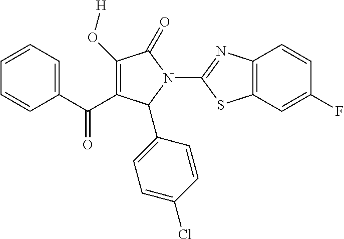 | *** | **** | |
| 289 | 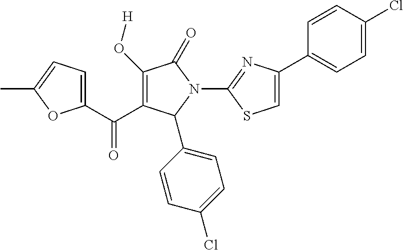 | **** | ** | |
| 290 | 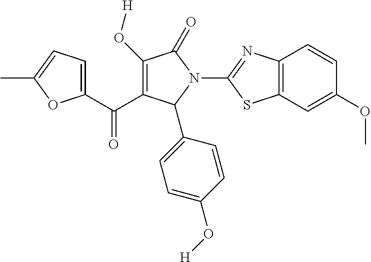 | * | ** | |
| 291 | 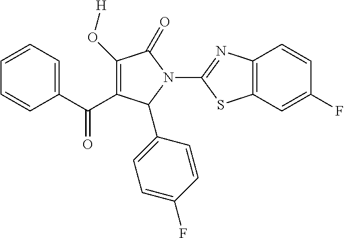 | *** | *** | |
| 292 | 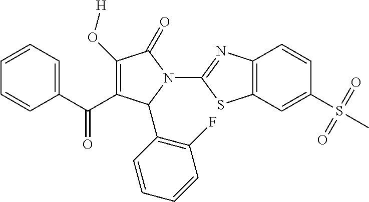 | * | * | |
| 293 | 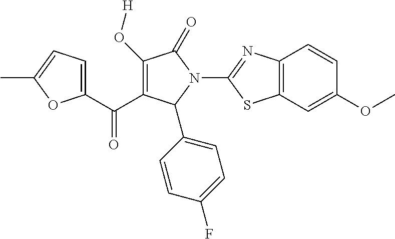 | * | **** | |
| 294 | 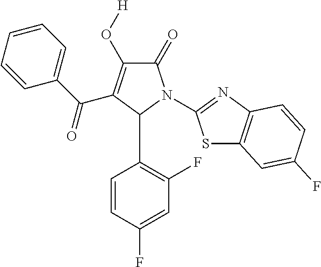 | *** | *** | |
| 295 | 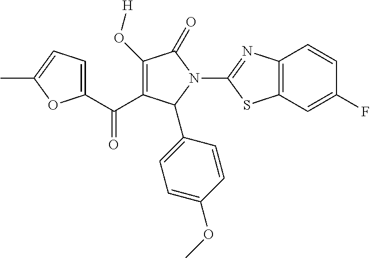 | **** | **** | |
| 296 | 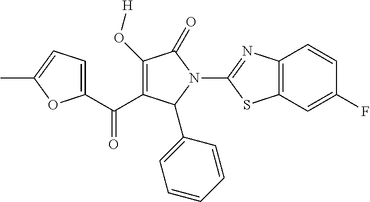 | *** | **** | |
| 297 | 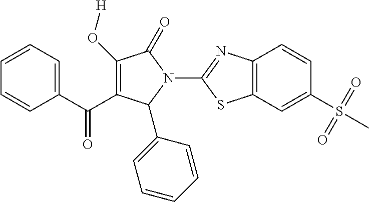 | * | * | |
| 298 | 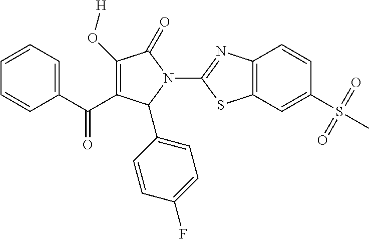 | * | * | |
| 299 | 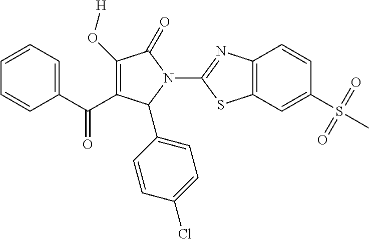 | * | * | |
| 300 | 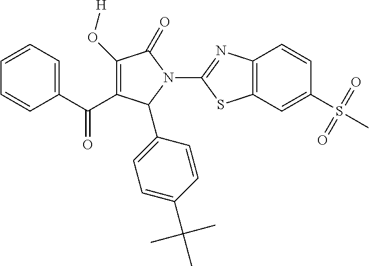 | ** | *** | |
| 301 | 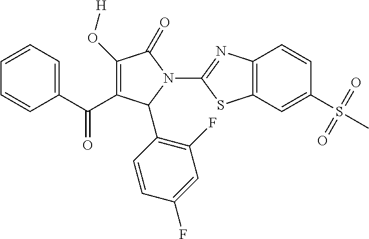 | * | * | |
| 302 | 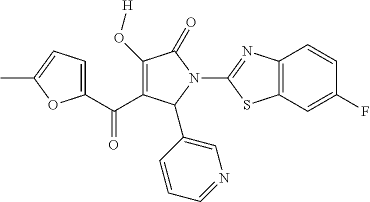 | * | * | |
| 303 | 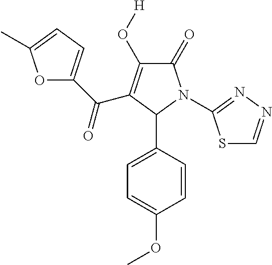 | * | * | |
| 304 | 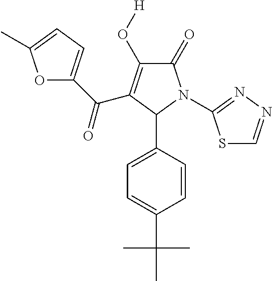 | ** | ** | |
| 305 | 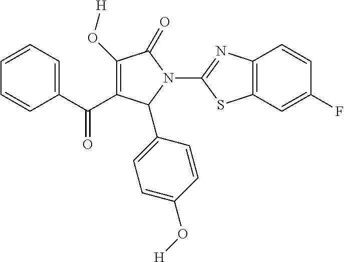 | * | * | |
| 306 | 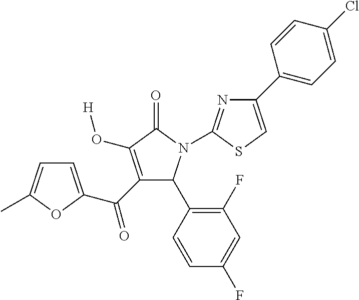 | **** | * | |
| 307 | 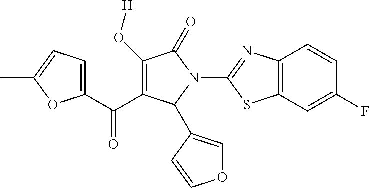 | ** | *** | |
| 308 | 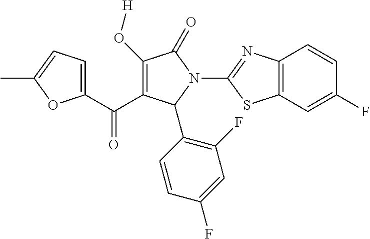 | **** | **** | |
| 309 | 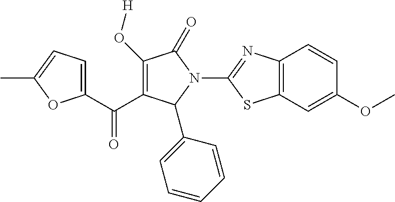 | * | **** | |
| 310 | 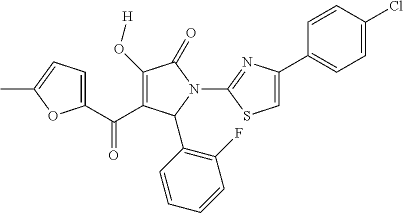 | **** | **** | |
| 311 | 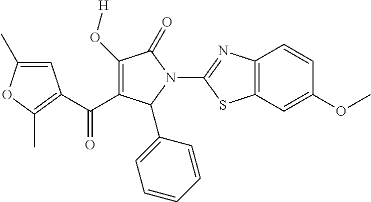 | * | *** | |
| 312 | 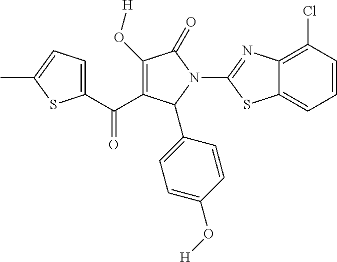 | ** | *** | |
| 313 | 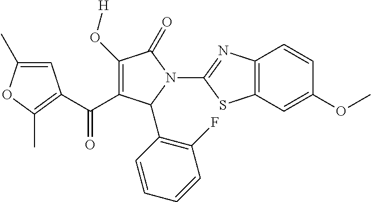 | * | *** | |
| 314 | 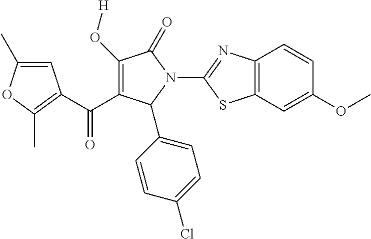 | * | **** | |
| 315 | 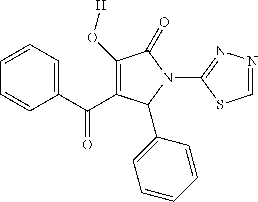 | * | * | |
| 316 | 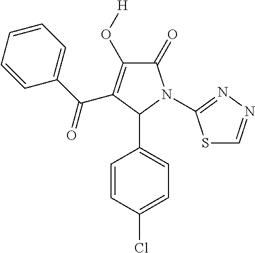 | * | * | |
| 317 | 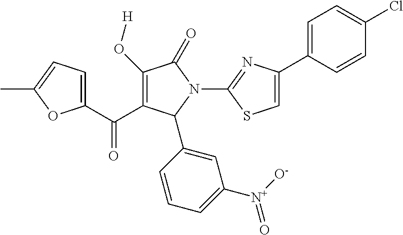 | **** | ***** | |
| 318 | 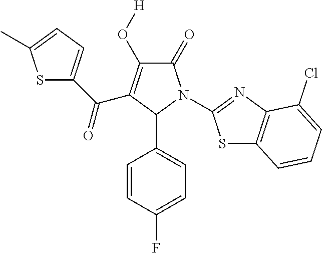 | **** | **** | |
| 319 | 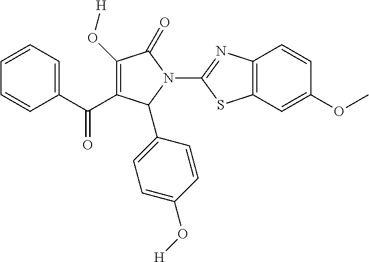 | * | * | |
| 320 | 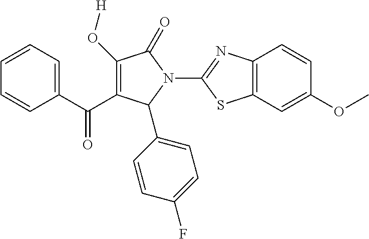 | * | ** | |
| 321 | 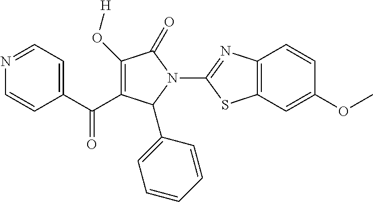 | * | * | |
| 322 | 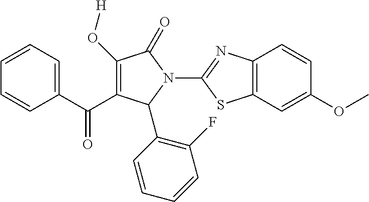 | ** | * | |
| 323 | 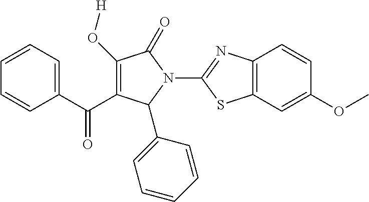 | * | * | |
| 324 | 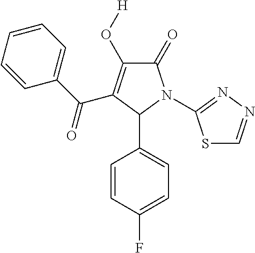 | * | * | |
| 325 | 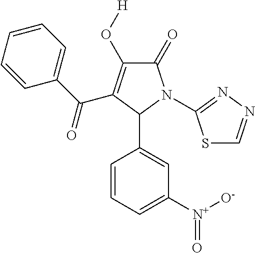 | * | * | |
| 326 | 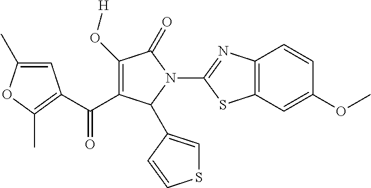 | * | *** | |
| 327 | 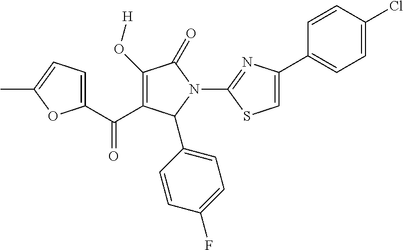 | **** | **** | |
| 328 | 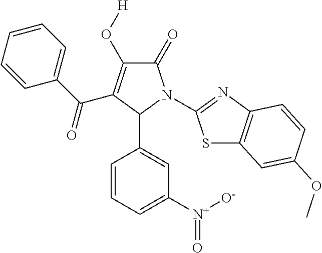 | * | * | |
| 329 | 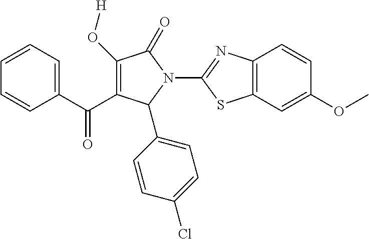 | *** | *** | |
| 330 | 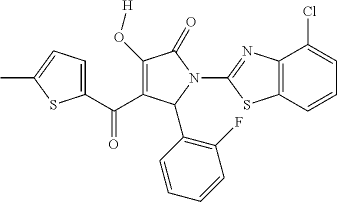 | **** | **** | |
| 331 | 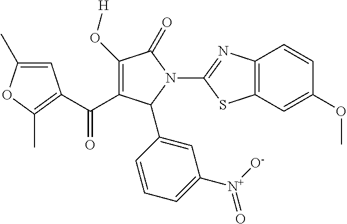 | ** | **** | |
| 332 | 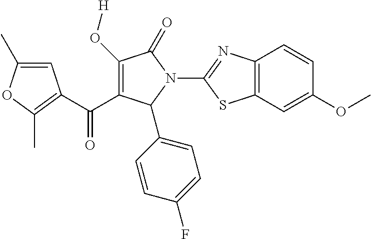 | * | **** | |
| 333 | 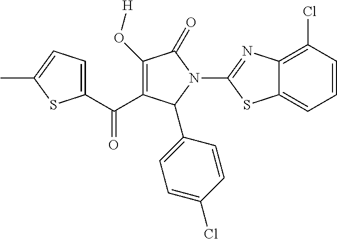 | **** | **** | |
| 334 | 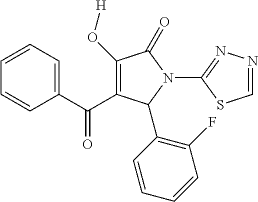 | * | * | |
| 335 | 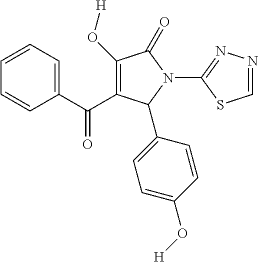 | * | * | |
| 336 | 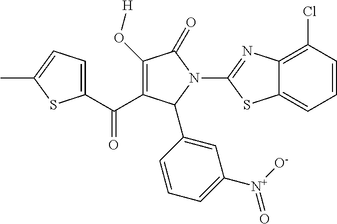 | **** | **** | |
| 337 | 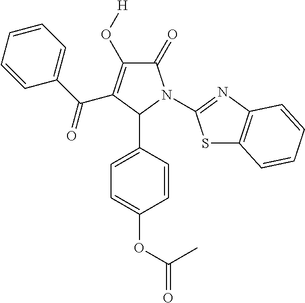 | * | * | |
| 338 | 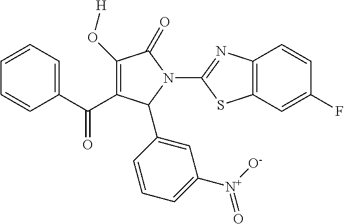 | ** | ** | |
| 339 | 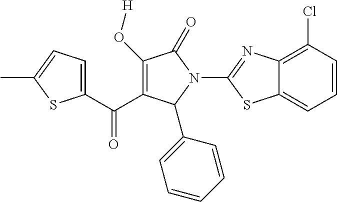 | **** | **** | |
| 340 | 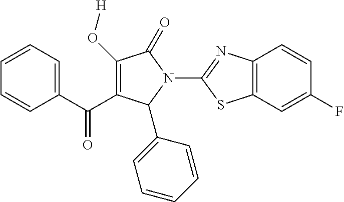 | ** | ** | |
| 341 | 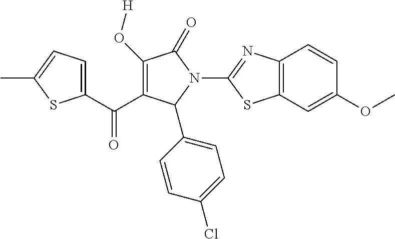 | *** | **** | |
| 342 | 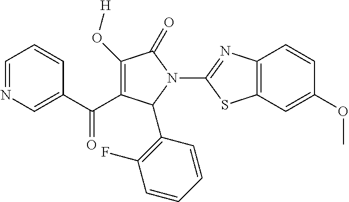 | * | * | |
| 343 | 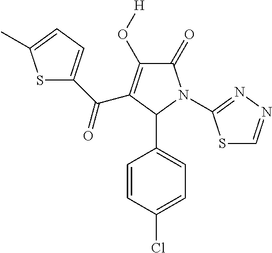 | * | * | |
| 344 | 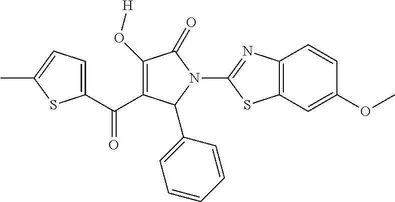 | * | *** | |
| 345 | 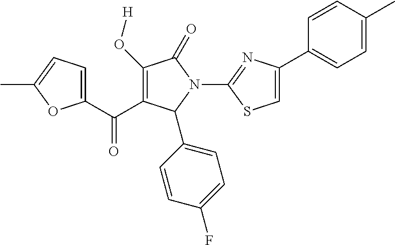 | **** | **** | |
| 346 | 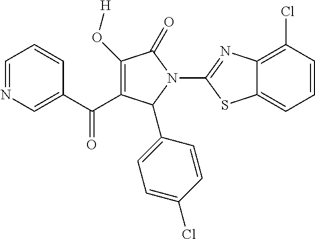 | * | * | |
| 347 | 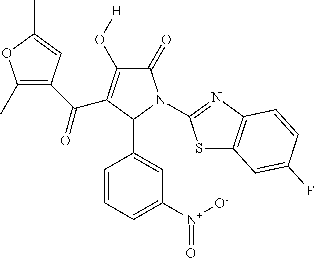 | ** | *** | |
| 348 | 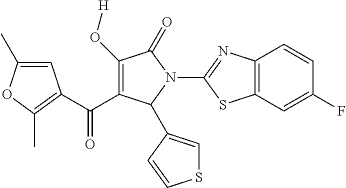 | ** | **** | |
| 349 | 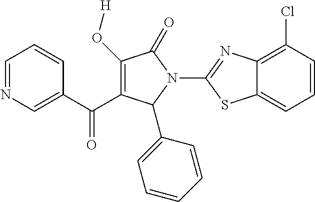 | * | * | |
| 350 | 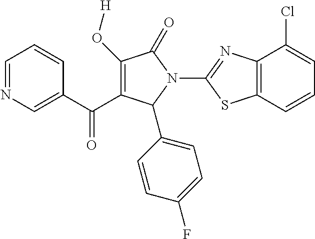 | * | * | |
| 351 | 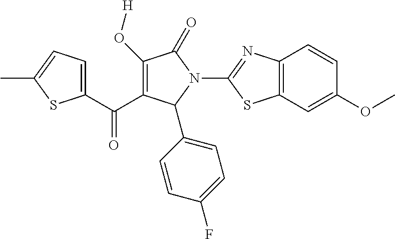 | ** | *** | |
| 352 | 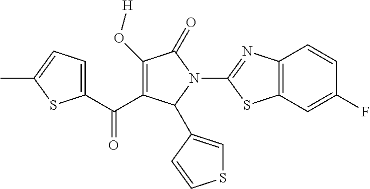 | *** | **** | |
| 353 | 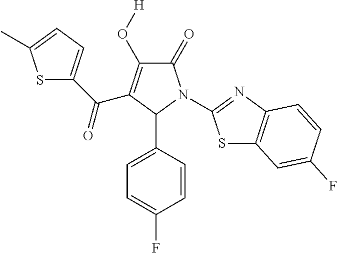 | *** | *** | |
| 354 | 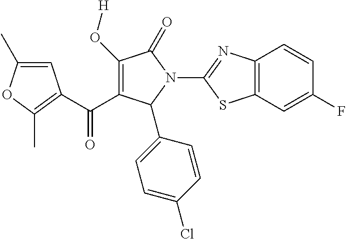 | *** | **** | |
| 355 | 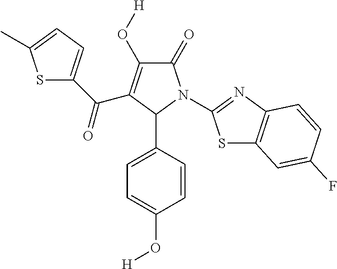 | * | * | |
| 356 | 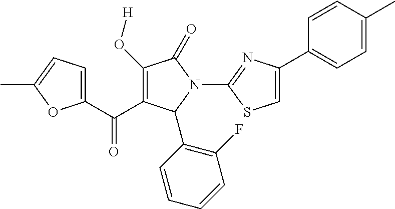 | **** | **** | |
| 357 | 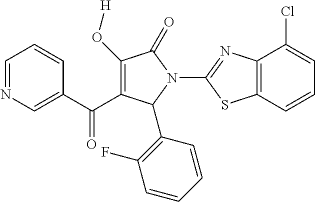 | * | * | |
| 358 | 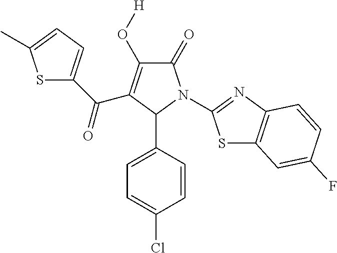 | **** | **** | |
| 359 | 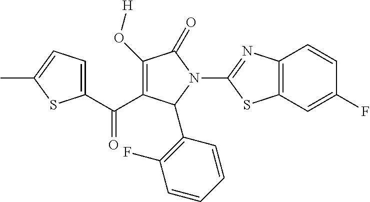 | *** | **** | |
| 362 | 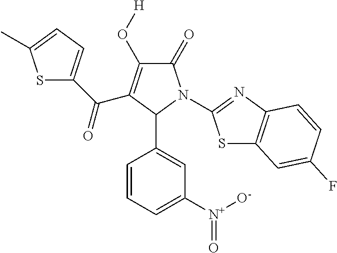 | *** | *** | |
| 361 | 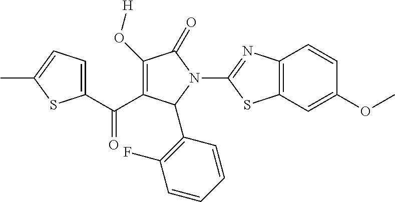 | ** | *** | |
| 362 | 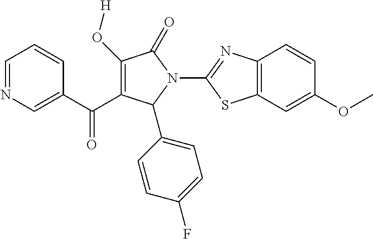 | * | * | |
| 363 | 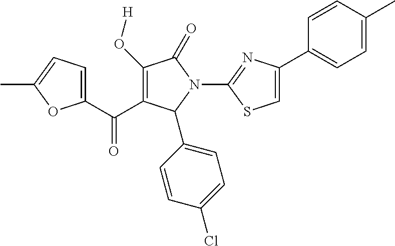 | **** | **** | |
| 364 | 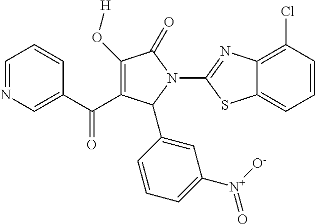 | * | * | |
| 365 | 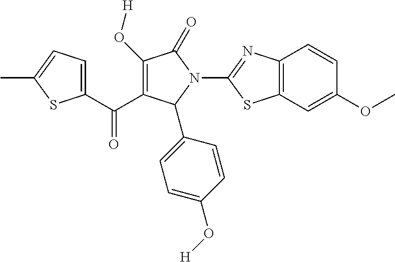 | * | * | |
| 366 | 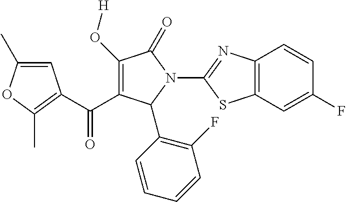 | * | *** | |
| 367 | 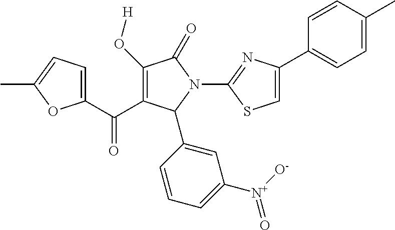 | **** | ***** | |
| 368 | 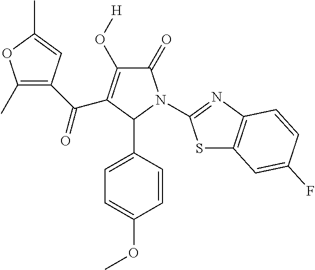 | ** | **** | |
| 369 | 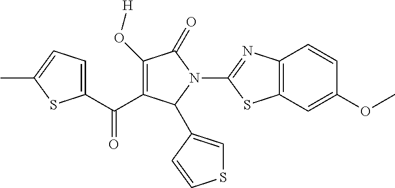 | * | *** | |
| 370 | 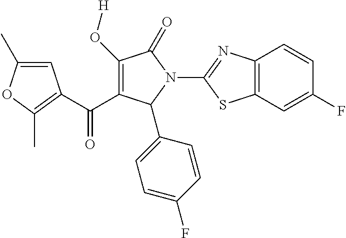 | * | **** | |
| 371 | 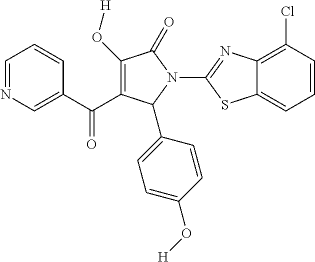 | * | * | |
| 372 | 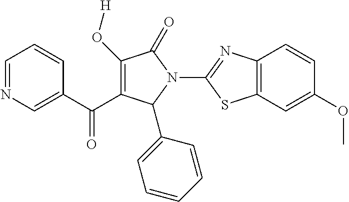 | * | * | |
| 373 | 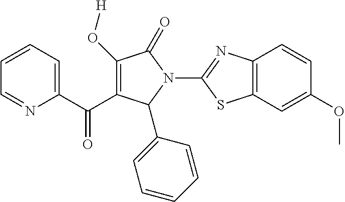 | * | * | |
| 374 | 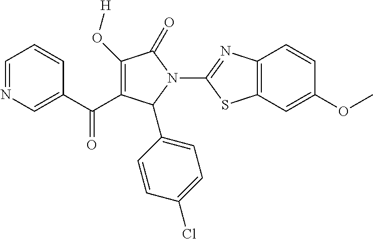 | * | * | |
| 375 | 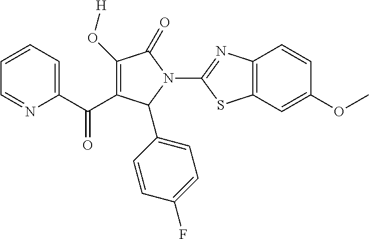 | * | * | |
| 376 | 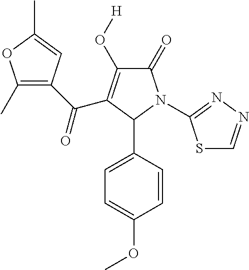 | * | * | |
| 377 | 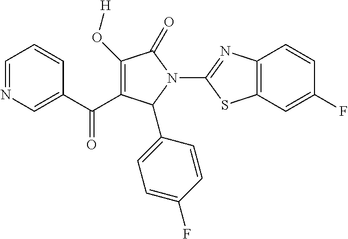 | * | * | |
| 378 | 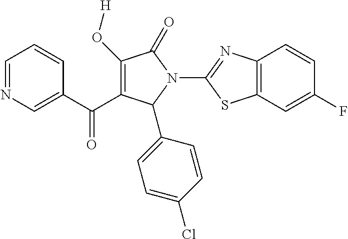 | * | * | |
| 379 | 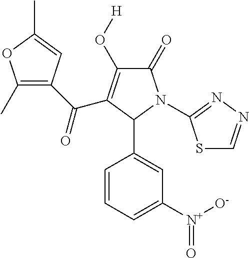 | * | * | |
| 380 | 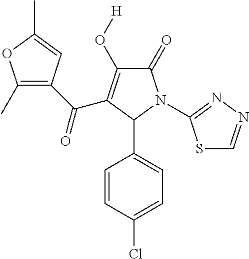 | * | * | |
| 381 | 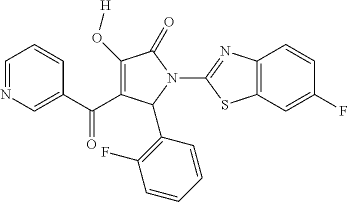 | * | * | |
| 382 | 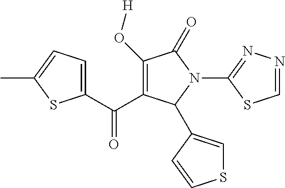 | * | * | |
| 383 | 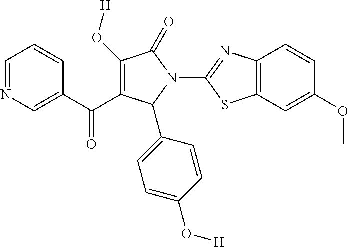 | * | * | |
| 384 | 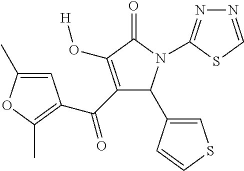 | * | * | |
| 385 | 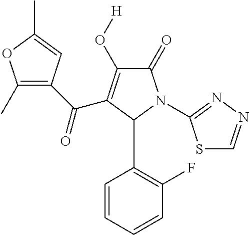 | * | * | |
| 386 | 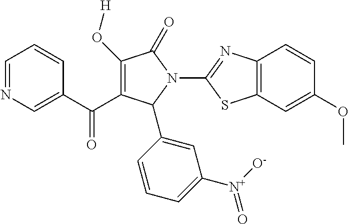 | * | * | |
| 387 | 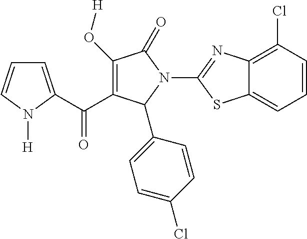 | * | * | |
| 388 | 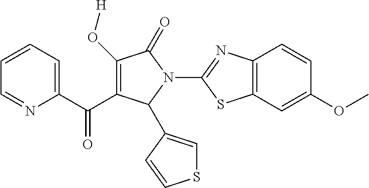 | * | * | |
| 389 | 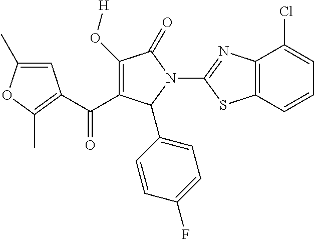 | *** | **** | |
| 390 | 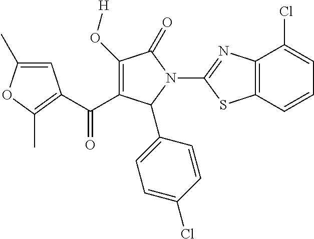 | *** | **** | |
| 391 | 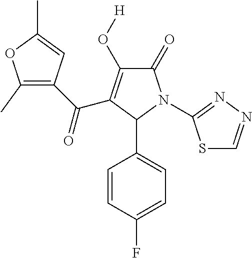 | * | * | |
| 392 | 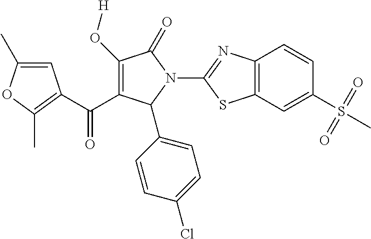 | * | * | |
| 393 | 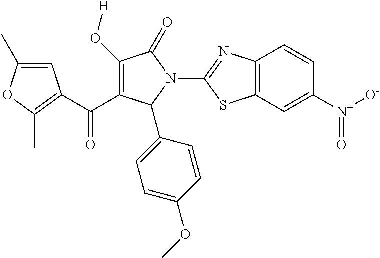 | *** | **** | |
| 394 | 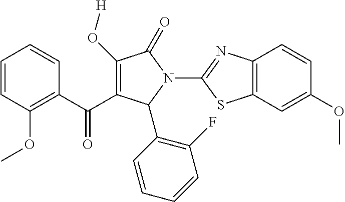 | ** | *** | |
| 395 | 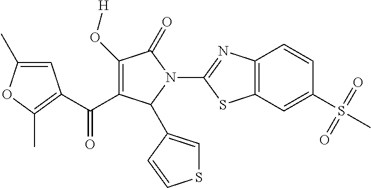 | * | * | |
| 396 | 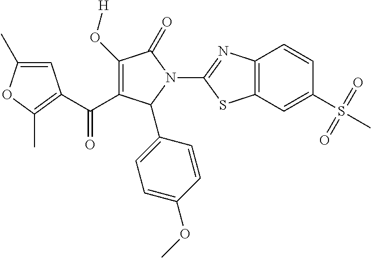 | * | * | |
| 397 | 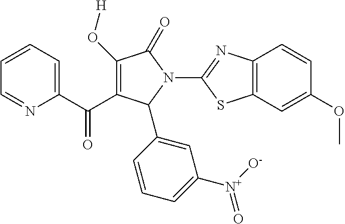 | * | * | |
| 398 | 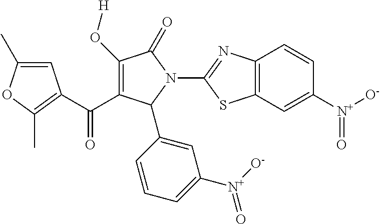 | * | ** | |
| 399 | 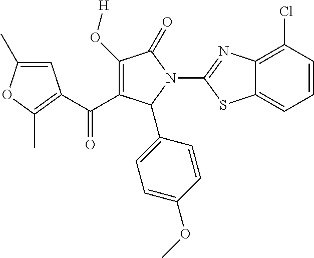 | **** | **** | |
| 400 | 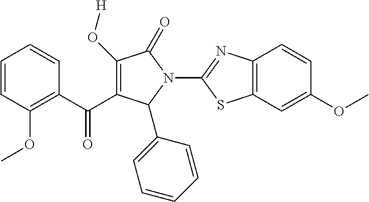 | ** | *** | |
| 401 | 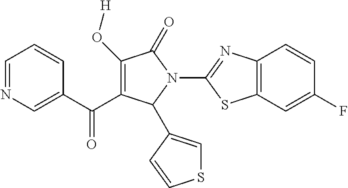 | * | * | |
| 402 | 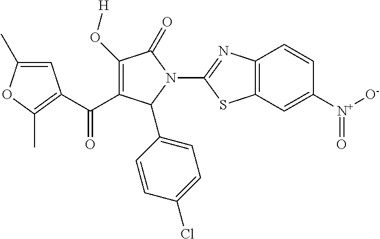 | * | **** | |
| 403 | 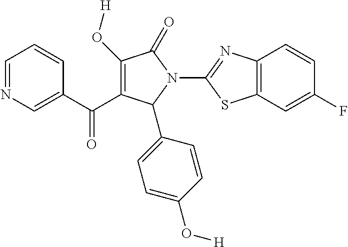 | * | * | |
| 404 | 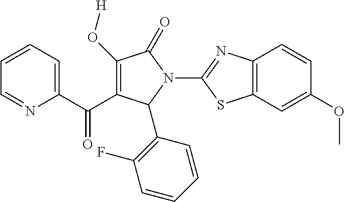 | * | * | |
| 405 | 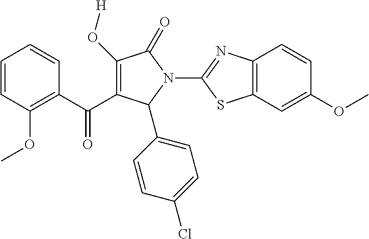 | * | **** | |
| 406 | 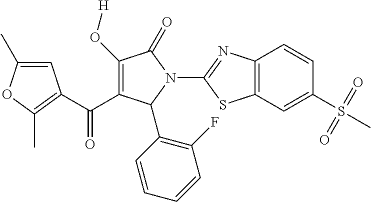 | * | * | |
| 407 | 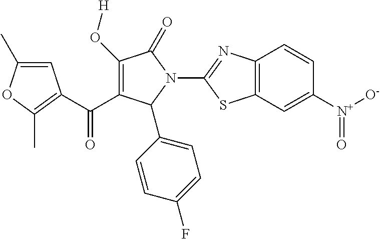 | * | **** | |
| 408 | 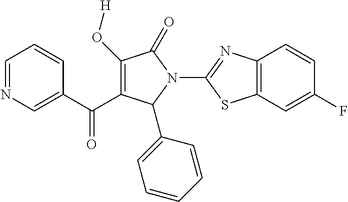 | * | * | |
| 409 | 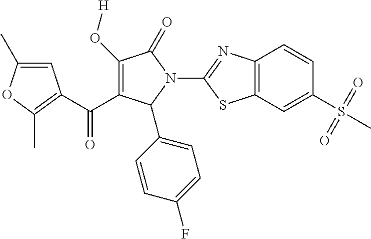 | * | * | |
| 410 | 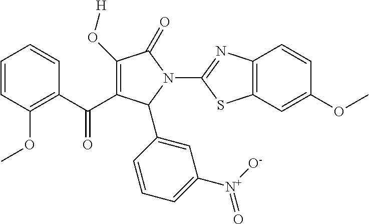 | ** | ** | |
| 411 | 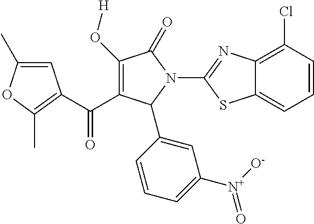 | ** | **** | |
| 412 | 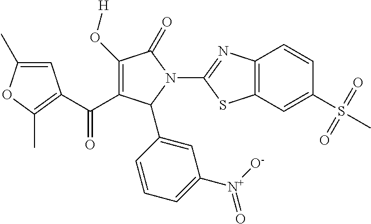 | * | * | |
| 413 | 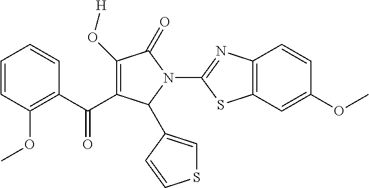 | * | ** | |
| 414 | 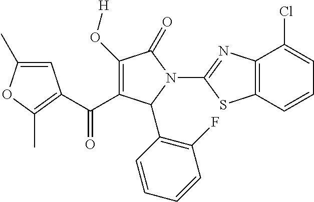 | ** | **** | |
| 415 | 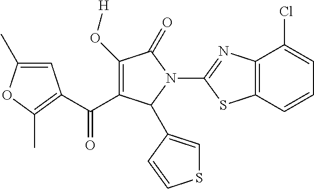 | **** | **** | |
| 416 | 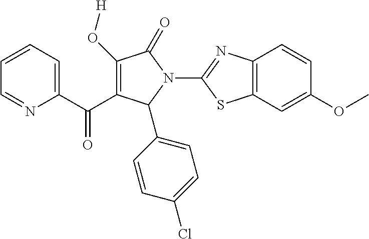 | * | * | |
| 417 | 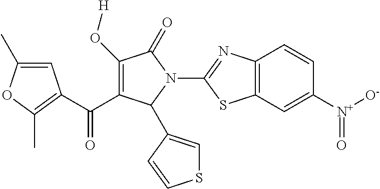 | * | **** | |
| 418 | 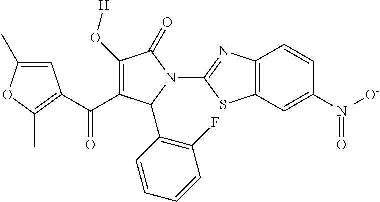 | * | **** | |
| 419 | 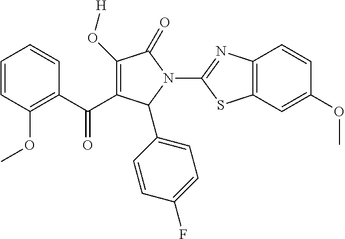 | * | *** | |
| 420 | 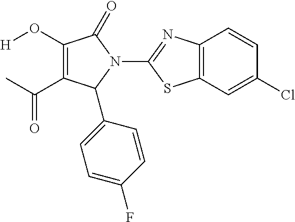 | **** | ***** | |
| 421 | 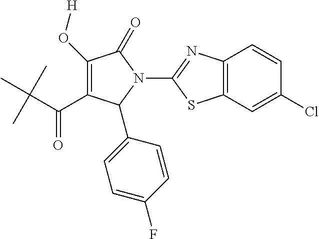 | ** | * | |
| 422 | 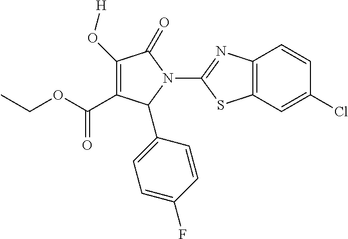 | *** | *** | |
| 423 | 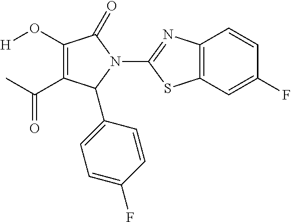 | **** | **** | |
| 424 | 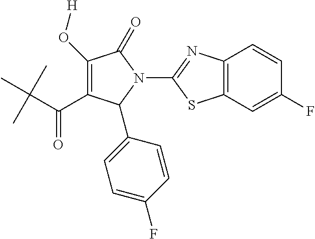 | ** | ** | |
| 425 | 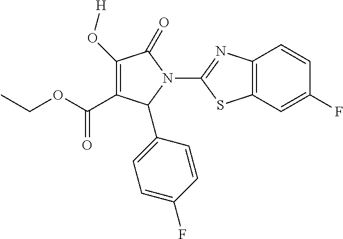 | * | * | |
| 426 | 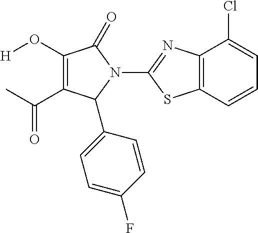 | ** | ** | |
| 427 | 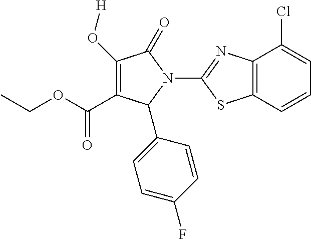 | ** | * | |
| 428 | 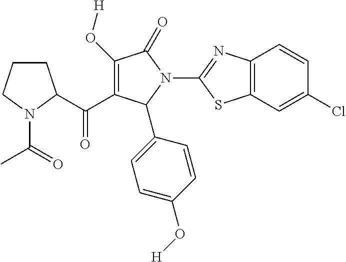 | * | * | |
| 429 | 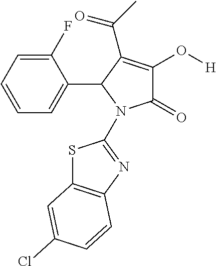 | * | * | |
| 430 | 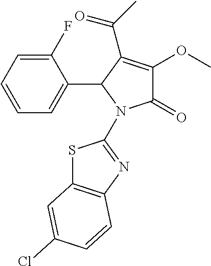 | * | * | |
| 431 | 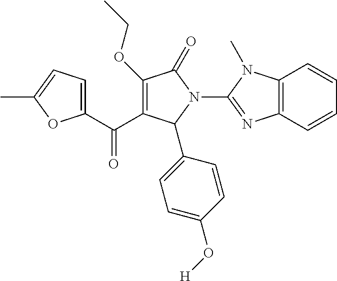 | * | * | |
| 432 | 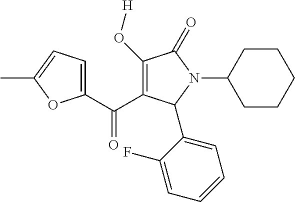 | * | * | |
| 433 | 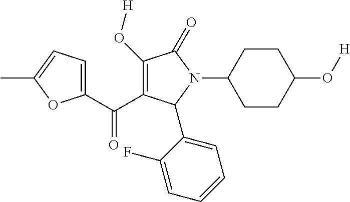 | ** | ** | |
| 434 | 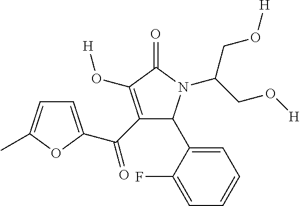 | * | * | |
| 435 | 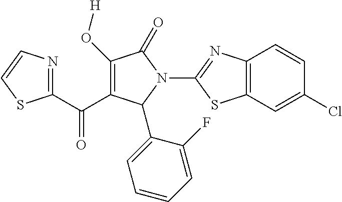 | **** | ***** | |
| 436 | 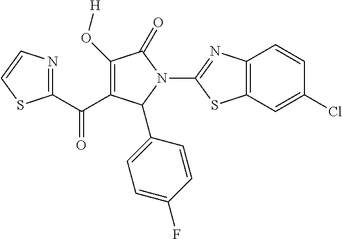 | **** | **** | |
| 437 | 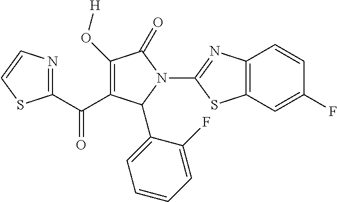 | **** | **** | |
| 438 | 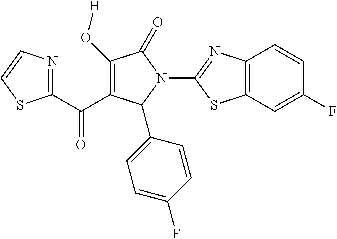 | * | *** | |
| 439 | 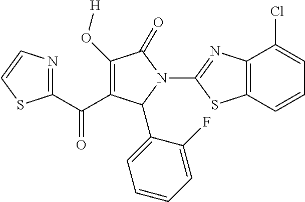 | *** | **** | |
| 440 | 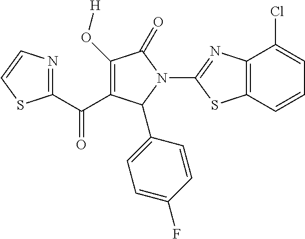 | **** | **** | |
| 441 | 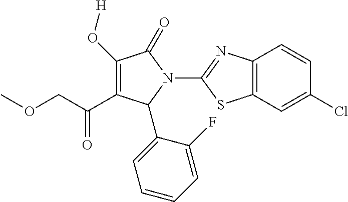 | **** | **** | |
| 442 | 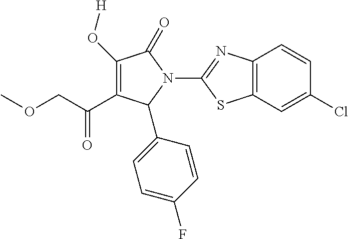 | * | * | |
| 443 | 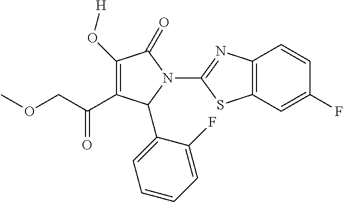 | * | * | |
| 444 | 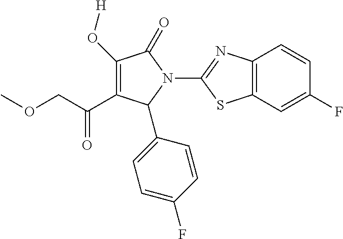 | * | * | |
| 445 | 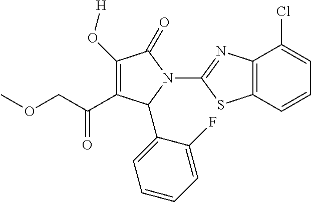 | * | * | |
| 446 | 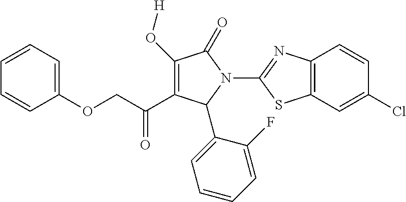 | * | * | |
| 447 | 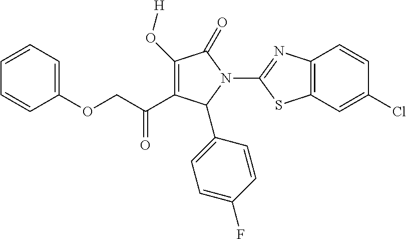 | ** | ** | |
| 448 | 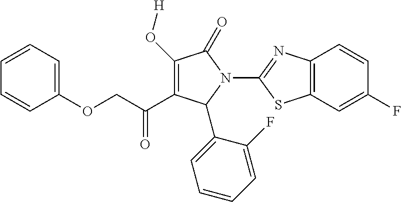 | ** | *** | |
| 449 | 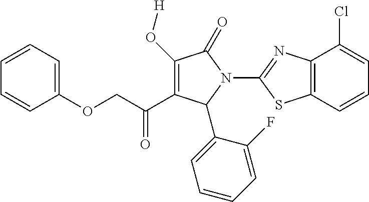 | * | * | |
| 450 | 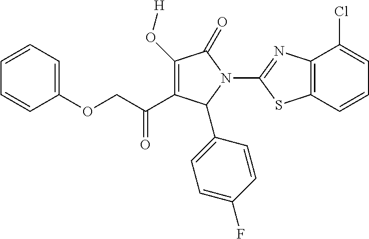 | * | * | |
| 451 | 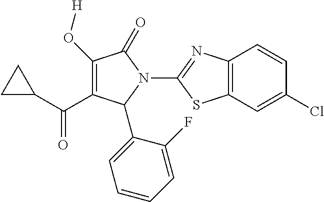 | * | *** | |
| 452 | 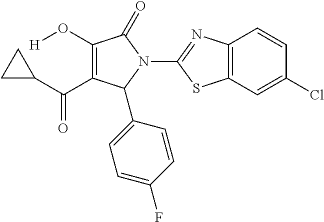 | *** | *** | |
| 453 | 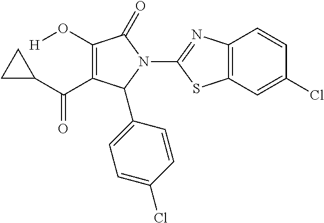 | **** | *** | |
| 454 | 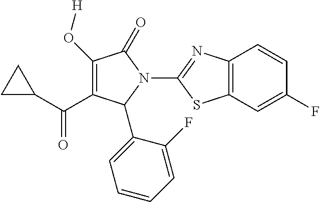 | **** | **** | |
| 455 | 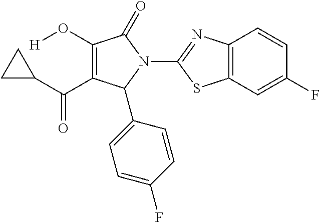 | * | ** | |
| 456 | 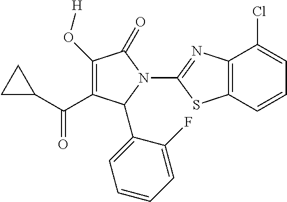 | **** | * | |
| 457 | 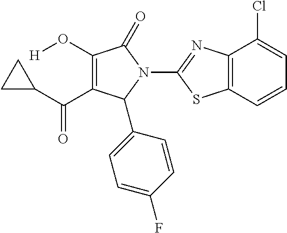 | ** | *** | |
| 458 | 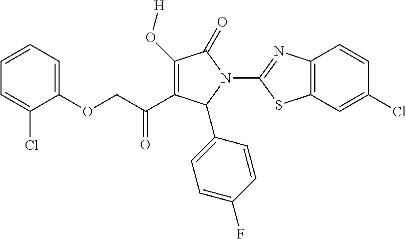 | ** | *** | |
| 459 | 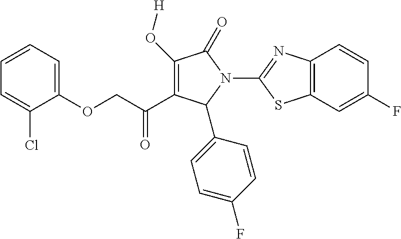 | *** | *** | |
| 460 | 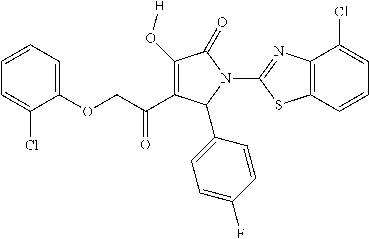 | ** | ** | |
| 461 | 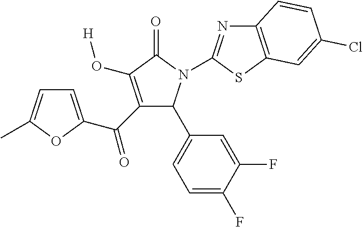 | * | *** | |
| 462 | 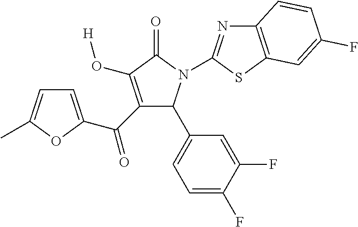 | **** | **** | |
| 463 | 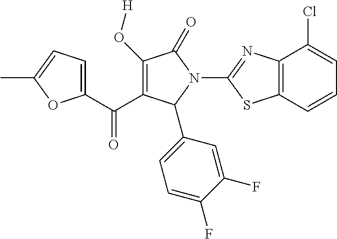 | **** | **** | |
| 464 | 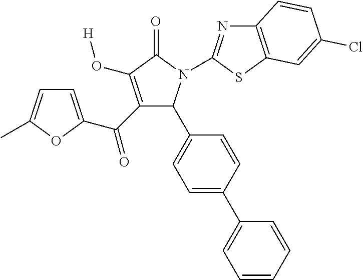 | * | * | |
| 465 | 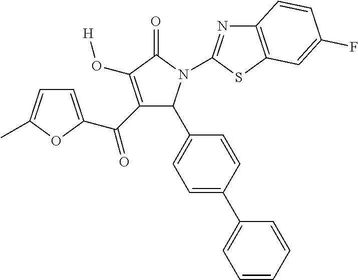 | * | **** | |
| 466 | 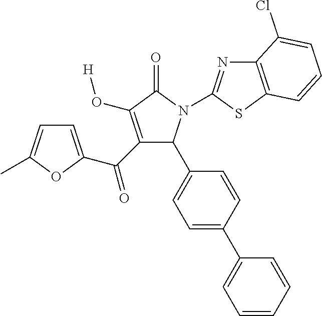 | * | **** | |
| 467 | 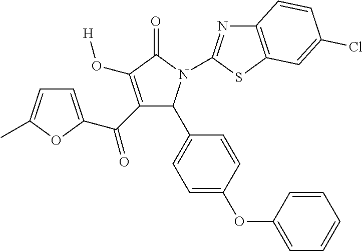 | * | *** | |
| 468 | 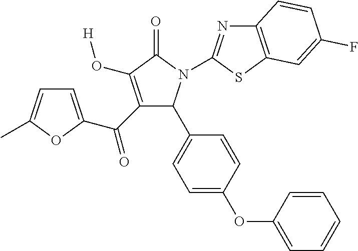 | * | * | |
| 469 | 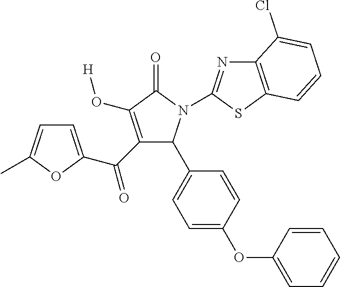 | * | **** | |
| 470 | 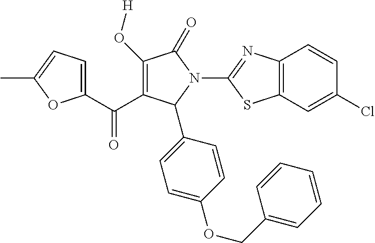 | * | * | |
| 471 | 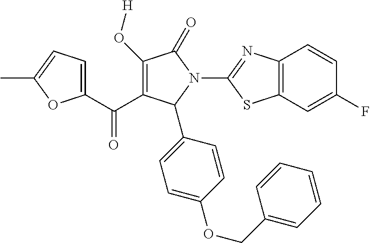 | * | * | |
| 472 | 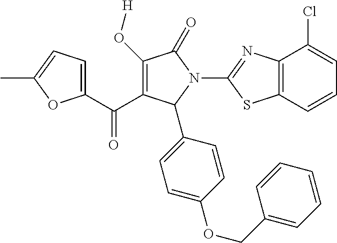 | * | **** | |
| 473 | 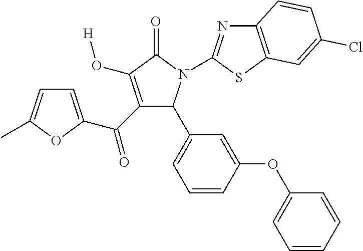 | * | * | |
| 474 | 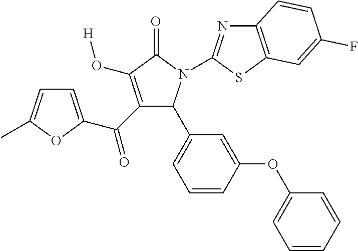 | * | * | |
| 475 | 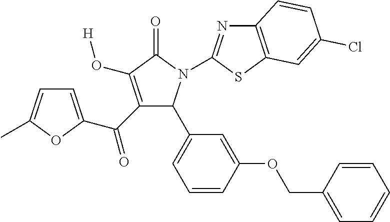 | * | **** | |
| 476 | 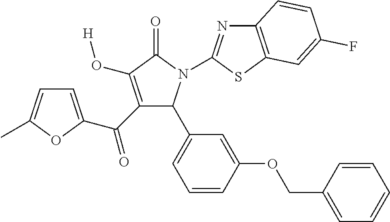 | * | *** | |
| 477 | 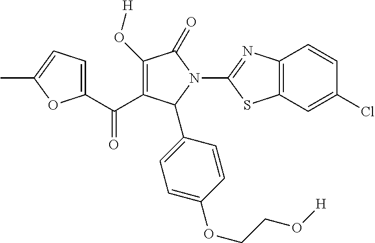 | *** | **** | |
| 478 | 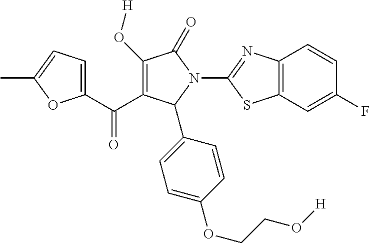 | *** | **** | |
| 479 | 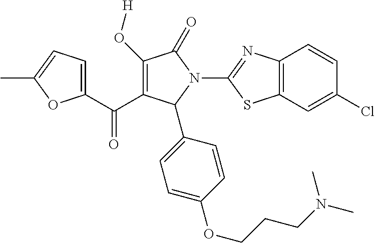 | *** | **** | |
| 480 | 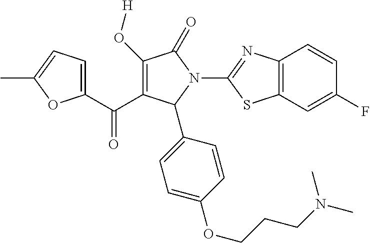 | **** | **** | |
| 481 | 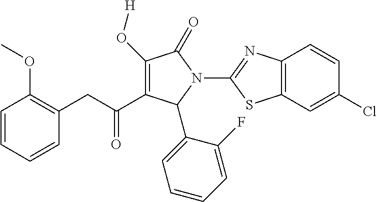 | **** | **** | |
| 482 | 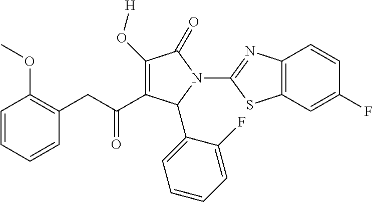 | * | ** | |
| 483 | 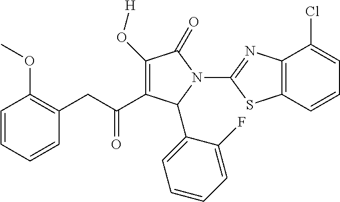 | ** | *** | |
| 484 | 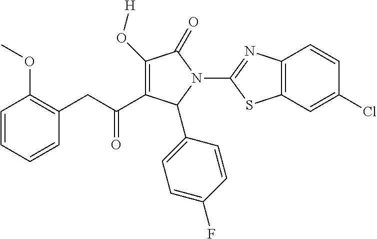 | ** | **** | |
| 485 | 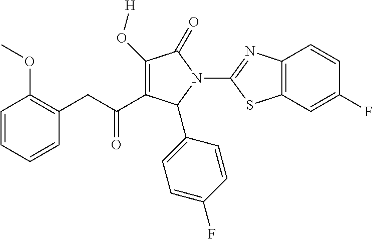 | *** | **** | |
| 486 | 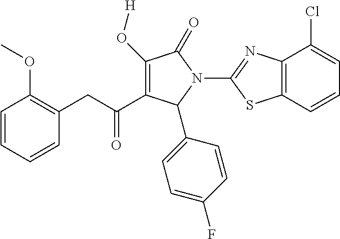 | *** | *** | |
| 487 | 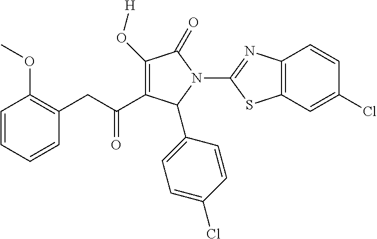 | *** | **** | |
| 488 | 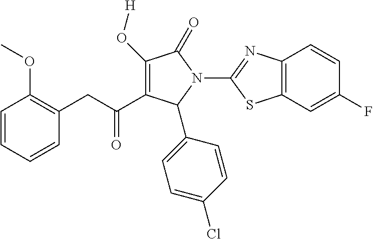 | *** | **** | |
| 489 | 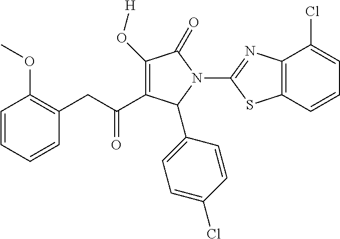 | **** | **** | |
| 490 | 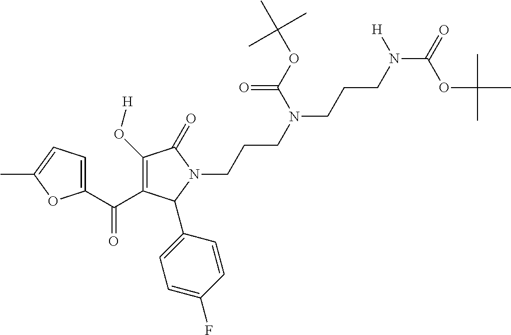 | * | * | |
| 491 | 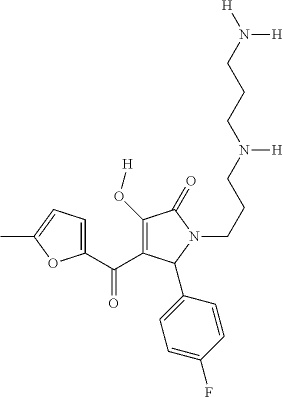 | * | * | |
| 492 | 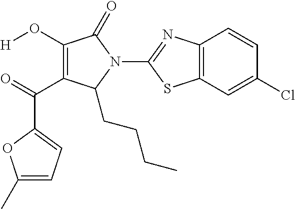 | **** | **** | |
| 493 | 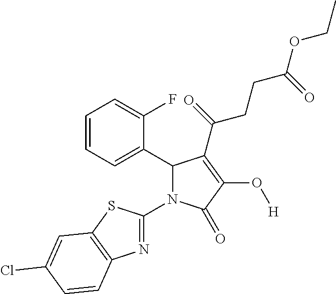 | * | **** | |
| 494 | 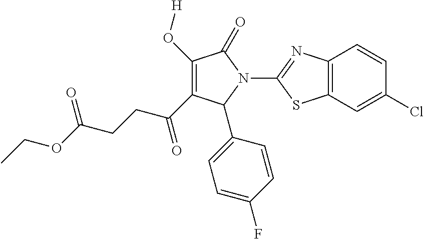 | * | * | |
| 495 | 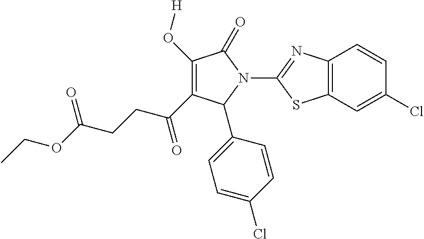 | * | * | |
| 496 | 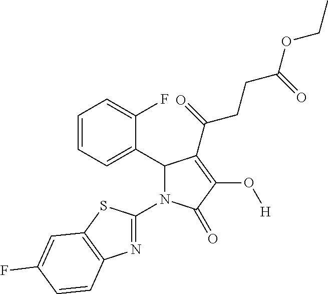 | ** | ** | |
| 497 | 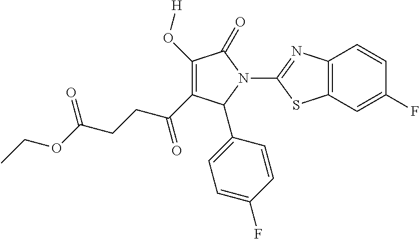 | * | * | |
| 498 | 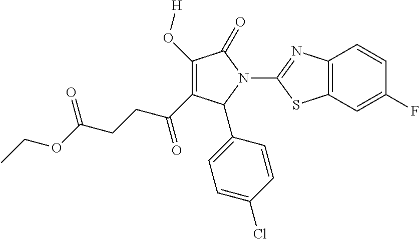 | * | * | |
| 499 | 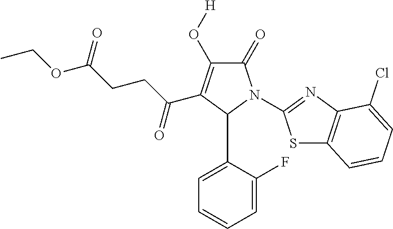 | * | * | |
| 500 | 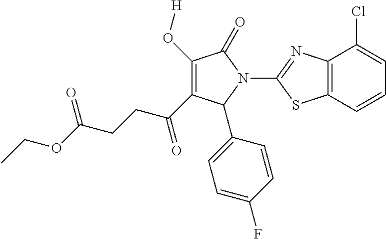 | * | * | |
| 501 | 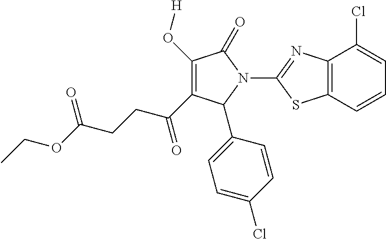 | * | * | |
| 502 | 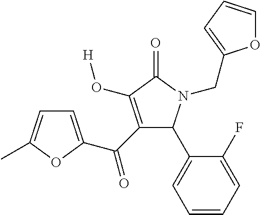 | * | ** | |
| 503 | 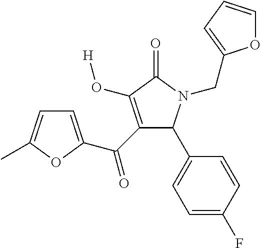 | * | * | |
| 504 | 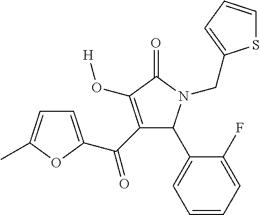 | * | * | |
| 505 | 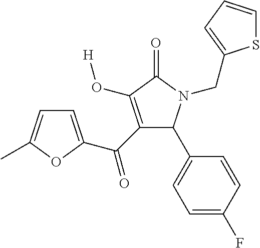 | ** | ** | |
| 506 | 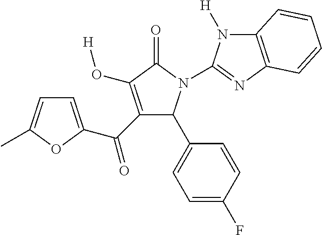 | * | ***** | |
| 507 | 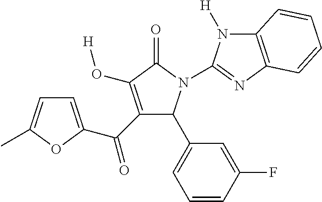 | * | **** | |
| 508 | 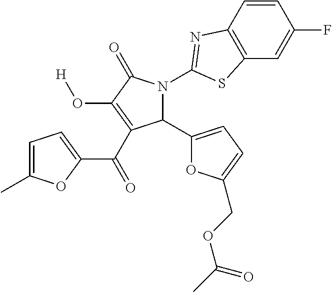 | * | *** | |
| 509 | 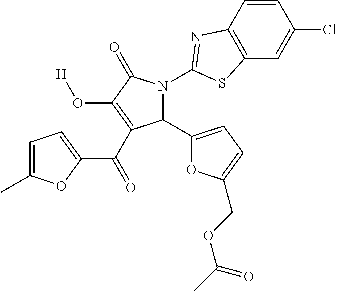 | * | * | |
| 510 | 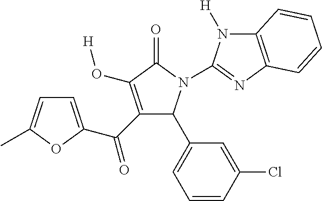 | * | *** | |
| 511 | 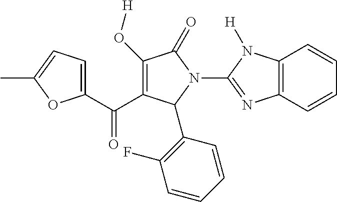 | ** | **** | |
| 512 | 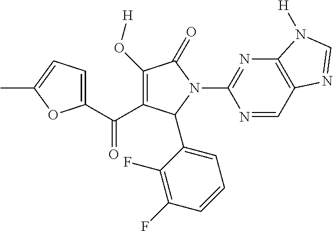 | * | * | |
| 513 | 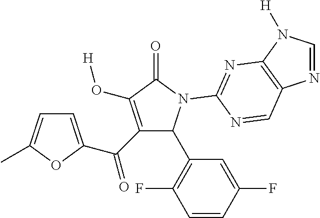 | * | * | |
| 514 | 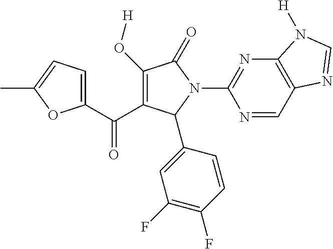 | * | * | |
| 515 | 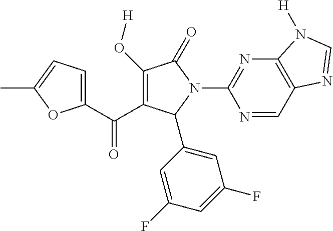 | * | * | |
| 516 | 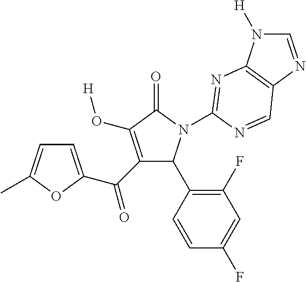 | * | * | |
| 517 | 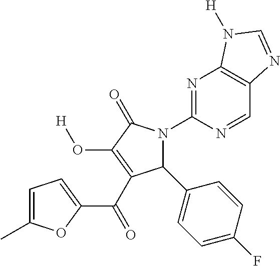 | * | * | |
| 518 | 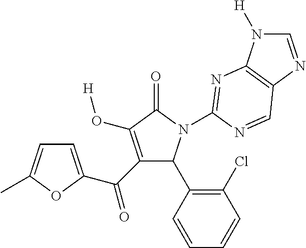 | * | * | |
| 519 | 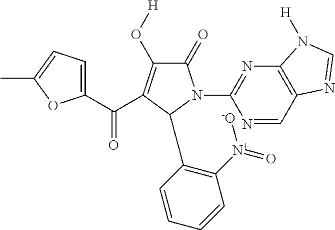 | * | * | |
| 520 | 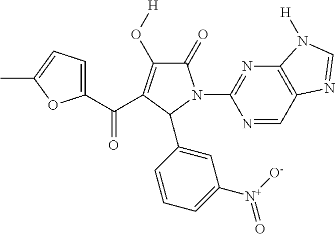 | * | * | |
| 521 | 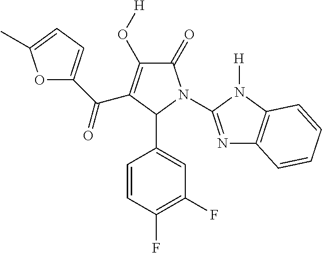 | * | * | |
| 522 | 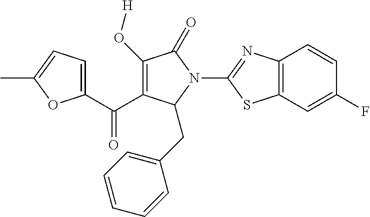 | ** | **** | |
| 523 | 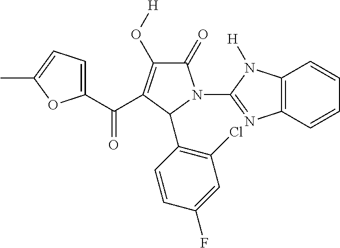 | * | *** | |
| 524 | 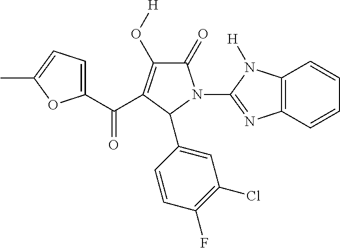 | * | ** | |
| 525 | 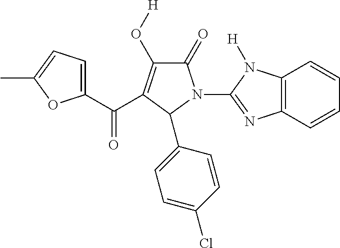 | *** | **** | |
| 526 | 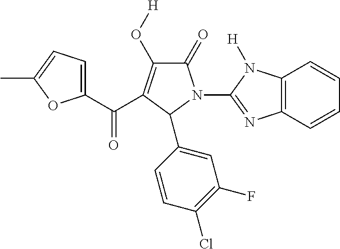 | * | * | |
| 527 | 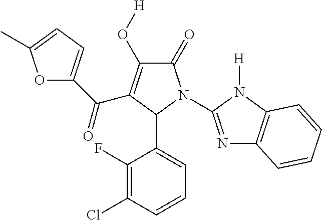 | *** | **** | |
| 528 | 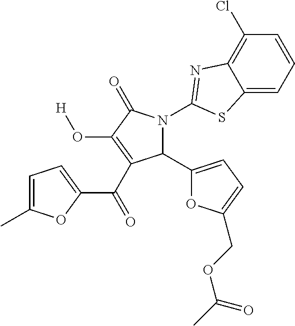 | *** | **** | |
| 529 | 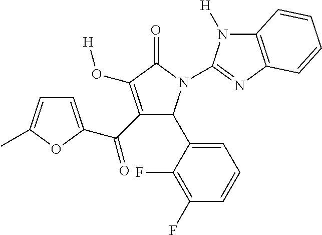 | * | *** | |
| 530 | 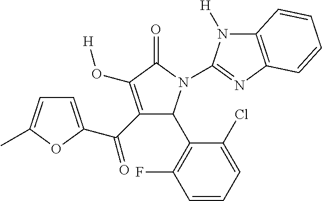 | ** | *** | |
| 531 | 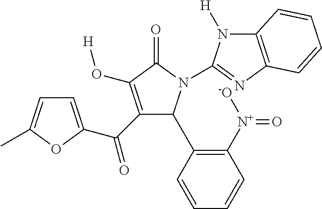 | * | * | |
| 532 | 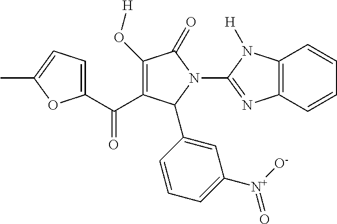 | * | * | |
| 533 | 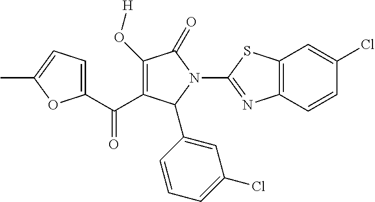 | * | ** | |
| 534 | 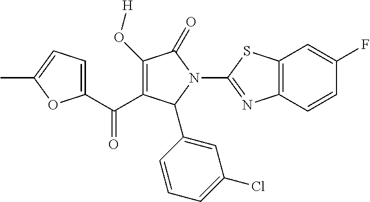 | **** | **** | |
| 535 | 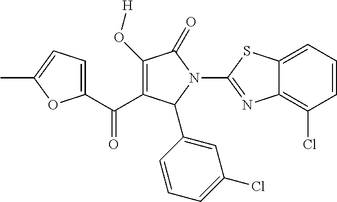 | *** | **** | |
| 536 | 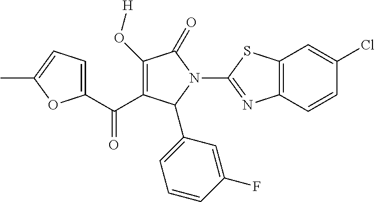 | **** | **** | |
| 537 | 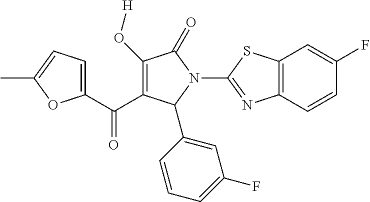 | *** | **** | |
| 538 | 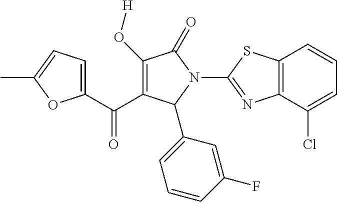 | *** | **** | |
| 539 | 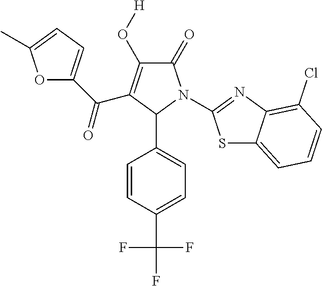 | **** | **** | |
| 540 | 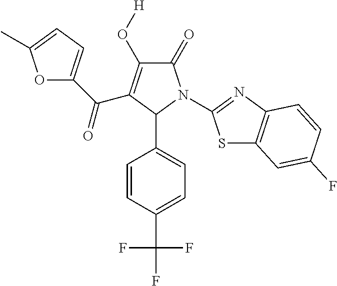 | **** | **** | |
| 541 | 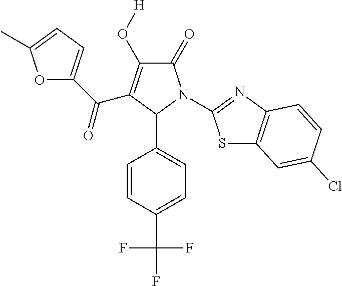 | **** | **** | |
| 542 | 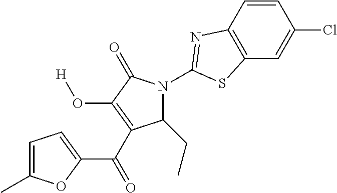 | **** | **** | |
| 543 | 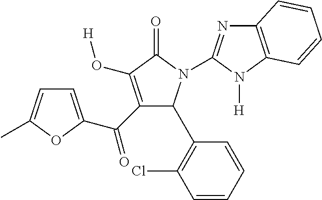 | ** | **** | |
| 544 | 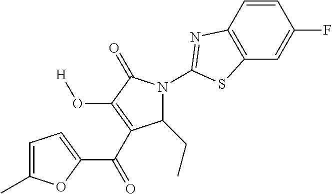 | * | * | |
| 545 | 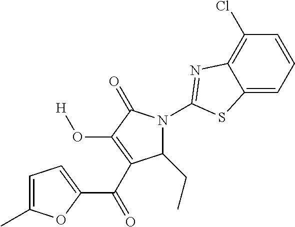 | ** | ** | |
| 546 | 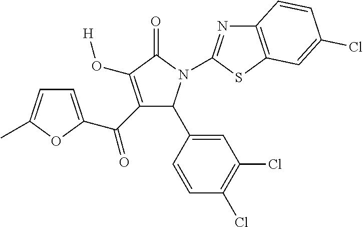 | ** | **** | |
| 547 | 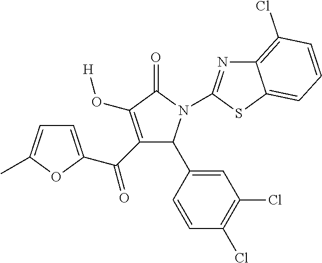 | *** | **** | |
| 548 | 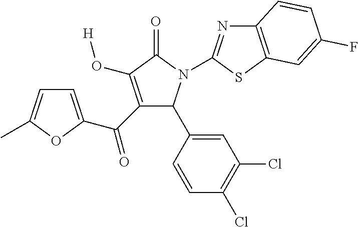 | *** | **** | |
| 549 | 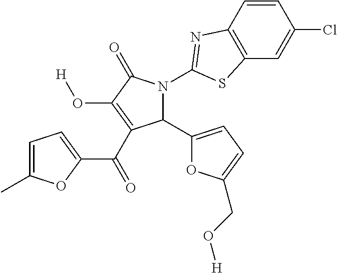 | **** | **** | |
| 550 | 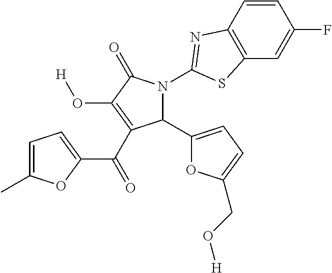 | * | * | |
| 551 | 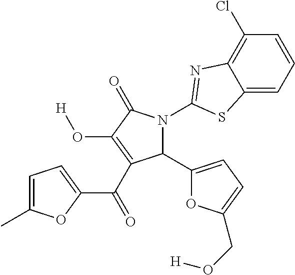 | * | * | |
| 552 | 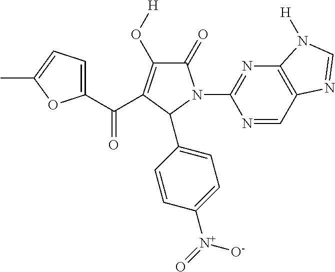 | **** | ***** | |
| 553 | 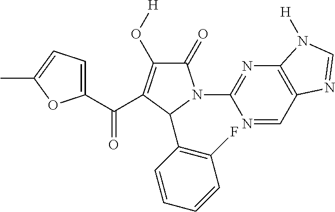 | * | * | |
| 554 | 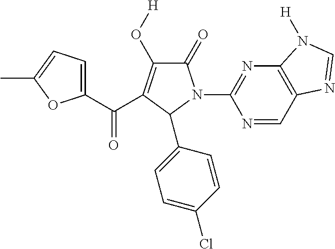 | * | * | |
| 555 | 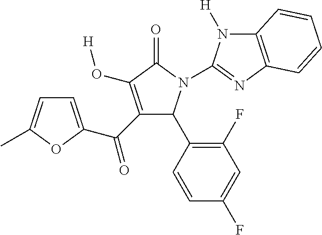 | * | * | |
| 556 | 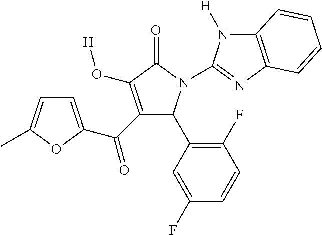 | * | **** | |
| 557 | 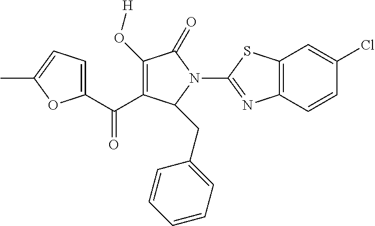 | * | * | |
| 558 | 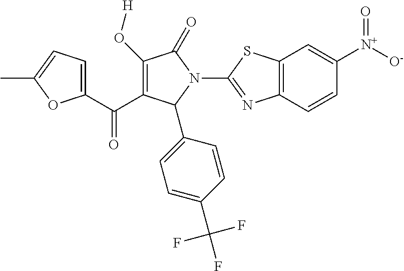 | * | **** | |
| 559 | 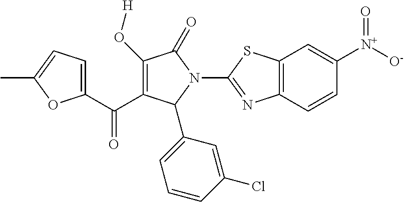 | * | **** | |
| 560 | 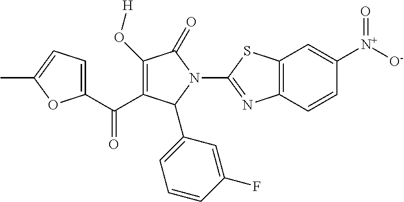 | * | **** | |
| 561 | 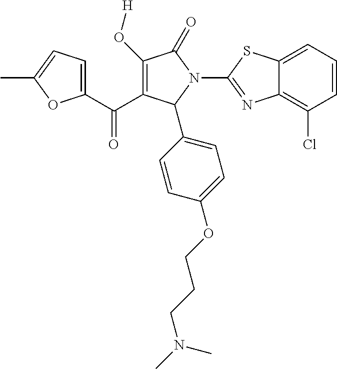 | * | **** | |
| 562 | 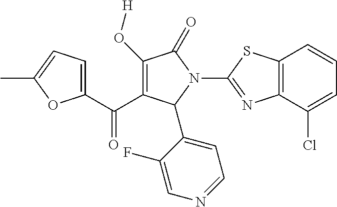 | * | ** | |
| 563 | 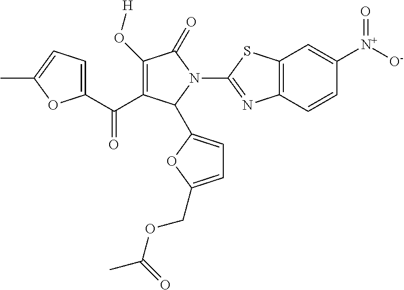 | * | * | |
| 564 | 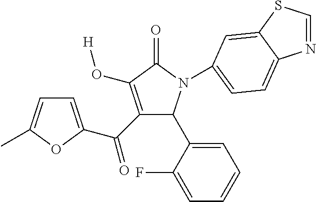 | * | * | |
| 565 | 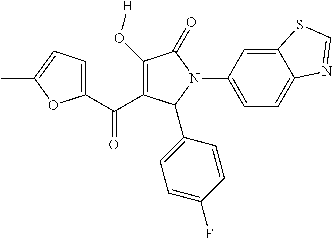 | * | ** | |
| 566 | 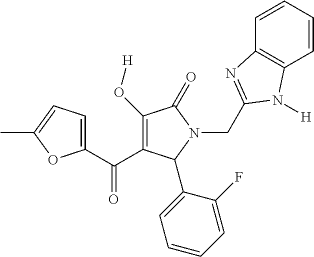 | * | * | |
| 567 | 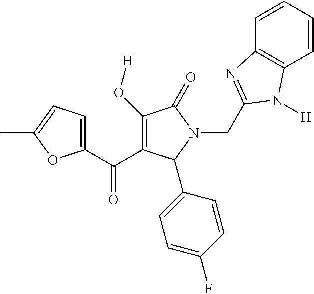 | * | * | |
| 568 | 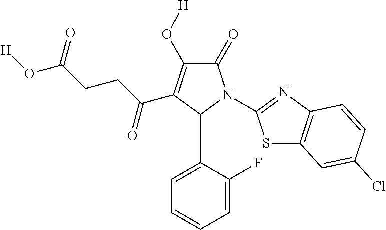 | * | * | |
| 569 | 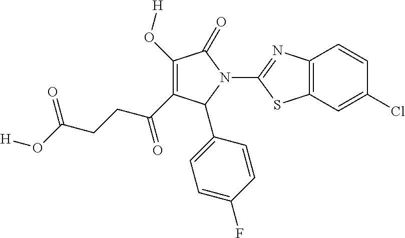 | * | * | |
| 570 | 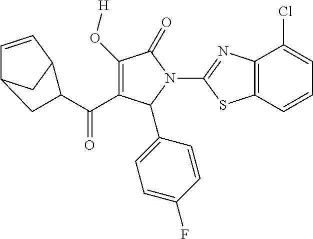 | * | * | |
| 571 | 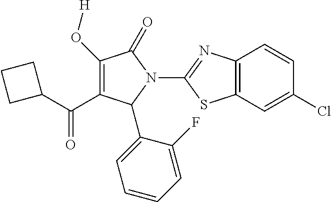 | ** | **** | |
| 572 | 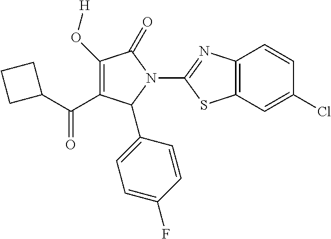 | ** | **** | |
| 573 | 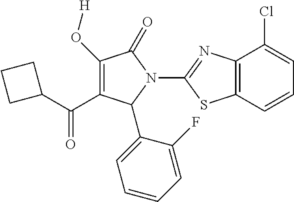 | ** | **** | |
| 574 | 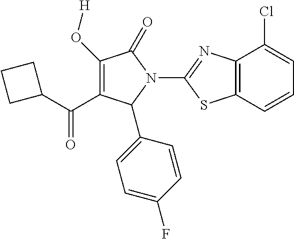 | ** | **** | |
| 575 | 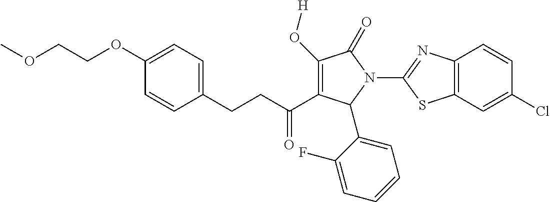 | ** | **** | |
| 576 | 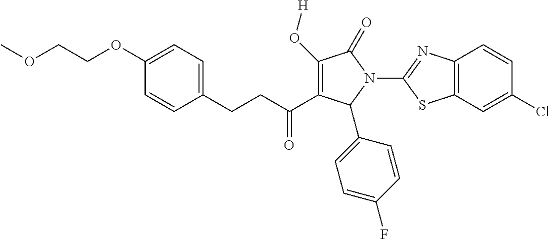 | * | *** | |
| 577 | 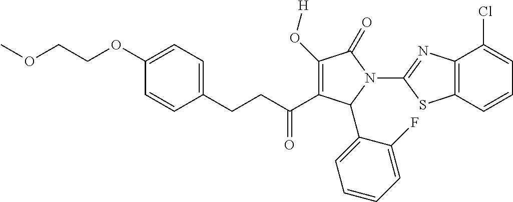 | * | *** | |
| 578 | 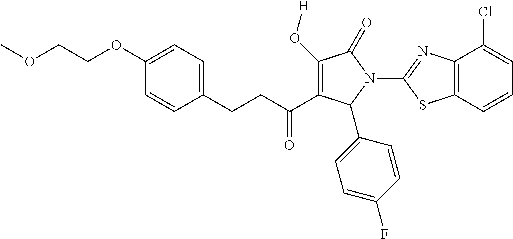 | * | *** | |
| 579 | 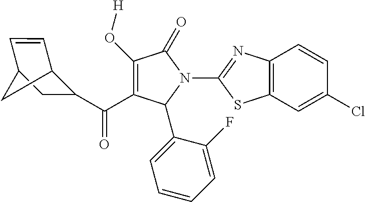 | * | *** | |
| 580 | 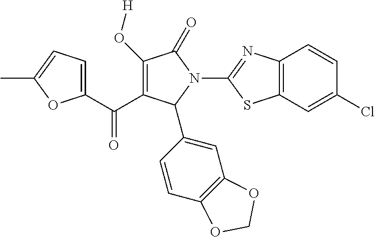 | *** | **** | |
| 581 | 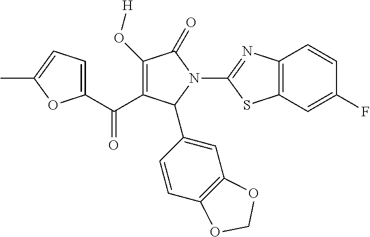 | *** | **** | |
| 582 | 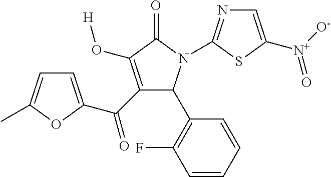 | ** | * | |
| 583 | 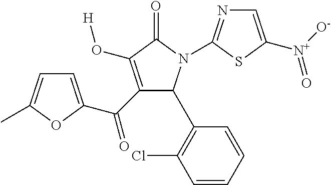 | * | * | |
| 584 | 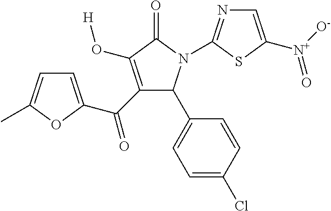 | ** | * | |
| 585 | 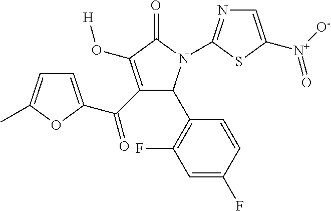 | ** | * | |
| 586 | 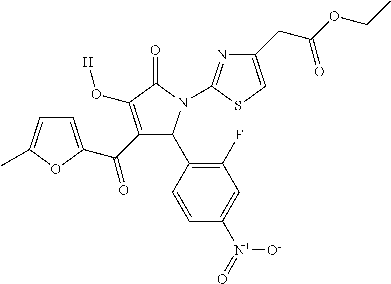 | * | * | |
| 587 | 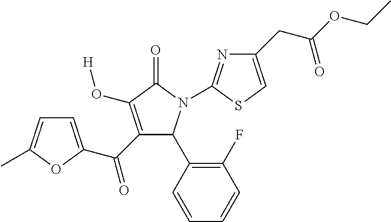 | * | * | |
| 588 | 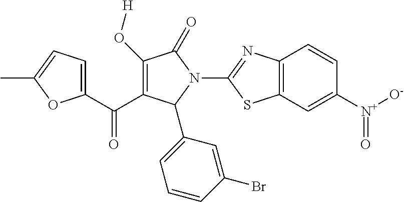 | * | **** | |
| 589 | 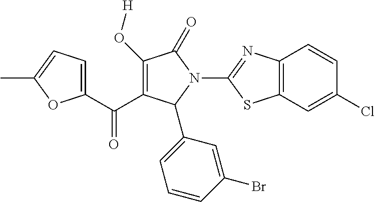 | **** | **** | |
| 590 | 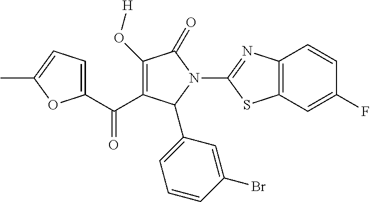 | *** | **** | |
| 591 | 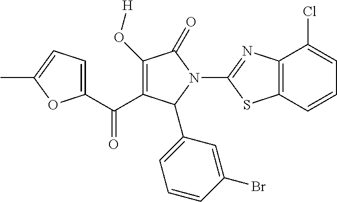 | *** | **** | |
| 592 | 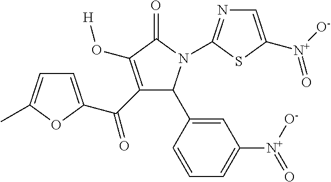 | * | * | |
| 593 | 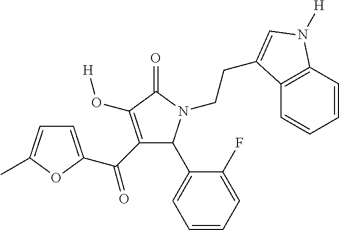 | * | **** | |
| 594 | 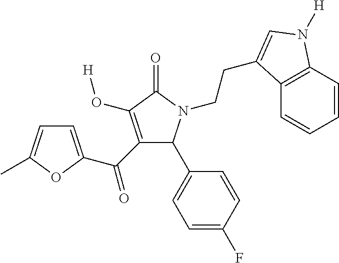 | *** | **** | |
| 595 | 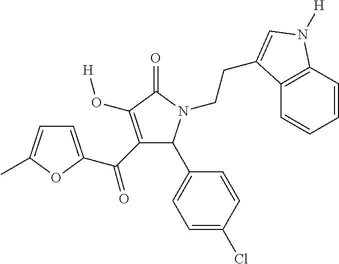 | *** | **** | |
| 596 | 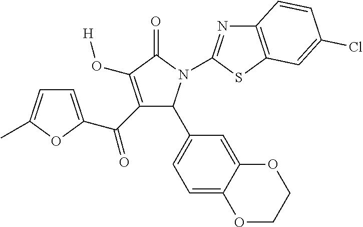 | **** | **** | |
| 597 | 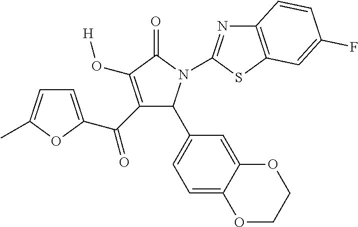 | *** | **** | |
| 598 | 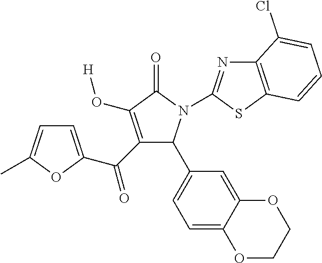 | *** | **** | |
| 599 | 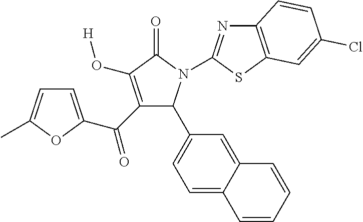 | *** | **** | |
| 600 | 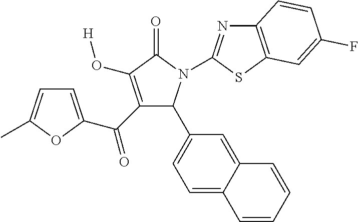 | **** | ***** | |
| 601 | 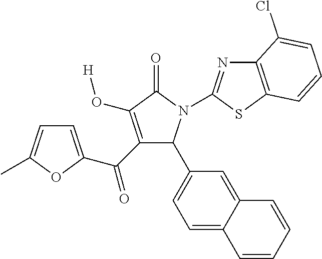 | *** | **** | |
| 602 | 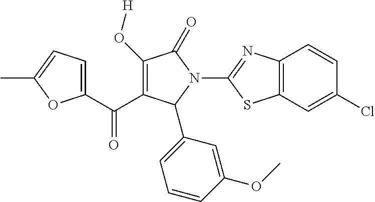 | *** | **** | |
| 603 | 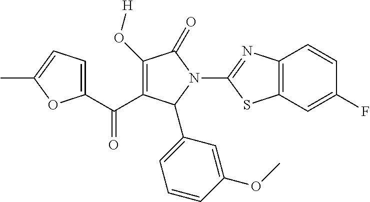 | * | **** | |
| 604 | 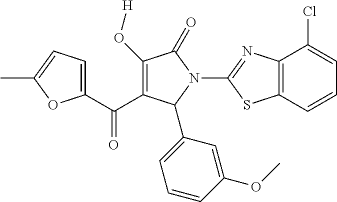 | * | **** | |
| 605 | 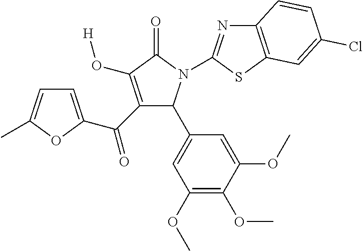 | * | **** | |
| 606 | 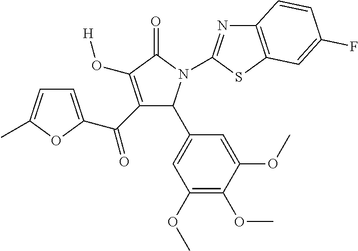 | * | **** | |
| 607 | 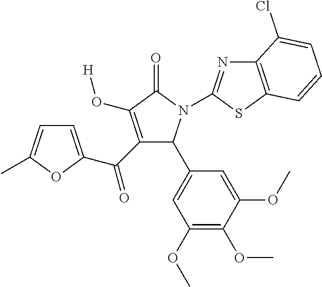 | * | **** | |
| 608 | 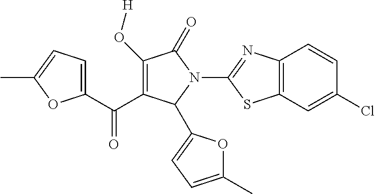 | * | **** | |
| 609 | 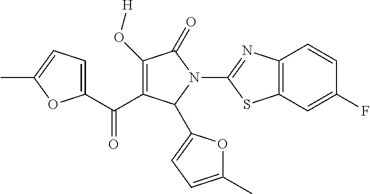 | * | **** | |
| 610 | 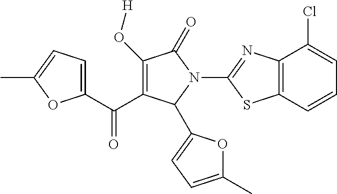 | ** | **** | |
| 611 | 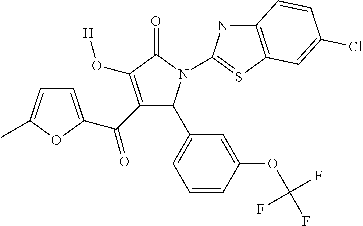 | * | ** | |
| 612 | 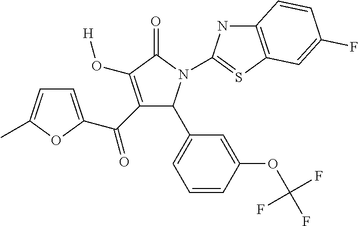 | ** | **** | |
| 613 | 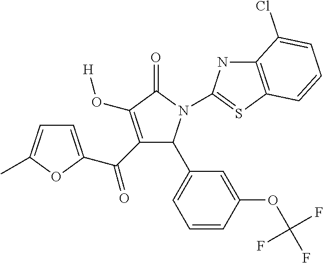 | *** | **** | |
| 614 | 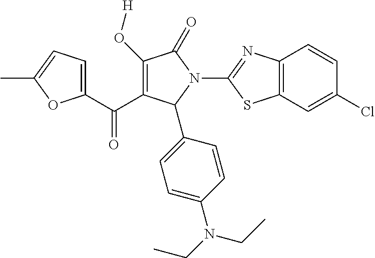 | *** | **** | |
| 615 | 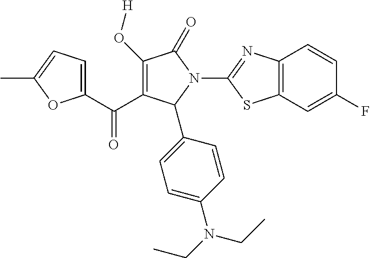 | **** | **** | |
| 616 | 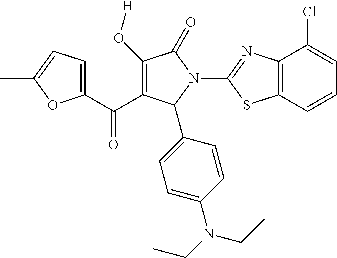 | **** | **** | |
| 617 | 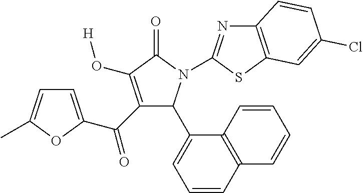 | *** | **** | |
| 618 | 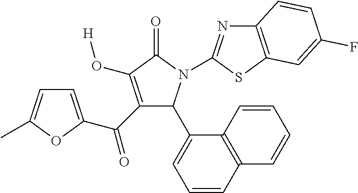 | *** | **** | |
| 619 | 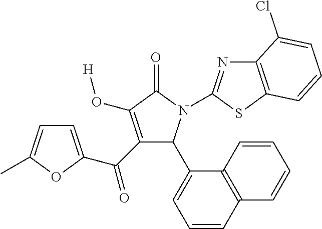 | * | ***** | |
| 620 | 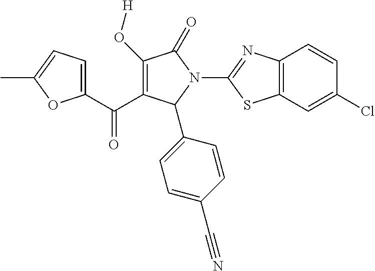 | ** | **** | |
| 621 | 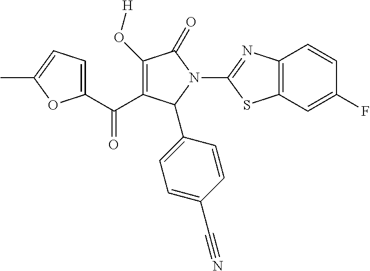 | ** | *** | |
| 622 | 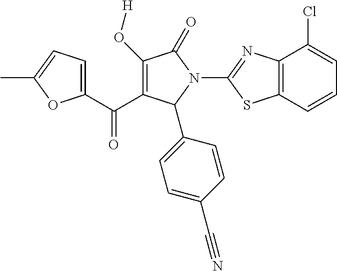 | *** | **** | |
| 623 | 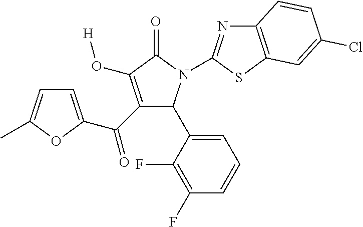 | **** | ***** | |
| 624 | 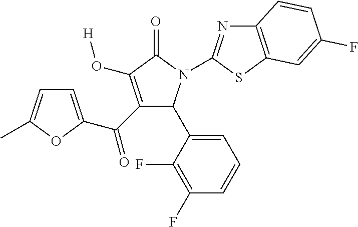 | **** | **** | |
| 625 | 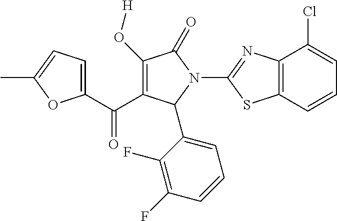 | **** | **** | |
| 626 | 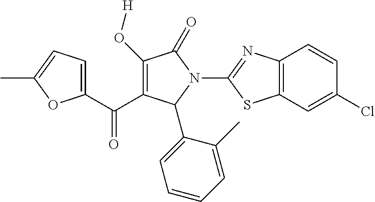 | ** | **** | |
| 627 | 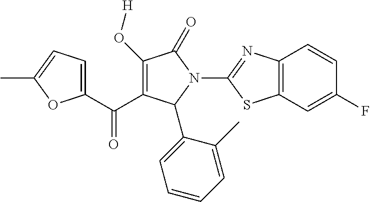 | ** | **** | |
| 628 | 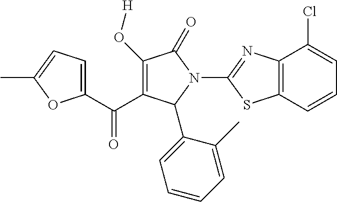 | *** | **** | |
| 629 | 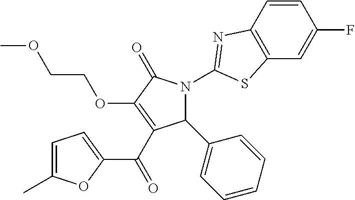 | * | * | |
| 630 | 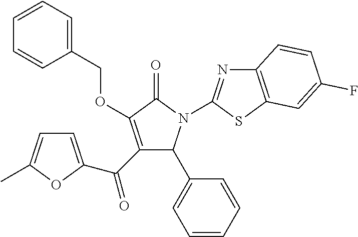 | * | * | |
| 631 | 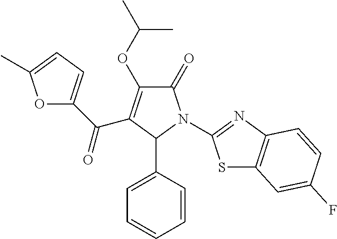 | * | * | |
| 632 | 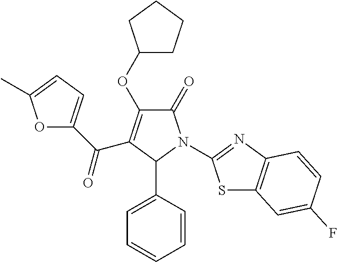 | * | * | |
| 633 | 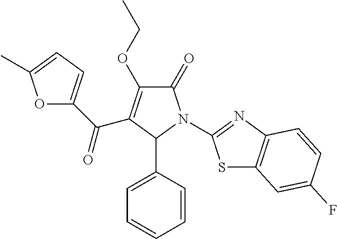 | * | * | |
| 634 | 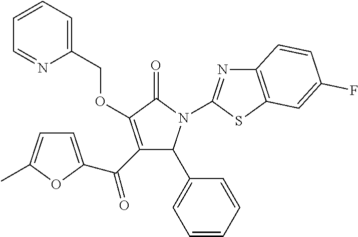 | * | * | |
| 635 | 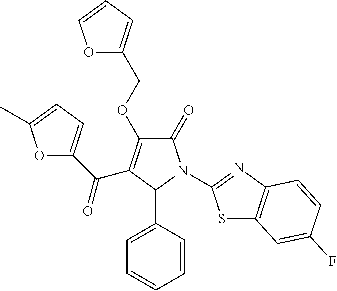 | * | * | |
| 636 | 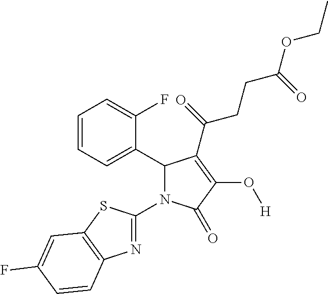 | ** | ** | |
| 637 | 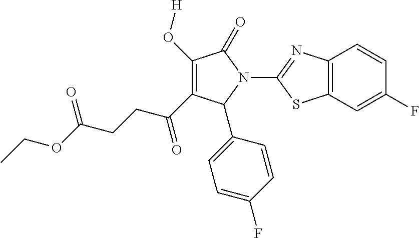 | * | * | |
| 638 | 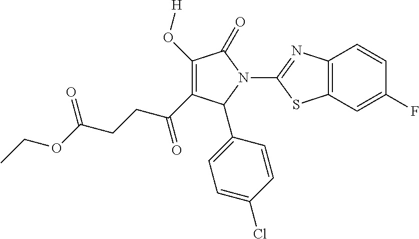 | * | * | |
| 639 | 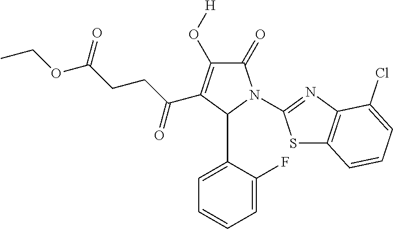 | * | * | |
| 640 | 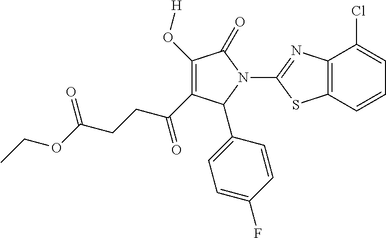 | * | * | |
| 641 | 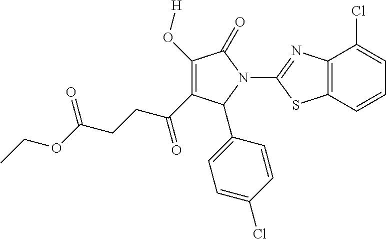 | * | * | |
| 642 | 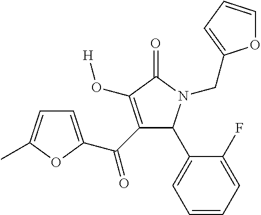 | * | ** | |
| 643 | 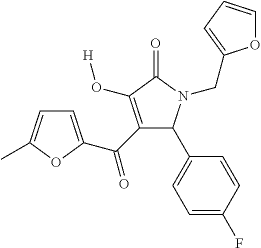 | * | * | |
| 644 | 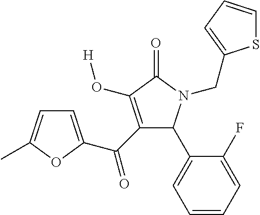 | * | * | |
| 645 | 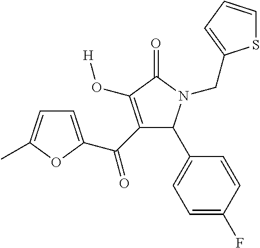 | ** | ** | |
| 646 | 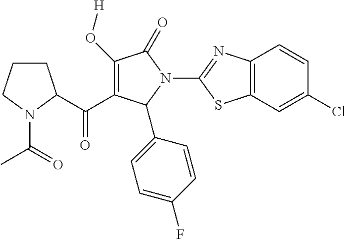 | * | * | |
| 647 | 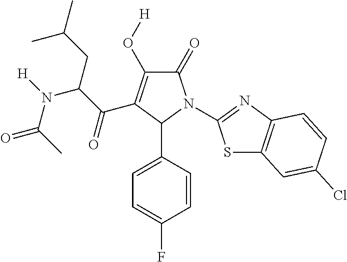 | * | * | |
| 648 | 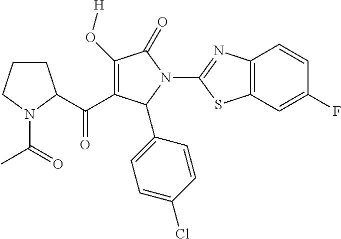 | * | * | |
| 649 | 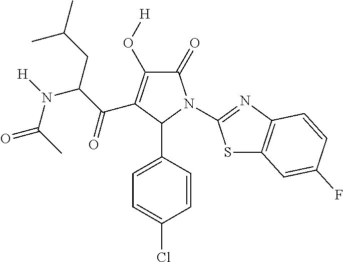 | * | * | |

0.1-1 μg/ml = 5 stars
1.1-10 μg/ml = 4 stars
10.1-20 μg/ml = 3 stars
20.1-50 μg/ml = 2 stars
>50 μg/ml = 1 star

Any of the compounds disclosed herein can be used in the uses described in section 5.5.

In one embodiment, provided herein are the disclosed compounds being isotopically-labelled (i.e., having one or more atoms replaced by an atom having a different atomic mass or mass number). Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

### 5.3 Methods for Making Compounds

Compounds disclosed herein can be made by one skilled in the art using conventional organic syntheses and commercially available materials. By way of example and not limitation, compounds disclosed herein can be prepared as outlined in Schemes 1-8 shown below, as well as in the accompanying description. It should be noted that one skilled in the art can modify the procedures set forth in the illustrative schemes and examples to obtain the desired product.

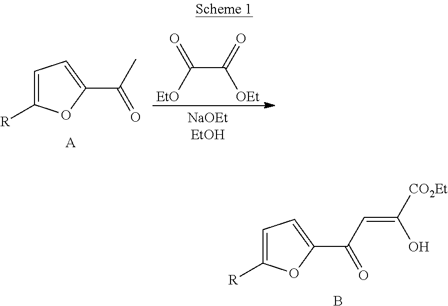

Sodium metal (1.1 eq. based on ketone) is washed free of oil and added portionwise to ethanol (2 mL/mmol substrate). After the evolution of H₂ gas is complete, a solution of the furyl ketone (1.0 eq.) and diethyl oxalate (1.05 eq.) in THF is added dropwise. After the addition is complete, the mixture is heated to reflux overnight, then cooled and partially evaporated. The solution is poured into 4 volumes 0.5 N aq. HCl, and this is extracted twice with ethyl acetate. The extracts are washed with satd. aq. brine, combined, dried over anhydrous magnesium sulfate, filtered and evaporated. The product may be a solid, and may be further purified by recrystallization.

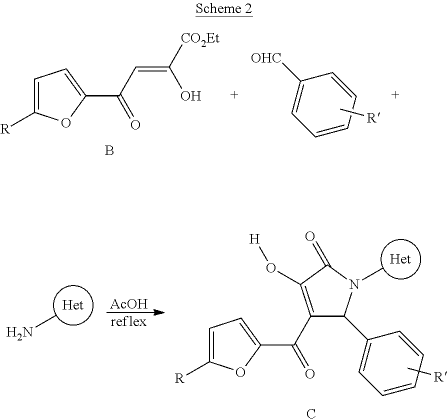

A mixture of diketoester B, substituted benzaldehyde and amine (all 1.0 eq.) in acetic acid (1 M) is heated to reflux overnight. The mixture is cooled, and the resulting precipitate is collected by filtration, washed with ethyl acetate, washed with ether and dried under vacuum to afford racemic product C. Further purification if necessary may be attempted through recrystallization or HPLC. Separation of the enantiomers may be accomplished by chiral HPLC.

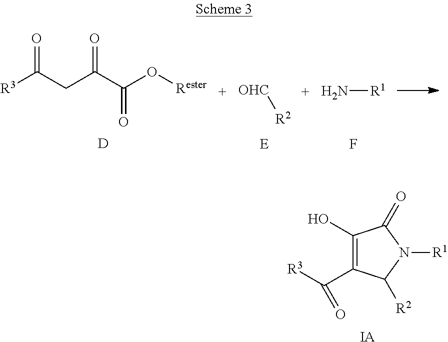

Scheme 3 shows a three-component condensation reaction which may be used in the preparation of compounds of formula IA. A ketoester compound D is mixed with an aldehyde E and an amine F in a suitable solvent. The group R^{ester} may be a small alkyl group (methyl, ethyl, and the like), or may be a group designed to function as an efficient leaving group (along with the ester oxygen atom), such as 4-nitrophenyl, 2,4,6-trichlorophenyl, 1-imidazolyl, and the like. Compounds E and F may be commercial reagents, or may be more complicated or rare compounds made in procedures familiar to those skilled in the art of organic and heterocyclic synthesis. The condensation reaction may be performed in a variety of solvents including but not limited to, methanol, ethanol, isopropanol, 1-methoxy-2-propanol, tetrahydrofuran, acetic acid, toluene, dimethylformamide or dimethylsulfoxide. The presence of an acid catalyst may prove beneficial to the rate of reaction. Appropriate catalysts include acetic acid, trifluoroacetic acid, hydrochloric acid, boron trifluoride etherate, and the like, as well as certain surface-acting catalysts such as silica gel or montmorillonite K-10 clay. The reaction may be performed at ambient temperature, or heated to the reflux temperature of the solvent system being used, or heated to some intermediate temperature. The reaction may also be performed in the presence of microwave radiation.

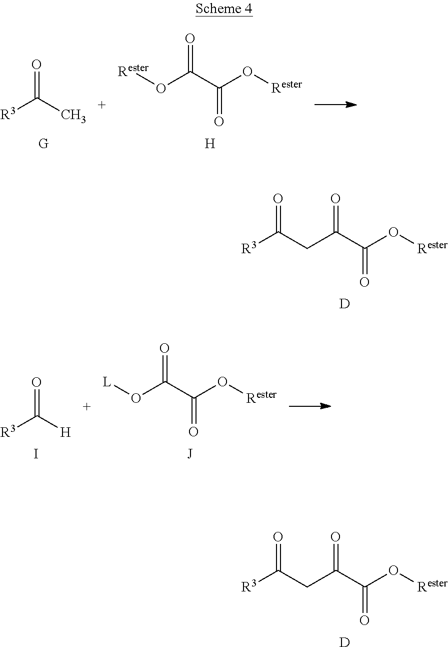

The synthesis of compound D may be accomplished by the reactions shown in Scheme 4. A reagent containing the C(═O) group, which acidifies an attached methyl group, is condensed with an oxalate ester compound H to afford compound D. This reaction can performed in the presence or with the pretreatment with a base reagent. In one embodiment, a modestly basic reagent may be used, such as sodium or potassium ethoxide, potassium tert-butoxide, and the like. Solvents useful for this type of reaction include anhydrous methanol, ethanol, tert-butanol, dioxane, and the like. The reagent of formula H may be a commercially-available oxalate reagent, such as diethyl oxalate, or may be a compound employing O—R^{ester} groups chosen for their reactivity (as discussed above). The two ester groups may be the same or may be different, where one R^{ester} group is more reactive than the other, to better ensure that only one G unit is incorporated into the product.

An alternative method is also shown in Scheme 4. A compound of formula I is allowed to react with a pyruvate ester compound J to afford compound D. The group L denotes a leaving group, such as halogen (chloride, bromide, and the like) or a sulfonate (methanesulfonate, trifluoromethanesulfonate, toluenesulfonate, and the like). This reaction may be performed in the presence of a basic reagent whose purpose is to neutralize the molecule of L-H acid that is formed in the course of the reaction. Such heterogeneous base reagents include potassium carbonate, cesium carbonate, “proton sponge”, etc. Alternatively, the hydrogen atom in compound I may first be removed by the treatment of a stronger base, such as sodium methoxide, sodium hydride or n-butyllithium, and the in situ-prepared salt form of compound I is then allowed to react with pyruvate compound J to afford the desired product.

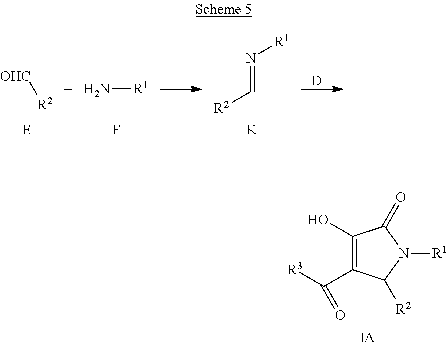

An alternative method for the preparation of compounds of formula IA is shown in Scheme 5. The reagents E and F are reacted together to give a Schiff base compound of formula K. This reaction involves the removal of water and thus may be performed in an apparatus designed for distillative water removal, such as a Dean-Stark still head. Chemical removal of water may be accomplished by adding a dehydrating reagent such as an orthoester, and the like, or the water may be removed by physical adsorption by such additives as molecular sieves or magnesium sulfate. The presence of an acid catalyst may also prove advantageous; such catalysts as toluenesulfonic acid or camphorsulfonic acid may be employed in small amounts to aid in the formation of the Schiff base. This compound is then allowed to react with the previously described ketoester reagent D to give the desired compound. This reaction may be performed in a manner analogous to that of the three-component condensation reaction described previously. Alternatively, the use of base reagents may be employed to effect the condensation. Moderate to strong bases may be used to deprotonate the methylene group between the ketones, and then the imine compound K is added. A separate acid- or base-catalyzed step may be required for ring closure and elimination of the O—R^{ester} group.

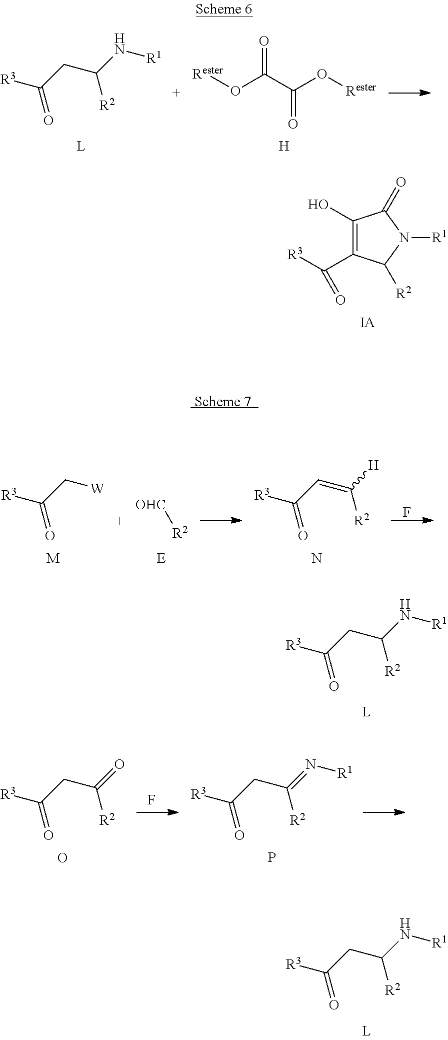

A third general route to the compounds of formula IIA is depicted in Scheme 6. An amine compound of formula L is allowed to react with the oxalate reagent H previously discussed. This reaction is typically performed under basic conditions. Compound L is prepared as shown in Scheme 7. Compound M is allowed to condense with the aldehyde reagent E to afford the olefin compound N. The group W is chosen to activate the coupling reaction with the elimination of W—OH as a byproduct. Thus, W groups that are useful here include triaryl- or trialkylphosphonium (Wittig-type), phosphonate (Horner-Emmons, and the like), or silicon. Hydrogen as the W group may also be used. The resulting olefin compound N is then condensed with the amine compound F to afford the final product. This reaction can be performed in the presence of an acid catalyst or can be done with microwave radiation. Alternatively, a ketone compound of formula O may be condensed with the amine F in a manner similar to the preparation of compound K to afford the Schiff base compound P. The imine group may be reduced with typical reducing agents, including catalytic hydrogenation, sodium cyanoborohydride, sodium triacetoxyborohydride, sodium borohydride, lithium aluminum hydride, borane, etc. The choice of reducing agent is made based on the presence of other reactive functionality in the molecule; reagents selective for the imine bond will give the amine product in cleaner fashion.

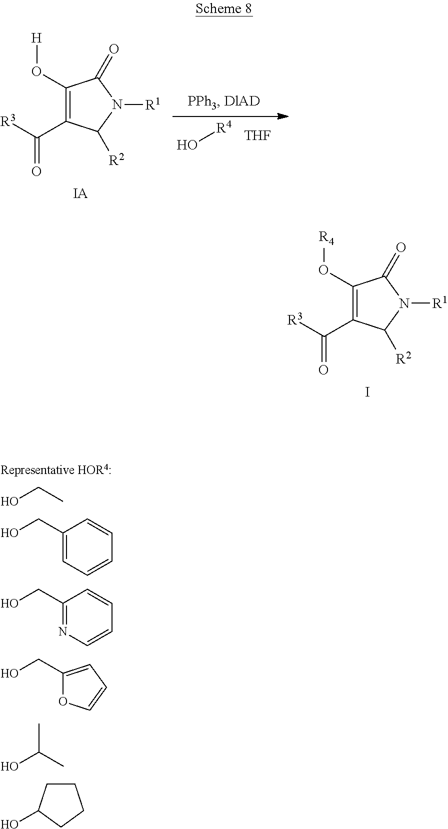

Compounds of formula I can be prepared from compounds of formula IA as set forth above in Scheme 8.

Pharmaceutically acceptable salts of the compounds provided herein can be formed by conventional and known techniques, such as by reacting a compound provided herein with a suitable acid or base.

### 5.4 Compositions

Any of the compounds provided herein, including the compounds described in Section 5.2 and Table 1 and compounds identified by the methods provided herein, can optionally be in the form of a composition comprising the compound or its pharmaceutically acceptable salt, solvate, hydrate, prodrug or stereoisomer thereof.

In some embodiments, provided herein are compositions (including pharmaceutical compositions) comprising a compound and a pharmaceutically acceptable carrier, excipient, or diluent.

In certain embodiments, provided herein are pharmaceutical compositions comprising an effective amount of a compound and a pharmaceutically acceptable carrier, excipient, or diluent. The pharmaceutical compositions are suitable for veterinary and/or human administration.

The pharmaceutical compositions provided herein can be in any form that allows for the composition to be administered to a subject, said subject being an animal in one embodiment, including, but not limited to a human, or non-human animal.

In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is an exemplary carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical compositions and dosage forms comprise one or more excipients. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

These and other ways in which specific dosage forms will vary from one another will be readily apparent to those skilled in the art. See, e.g., Remington's Pharmaceutical Sciences, 18th ed., Mack Publishing, Easton Pa. (1990).

Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

### 5.5 Uses of Compounds

Compounds provided herein are useful generally as inhibitors of protein synthesis. Specifically, compounds provided herein are useful as inhibitors of a peptidyl tRNA hydrolase, in one embodiment a bacterial peptidyl hydrolase. In certain embodiments, the compounds provided herein exhibit specificity for bacterial peptidyl tRNA hydrolase enzymes compared to eukaryotic peptidyl tRNA hydrolase enzymes and in particular, mammalian peptidyl tRNA hydrolase enzymes. In a specific embodiment, a compound provided herein is an inhibitor of bacterial cell proliferation. In another embodiment, a compound provided herein is cytotoxic to bacterial cells and has comparatively low cytotoxicity in eukaryotic cells, in one embodiment mammalian cells. In alternative embodiment, a compound provided herein is cytostatic to bacterial cells and has comparatively low cytotoxicity in eukaryotic cells, in one embodiment mammalian cells.

As used in this context, the term low toxicity refers to a therapeutic window between effective dose whereby bacterial growth is inhibited, and non-specific cytotoxicity is observed having a detrimental effect on mammalian cell growth. The difference targeted for hit-to-lead molecules are greater than 5 fold between MIC and CC540. Development candidates are greater than 50 fold.

In one embodiment, a compound provided herein reduces or inhibits a bacterial infection. In a specific embodiment, a compound eliminates or reduces the amount of bacteria by 75%, 80%, 85%, 90%, 95%, 98%, 99%, 75-99.5%, 85-99.5%, or 90-99.8% in a subject as determined by an assay described herein or known to one of skill in the art. Accordingly, compounds provided herein are useful in methods of preventing, treating and/or managing bacterial infections. In a particular embodiment, a compound provided herein is useful in preventing, treating and/or managing a bacterial infection caused by a strain of bacteria that exhibits resistance to other antibacterial agents.

In certain embodiments, a compound provided herein inhibits or reduces bacterial protein synthesis by at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% as measured by a standard assay (e.g., an in vitro protein translation assay, or other inhibition assay) known to one of skill in the art, or an assay described herein.

In some embodiments, a compound provided herein inhibits or reduces bacterial proliferation by at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% as measured by an assay to determine the minimal inhibitory concentration (e.g., by microbroth dilution or agar diffusion) known to one of skill in the art, or an assay described herein.

In some embodiments, a compound provided herein eliminates or reduces the amount of bacteria by at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% as measured by bacterial assays known to one of skill in the art, or an assay described herein.

Bacterial infections reduced or inhibited in accordance with the methods provided herein include infections caused by gram negative bacteria and gram positive bacteria. In a specific embodiment, the bacterial infection reduced or inhibited is caused by an intracellular bacteria. In another embodiments, the bacterial infections reduced or inhibited are resistant to one or more currently available antibiotics. Nonlimiting examples of bacterial infections that can be reduced or inhibited in accordance with the methods provided herein include, but are not limited to infections caused by bacteria such as, Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., and Helicobacter pylori.

In certain embodiments, a compound provided herein reduces or inhibits a bacterial infection caused by one or more bacteria selected from the group consisting of Brucella, Bacillus, Yersinia, Coxiella, Francisella, Mycobacterium, Shigella, Salmonella, Vibrio, and Campylobacter.

In certain embodiments, a compound provided herein reduces or inhibits a bacterial infection caused by one or more bacteria selected from the group consisting of Haemophilus influenzae, Neisseria meningitidis, and Streptococcus pneumoniae.

In some embodiments, a compound provided herein reduces or inhibits a bacterial infection caused by one or more bacteria selected from the group consisting of Staphylococcus aureus, Staphylococcus epidermidis, Enterococcus faecium, Enterococcus faecalis, and Pseudomonas aeruginosa.

5.5.1 Prophylactic and Therapeutic Methods

Provided herein are methods of preventing, treating and/or managing a bacterial infection, said methods comprising administering to a subject in need thereof one or more compounds provided herein, such as a compound identified in accordance with the methods provided herein. In one embodiment, provided herein are methods of preventing, treating/and or managing a bacterial infection, said methods comprising administering to a subject having a bacterial infection a dose of a prophylactically or therapeutically effective amount of one or more compounds provided herein.

Further provided herein are methods of preventing, treating and/or managing a bacterial infection, said methods comprising administering to a subject in need thereof one or more compounds provided herein, and one or more other therapies (e.g., prophylactic or therapeutic agents). In a specific embodiment, the other therapies are currently being used, have been used or are known to be useful in the prevention, treatment and/or management of a bacterial infection. Non-limiting examples of such prophylactic or therapeutics are provided in §5.6, infra.

The combination therapies provided herein can be administered sequentially or concurrently. In one embodiment, the combination therapies provided herein comprise a compound provided herein and at least one other therapy which has the same mechanism of action. In another embodiment, the combination therapies provided herein comprise a compound provided herein and at least one other therapy which has a different mechanism of action than the compound.

In a specific embodiment, the combination therapies provided herein improve the prophylactic and/or therapeutic effect of a compound provided herein by functioning together with the compound to have an additive or synergistic effect. In another embodiment, the combination therapies provided herein reduce the side effects associated with each therapy taken alone.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

In certain embodiments, provided herein are methods for treating and/or managing a bacterial infection, in a subject refractory to conventional therapies for such an infection, the methods comprising administering to said subject a dose of a prophylactically or therapeutically effective amount of a compound provided herein. An infection may be determined to be refractory to a therapy means when at least some significant portion of the bacterial cells are not killed or their cell division arrested in response to the therapy. Such a determination can be made either in vivo or in vitro by any method known in the art for assaying the effectiveness of treatment on bacterial cells, using the art-accepted meanings of “refractory” in such a context.

In specific embodiments, the bacterial infection prevented, treated, and/or managed is caused by gram negative bacteria, gram positive bacteria, or intracellular bacteria, examples of which are described herein.

5.5.2 Use as Disinfectant

Further provided herein are methods for the use of the compounds provided herein as active ingredients in products having antibacterial properties or in products in which it is desirable to have antibacterial activity. In one embodiment, one or more of the compounds provided herein is used as an additive in a cosmetic product, a personal hygiene product, or a household or industrial cleaning product. In another embodiment, one or more of the compounds provided herein is used as an additive in an antibacterial ointment or cream. In another embodiment one or more compounds provided herein is used as an additive to soap.

### 5.6 Agents Useful in Combination with Compounds

Therapeutic or prophylactic agents that can be used in combination with the compounds provided herein for the prevention, treatment and/or management of a bacterial infection include, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules. Specific examples of such agents include, but are not limited to, immunomodulatory agents (e.g., interferon), anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, and non-steriodal anti-inflammatory drugs (e.g., aspirin, ibuprofen, diclofenac, and COX-2 inhibitors), pain relievers, leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents (e.g., nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, and AZT) and antibiotics (e.g., dactinomycin (formerly actinomycin), bleomycin, erythomycin, penicillin, mithramycin, and anthramycin (AMC)).

Any therapy which is known to be useful, or which has been used or is currently being used for the prevention, management, and/or treatment of a bacterial infection or can be used in combination with the compounds provided herein in accordance with the invention described herein. See, e.g., Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W.B. Saunders, Philadelphia, 1996 for information regarding therapies (e.g., prophylactic or therapeutic agents) which have been or are currently being used for preventing, treating and/or managing bacterial infections.

5.6.1 Antibacterial Agents

Antibacterial agents, including antibiotics, that can be used in combination with the compounds provided herein include, but are not limited to, aminoglycoside antibiotics, glycopeptides, amphenicol antibiotics, ansamycin antibiotics, cephalosporins, cephamycins oxazolidinones, penicillins, quinolones, streptogamins, tetracyclins, and analogs thereof.

In a specific embodiment, the compounds provided herein are used in combination with other protein synthesis inhibitors, including but not limited to, streptomycin, neomycin, erythromycin, carbomycin, and spiramycin.

In one embodiment, the antibacterial agent is selected from the group consisting of ampicillin, amoxicillin, ciprofloxacin, gentamycin, kanamycin, neomycin, penicillin G, streptomycin, sulfanilamide, and vancomycin. In another embodiment, the antibacterial agent is selected from the group consisting of azithromycin, cefonicid, cefotetan, cephalothin, cephamycin, chlortetracycline, clarithromycin, clindamycin, cycloserine, dalfopristin, doxycycline, erythromycin, linezolid, mupirocin, oxytetracycline, quinupristin, rifampin, spectinomycin, and trimethoprim

Additional, non-limiting examples of antibacterial agents for use in combination with the compounds provided herein include the following: aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), folic acid analogs (e.g., trimethoprim), glycopeptides (e.g., vancomycin), lincosamides (e.g., clindamycin, and lincomycin), macrolides (e.g., azithromycin, carbomycin, clarithomycin, dirithromycin, erythromycin, and erythromycin acistrate), monobactams (e.g., aztreonam, carumonam, and tigemonam), nitrofurans (e.g., furaltadone, and furazolium chloride), oxacephems (e.g., flomoxef, and moxalactam), oxazolidinones (e.g., linezolid), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o benethamine, penicillin 0, penicillin V, penicillin V benzathine, penicillin V hydrabamine, penimepicycline, and phencihicillin potassium), quinolones and analogs thereof (e.g., cinoxacin, ciprofloxacin, clinafloxacin, flumequine, grepagloxacin, levofloxacin, and moxifloxacin), streptogramins (e.g., quinupristin and dalfopristin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), and tetracyclines (e.g., apicycline, chlortetracycline, clomocycline, and demeclocycline). Additional examples include cycloserine, mupirocin, tuberin amphomycin, bacitracin, capreomycin, colistin, enduracidin, enviomycin, and 2,4 diaminopyrimidines (e.g., brodimoprim).

5.6.2 Antiviral Agents

Antiviral agents that can be used in combination with the compounds provided herein include, but are not limited to, non-nucleoside reverse transcriptase inhibitors, nucleoside reverse transcriptase inhibitors, protease inhibitors, and fusion inhibitors. In one embodiment, the antiviral agent is selected from the group consisting of amantadine, oseltamivir phosphate, rimantadine, and zanamivir. In another embodiment, the antiviral agent is a non-nucleoside reverse transcriptase inhibitor selected from the group consisting of delavirdine, efavirenz, and nevirapine. In another embodiment, the antiviral agent is a nucleoside reverse transcriptase inhibitor selected from the group consisting of abacavir, didanosine, emtricitabine, emtricitabine, lamivudine, stavudine, tenofovir DF, zalcitabine, and zidovudine. In another embodiment, the antiviral agent is a protease inhibitor selected from the group consisting of amprenavir, atazanavir, fosamprenav, indinavir, lopinavir, nelfinavir, ritonavir, and saquinavir. In another embodiment, the antiviral agent is a fusion inhibitor such as enfuvirtide.

Additional, non-limiting examples of antiviral agents for use in combination with the compounds provided herein include the following: rifampicin, nucleoside reverse transcriptase inhibitors (e.g., AZT, ddI, ddC, 3TC, d4T), non-nucleoside reverse transcriptase inhibitors (e.g., delavirdine efavirenz, nevirapine), protease inhibitors (e.g., aprenavir, indinavir, ritonavir, and saquinavir), idoxuridine, cidofovir, acyclovir, ganciclovir, zanamivir, amantadine, and palivizumab. Other examples of anti-viral agents include but are not limited to acemannan; acyclovir; acyclovir sodium; adefovir; alovudine; alvircept sudotox; amantadine hydrochloride (SYMMETREL™); aranotin; arildone; atevirdine mesylate; pyridine; cidofovir; cipamfylline; cytarabine hydrochloride; delavirdine mesylate; desciclovir; didanosine; disoxaril; edoxudine; enviradene; enviroxime; famciclovir; famotine hydrochloride; fiacitabine; fialuridine; fosarilate; foscamet sodium; fosfonet sodium; ganciclovir; ganciclovir sodium; idoxuridine; kethoxal; lamivudine; lobucavir; memotine hydrochloride; methisazone; nevirapine; oseltamivir phosphate (TAMIFLU™); penciclovir; pirodavir; ribavirin; rimantadine hydrochloride (FLUMADINE™); saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zanamivir (RELENZA™); zidovudine; and zinviroxime.

### 5.7 Dosages and Frequency

The amount of a compound provided herein, or the amount of a composition comprising the compound, that will be effective in the prevention, treatment and/or management of a bacterial infection can be determined by standard clinical techniques. In vitro or in vivo assays may optionally be employed to help identify optimal dosage ranges. The precise dose to be employed will also depend, e.g., on the route of administration, the type of infection, and the seriousness of the infection, and should be decided according to the judgment of the practitioner and each patient's circumstances.

Exemplary doses of the compounds or compositions provided herein include milligram or microgram amounts per kilogram of subject or sample weight (e.g., about 1 microgram per kilogram to about 500 milligrams per kilogram, about 5 micrograms per kilogram to about 100 milligrams per kilogram, or about 1 microgram per kilogram to about 50 micrograms per kilogram). In specific embodiments, a daily dose is at least 50 mg, 75 mg, 100 mg, 150 mg, 250 mg, 500 mg, 750 mg, or at least 1 g.

In one embodiment, the dosage is a concentration of 0.01 to 5000 mM, 1 to 300 mM, 10 to 100 mM and 10 mM to 1 M. In another embodiment, the dosage is a concentration of at least 5 μM, at least 10 μM, at least 50 μM, at least 100 μM, at least 500 μM, at least 1 mM, at least 5 mM, at least 10 mM, at least 50 mM, at least 100 mM, or at least 500 mM.

In a specific embodiment, the dosage is 0.25 μg/kg or more, 0.5 μg/kg or more, 1 μg/kg or more, 2 μg/kg or more, 3 μg/kg or more, 4 μg/kg or more, 5 μg/kg or more, 6 μg/kg or more, 7 μg/kg or more, 8 μg/kg or more, 9 μg/kg or more, or 10 μg/kg or more, 25 μg/kg or more, 50 μg/kg or more, 100 μg/kg or more, 250 μg/kg or more, 500 μg/kg or more, 1 mg/kg or more, 5 mg/kg or more, 6 mg/kg or more, 7 mg/kg or more, 8 mg/kg or more, 9 mg/kg or more, or 10 mg/kg or more of a patient's body weight.

The dosages of prophylactic or therapeutic agents other than a compound provided herein or composition provided herein which have been or are currently being used for the prevention, treatment and/or management of a bacterial infection can be determined using references available to a clinician such as, e.g., the Physicians' Desk Reference (55th ed. 2001). In one embodiment, dosages lower than those which have been or are currently being used to prevent, treat and/or manage the infection are utilized in combination with one or more compounds or compositions provided herein.

The above-described administration schedules are provided for illustrative purposes only and should not be considered limiting. A person of ordinary skill in the art will readily understand that all doses are within the scope of the embodiments provided herein.

### 6. EXAMPLES

The methods provided herein provide assays designed to identify novel, broad spectrum antibacterial compounds. In particular, the methods provided herein identify compounds having inhibitory activity against a bacterial peptidyl tRNA hydrolase (“Pth”). Pth inhibitors are further screened in a series of secondary assays designed to select for the ability to specifically inhibit bacterial cell proliferation. The methods provided herein further provide for the synthesis of novel compounds based on the identified Pth inhibitors. The novel compounds are designed using structure activity relationship analyses combined with molecular modeling approaches. The novel compounds represent compounds optimized for their ability to inhibit bacterial cell proliferation while maintaining low toxicity with respect to eukaryotic cells, in one embodiment mammalian cells. The novel compounds are also optimized for their ability to minimize the emergence of bacterial resistance. In a specific embodiment, compounds for use in the prevention, treatment and/or management of bacterial infections include those having a 50% inhibitory concentration of less than 1 micromolar against bacterial Pth, a minimal inhibitory concentration (“MIC”) of less than 1 micromolar, preferably, less than 0.80, 0.75, 0.50, 0.25, or 0.15 micromolar in assays of bacterial cell proliferation, a fifty to one hundred fold therapeutic window between the MIC value and cytoxicity, less than 90% binding to serum proteins, and sustained serum protein levels at least 4-fold above the MIC value.

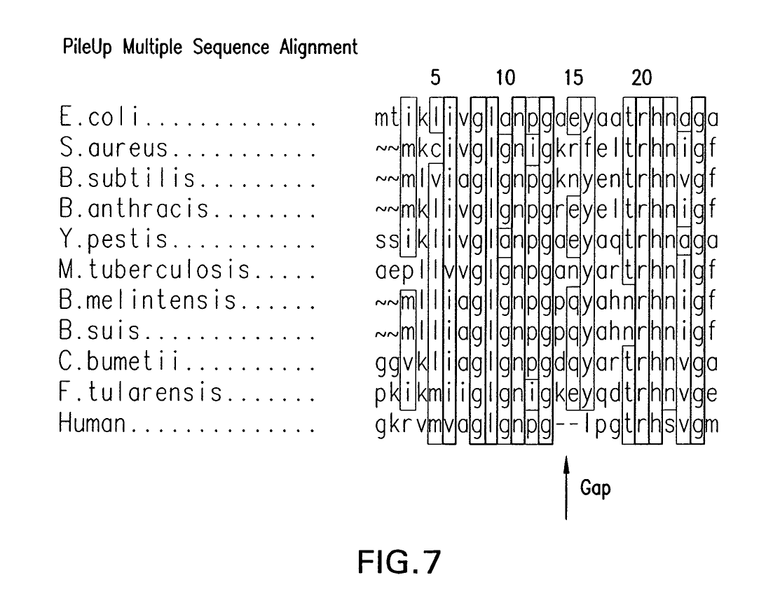

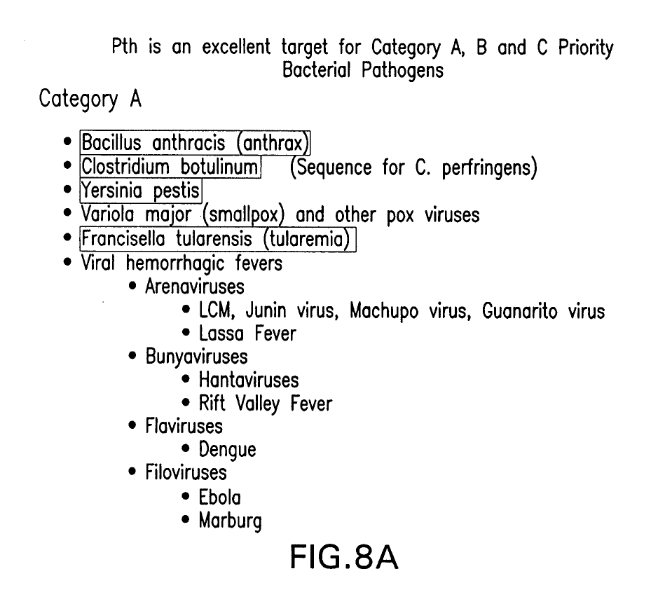

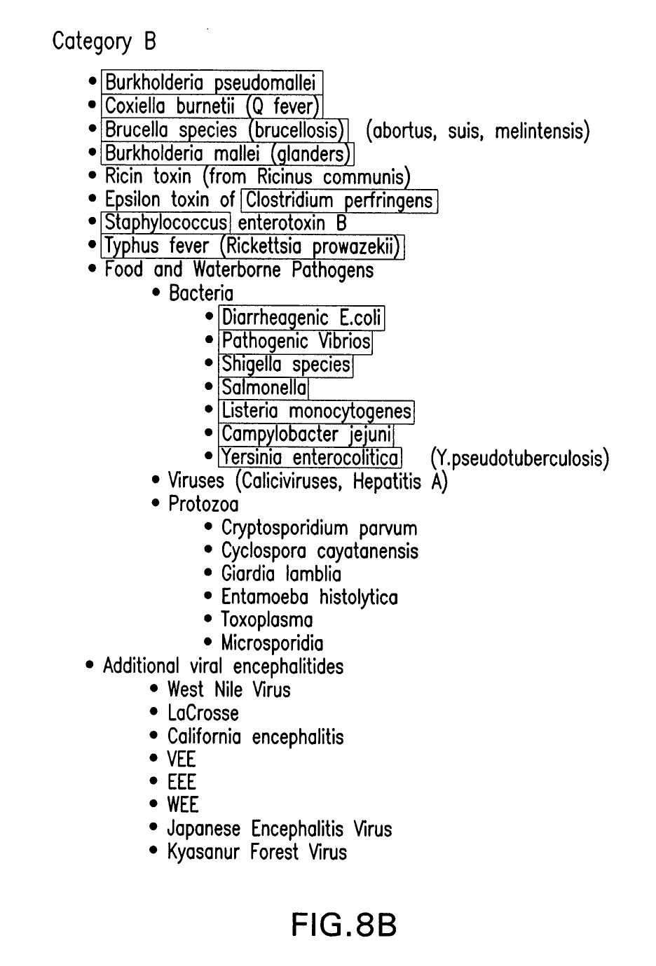

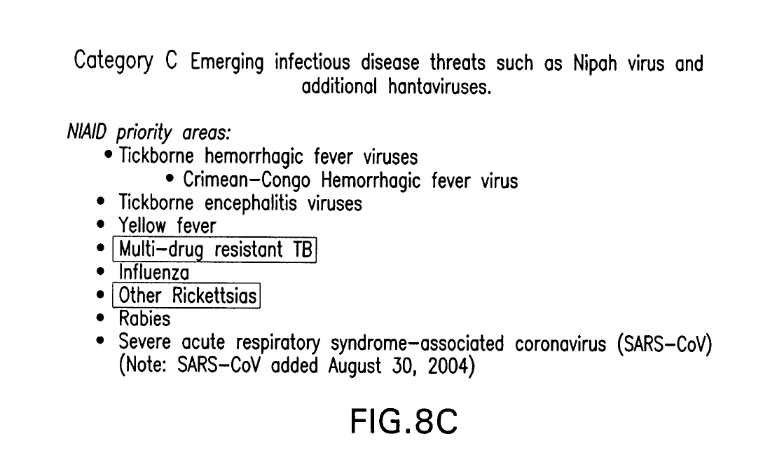

In certain embodiments, a compound provided herein is a highly selective inhibitor which binds preferentially to the loop region of the bacterial peptidyl tRNA hydrolase active site represented by the consensus sequence in FIG. 7. This active site sequence is conserved among various bacterial species. However, sequence alignment of the bacterial sequences with the human homolog introduces a two amino acid gap within this site (FIG. 7). Thus, this gap may introduce differences in the catalytic site between the human and bacterial peptidyl tRNA hydrolases.

In certain embodiments, a compound provided herein is a highly selective inhibitor which binds preferentially to the loop region of the bacterial peptidyl tRNA hydrolase active site represented by the consensus sequence in FIG. 7, and specifically interacts with the aromatic residue (tyrosine or phenylalanine) represented by amino acid number 15 in FIG. 7. While all bacterial species demonstrate variability in residues 13-17 of the loop region as represented by FIG. 7, the aromatic residue at position 15 is one residue that is highly conserved among bacteria. In the putative human enzyme, this residue is a leucine. Thus, this residue may identify a separate, targetable change in tRNA recognition elements.

### 6.1 Initial Screen for Inhibitory Activity Against RNA Hydrolase

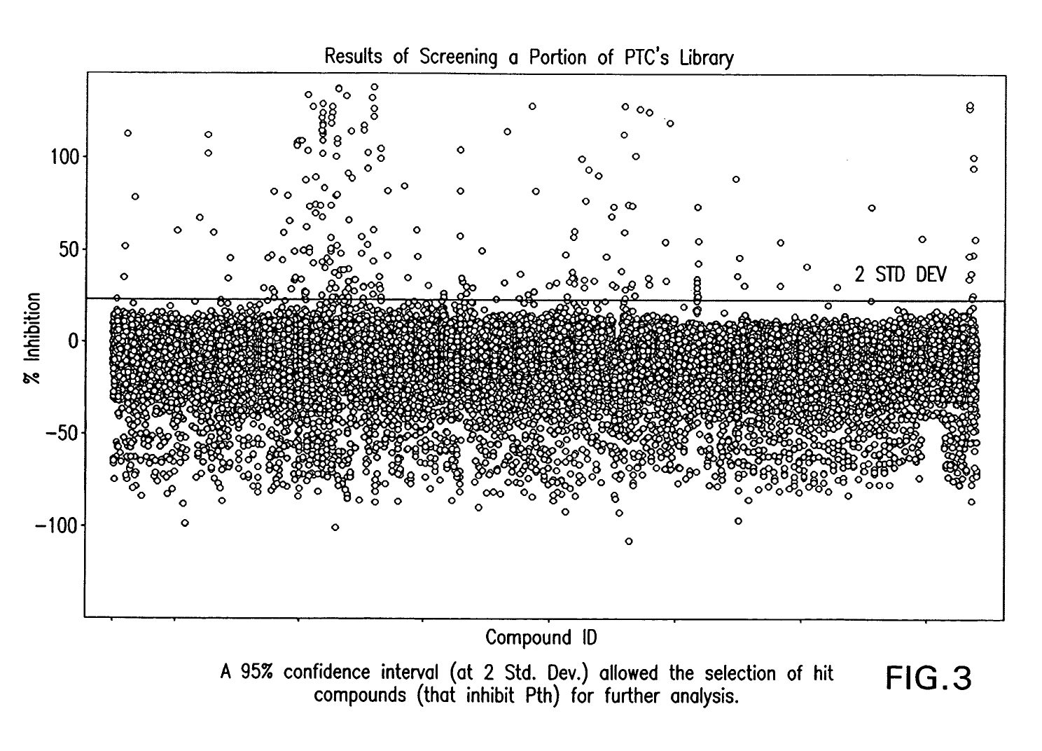

Using the E. coli peptidyl tRNA hydrolase as the prototype enzyme target and a 3,400-compound library subset, the fluorescence polarization assay for inhibition of peptidyl tRNA hydrolase activity was determined to be robust and sensitive, with Z′-values up to 0.65. FIG. 3 shows the analysis of percent inhibition profiles for 30,000 compounds screened against the E. coli peptidyl tRNA hydrolase enzyme.

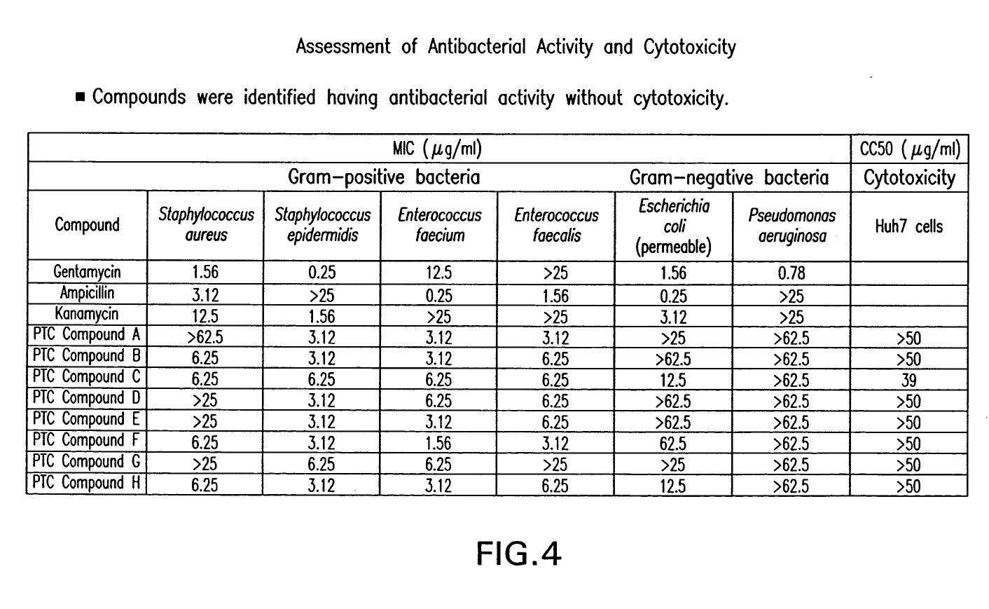

Compounds having inhibition greater than 25% were further evaluated for antibacterial activity (MIC), enzyme inhibition (IC₅₀) and cytotoxicity. The minimum inhibitory concentrations (MIC) of test compounds were determined using bacteria grown in brain heart infusion media (BHI). Logarithmically growing cells were diluted to approximately 5×105 CFU/ml and subjected to test compounds solubilized and serially diluted in DMSO. A 5% final DMSO concentration had no affect on cell viability or killing (2.5% final DMSO concentration routinely performed). After 18 hours at 37° C., the OD600 was determined by reading the ninety-six well microtiter plates on a microplate reader. For a given concentration, an MIC determination was made if: [OD600 Control−OD600 Test Conc.]/[OD₆₀₀ Control−OD600 Media]×100≧90%. All organisms are grown in a universal rich media to minimize media effects on the inhibition assay. All bacteria utilized in the MIC assay have been demonstrated to grow in Brain Heart Infusion (BHI) media (Difco, Detroit, Mich.). Library compounds are at a concentration of 2.5 to 10 mg/ml. The MICs for the antibiotics Ampicillin, Kanamycin, and Gentamicin are also shown in FIG. 4. Antibiotic concentrations varied from 25 ug/ml (1 mM stock) to 0.39 ug/ml. The bacteria tested were Enterococcus faecium (ATCC 49624), Enterococcus faecalis (ATCC 29212), Staphylococcus aureus (ATCC 29213), Staphylococcus epidermidis (ATCC 12228), Escherichia coli (BAS849—permeable) and Pseudomonas aeruginosa (ATCC 27853).

Novel antibacterial peptidyl tRNA hydrolase inhibitors were identified using the MIC assay (inhibition ranged from 32-100%). The inhibitors were also bacteria specific, as evidenced by the low cytotoxicity observed for the human Huh7 cells (FIG. 4). Cytotoxicity was determined according to manufacturer's directions (CellTiter 96 Aqueous Non-Radioactive Cell Proliferation Assay, Promega).

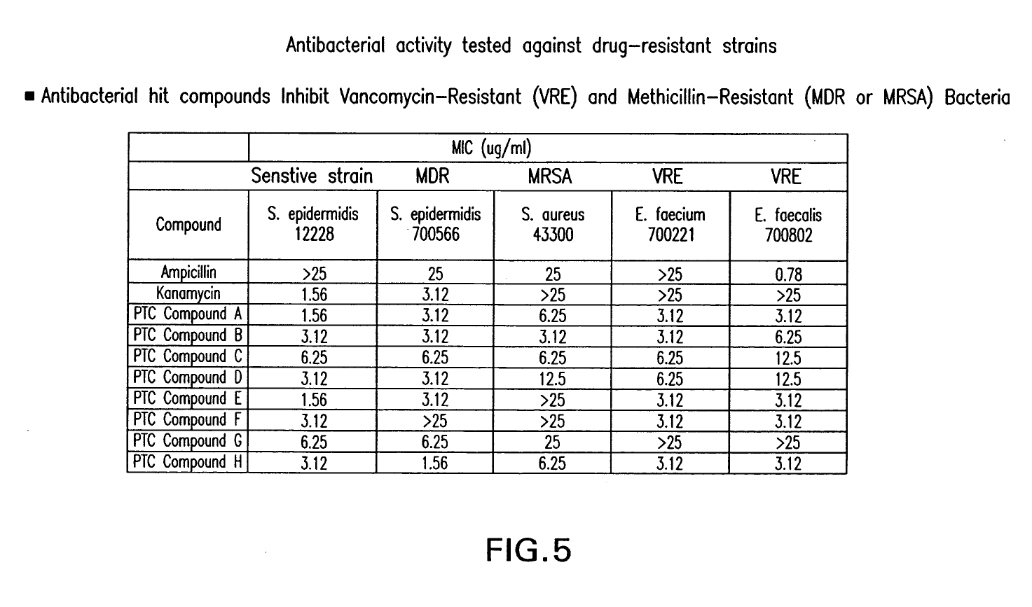

The inhibitors were also effective against antibiotic resistant strains of Staphylococcus epidermidis, Staphylococcus aureus, Enterococcus faecium, and Enterococcus faecalis (FIG. 5). Two unique classes of molecules have emerged from this initial screen. The significance of these compounds is two-fold. First, they can be used to standardize the assay for screening the full library. Second, these compounds serve as a starting point to evaluate analogs in a traditional drug discovery process. Analogs that exhibit activity will be analyzed in order to identify chemotypes that display the ideal behaviors of peptidyl tRNA hydrolase inhibition: antibacterial activity, limited cytotoxicity and good pharmacokinetic profiles. While the inhibitory concentrations are not sub-micromolar, it should be noted that the goal of the initial high throughput library screen is to identify compounds having the desired properties, which are then further optimized.

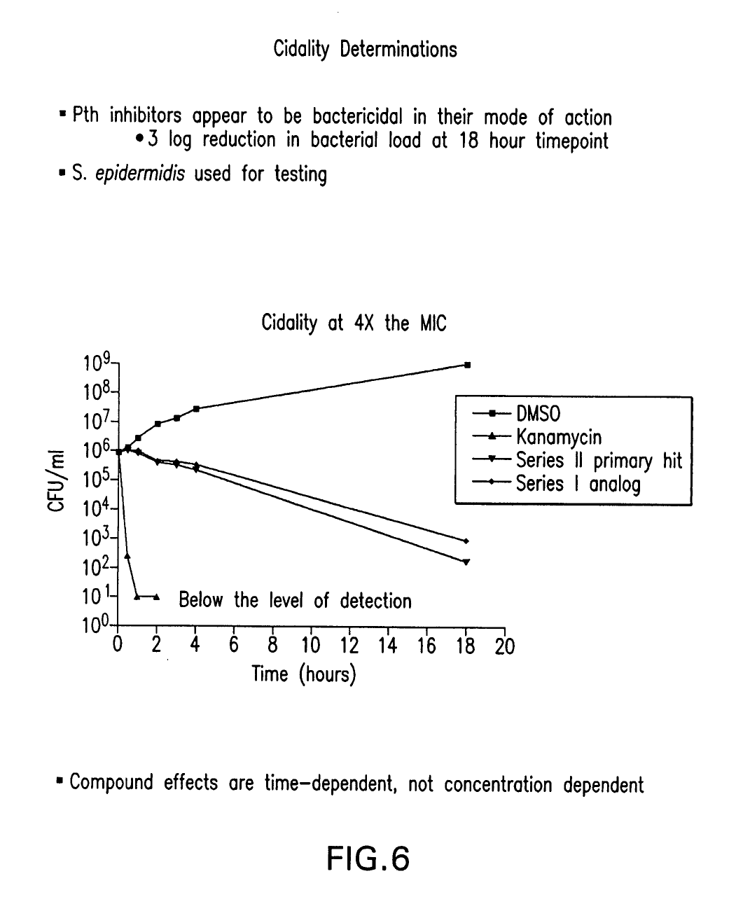

Preliminary testing of cidality on a representative compound against S. epidermidis suggests that the inhibitors identified were bactericidal (FIG. 6).

Further data for representative compounds is set forth in Table 1 (MIC and cytotoxicity). Compounds were tested against S. aureus (ATCC 29213) and E. coli (BAS849—permeable) in the MIC assays. Certain compounds were also tested against other bacteria, including S. epidermis (ATCC 12228), E. faecium (ATCC 49624), and E. faecalis (ATCC 29212). Cytotoxicity was determined using Hu7 cells and Hep G2 cells. The MIC and cytotoxicity assays were performed as described above.

The MIC results are presented according to the following scheme:
0.1-1 μg/ml=5 stars
1.1-10 μg/ml=4 stars
10.1-20 μg/ml=3 stars
20.1-50 μg/ml=2 stars
>50 μg/ml=1 star

### 6.2 Synthetic Examples

### 6.2.1 Example 1

### 1-(6-Chlorobenzo[d]thiazol-2-yl)-5-(3,4-difluorophenyl)-3-hydroxy-4-(5-methylfuran-2-carbonyl)-1H-pyrrol-2(5H)-one (Compound 461)

Part A.

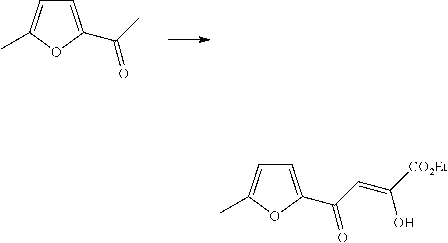

A round bottom flask was charged with ethanolic potassium ethoxide solution (35.0 mL, 2.55 M, 89.3 mmol) and 25 mL absolute ethanol. A solution of 2-acetyl-5-methylfuran (10.94 g, 88.1 mmol) and diethyl oxalate (12.1 mL, 88.3 mmol) in 30 mL ethanol was added dropwise over 30 minutes, and the resulting mixture was stirred for 12 hours. The resulting mixture was filtered to collect the precipitated potassium salt of the product, and the filter cake was washed with a small amount of ethanol. The moist solid was added to a beaker containing 500 mL of 0.5 N HCl with stirring. After stirring for 1 hour, the precipitate was collected by filtration, washed with water and dried under vacuum to afford the product, ethyl 2-hydroxy-4-(5-methylfuran-2-yl)-4-oxobut-2-enoate, as a pale yellow powder (16.57 g, 73.9 mmol, 84%), m.p. 88-92° C. ¹H NMR (300 MHz, DMSO-d₆): δ 7.60 (1H, br), 6.74 (1H, br), 6.44 (1H, d, J=2.6 Hz), 4.25 (2H, q, J=7.0 Hz), 2.39 (3H, s), 1.27 (3H, t, J=7.0 Hz). MS (ES−): m/e 224 (10), 223 (100).

Part B.

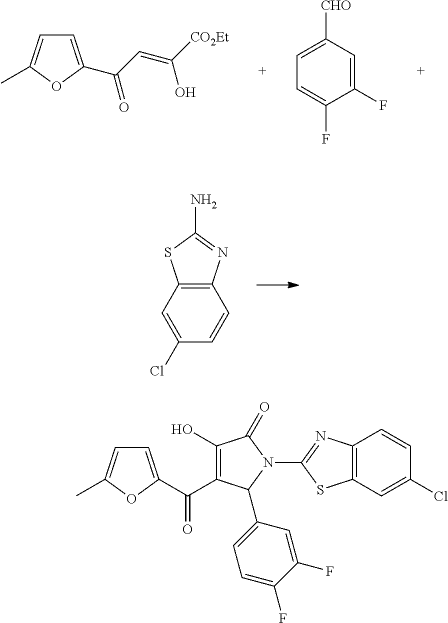

A mixture of ethyl 2-hydroxy-4-(5-methylfuran-2-yl)-4-oxobut-2-enoate (299 mg, 1.34 mmol), 3,4-difluorobenzaldehyde (152 μL, 1.32 mmol) and 2-amino-6-chlorobenzothiazole (197 mg, 1.07 mmol) was dissolved in 4 mL isopropanol and 1 mL acetic acid in a screw-top tube. The resulting solution was heated to 80° C. for 14 hours. The solution was cooled, and the precipitate was collected by filtration, washed well with diethyl ether and dried under high vacuum to afford the title product as a yellow solid (267 mg, 0.548 mmol, 51%), m.p. 235-236° C. ¹H NMR (300 MHz, DMSO-d₆): δ 8.14 (1H, s), 7.64-7.57 (3H, m), 7.41-7.24 (3H, m), 6.37 (1H, d, J=3.0 Hz), 6.24 (1H, s), 2.33 (3H, s). MS (ES+): m/e 489.0 (40), 487.1 (100). MS (ES−): m/e 487.2 (30), 485.2 (100).

The following compounds were prepared using these procedures and the appropriate starting materials.

Compound 50: m.p. 241-243° C. ¹H NMR (300 MHz, CDCl₃): δ 7.70-6.80 (15H, m), 5.57 (1H, br), 4.88 (2H, s), 2.10 (3H, s). MS (ES+): m/e 524 (30), 523 (100). MS (ES−): m/e 522 (20), 521 (100).

Compound 51: m.p. 213-214° C. ¹H NMR (300 MHz, DMSO-d₆): δ 8.12 (1H, s), 8.00 (1H, d, J=7.0 Hz), 7.85 (1H, d, J=7.6 Hz), 7.72-7.66 (2H, m), 7.53 (1H, td, J=8.3, 1.3 Hz), 7.44-7.24 (10H, m), 6.85 (2H, d, J=8.8 Hz), 6.34 (1H, s), 4.92 (2H, s). MS (ES+): m/e 560 (30), 559 (100). MS (ES−): m/e 558 (20), 557 (100).

Compound 52: m.p. 150-154° C. ¹H NMR (300 MHz, DMSO-d₆): δ 9.18 (1H, s), 8.73 (1H, s), 7.78 (1H, d, J=7.6 Hz), 7.59 (1H, d, J=7.9 Hz), 7.44-7.25 (10H, m), 6.83 (2H, d, J=8.8 Hz), 6.11 (1H, s), 4.96 (2H, s). MS (ES+): m/e 511 (30), 510 (100). MS (ES−): m/e 509 (40), 508 (100).

Compound 53: m.p. 193-195° C. ¹H NMR (300 MHz, DMSO-d₆): δ 9.11 (1H, s), 7.90-7.20 (14H, m), 6.90 (2H, d, J=8 Hz), 4.92 (2H, s). MS (ES+): m/e 510 (30), 509 (100). MS (ES−): m/e 508 (30), 507 (100).

Compound 54: m.p. 205-208° C. ¹H NMR (300 MHz, DMSO-d₆): δ 8.61 (1H, s), 7.79 (1H, d, J=7.6 Hz), 7.60 (1H, d, J=7.9 Hz), 7.43 (1H, td, J=7.0, 1.1 Hz), 7.39-7.09 (7H, m), 6.14 (1H, s), 2.44 (3H, s), 1.89 (3H, s). MS (ES+): m/e 460 (25), 459 (100). MS (ES−): m/e 458 (30), 457 (100).

Compound 55: m.p. 226-228° C. ¹H NMR (300 MHz, DMSO-d₆): δ 8.34 (1H, s), 7.83 (1H, d, J=7.9 Hz), 7.64 (1H, d, J=8.5 Hz), 7.59-7.26 (12H, m), 6.26 (1H, s), 2.46 (3H, s), 1.89 (3H, s). MS (ES+): m/e 536 (20), 535 (100). MS (ES−): m/e 534 (20), 533 (100).

Compound 56: m.p. 251-253° C. ¹H NMR (300 MHz, DMSO-d₆): δ 8.18 (1H, s), 7.84 (1H, d, J=7.3 Hz), 7.68 (1H, d, J=7.9 Hz), 7.52 (1H, td, J=8.5, 1.2 Hz), 7.43 (2H, d, J=8.8 Hz), 7.37-7.31 (2H, m), 7.28 (1H, t, J=7.2 Hz), 6.90 (2H, d, J=7.8 Hz), 6.82 (2H, d, J=8.8 Hz), 6.23 (1H, s), 2.47 (3H, s), 1.89 (3H, s). MS (ES+): m/e 552 (30), 551 (100). MS (ES−): m/e 550 (35), 549 (100).

Compound 57: m.p. 186-188° C. ¹H NMR (300 MHz, DMSO-d₆): δ 7.90-6.60 (10H, m), 3.80 (2H, m), 2.45 (3H, s), 2.15 (3H, s), 1.57 (2H, m), 1.33 (2H, m), 0.85 (3H, m). MS (ES+): m/e 532 (20), 531 (100). MS (ES−): m/e 530 (30), 529 (100).

Compound 10: m.p. 195-197° C. ¹H NMR (300 MHz, DMSO-d₆): δ 9.16 (1H, s), 7.81 (1H, s), 7.74 (1H, d, J=7.9 Hz), 7.60-7.20 (10H, m), 6.80 (2H, m), 5.99 (1H, s), 4.96 (2H, s), 2.45 (3H, s), 2.15 (3H, s). MS (ES+): m/e 566 (25), 565 (100). MS (ES−): m/e 564 (30), 563 (100).

Compound 58: m.p. 169-171° C. ¹H NMR (300 MHz, DMSO-d₆): δ 7.90-6.80 (17H, br), 5.05 (2H, s), 1.89 (3H, s).

Compound 117: m.p. 251-253° C. ¹H NMR (300 MHz, DMSO-d₆): δ 8.10 (1H, d, J=2.0 Hz), 8.08 (1H, br), 7.55 (1H, d, J=8.8 Hz), 7.36 (1H, dd, J=8.6, 2.2 Hz), 7.25-6.94 (4H, m), 6.38 (1H, narrow m), 6.25 (1H, narrow m), 2.28 (3H, s). ¹⁹F NMR (300 MHz, DMSO-d₆): δ −117.89 (1H, s). MS (ES+): m/e 471 (50), 469 (100). MS (ES−): m/e 469 (48), 467 (100).

Compound 120: m.p. 197-201° C. ¹H NMR (300 MHz, DMSO-d₆): δ 8.35-7.49 (7H, m), 7.22 (1H, td, J=8.2, 1.1 Hz), 6.38 (1H, s), 6.35 (1H, dd, J=3.5, 0.9 Hz), 2.59 (2H, q, J=6.8 Hz), 2.31 (3H, d, J=2 Hz), 1.16 (3H, t, J=6.8 Hz). MS (ES+): m/e 491 (30), 490 (100). MS (ES−): m/e 489 (25), 488 (100).

Compound 130: m.p. 204-206° C. ¹H NMR (300 MHz, CDCl₃): δ 7.80-6.80 (9H, m), 6.20 (1H, narrow m), 5.90 (1H, br), 2.60 (2H, q, J=7 Hz), 2.30 (3H, s), 1.20 (3H, t, J=7 Hz). MS (ES+): m/e 446 (25), 445 (100). MS (ES−): m/e 444 (25), 443 (100).

Compound 131: m.p. 222-225° C. ¹H NMR (300 MHz, CDCl₃): δ 7.74 (1H, d, J=8.2 Hz), 7.60-7.50 (3H, m), 7.32-7.21 (2H, m), 6.92 (1H, t, J=8.6 Hz), 6.75 (1H, s), 6.19 (1H, dd, J=2.9, 0.4 Hz), 5.98 (1H, br), 2.74 (2H, q, J=7.6 Hz), 2.45 (3H, s), 1.26 (3H, t, J=7.6 Hz). ¹⁹F NMR (300 MHz, CDCl₃): δ −113.14 (1F, s). MS (ES+): m/e 464 (25), 463 (100). MS (ES−): m/e 462 (25), 461 (100).

Compound 132: m.p. 192-194° C. ¹H NMR (300 MHz, CDCl₃): δ 7.75 (1H, d, J=8.2 Hz), 7.58 (1H, d, J=1.5 Hz), 7.49 (2H, d, J=8.8 Hz), 7.30 (1H, d, J=3.3 Hz), 7.25 (1H, dd, J=8.2, 1.5 Hz), 6.74 (2H, d, J=8.8 Hz), 6.71 (1H, s), 6.18 (1H, dd, J=3.3, 0.8 Hz), 3.69 (3H, s), 2.72 (2H, q, J=7.3 Hz), 2.45 (3H, s), 1.25 (3H, t, J=7.3 Hz). MS (ES+): m/e 476 (25), 475 (100).

Compound 133: m.p. 236-238° C. ¹H NMR (300 MHz, CDCl₃): δ 7.73 (1H, d, J=7.6 Hz), 7.59 (1H, s), 7.52 (2H, d, J=8.8 Hz), 7.33 (1H, d, J=3.2 Hz), 7.31-7.25 (1H, m), 7.21 (2H, d, J=8.8 Hz), 6.72 (1H, s), 6.21 (1H, d, J=2.9 Hz), 2.73 (2H, q, J=7.6 Hz), 2.46 (3H, s), 1.26 (3H, t, J=7.6 Hz). MS (ES+): m/e 481 (50), 479 (100). MS (ES−): m/e 479 (45), 477 (100).

Compound 134: m.p. 197-201° C. ¹H NMR (300 MHz, CDCl₃): δ 8.00-7.00 (7H, m), 6.22 (1H, br), 6.03 (1H, br), 2.73 (2H, q, J=7 Hz), 2.49 (3H, s), 1.21 (3H, t, J=7 Hz). MS (ES+): m/e 513 (100). MS (ES−): m/e 511 (100).

Compound 135: m.p. 220-222° C. ¹H NMR (300 MHz, CDCl₃): δ 7.76 (1H, d, J=8.2 Hz), 7.59 (1H, dd, J=1.8, 0.6 Hz), 7.47 (2H, d, J=8.2 Hz), 7.30-7.24 (2H, m), 7.06 (2H, d, J=8.2 Hz), 6.74 (1H, s), 6.18 (1H, dd, J=3.5, 0.9 Hz), 2.76 (1H, hp, J=7.0 Hz), 2.73 (2H, q, J=7.6 Hz), 2.45 (3H, s), 1.26 (3H, t, J=7.6 Hz), 1.12 (6H, d, J=7.0 Hz). MS (ES+): m/e 488 (25), 487 (100). MS (ES−): m/e 486 (25), 485 (100).

Compound 136: m.p. 174-177° C. ¹H NMR (300 MHz, CDCl₃): δ 9.99 (1H, s), 7.80-7.00 (8H, m), 6.72 (1H, s), 6.23 (1H, m), 2.74 (2H, q, J=7 Hz), 2.49 (3H, s), 1.26 (3H, t, J=7 Hz). MS (ES+): m/e 479 (100).

Compound 137: m.p. 138-140° C. ¹H NMR (300 MHz, CDCl₃): δ 8.00-7.20 (8H, m), 6.81 (1H, s), 6.22 (1H, m), 2.73 (2H, q, J=7 Hz), 2.45 (3H, s), 1.26 (3H, t, J=7 Hz). ¹⁹F NMR (300 MHz, CDCl₃): δ −63.11 (3F, s). MS (ES+): m/e 514 (20), 513 (100). MS (ES−): m/e 512 (20), 511 (100).

Compound 138: m.p. 165-169° C. ¹H NMR (300 MHz, CDCl₃): δ 7.90-7.00 (9H, m), 6.24 (1H, m), 2.75 (2H, q, J=7 Hz), 2.43 (3H, s), 1.23 (3H, t, J=7 Hz). MS (ES+): m/e 464 (20), 463 (100).

Compound 139: m.p.>300° C. ¹H NMR (300 MHz, DMSO-d₆): δ 8.20-6.80 (9H, br), 6.20 (1H, br), 2.40 (3H, br). MS (ES+): m/e 496 (100). MS (ES−): m/e 496 (40), 494 (100).

Compound 151: m.p. 268-269° C. ¹H NMR (300 MHz, DMSO-d₆): δ 7.58-7.55 (2H, m), 7.48 (1H, d, J=9.0 Hz), 7.34 (1H, td, J=7.6, 1.5 Hz), 7.25-7.06 (2H, m), 7.02 (1H, td, J=7.3, 1.1 Hz), 6.96 (1H, dd, J=8.8, 2.6 Hz), 6.48 (1H, s), 6.36 (1H, dd, J=3.7, 1.0 Hz), 3.76 (3H, s), 2.32 (3H, s). ¹⁹F NMR (300 MHz, DMSO-d₆): δ −117.97 (1F, s). MS (ES+): m/e 466 (25), 465 (100). MS (ES−): m/e 463 (100).

Compound 152: m.p. 255-257° C. ¹H NMR (300 MHz, DMSO-d₆): δ 9.06 (1H, d, J=2.3 Hz), 8.20 (1H, dd, J=9.1, 2.6 Hz), 7.72 (1H, d, J=9.1 Hz), 7.58 (1H, d, J=3.5 Hz), 7.43-7.37 (1H, m), 7.27-7.19 (1H, m), 7.16-7.08 (1H, m), 7.02 (1H, td, J=7.6, 1.1 Hz), 6.53 (1H, s), 6.37 (1H, dd, J=3.5, 0.8 Hz), 2.33 (3H, s). ¹⁹F NMR (300 MHz, DMSO-d₆): δ −117.99 (1F, s). MS (ES+): m/e 481 (25), 480 (100). MS (ES−): m/e 479 (25), 478 (100).

Compound 117: m.p. 264-265° C. ¹H NMR (300 MHz, DMSO-d₆): δ 8.13 (1H, d, J=2.3 Hz), 7.58 (1H, d, J=3.6 Hz), 7.56 (1H, d, J=8.8 Hz), 7.40-7.32 (2H, m), 7.26-7.00 (4H, m), 6.49 (1H, s), 6.36 (1H, dd, J=3.6, 0.7 Hz), 2.32 (3H, s). ¹⁹F NMR (300 MHz, DMSO-d₆): δ −117.99 (1F, s). MS (ES+): m/e 471 (45), 469 (100). MS (ES−): m/e 469 (50), 467 (100).

Compound 153: m.p. 240-242° C. ¹H NMR (300 MHz, DMSO-d₆): δ 7.65 (1H, d, j=8.8 Hz), 7.60 (1H, d, J=2.6 Hz), 7.57 (1H, d, J=3.5 Hz), 7.44 (1H, d, J=1.2 Hz), 7.01 (1H, dd, J=8.8, 2.6 Hz), 6.56 (1H, d, J=2.7 Hz), 6.45 (1H, s), 6.39 (1H, dd, J=3.5, 0.9 Hz), 6.30 (1H, dd, J=3.2, 1.9 Hz), 3.78 (3H, s), 2.36 (3H, s). MS (ES+): m/e 437 (100).

Compound 154: m.p. 250-252° C. ¹H NMR (300 MHz, DMSO-d₆): δ 7.98 (1H, s), 7.76 (1H, d, J=7.9 Hz), 7.68 (1H, d, J=7.9 Hz), 7.58-7.54 (2H, m), 7.49 (1H, d, J=8.8 Hz), 7.38 (1H, t, J=7.7 Hz), 6.95 (1H, dd, J=8.8, 2.5 Hz), 6.37-6.32 (2H, m), 3.81 (3H, s), 3.75 (3H, s), 2.32 (3H, s). MS (ES+): m/e 505 (100).

Compound 155: m.p. 263-264° C. ¹H NMR (300 MHz, DMSO-d₆): δ 9.34 (1H, s), 7.58 (1H, d, J=2.7 Hz), 7.55 (1H, d, J=8.8 Hz), 7.47 (1H, d, J=3.5 Hz), 7.00 (1H, t, J=8 Hz), 6.97 (1H, dd, J=9.2, 2.7 Hz), 6.75 (1H, d, J=7.9 Hz), 6.73 (1H, t, J=1.9 Hz), 6.52 (1H, ddd, J=8.2, 2.3, 0.9 Hz), 6.36 (1H, dd, J=3.5, 1.0 Hz), 6.19 (1H, s), 3.77 (3H, s), 2.33 (3H, s). MS (ES−): m/e 462 (20), 461 (100).

Compound 156: m.p. 241-242° C. ¹H NMR (300 MHz, DMSO-d₆): δ 8.92 (1H, s), 7.58 (1H, s), 7.56 (1H, d, J=5.6 Hz), 7.48 (1H, d, J=3.5 Hz), 6.98 (1H, dd, J=9.0, 2.6 Hz), 6.78-6.72 (3H, m), 6.36 (1H, dd, J=3.5, 0.9 Hz), 6.16 (1H, s), 3.77 (3H, s), 3.63 (3H, s), 2.34 (3H, s). MS (ES+): m/e 493 (100).

Compound 157: m.p. 240-241° C. ¹H NMR (300 MHz, DMSO-d₆): δ 8.63 (1H, d, J=3.2 Hz), 8.18 (1H, dd, J=4.7, 0.9 Hz), 7.72 (1H, d, J=7.6 Hz), 7.63 (1H, td, J=7.6, 1.8 Hz), 7.49 (1H, d, J=2.6 Hz), 7.43 (1H, d, J=8.8 Hz), 7.02 (1H, ddd, J=7.3, 4.7, 1.2 Hz), 6.89 (1H, dd, J=8.8, 2.6 Hz), 6.13-6.09 (2H, m), 3.75 (3H, s), 2.24 (3H, s). MS (ES+): m/e 449 (32), 448 (100).

Compound 158: m.p. 291-292° C. ¹H NMR (300 MHz, DMSO-d₆): δ 8.79 (1H, d, J=1.8 Hz), 8.40 (1H, dd, J=4.8, 1.3 Hz), 7.90 (1H, d, J=8.2 Hz), 7.77 (1H, d, J=3.2 Hz), 7.57 (1H, d, J=2.6 Hz), 7.52 (1H, d, J=8.8 Hz), 7.32 (1H, dd, J=7.9, 5.0 Hz), 6.96 (1H, dd, J=8.8, 2.6 Hz), 6.34 (1H, d, J=2.9 Hz), 6.25 (1H, s), 3.76 (3H, s), 2.31 (3H, s). MS (ES+): m/e 448 (100). MS (ES−): m/e 446 (100).

Compound 159: m.p. 244-245° C. ¹H NMR (300 MHz, DMSO-d₆): δ 7.57 (1H, d, J=3.5 Hz), 7.32-7.19 (2H, m), 7.13-6.98 (2H, m), 6.40 (1H, s), 6.36 (1H, dd, J=3.5, 0.9 Hz), 2.44 (3H, s), 2.40 (3H, s), 2.31 (3H, s). ¹⁹F NMR (300 MHz, DMSO-d₆): δ −118.01 (1F, s). MS (ES+): m/e 441 (100).

Compound 160: m.p. 244-245° C. ¹H NMR (300 MHz, DMSO-d₆): δ 7.57 (1H, d, J=3.5 Hz), 7.32-7.19 (2H, m), 7.13-6.98 (2H, m), 6.40 (1H, s), 6.36 (1H, dd, J=3.5, 0.9 Hz), 2.44 (3H, s), 2.40 (3H, s), 2.31 (3H, s). ¹⁹F NMR (300 MHz, DMSO-d₆): δ −118.01 (1F, s). MS (ES+): m/e 441 (100).

Compound 161: m.p. 251-252° C. ¹H NMR (300 MHz, DMSO-d₆): δ 7.53 (1H, d, J=3.3 Hz), 7.28-7.16 (2H, m), 7.10-6.98 (3H, m), 6.37-6.34 (2H, m), 2.31 (3H, s), 2.29 (3H, d, J=1.2 Hz). ¹⁹F NMR (300 MHz, DMSO-d₆): δ −118.25 (1F, s). MS (ES−): m/e 397 (100).

Compound 162: m.p.>350° C. ¹H NMR (300 MHz, DMSO-d₆): δ 8.35 (1H, br), 8.00 (1H, s), 7.55 (1H, dd, J=3.5, 0.6 Hz), 7.32 (1H, t, J=7.9 Hz), 7.30-7.00 (3H, m), 6.37 (1H, s), 6.35 (1H, d, J=3.5 Hz), 2.31 (3H, s). MS (ES+): m/e 436 (100).

Compound 163: m.p. 253-254° C. ¹H NMR (300 MHz, DMSO-d₆): δ 7.98 (1H, dd, J=8.4, 1.1 Hz), 7.60-7.57 (2H, m), 7.40-7.00 (6H, m), 6.52 (1H, s), 6.36 (1H, d, J=3.5 Hz), 2.33 (3H, s). ¹⁹F NMR (300 MHz, DMSO-d₆): δ −117.98 (1F, s). MS (ES+): m/e 435 (100).

Compound 178: m.p. 233-234° C. ¹H NMR (300 MHz, DMSO-d₆): δ 7.56 (1H, d, J=3.8 Hz), 6.40 (1H, dd, J=3.8, 0.9 Hz), 5.00 (2H, s), 4.28 (2H, s), 2.37 (3H, s). MS (ES+): m/e 290.11 (100). MS (ES−): m/e 289.34 (10), 288.02 (100).

Compound 179: m.p. 208-209° C. ¹H NMR (300 MHz, DMSO-d₆): δ 8.71 (1H, d, J=1.8 Hz), 8.08 (1H, d, J=8.5 Hz), 7.96 (1H, dd, J=8.5, 1.8 Hz), 7.50 (1H, d, J=3.3 Hz), 6.46 (1H, d, J=3.3 Hz), 5.41 (1H, d, J=2.9 Hz), 3.24 (3H, s), 2.72-2.62 (1H, m), 2.41 (3H, s), 1.78-1.44 (4H, m), 1.30-0.72 (6H, m). MS (ES+): m/e 502.50 (20), 501.10 (100). MS (ES−): m/e 500.57 (20), 499.06 (100).

Compound 180: m.p. 199-200° C. ¹H NMR (300 MHz, DMSO-d₆): δ 8.72 (0.5H, d, J=2.0 Hz), 8.71 (0.5H, d, J=2.0 Hz), 8.08 (0.5H, d, J=8.5 Hz), 8.07 (0.5H, d, J=8.5 Hz), 7.97 (0.5H, d, J=8.5 Hz), 7.96 (0.5H, d, J=8.5 Hz), 7.51 (0.5H, d, J=3.5 Hz), 7.45 (0.5H, d, J=3.5 Hz), 6.43 (0.5H, d, J=3.5 Hz), 6.40 (0.5H, d, J=3.5 Hz), 5.60 (0.5H, d, J=3.0 Hz), 5.57 (0.5H, d, J=2.5 Hz), 3.75-3.30 (5H, m), 3.24 (3H, s), 2.40 (1.5H, s), 2.39 (1.5H, s), 1.80-1.40 (2H, s). MS (ES+): m/e 490.46 (15), 489.06 (100). MS (ES−): m/e 488.47 (20), 487.01 (100).

Compound 181: m.p. 171-173° C. ¹H NMR (300 MHz, DMSO-d₆): δ 8.70 (1H, narrow m), 8.08 (1H, d, J=8 Hz), 7.96 (1H, d, J=8 Hz), 7.22 (1H, d, J=3 Hz), 6.42 (1H, d, J=3 Hz), 5.66 (1H, m), 3.25 (3H, s), 2.47 (3H, s), 1.40-0.70 (11H, m). MS (ES+): m/e 490.56 (20), 489.10 (100). MS (ES−): m/e 488.51 (20), 487.09 (100).

Compound 182: m.p. 247-248° C. ¹H NMR (300 MHz, DMSO-d₆): δ 7.55-7.50 (3H, m), 7.40-7.36 (2H, m), 7.25-7.12 (3H, m), 6.94 (1H, dd, J=8.9, 2.5 Hz), 6.34 (1H, dd, J=3.7, 1.0 Hz), 6.25 (1H, s), 4.01 (2H, q, J=7.1 Hz), 2.32 (3H, s), 1.30 (3H, t, J=7.1 Hz). MS (ES+): m/e 462.51 (20), 461.11 (100). MS (ES−): m/e 460.48 (15), 459.11 (100).

Compound 183: m.p. 251-252° C. ¹H NMR (300 MHz, DMSO-d₆): δ 7.57 (1H, d, J=3.2 Hz), 7.55 (1H, d, J=2.6 Hz), 7.46 (1H, d, J=8.8 Hz), 7.33 (1H, td, J=7.6, 1.5 Hz), 7.25-7.17 (1H, m), 7.10 (1H, ddd, J=17, 8, 1 Hz), 7.02 (1H, td, J=7.6, 1.1 Hz), 6.94 (1H, dd, J=8.8, 2.6 Hz), 6.47 (1H, s), 6.36 (1H, dd, J=3.5, 0.9 Hz), 4.01 (2H, q, J=7.1 Hz), 2.32 (3H, s), 1.30 (3H, t, J=7.1 Hz). MS (ES+): m/e 480.51 (20), 479.12 (100). MS (ES−): m/e 478.42 (20), 477.08 (100).

Compound 184: m.p. 256-257° C. ¹H NMR (300 MHz, DMSO-d₆): δ 8.32 (1H, t, J=1.9 Hz), 8.04 (1H, ddd, J=8.2, 2.3, 0.9 Hz), 7.90 (1H, dt, J=8.2, 1.3 Hz), 7.63 (1H, d, J=3.2 Hz), 7.56-7.46 (3H, m), 6.94 (1H, dd, J=8.8, 2.6 Hz), 6.38 (1H, s), 6.35 (1H, dd, J=3.5, 0.9 Hz), 4.00 (2H, q, J=7.0 Hz), 2.32 (3H, s), 1.30 (3H, t, J=7.0 Hz). MS (ES+): m/e 507.50 (20), 506.12 (100). MS (ES−): m/e 505.50 (15), 504.10 (100).

Compound 185: m.p. 166-167° C. ¹H NMR (300 MHz, DMSO-d₆): δ 7.80-7.00 (4H, m), 6.40-5.95 (2H, m), 4.07 (2H, q, J=7 Hz), 2.47 (1H, m), 2.31 (3H, s), 1.75-1.40 (10H, m), 1.34 (3H, t, J=7 Hz). MS (ES+): m/e 468.55 (20), 467.15 (100). MS (ES−): m/e 466.51 (20), 465.16 (100).

Compound 186: m.p. 214-215° C. ¹H NMR (300 MHz, DMSO-d₆): δ 7.56-7.50 (4H, m), 7.39-7.28 (5H, m), 7.14 (1H, t, J=7.9 Hz), 7.02-6.94 (2H, m), 6.81 (1H, dd, J=8.4, 1.9 Hz), 6.35 (1H, dd, J=3.5, 0.9 Hz), 6.22 (1H, s), 5.00 (1H, d, J=11.7 Hz), 4.95 (1H, d, J=11.7 Hz), 4.02 (2H, q, J=7.0 Hz), 2.33 (3H, s), 1.31 (3H, t, J=7.0 Hz). MS (ES+): m/e 568.54 (20), 567.25 (100). MS (ES−): m/e 566.49 (20), 565.20 (100).

Compound 187: m.p. 191-192° C. ¹H NMR (300 MHz, DMSO-d₆): δ 7.55-7.52 (3H, m), 7.29 (2H, d, J=8.5 Hz), 6.95 (1H, dd, J=8.8, 2.7 Hz), 6.74 (2H, d, J=8.5 Hz), 6.34 (1H, dd, J=3.5, 0.6 Hz), 6.21 (1H, s), 4.01 (2H, q, J=7.0 Hz), 3.85-3.75 (2H, m), 2.33 (3H, s), 1.62-1.53 (2H, m), 1.39-1.27 (5H, m), 0.85 (3H, t, J=7.4 Hz). MS (ES+): m/e 534.55 (20), 533.23 (100). MS (ES−): m/e 532.52 (20), 531.18 (100).

Compound 188: m.p. 249-250° C. ¹H NMR (300 MHz, DMSO-d₆): δ 12.07 (1H, br s), 8.65 (2H, d, J=4.7 Hz), 7.53 (1H, d, J=3.5 Hz), 7.25 (1H, td, J=7.6, 1.5 Hz), 7.18 (1H, t, J=4.7 Hz), 7.17-7.11 (1H, m), 7.06-6.95 (2H, m), 6.48 (1H, s), 6.34 (1H, dd, J=3.5, 0.9 Hz), 2.31 (3H, s). ¹⁹F NMR (300 MHz, DMSO-d₆): δ −118.15 (1F, s). MS (ES+): m/e 381.34 (15), 380.11 (100). MS (ES−): m/e 379.37 (15), 378.07 (100).

Compound 189: m.p. 235-236° C. ¹H NMR (300 MHz, DMSO-d₆): δ 8.77 (1H, d, J=1.2 Hz), 8.68 (1H, d, J=5.8 Hz), 8.25 (1H, dd, J=5.8, 1.2 Hz), 7.56 (1H, d, J=3.5 Hz), 7.27-7.14 (1H, m), 7.22-7.14 (1H, m), 7.08-6.96 (2H, m), 6.45 (1H, s), 6.34 (1H, dd, J=3.8, 1.2 Hz), 2.31 (3H, s). MS (ES+): m/e 381.35 (15), 380.11 (100). MS (ES−): m/e 379.35 (10), 378.07 (100).

Compound 190: m.p. 185-186° C. ¹H NMR (300 MHz, DMSO-d₆): δ 7.80-7.00 (4H, m), 6.40 (1H, m), 6.18 (1H, m), 4.06 (2H, q, J=7.0 Hz), 3.60-3.35 (5H, m), 2.33 (3H, s), 1.80-1.65 (2H, m), 1.34 (3H, t, J=7.0 Hz). MS (ES+): m/e 456.55 (15), 455.12 (100). MS (ES−): m/e 454.47 (15), 453.13 (100).

Compound 217: m.p. 106-109° C. MS (ES+): m/e 420.17 (100).

Compound 218: m.p. 256-258° C. ¹H NMR (300 MHz, DMSO-d₆): δ 8.57 (1H, s), 7.61 (1H, d, J=3.5 Hz), 7.40-6.93 (4H, m), 6.69 (1H, s), 6.35 (1H, dd, J=3.5, 0.8 Hz), 2.32 (3H, s). ¹⁹F NMR (282 MHz, DMSO-d₆): δ −118.01 (1F, s). MS (ES+): m/e 421.39 (20), 420.17 (100). MS (ES−): m/e 419.42 (20), 418.12 (100).

Compound 219: m.p. 170-173° C. ¹H NMR (300 MHz, DMSO-d₆): δ 8.63 (1H, s), 8.55 (1H, s), 7.56 (1H, d, J=3.2 Hz), 7.35-6.68 (5H, m), 6.36 (1H, d, J=3.2 Hz), 2.32 (3H, s). MS (ES+): m/e 421.49 (20), 420.29 (100). MS (ES−): m/e 419.52 (20), 418.29 (100).

Compound 220: m.p. 226-227° C. ¹H NMR (300 MHz, DMSO-d₆): δ 7.99 (1H, t, J=1.7 Hz), 7.80 (1H, t, J=7.9, 1 Hz), 7.66-7.62 (2H, m), 7.55 (1H, d, J=2.6 Hz), 7.53 (1H, d, J=11.4 Hz), 7.45 (1H, t, J=7.8 Hz), 6.95 (1H, dd, J=8.8, 2.6 Hz), 6.36 (1H, dd, J=3.5, 0.9 Hz), 6.26 (1H, s), 4.01 (2H, q, J=7.0 Hz), 2.32 (3H, s), 1.31 (3H, t, J=7.0 Hz). MS (ES+): m/e 487.56 (25), 486.21 (100). MS (ES−): m/e 485.51 (25), 484.21 (100).

Compound 221: m.p. 259-260° C. ¹H NMR (300 MHz, DMSO-d₆): δ 8.84 (2H, br s), 7.56 (1H, d, J=5.3 Hz), 7.55 (1H, s), 7.46 (1H, d, J=3.5 Hz), 6.96 (1H, dd, J=8.8, 2.5 Hz), 6.70 (1H, d, J=2.0 Hz), 6.62 (1H, dd, J=8.2, 2.0 Hz), 6.53 (1H, d, J=8.2 Hz), 6.36 (1H, dd, J=3.5, 0.9 Hz), 6.12 (1H, s), 4.02 (2H, q, J=7.0 Hz), 2.34 (3H, s), 1.31 (3H, t, J=7.0 Hz). MS (ES+): m/e 494.61 (15), 493.21 (100). MS (ES−): m/e 492.40 (10), 491.22 (100).

Compound 222: m.p. 248-249° C. ¹H NMR (300 MHz, DMSO-d₆): δ 7.57-7.50 (3H, m), 7.30-7.23 (3H, m), 7.03-6.93 (2H, m), 6.35 (1H, d, J=3.5 Hz), 6.24 (1H, s), 4.01 (2H, q, J=7.0 Hz), 2.32 (3H, s), 1.31 (3H, t, J=7.0 Hz). ¹⁹F NMR (282 MHz, DMSO-d₆): δ 113.82 (1F, m). MS (ES+): m/e 480.60 (20), 479.20 (100). MS (ES−): m/e 478.56 (20), 477.23 (100).

Compound 223: m.p. 254-255° C. ¹H NMR (300 MHz, DMSO-d₆): δ 9.34 (1H, br s), 7.56 (1H, d, J=3 Hz), 7.54 (1H, d, J=9 Hz), 7.47 (1H, d, J=3.5 Hz), 7.00 (1H, t, J=7.6 Hz), 6.96 (1H, dd, J=8.8, 2.6 Hz), 6.75 (1H, d, J=7.9 Hz), 6.72 (1H, t, J=1.9 Hz), 6.52 (1H, ddd, J=8.2, 2.3, 0.9 Hz), 6.35 (1H, dd, J=3.8, 0.9 Hz), 6.19 (1H, s), 4.02 (2H, q, J=7.0 Hz), 2.33 (3H, s), 1.31 (3H, t, J=7.0 Hz). MS (ES+): m/e 478.59 (20), 477.18 (100). MS (ES−): m/e 476.48 (20), 475.23 (100).

Compound 224: m.p. 266-267° C. ¹H NMR (300 MHz, DMSO-d₆): δ 8.65 (1H, s), 8.59 (1H, s), 8.19 (1H, t, J=1.8 Hz), 7.95 (1H, ddd, J=8.2, 2.3, 0.9 Hz), 7.83 (1H, d, J=7.6 Hz), 7.60 (1H, d, J=3.5 Hz), 7.46 (1H, t, J=8.0 Hz), 6.71 (1H, br s), 6.36 (1H, dd, J=3.5, 0.9 Hz), 2.32 (3H, s). MS (ES+): m/e 448.47 (15), 447.19 (100). MS (ES−): m/e 446.47 (15), 445.21 (100).

Compound 225: m.p. 222-223° C. ¹H NMR (300 MHz, DMSO-d₆): δ 8.76 (1H, s), 8.58 (1H, s), 8.39 (1H, s), 8.00-7.81 (3H, m), 7.41 (1H, t, J=8.2 Hz), 6.75 (1H, s), 6.26 (1H, d, J=3 Hz), 2.37 (3H, s). MS (ES+): m/e 448.47 (15), 447.19 (100). MS (ES−): m/e 446.49 (15), 445.21 (100).

Compound 244: m.p. 248-249° C. ¹H NMR (300 MHz, DMSO-d₆): δ 8.44 (1H, d, J=4.1 Hz), 8.34 (1H, s), 8.05 (1H, dd, J=8.2, 2.3 Hz), 7.97 (1H, dd, J=8.2, 0.6 Hz), 7.94 (1H, d, J=7.6 Hz), 7.66 (1H, d, J=3.5 Hz), 7.54 (1H, t, J=7.6 Hz), 7.42 (1H, dd, J=8.3, 4.5 Hz), 6.41 (1H, s), 6.36 (1H, d, J=3.5 Hz), 2.32 (3H, s). MS (ES+): m/e 463.17 (100). MS (ES−): m/e 462.51 (20), 461.21 (100).

Compound 245: m.p. 227-229° C. ¹H NMR (300 MHz, DMSO-d₆): δ 8.44 (1H, dd, J=4.7, 1.2 Hz), 7.97 (1H, dd, J=8.2, 1.2 Hz), 7.59 (1H, d, J=3.5 Hz), 7.45-7.35 (2H, m), 7.26-7.19 (1H, m), 7.11 (1H, t, J=9 Hz), 7.03 (1H, t, J=7.3 Hz), 6.51 (1H, s), 6.36 (1H, d, J=3.5 Hz), 2.32 (3H, s). ¹⁹F NMR (282 MHz, DMSO-d₆): δ −117.99 (1F, s). MS (ES+): m/e 437.59 (15), 436.21 (100). MS (ES−): m/e 435.51 (20), 434.22 (100).

Compound 246: m.p. 251-252° C. ¹H NMR (300 MHz, DMSO-d₆): δ 8.44 (1H, dd, J=4.7, 1.4 Hz), 8.03 (1H, s), 7.99 (1H, dd, J=8.2, 1.4 Hz), 7.83 (1H, d, J=7.9 Hz), 7.67-7.62 (2H, m), 7.49-7.41 (2H, m), 6.36 (1H, d, J=3.5 Hz), 6.30 (1H, s), 2.32 (3H, s). MS (ES+): m/e 444.56 (20), 443.20 (100). MS (ES−): m/e 442.53 (20), 441.23 (100).

Compound 247: m.p. 248-249° C. ¹H NMR (300 MHz, DMSO-d₆): δ 8.45 (1H, dd, J=4.7, 1.2 Hz), 8.04 (1H, dd, J=8.2, 1.5 Hz), 7.58 (1H, d, J=3.5 Hz), 7.43 (1H, dd, J=8.2, 4.7 Hz), 6.99 (1H, d, J=1.4 Hz), 6.93 (1H, dd, J=8.2, 1.4 Hz), 6.76 (1H, d, J=8.2 Hz), 6.36 (1H, d, J=3.5 Hz), 6.21 (1H, s), 5.92 (1H, s), 5.88 (1H, s), 2.33 (3H, s). MS (ES+): m/e 463.54 (15), 462.20 (100).

Compound 248: m.p. 243-245° C. ¹H NMR (300 MHz, DMSO-d₆): δ 8.45 (1H, dd, J=4.7, 0.9 Hz), 8.00 (1H, dd, J=8.2, 1.3 Hz), 7.85 (1H, d, J=2.3 Hz), 7.58-7.55 (2H, m), 7.44 (1H, dd, J=8.2, 4.7 Hz), 6.80 (1H, d, J=8.2 Hz), 6.37 (1H, d, J=3.5 Hz), 6.27 (1H, s), 2.33 (3H, s). MS (ES+): m/e 479.56 (20), 478.18 (100). MS (ES−): m/e 477.53 (20), 476.21 (100).

Compound 249: m.p. 220-221° C. ¹H NMR (300 MHz, DMSO-d₆): δ 8.44 (1H, dd, J=4.7, 1.5 Hz), 8.01 (1H, t, J=1.6 Hz), 7.97 (1H, dd, J=8.2, 1.5 Hz), 7.76 (1H, dt, J=7.6, 1.0 Hz), 7.70 (1H, d, J=7.9 Hz), 7.58 (1H, d, J=3.5 Hz), 7.44-7.36 (2H, m), 6.36 (1H, s), 6.35 (1H, d, J=3.5 Hz), 3.81 (3H, s), 2.32 (3H, s). MS (ES+): m/e 477.60 (20), 476.21 (100). MS (ES−): m/e 475.47 (20), 474.20 (100).

Compound 250: m.p. 227-228° C. ¹H NMR (300 MHz, DMSO-d₆): δ 7.73 (1H, d, J=8 Hz), 7.70 (1H, d, J=8 Hz), 7.60 (1H, d, J=3.5 Hz), 7.55 (1H, d, J=4.4 Hz), 7.53 (1H, d, J=2.0 Hz), 7.33 (1H, t, J=7.4 Hz), 7.24 (1H, t, J=7.5 Hz), 6.95 (1H, dd, J=9.1, 2.3 Hz), 6.78 (1H, s), 6.37 (1H, d, J=3.5 Hz), 3.99 (2H, q, J=7.0 Hz), 2.70 (3H, s), 2.31 (3H, s), 1.29 (3H, t, J=7.0 Hz). MS (ES+): m/e 532.62 (20), 531.21 (100). MS (ES−): m/e 530.46 (20), 529.24 (100).

Compound 251: m.p. 230-231° C. ¹H NMR (300 MHz, DMSO-d₆): δ 7.58-7.53 (3H, m), 6.98-6.89 (3H, m), 6.75 (1H, d, J=7.9 Hz), 6.36 (1H, dd, J=3.5, 0.8 Hz), 6.17 (1H, s), 5.91 (1H, s), 5.88 (1H, s), 4.02 (2H, q, J=6.7 Hz), 2.33 (3H, s), 1.31 (3H, t, J=6.7 Hz). MS (ES+): m/e 505.22 (100). MS (ES−): m/e 504.51 (15), 503.26 (100).

Compound 252: m.p. 260-261° C. ¹H NMR (300 MHz, DMSO-d₆): δ 8.33 (1H, d, J=8.8 Hz), 7.91-7.79 (2H, m), 7.70 (1H, d, J=8.8 Hz), 7.65-7.60 (2H, m), 7.55-7.49 (1H, m), 7.39 (1H, d, J=8.8 Hz), 7.06 (1H, dd, J=8.9, 2.5 Hz), 6.88 (1H, dd, J=8.9, 2.6 Hz), 6.52 (1H, s), 6.31 (1H, dd, J=3.5, 0.9 Hz), 4.07 (2H, q, J=7.0 Hz), 2.28 (3H, s), 1.33 (3H, t, J=7.0 Hz). MS (ES+): m/e 513.61 (20), 512.24 (100). MS (ES−): m/e 511.56 (20), 510.24 (100).

Compound 253: m.p. 114-115° C. ¹H NMR (300 MHz, DMSO-d₆): δ 7.57 (1H, d, J=2.3 Hz), 7.53 (1H, d, J=8.8 Hz), 7.48 (1H, d, J=3.8 Hz), 7.32 (2H, d, J=8.8 Hz), 6.96 (1H, dd, J=8.8, 2.6 Hz), 6.84 (2H, d, J=8.8 Hz), 6.48 (1H, s), 6.44 (1H, d, J=3.5 Hz), 4.24-4.20 (2H, m), 4.08-4.04 (2H, m), 4.02 (2H, q, J=7.0 Hz), 2.35 (3H, s), 2.28 (3H, s), 1.93 (3H, s), 1.31 (3H, t, J=7.0 Hz). MS (ES+): m/e 606.69 (30), 605.37 (100). MS (ES−): m/e 603.44 (25), 561.30 (100).

Compound 254: m.p. 251-252° C. ¹H NMR (300 MHz, DMSO-d₆): δ 7.84 (1H, d, J=2.3 Hz), 7.56-7.50 (4H, m), 6.96 (1H, dd, J=8.8, 2.6 Hz), 6.79 (1H, d, J=8.5 Hz), 6.36 (1H, d, J=3.5 Hz), 6.23 (1H, s), 4.01 (2H, q, J=7.0 Hz), 2.33 (3H, s), 1.31 (3H, t, J=7.0 Hz). MS (ES+): m/e 522.58 (20), 521.22 (100). MS (ES−): m/e 520.42 (20), 519.17 (100).

Compound 255: m.p. 228-229° C. ¹H NMR (300 MHz, DMSO-d₆): δ 7.98 (1H, t, J=1.5 Hz), 7.75 (1H, d, J=7.6 Hz), 7.67 (1H, d, J=7.9 Hz), 7.55 (1H, d, J=3.5 Hz), 7.54 (1H, d, J=2.6 Hz), 7.47 (1H, d, J=9.0 Hz), 7.38 (1H, t, J=7.8 Hz), 6.93 (1H, dd, J=9.0, 2.6 Hz), 6.34 (1H, d, J=3.5 Hz), 6.32 (1H, s), 4.00 (2H, q, J=7.0 Hz), 3.81 (3H, s), 2.32 (3H, s), 1.30 (3H, t, J=7.0 Hz). MS (ES+): m/e 520.64 (20), 519.24 (100). MS (ES−): m/e 518.53 (15), 517.15 (100).

Compound 256: m.p. 187-188° C. ¹H NMR (300 MHz, DMSO-d₆): δ 8.29 (1H, s), 8.06 (1H, dd, J=7.9, 1 Hz), 7.97 (1H, d, J=7.9 Hz), 7.76 (1H, br s), 7.67-7.60 (2H, m), 7.57 (1H, d, J=2.3 Hz), 7.08 (1H, d, J=3.2 Hz), 6.99 (1H, dd, J=8.8, 2.3 Hz), 6.72 (1H, d, J=3.2 Hz), 6.50 (1H, s), 6.37 (1H, d, J=2.7 Hz), 4.02 (2H, q, J=6.8 Hz), 2.33 (3H, s), 1.31 (3H, t, J=6.8 Hz). MS (ES+): m/e 573.59 (20), 572.27 (100). MS (ES−): m/e 571.59 (20), 570.17 (100).

Compound 257: m.p. 235-236° C. ¹H NMR (300 MHz, DMSO-d₆): δ 7.66 (1H, d, J=8.8 Hz), 7.58-7.55 (2H, m), 7.33 (1H, ddd, J=5.0, 1.2, 0.6 Hz), 7.28 (1H, ddd, J=3.5, 1.2, 0.6 Hz), 7.00 (1H, dd, J=8.8, 2.6 Hz), 6.86 (1H, dd, J=5.0, 3.5 Hz), 6.62 (1H, s), 6.38 (1H, dd, J=3.5, 0.9 Hz), 4.03 (2H, q, J=7.0 Hz), 2.35 (3H, s), 1.32 (3H, t, J=7.0 Hz). MS (ES+): m/e 468.64 (20), 467.17 (100). MS (ES−): m/e 466.48 (15), 465.19 (100).

Compound 283: m.p. 291-292° C. ¹H NMR (300 MHz, DMSO-d₆): δ 13.09 (1H, s), 8.02 (1H, s), 7.46 (1H, d, J=3.5 Hz), 7.28-7.05 (5H, m), 6.32 (1H, d, J=3.5 Hz), 6.24 (1H, s), 4.11 (4H, br), 3.63 (4H, br), 2.30 (3H, s). MS (ES+): m/e 488.58 (20), 487.30 (100). MS (ES−): m/e 486.55 (20), 485.34 (100).

Compound 284: m.p. 280-282° C. ¹H NMR (300 MHz, DMSO-d₆): δ 13.10 (1H, s), 8.16 (1H, t, J=1.9 Hz), 8.01 (1H, s), 7.98 (1H, dd, J=8.2, 2.3 Hz), 7.75 (1H, d, J=8.2 Hz), 7.62 (1H, narrow m), 7.48 (1H, t, J=7.9 Hz), 6.34 (1H, s), 6.32 (1H, d, J=3.4 Hz), 4.10 (4H, br), 3.60 (4H, br), 2.30 (3H, s). MS (ES+): m/e 533.52 (20), 532.29 (100). MS (ES−): m/e 531.50 (25), 530.31 (100).

Compound 285: m.p. 297-299° C. ¹H NMR (300 MHz, DMSO-d₆): δ 13.09 (1H, br s), 11.96 (1H, br s), 8.02 (1H, s), 7.50 (1H, d, J=3.5 Hz), 7.26-7.11 (2H, m), 7.06-6.96 (2H, m), 6.47 (1H, s), 6.33 (1H, dd, J=3.5, 0.9 Hz), 4.13 (4H, br), 3.64 (4H, br), 2.30 (3H, s). MS (ES+): m/e 506.56 (20), 505.31 (100). MS (ES−): m/e 504.55 (20), 503.30 (100).

Compound 420: m.p. 238-239° C. ¹H NMR (300 MHz, DMSO-d₆): δ 8.14 (1H, d, J=2.0 Hz), 7.63 (1H, d, J=8.5 Hz), 7.46-7.38 (3H, m), 7.07 (2H, t, J=7.8 Hz), 6.03 (1H, s), 2.35 (3H, s). MS (ES+): m/e 405.2 (35), 403.3 (100). MS (ES−): m/e 403.0 (40), 401.0 (100).

Compound 421: m.p. 239-240° C. ¹H NMR (300 MHz, DMSO-d₆): δ 8.15 (1H, s), 7.64 (1H, d, J=7.9 Hz), 7.41-7.30 (3H, m), 7.10-7.00 (2H, m), 6.09 (1H, s), 1.08 (9H, s). MS (ES+): m/e 447.2 (40), 445.3 (100). MS (ES−): m/e 445.0 (40), 443.1 (100).

Compound 422: m.p. 236-237° C. ¹H NMR (300 MHz, DMSO-d₆): δ 8.09 (1H, d, J=2.0 Hz), 7.60 (1H, d, J=8.8 Hz), 7.39-7.32 (3H, m), 7.04 (2H, t, J=8.8 Hz), 5.90 (1H, s), 3.91 (2H, m), 1.03 (3H, t, J=7.0 Hz). MS (ES+): m/e 434.86 (45), 433.03 (100). MS (ES−): m/e 433.00 (40), 431.10 (100).

Compound 423: m.p. 238-239° C. ¹H NMR (300 MHz, DMSO-d₆): δ 7.90 (1H, dd, J=8.8, 2.6 Hz), 7.65 (1H, dd, J=8.9, 4.8 Hz), 7.45-7.39 (2H, m), 7.23 (1H, td, J=8.8, 2.6 Hz), 7.07 (2H, t, J=8.8 Hz), 6.03 (1H, s), 2.35 (3H, s). MS (ES+): m/e 388.47 (20), 387.12 (100). MS (ES−): m/e 386.38 (20), 385.15 (100).

Compound 424: m.p. 232-233° C. ¹H NMR (300 MHz, DMSO-d₆): δ 7.90 (1H, dd, J=8.8, 2.6 Hz), 7.66 (1H, dd, J=8.8, 4.8 Hz), 7.41-7.36 (2H, m), 7.24 (1H, td, J=9.3, 2.6 Hz), 7.07 (2H, t, J=8.8 Hz), 6.09 (1H, s), 1.08 (9H, s). MS (ES+): m/e 430.5 (20), 429.3 (100). MS (ES−): m/e 428.1 (20), 427.0 (100).

Compound 425: m.p. 218-219° C. ¹H NMR (300 MHz, DMSO-d₆): δ 7.91 (1H, dd, J=8.6, 2.5 Hz), 7.64 (1H, dd, J=8.9, 4.6 Hz), 7.47-7.42 (2H, m), 7.24 (1H, td, J=8.9, 2.5 Hz), 7.10 (2H, t, J=8.8 Hz), 6.06 (1H, s), 4.04 (2H, m), 1.09 (3H, t, J=7.0 hz). MS (ES−): m/e 416.2 (20), 415.0 (100).

Compound 426: m.p. 231-232° C. ¹H NMR (300 MHz, DMSO-d₆): δ 7.96 (1H, d, J=7.9 Hz), 7.52-7.46 (3H, m), 7.29 (1H, t, J=7.9 Hz), 7.07 (2H, t, J=8.8 Hz), 6.05 (1H, s), 2.37 (3H, s). MS (ES+): m/e 404.87 (50), 403.04 (100). MS (ES−): m/e 402.99 (40), 401.08 (100).

Compound 428: ¹H NMR (300 MHz, DMSO-d₆): δ 9.37 (1H, br s), 8.14 (1H, d, J=2.0 Hz), 7.68 (1H, d, J=8.5 Hz), 7.41 (1H, dd, J=8.8, 2.0 Hz), 7.17 (2H, d, J=8.5 Hz), 6.61 (2H, d, J=8.5 Hz), 5.93 (1H, s), 5.22-5.19 (1H, m), 3.45-3.29 (2H, m), 2.20-2.04 (1H, m), 1.93 (3H, s), 1.80-1.70 (1H, m), 1.60-1.50 (1H, m), 1.35-1.20 (1H, m). MS (ES+): m/e 499.96 (50), 498.16 (100). MS (ES−): m/e 497.99 (50), 496.20 (100).

Compound 429: m.p. 255-256° C. ¹H NMR (300 MHz, DMSO-d₆): δ 8.13 (1H, s), 7.56 (1H, d, J=8.8 Hz), 7.38 (1H, d, J=8.5 Hz), 7.24-6.99 (4H, m), 6.24 (1H, s), 2.35 (3H, s). MS (ES+): m/e 404.8 (40), 403.0 (100). MS (ES−): m/e 402.9 (45), 401.0 (100).

Compound 430: m.p. 235-236° C. ¹H NMR (300 MHz, CDCl₃): δ 7.74 (1H, d, J=2.0 Hz), 7.60 (1H, d, J=8.8 Hz), 7.33-7.19 (3H, m), 7.06-6.95 (2H, m), 6.32 (1H, s), 4.51 (3H, s), 2.43 (3H, s). MS (ES+): m/e 417.0 (100). MS (ES−): m/e 415.1 (100).

Compound 431: m.p. 254-255° C. ¹H NMR (300 MHz, DMSO-d₆): δ 9.43 (1H, s), 7.37 (1H, d, J=7.6 Hz), 7.24-7.04 (5H, m), 6.81 (1H, d, J=3.2 Hz), 6.71 (1H, s), 6.59 (2H, d, J=8.5 Hz), 6.19 (1H, dd, J=3.2, 0.8 Hz), 3.94-3.78 (2H, m), 3.57 (3H, s), 2.29 (3H, s), 1.01 (3H, t, J=7.0 Hz). MS (ES+): m/e 459.2 (35), 458.2 (100). MS (ES−): m/e 457.4 (15), 456.2 (100).

Compound 432: m.p. 195-196° C. ¹H NMR (300 MHz, DMSO-d₆): δ 7.48 (1H, d, J=3.2 Hz), 7.32-7.06 (4H, m), 6.31 (1H, d, J=3.2 Hz), 5.72 (1H, s), 3.54 (1H, m), 2.28 (3H, s), 1.69-1.47 (6H, m), 1.14-0.80 (4H, m). MS (ES+): m/e 384.4 (100). MS (ES−): m/e 383.2 (10), 382.0 (100).

Compound 433: m.p. 204-206° C. ¹H NMR (300 MHz, DMSO-d₆): δ 7.48 (1H, d, J=3.5 Hz), 7.37-7.20 (2H, m), 7.18-7.02 (3H, m), 6.31 (1H, d, J=3.5 Hz), 5.70 (1H, s), 4.51 (1H, br), 3.49 (1H, br), 3.11 (2H, m), 2.29 (3H, s), 1.90-1.40 (6H, m). MS (ES+): m/e 400.3 (100). MS (ES−): m/e 399.1 (20), 398.0 (100).

Compound 434: m.p. 74-75° C. ¹H NMR (300 MHz, DMSO-d₆): δ 7.49 (1H, d, J=3 Hz), 7.30-7.00 (4H, m), 6.30 (1H, d, J=3 Hz), 5.78 (1H, s), 4.80 (1H, br), 3.75-3.30 (6H, m), 2.27 (3H, s). MS (ES−): m/e 373.9 (100).

Compound 435: m.p. 309-310° C. ¹H NMR (300 MHz, DMSO-d₆): δ 8.42 (2H, s), 8.15 (1H, d, J=2.0 Hz), 7.61 (1H, d, J=8.8 Hz), 7.43-7.11 (4H, m), 7.02 (1H, t, J=7.5 Hz), 6.47 (1H, s). MS (ES−): m/e 471.7 (45), 469.8 (100).

Compound 436: m.p. 310-311° C. ¹H NMR (300 MHz, DMSO-d₆): δ 8.43 (1H, d, J=3.2 Hz), 8.41 (1H, d, J=3.2 Hz), 8.16 (1H, d, J=2.0 Hz), 7.67 (1H, d, J=8.8 Hz), 7.55 (2H, dd, J=8.8, 5.3 Hz), 7.42 (1H, dd, J=8.8, 2.3 Hz), 7.07 (2H, t, J=8.8 Hz), 6.24 (1H, s). MS (ES−): m/e 471.5 (35), 469.7 (100).

Compound 437: m.p. 298-299° C. ¹H NMR (300 MHz, DMSO-d₆): δ 8.43 (1H, d, J=3.3 Hz), 8.42 (1H, d, J=3.3 Hz), 7.92 (1H, dd, J=8.8, 2.6 Hz), 7.62 (1H, dd, J=8.8, 4.8 Hz), 7.40-7.00 (5H, m), 6.46 (1H, s). MS (ES+): m/e 456.0 (100). MS (ES−): m/e 454.9 (20), 453.6 (100).

Compound 438: m.p. 320-321° C. ¹H NMR (300 MHz, DMSO-d₆): δ 8.43 (1H, d, J=3.2 Hz), 8.41 (1H, d, J=3.2 Hz), 7.92 (1H, dd, J=8.4, 2.6 Hz), 7.69 (1H, dd, J=8.8, 4.8 Hz), 7.54 (2H, dd, J=9.0, 5.6 Hz), 7.26 (1H, td, J=9.0, 2.6 Hz), 7.07 (2H, t, J=8.8 Hz), 6.24 (1H, s). MS (ES+): m/e 457.7 (25), 456.0 (100). MS (ES−): m/e 454.9 (20), 453.7 (100).

Compound 439: m.p. 320-321° C. ¹H NMR (300 MHz, DMSO-d₆): δ 8.44 (1H, d, J=3.2 Hz), 8.42 (1H, d, J=3.2 Hz), 7.98 (1H, dd, J=8.1, 0.8 Hz), 7.48 (1H, dd, J=7.8, 1.0 Hz), 7.34-7.12 (4H, m), 7.00 (1H, t, J=6.9 Hz), 6.50 (1H, s). MS (ES−): m/e 471.7 (25), 469.7 (100).

Compound 440: m.p. 303-304° C. ¹H NMR (300 MHz, DMSO-d₆): δ 8.44 (1H, d, J=3.1 Hz), 8.42 (1H, d, J=3.1 Hz), 7.98 (1H, dd, J=7.9, 1.1 Hz), 7.61 (2H, dd, J=9.0, 5.6 Hz), 7.50 (1H, dd, J=7.9, 1.2 Hz), 7.30 (1H, t, J=7.9 Hz), 7.07 (2H, t, J=8.8 Hz), 6.26 (1H, s). MS (ES−): m/e 471.6 (40), 469.7 (100).

Compound 441: m.p. 253-254° C. ¹H NMR (300 MHz, DMSO-d₆): δ 8.13 (1H, d, J=2.0 Hz), 7.58 (1H, d, J=9.0 Hz), 7.39 (1H, dd, J=8.6, 2.2 Hz), 7.30-7.00 (4H, m), 6.27 (1H, s), 4.47 (1H, d, J=18.1 Hz), 4.39 (1H, d, J=18.1 Hz), 3.23 (3H, s). MS (ES+): m/e 435.0 (50), 433.1 (100). MS (ES−): m/e 432.8 (30), 430.7 (100).

Compound 442: m.p. 263-264° C. ¹H NMR (300 MHz, DMSO-d₆): δ 8.14 (1H, d, J=2.0 Hz), 7.64 (1H, d, J=8.8 Hz), 7.47-7.38 (3H, m), 7.07 (2H, t, J=8.8 Hz), 6.06 (1H, s), 4.46 (1H, d, J=18.1 Hz), 4.37 (1H, d, J=18.1 Hz), 3.23 (3H, s). MS (ES+): m/e 435.0 (45), 433.0 (100). MS (ES−): m/e 432.8 (35), 430.8 (100).

Compound 443: m.p. 228-229° C. ¹H NMR (300 MHz, DMSO-d₆): δ 7.90 (1H, dd, J=8.6, 2.8 Hz), 7.59 (1H, dd, J=8.9, 4.8 Hz), 7.28-7.00 (5H, m), 6.27 (1H, s), 4.47 (1H, d, J=18.1 Hz), 4.39 (1H, d, J=18.1 Hz), 3.23 (3H, s). MS (ES+): m/e 418.3 (20), 417.1 (100). MS (ES−): m/e 415.9 (20), 414.7 (100).

Compound 444: m.p. 249-250° C. ¹H NMR (300 MHz, DMSO-d₆): δ 7.90 (1H, dd, J=8.6, 2.7 Hz), 7.66 (1H, dd, J=8.9, 4.8 Hz), 7.44 (2H, dd, J=8.8, 5.4 Hz), 7.24 (1H, td, J=8.9, 2.7 Hz), 7.07 (2H, t, J=8.8 Hz), 6.06 (1H, s), 4.46 (1H, d, J=18.1 Hz), 4.37 (1H, d, J=18.1 Hz), 3.23 (3H, s). MS (ES+): m/e 418.6 (15), 417.0 (100). MS (ES−): m/e 416.1 (15), 414.8 (100).

Compound 445: m.p. 231-233° C. ¹H NMR (300 MHz, DMSO-d₆): δ 7.88 (1H, d, J=7.9 Hz), 7.41 (1H, d, J=7.9 Hz), 7.21 (1H, t, J=7.9 Hz), 7.14-6.91 (4H, m), 6.12 (1H, s), 4.45 (1H, d, J=16.7 Hz), 4.29 (1H, d, J=16.7 Hz), 3.20 (3H, s). MS (ES+): m/e 434.7 (40), 433.0 (100). MS (ES−): m/e 432.7 (40), 430.7 (100).

Compound 446: m.p. 218-219° C. ¹H NMR (300 MHz, DMSO-d₆): δ 8.14 (1H, d, J=2.0 Hz), 7.58 (1H, dd, J=8.7, 0.6 Hz), 7.39 (1H, dd, J=8.7, 2.0 Hz), 7.36-7.00 (6H, m), 6.90 (1H, t, J=7.3 Hz), 6.71 (2H, dd, J=8.8, 1.2 Hz), 6.30 (1H, s), 5.18 (1H, d, J=17.8 Hz), 5.08 (1H, d, J=17.8 Hz). MS (ES+): m/e 496.9 (40), 495.1 (100). MS (ES−): m/e 494.7 (40), 492.8 (100).

Compound 447: m.p. 203-204° C. ¹H NMR (300 MHz, DMSO-d₆): δ 8.14 (1H, dd, J=1.8, 1.1 Hz), 7.64 (1H, d, J=8.8 Hz), 7.48 (2H, dd, J=9.0, 5.6 Hz), 7.40 (1H, dd, J=8.8, 2.0 Hz), 7.18 (2H, dd, J=8.8, 7.3 Hz), 7.07 (2H, t, J=8.8 Hz), 6.91 (1H, t, J=8.4 Hz), 6.70 (2H, dd, J=7.8, 1.2 Hz), 6.09 (1H, s), 5.17 (1H, d, J=17.8 Hz), 5.08 (1H, d, J=17.8 Hz). MS (ES+): m/e 496.5 (25), 495.0 (100). MS (ES−): m/e 494.7 (45), 492.8 (100).

Compound 448: m.p. 197-198° C. ¹H NMR (300 MHz, DMSO-d₆): δ 7.91 (1H, dd, J=8.8, 2.7 Hz), 7.60 (1H, dd, J=9.1, 5.0 Hz), 7.34-7.02 (7H, m), 6.90 (1H, t, J=7.3 Hz), 6.71 (2H, dd, J=9.0, 1.2 Hz), 6.30 (1H, s), 5.18 (1H, d, J=17.9 Hz), 5.08 (1H, d, J=17.9 Hz). MS (ES+): m/e 480.4 (20), 479.0 (100). MS (ES−): m/e 478.0 (30), 476.8 (100).

Compound 449: m.p. 131-133° C. ¹H NMR (300 MHz, DMSO-d₆): δ 7.96 (1H, dd, J=8.2, 0.9 Hz), 7.50-6.70 (11H, m), 6.34 (1H, s), 5.18 (1H, d, J=17.6 Hz), 5.10 (1H, d, J=17.6 Hz). MS (ES+): m/e 496.6 (35), 495.1 (100). MS (ES−): m/e 494.9 (35), 492.8 (100).

Compound 450: m.p. 204-206° C. ¹H NMR (300 MHz, DMSO-d₆): δ 7.97 (1H, dd, J=8.0, 1.0 Hz), 7.60-7.03 (8H, m), 6.88 (1H, t, J=7.3 Hz), 6.72 (2H, dd, J=8.8, 0.9 Hz), 6.11 (1H, s), 5.18 (1H, d, J=18.7 Hz), 5.17 (1H, d, J=18.7 Hz). MS (ES+): m/e 496.8 (50), 495.0 (100). MS (ES−): m/e 494.8 (30), 492.8 (100).

Compound 451: m.p. 246-247° C. ¹H NMR (300 MHz, DMSO-d₆): δ 8.13 (1H, d, J=2.0 Hz), 7.56 (1H, d, J=8.5 Hz), 7.37 (1H, dd, J=8.5, 2.0 Hz), 7.28-7.18 (2H, m), 7.14-7.00 (2H, m), 6.25 (1H, s), 2.94-2.85 (1H, m), 0.98-0.69 (4H, m). MS (ES+): m/e 431.0 (35), 429.1 (100). MS (ES−): m/e 429.0 (30), 427.1 (100).

Compound 452: m.p. 229-230° C. ¹H NMR (300 MHz, DMSO-d₆): δ 8.13 (1H, d, J=2.0 Hz), 7.62 (1H, d, J=8.5 Hz), 7.45-7.37 (3H, m), 7.05 (2H, t, J=8.8 Hz), 6.04 (1H, s), 2.88-2.82 (1H, m), 0.95-0.71 (4H, m). MS (ES+): m/e 431.0 (35), 429.1 (100). MS (ES−): m/e 429.0 (35), 427.0 (100).

Compound 453: m.p. 245-246° C. ¹H NMR (300 MHz, DMSO-d₆): δ 8.13 (1H, d, J=1.8 Hz), 7.62 (1H, d, J=8.5 Hz), 7.42-7.37 (3H, m), 7.29 (2H, d, J=8.5 Hz), 6.03 (1H, s), 2.90-2.48 (1H, m), 0.96-0.69 (4H, m). MS (ES+): m/e 445.0 (100). MS (ES−): m/e 443.0 (100).

Compound 454: m.p. 234-235° C. ¹H NMR (300 MHz, DMSO-d₆): δ 7.89 (1H, dd, J=8.8, 2.6 Hz), 7.58 (1H, dd, J=8.8, 4.7 Hz), 7.27-6.99 (5H, m), 6.25 (1H, s), 2.94-2.85 (1H, m), 0.98-0.69 (4H, m). MS (ES+): m/e 414.2 (20), 413.1 (100). MS (ES−): m/e 412.2 (20), 411.1 (100).

Compound 455: m.p. 221-223° C. ¹H NMR (300 MHz, DMSO-d₆): δ 7.90 (1H, dd, J=8.8, 2.6 Hz), 7.64 (1H, dd, J=8.9, 4.8 Hz), 7.42 (2H, dd, J=9.0, 5.6 Hz), 7.23 (1H, td, J=9.0, 2.6 Hz), 7.05 (2H, t, J=9.0 Hz), 6.04 (1H, s), 2.91-2.83 (1H, m), 0.97-0.69 (4H, m). MS (ES+): m/e 414.3 (20), 413.1 (100). MS (ES−): m/e 412.2 (20), 411.1 (100).

Compound 456: m.p. 253-254° C. ¹H NMR (300 MHz, DMSO-d₆): δ 7.95 (1H, dd, J=7.9, 1.2 Hz), 7.45 (1H, dd, J=7.9, 1.2 Hz), 7.27 (1H, t, J=8.2 Hz), 7.26-7.18 (3H, m), 7.14-7.06 (1H, m), 7.00 (1H, td, J=7.6, 1.0 Hz), 6.29 (1H, s), 2.95-2.86 (1H, m), 0.98-0.70 (4H, m). MS (ES+): m/e 431.0 (40), 429.1 (100). MS (ES−): m/e 429.0 (40), 427.1 (100).

Compound 457: m.p. 218-219° C. ¹H NMR (300 MHz, DMSO-d₆): δ 7.95 (1H, dd, J=7.9, 0.9 Hz), 7.50-7.45 (3H, m), 7.28 (1H, t, J=7.9 Hz), 7.06 (2H, t, J=8.8 Hz), 6.06 (1H, s), 2.93-2.84 (1H, m), 0.98-0.71 (4H, m). MS (ES+): m/e 430.9 (40), 429.1 (100). MS (ES−): m/e 429.0 (40), 427.1 (100).

Compound 458: m.p. 196-197° C. ¹H NMR (300 MHz, DMSO-d₆): δ 8.14 (1H, d, J=2.0 Hz), 7.64 (1H, d, J=8.8 Hz), 7.49 (2H, dd, J=8.8, 5.5 Hz), 7.39 (2H, td, J=8.4, 2.0 Hz), 7.14-7.07 (1H, m), 7.07 (2H, t, J=9.0 Hz), 6.89 (1H, td, J=7.6, 1.2 Hz), 6.69 (1H, dd, J=8.2, 0.9 Hz), 6.09 (1H, s), 5.29 (1H, d, J=17.8 Hz), 5.23 (1H, d, J=17.8 Hz). MS (ES+): m/e 529.0 (100). MS (ES−): m/e 527.1 (100).

Compound 459: m.p. 234-235° C. ¹H NMR (300 MHz, DMSO-d₆): δ 7.91 (1H, dd, J=8.6, 2.4 Hz), 7.67 (1H, dd, J=8.9, 4.8 Hz), 7.48 (2H, dd, J=8.6, 5.4 Hz), 7.38 (1H, dd, J=7.9, 1.5 Hz), 7.25 (1H, td, J=8.9, 2.8 Hz), 7.14-7.04 (3H, m), 6.89 (1H, td, J=7.3, 1 Hz), 6.69 (1H, d, J=8.2 Hz), 6.08 (1H, s), 5.29 (1H, d, J=18.1 Hz), 5.22 (1H, d, J=18.1 Hz). MS (ES+): m/e 515.0 (40), 513.1 (100). MS (ES−): m/e 513.1 (30), 511.1 (100).

Compound 460: m.p. 143-145° C. ¹H NMR (300 MHz, DMSO-d₆): δ 7.97 (1H, d, J=7.9 Hz), 7.56 (2H, dd, J=8.5, 5.3 Hz), 7.50 (1H, d, J=7.9 Hz), 7.38 (1H, dd, J=7.9, 1.4 Hz), 7.30 (1H, t, J=7.9 Hz), 7.18-7.04 (3H, m), 6.90 (1H, t, J=7.6 Hz), 6.71 (1H, d, J=7.6 Hz), 6.11 (1H, s), 5.29 (2H, s). MS (ES+): m/e 529.0 (100). MS (ES−): m/e 527.1 (100).

Compound 461: m.p. 235-236° C. ¹H NMR (300 MHz, DMSO-d₆): δ 8.14 (1H, s), 7.64-7.57 (3H, m), 7.41-7.24 (3H, m), 6.37 (1H, d, J=3.0 Hz), 6.24 (1H, s), 2.33 (3H, s). MS (ES+): m/e 489.0 (40), 487.1 (100). MS (ES−): m/e 487.2 (30), 485.2 (100).

Compound 462: m.p. 223-224° C. ¹H NMR (300 MHz, DMSO-d₆): δ 7.91 (1H, dd, J=8.8, 2.7 Hz), 7.67-7.56 (3H, m), 7.37-7.19 (3H, m), 6.37 (1H, dd, J=3.5, 0.8 Hz), 6.24 (1H, s), 2.33 (3H, s). MS (ES+): m/e 472.2 (25), 471.1 (100). MS (ES−): m/e 470.2 (20), 469.1 (100).

Compound 463: m.p. 269-270° C. ¹H NMR (300 MHz, DMSO-d₆): δ 7.97 (1H, dd, J=8.0, 1.2 Hz), 7.70 (1H, dd, J=10.2, 2.0 Hz), 7.66 (1H, dd, J=10.2, 2.0 Hz), 7.49 (1H, dd, J=7.9, 0.9 Hz), 7.42-7.25 (3H, m), 6.39 (1H, dd, J=3.5, 0.9 Hz), 2.34 (3H, s). MS (ES+): m/e 489.0 (40), 487.0 (100). MS (ES−): m/e 487.1 (35), 485.0 (100).

Compound 464: m.p. 244-245° C. ¹H NMR (300 MHz, DMSO-d₆): δ 8.14 (1H, d, J=2.4 Hz), 7.65-7.29 (12H, m), 6.35 (1H, dd, J=3.5, 0.4 Hz), 6.32 (1H, s), 2.33 (3H, s). MS (ES+): m/e 529.2 (50), 527.3 (100). MS (ES−): m/e 527.0 (30), 525.1 (100).

Compound 465: m.p. 228-231° C. ¹H NMR (300 MHz, DMSO-d₆): δ 7.91 (1H, dd, J=8.8, 2.6 Hz), 7.66 (1H, dd, J=8.8, 5.0 Hz), 7.60-7.48 (5H, m), 7.40-7.20 (6H, m), 6.36 (1H, d, J=3.2 Hz), 6.32 (1H, s), 2.33 (3H, s). MS (ES+): m/e 512.4 (25), 511.3 (100). MS (ES−): m/e 510.1 (30), 509.0 (100).

Compound 466: m.p. 222-223° C. ¹H NMR (300 MHz, DMSO-d₆): δ 10.04 (1H, s), 7.97 (1H, d, J=7.9 Hz), 7.78-7.25 (12H, m), 6.39 (1H, d, J=3.0 Hz), 6.35 (1H, s), 2.33 (3H, s). MS (ES+): m/e 529.2 (35), 527.3 (100). MS (ES−): m/e 527.2 (45), 525.2 (100).

Compound 467: m.p. 238-239° C. ¹H NMR (300 MHz, DMSO-d₆): δ 8.15 (1H, d, J=2.0 Hz), 7.66 (1H, d, J=8.4 Hz), 7.57 (1H, d, J=3.5 Hz), 7.46-7.40 (3H, m), 7.33 (2H, t, J=7.9 Hz), 7.12 (1H, t, J=7.3 Hz), 6.92 (2H, d, J=7.9 Hz), 6.81 (2H, d, J=8.8 Hz), 6.37 (1H, d, J=3.5 Hz), 6.26 (1H, s), 2.34 (3H, s). MS (ES+): m/e 545.3 (25), 543.3 (100). MS (ES−): m/e 543.0 (40), 541.1 (100).

Compound 468: m.p. 172-173° C. ¹H NMR (300 MHz, DMSO-d₆): δ 7.91 (1H, dd, J=8.8, 2.6 Hz), 7.68 (1H, dd, J=8.8, 4.7 Hz), 7.56 (1H, d, J=3.5 Hz), 7.42 (2H, d, J=8.5 Hz), 7.33 (2H, t, J=7.9 Hz), 7.25 (1H, td, J=8.8, 2.6 Hz), 7.11 (1H, t, J=7.3 Hz), 6.92 (2H, d, J=7.6 Hz), 6.81 (2H, d, J=8.8 Hz), 6.37 (1H, d, J=3.5 Hz), 6.26 (1H, s), 2.34 (3H, s). MS (ES+): m/e 528.4 (25), 527.3 (100). MS (ES−): m/e 526.2 (20), 525.1 (100).

Compound 469: m.p. 208-209° C. ¹H NMR (300 MHz, DMSO-d₆): δ 7.97 (1H, dd, J=8.0, 1.0 Hz), 7.62 (1H, d, J=3.5 Hz), 7.51 (2H, d, J=8.8 Hz), 7.31 (1H, d, J=7.9 Hz), 7.27 (1H, d, J=7.9 Hz), 7.11 (1H, t, J=7.6 Hz), 6.92 (2H, br d, J=8 Hz), 6.82 (2H, d, J=8.5 Hz), 6.39 (1H, dd, J=3.5, 0.9 Hz), 6.29 (1H, s), 2.35 (3H, s). MS (ES+): m/e 545.3 (35), 543.3 (100). MS (ES−): m/e 543.1 (40), 541.0 (100).

Compound 470: m.p. 196-197° C. ¹H NMR (300 MHz, DMSO-d₆): δ 8.14 (1H, d, J=2.0 Hz), 7.65 (1H, d, J=8.8 Hz), 7.55 (1H, d, J=3.5 Hz), 7.50-7.27 (8H, m), 6.86 (2H, d, J=8.5 Hz), 6.36 (1H, d, J=3.5 Hz), 6.24 (1H, s), 4.95 (2H, s), 2.33 (3H, s). MS (ES+): m/e 557.3 (40), 379.3 (100). MS (ES−): m/e 557.0 (40), 555.1 (100).

Compound 471: m.p. 191-193° C. ¹H NMR (300 MHz, DMSO-d₆): δ 7.90 (1H, dd, J=8.5, 2.6 Hz), 7.67 (1H, dd, J=8.9, 4.8 Hz), 7.55 (1H, d, J=3.5 Hz), 7.38-7.21 (8H, m), 6.86 (2H, d, J=8.8 Hz), 6.36 (1H, d, J=3.5 Hz), 6.24 (1H, s), 4.95 (2H, s), 2.34 (3H, s). MS (ES+): m/e 542.4 (25), 541.4 (100). MS (ES−): m/e 540.2 (25), 539.1 (100).

Compound 472: m.p. 240-241° C. ¹H NMR (300 MHz, DMSO-d₆): δ 7.96 (1H, dd, J=7.9, 1.1 Hz), 7.61 (1H, d, J=3.5 Hz), 7.50 (1H, dd, J=7.9, 2.2 Hz), 7.45 (2H, d, J=8.8 Hz), 7.38-7.25 (6H, m), 6.86 (2H, d, J=8.8 Hz), 6.38 (1H, dd, J=3.5, 0.8 Hz), 6.27 (1H, s), 4.97 (1H, d, J=11.7 Hz), 4.93 (1H, d, J=11.7 Hz), 2.34 (3H, s). MS (ES+): m/e 559.3 (45), 557.3 (100). MS (ES−): m/e 557.0 (35), 555.1 (100).

Compound 473: m.p. 226-227° C. ¹H NMR (300 MHz, DMSO-d₆): δ 8.16 (1H, d, J=2.3 Hz), 7.60 (1H, d, J=8.8 Hz), 7.52 (1H, d, J=3.5 Hz), 7.44 (1H, dd, J=8.8, 2.0 Hz), 7.32-7.22 (3H, m), 7.15-7.08 (3H, m), 6.86-6.80 (3H, m), 6.38 (1H, dd, J=3.5, 0.9 Hz), 6.24 (1H, s), 2.33 (3H, s). MS (ES+): m/e 545.2 (45), 543.3 (100). MS (ES−): m/e 543.0 (45), 541.1 (100).

Compound 475: m.p. 213-214° C. ¹H NMR (300 MHz, DMSO-d₆): δ 7.92 (1H, dd, J=8.8, 2.6 Hz), 7.62 (1H, dd, J=8.9, 5.0 Hz), 7.52 (1H, d, J=3.5 Hz), 7.32-7.22 (4H, m), 7.16-7.08 (3H, m), 6.86-6.80 (3H, m), 6.38 (1H, dd, J=3.5, 0.9 Hz), 6.24 (1H, s), 2.33 (3H, s). MS (ES+): m/e 528.4 (25), 527.3 (100). MS (ES−): m/e 526.2 (25), 525.1 (100).

Compound 475: m.p. 215-216° C. ¹H NMR (300 MHz, DMSO-d₆): δ 8.14 (1H, d, J=2.0 Hz), 7.62 (1H, d, J=8.8 Hz), 7.53 (1H, d, J=3.5 Hz), 7.42-7.29 (6H, m), 7.15 (1H, t, J=7.9 Hz), 7.05-6.96 (2H, m), 6.82 (1H, dd, J=8.2, 2.0 Hz), 6.36 (1H, d, J=3.5 Hz), 6.24 (1H, s), 5.01 (1H, d, J=12.0 Hz), 4.96 (1H, d, J=12.0 Hz), 2.33 (3H, s). MS (ES+): m/e 559.2 (45), 557.3 (100). MS (ES−): m/e 557.0 (30), 555.0 (100).

Compound 476: m.p. 187-188° C. ¹H NMR (300 MHz, DMSO-d₆): δ 7.91 (1H, dd, J=8.8, 2.6 Hz), 7.64 (1H, dd, J=8.8, 5.0 Hz), 7.52 (1H, d, J=3.5 Hz), 7.39-7.20 (6H, m), 7.15 (1H, t, J=7.9 Hz), 7.04-6.97 (2H, m), 6.82 (1H, dd, J=7.9, 2.6 Hz), 6.36 (1H, d, J=3.5 Hz), 6.24 (1H, s), 5.02 (1H, d, J=12.0 Hz), 4.96 (1H, d, J=12.0 Hz), 2.33 (3H, s). MS (ES+): m/e 542.4 (30), 541.3 (100). MS (ES−): m/e 540.2 (30), 539.1 (100).

Compound 477: m.p. 209-210° C. ¹H NMR (300 MHz, DMSO-d₆): δ 8.14 (1H, d, J=2.2 Hz), 7.64 (1H, d, J=8.8 Hz), 7.54 (1H, d, J=3.5 Hz), 7.40 (1H, dd, J=8.8, 2.2 Hz), 7.32 (2H, d, J=8.8 Hz), 6.77 (2H, d, J=8.8 Hz), 6.36 (1H, d, J=3.5 Hz), 6.23 (1H, s), 3.85 (2H, narrow m), 3.60 (2H, narrow m), 2.33 (3H, s). MS (ES+): m/e 513.3 (40), 511.3 (100). MS (ES−): m/e 511.0 (40), 509.0 (100).

Compound 478: m.p. 134-135° C. ¹H NMR (300 MHz, DMSO-d₆): δ 7.90 (1H, dd, J=8.8, 2.6 Hz), 7.66 (1H, dd, J=8.8, 4.7 Hz), 7.53 (1H, d, J=3.5 Hz), 7.32 (2H, d, J=8.8 Hz), 7.23 (1H, td, J=9.0, 2.6 Hz), 6.77 (2H, d, J=8.8 Hz), 6.36 (1H, d, J=3.5 Hz), 6.23 (1H, s), 3.85 (2H, td, J=6.7, 2.1 Hz), 3.60 (2H, t, J=5.0 Hz), 2.33 (3H, s). MS (ES+): m/e 496.4 (25), 495.3 (100). MS (ES−): m/e 494.1 (25), 493.1 (100).

Compound 479: m.p. 211-213° C. ¹H NMR (300 MHz, DMSO-d₆): δ 8.62 (1H, d, J=3.2 Hz), 8.07 (1H, d, J=2.0 Hz), 7.61 (1H, d, J=8.8 Hz), 7.36 (1H, dd, J=8.8, 2.0 Hz), 7.26 (2H, d, J=8.5 Hz), 6.72 (2H, d, J=8.5 Hz), 6.13 (1H, d, J=3.2 Hz), 6.01 (1H, s), 3.91 (2H, narrow m), 3.14 (2H, t, J=7.9 Hz), 2.74 (6H, s), 2.24 (3H, s), 2.07-1.97 (2H, m). MS (ES+): m/e 554.3 (40), 552.4 (100). MS (ES−): m/e 552.1 (40), 550.1 (100).

Compound 480: m.p. 209-211° C. ¹H NMR (300 MHz, DMSO-d₆): δ 8.63 (1H, d, J=3.2 Hz), 7.84 (1H, dd, J=8.8, 2.4 Hz), 7.63 (1H, dd, J=8.8, 4.7 Hz), 7.26 (2H, d, J=8.8 Hz), 7.25-7.16 (1H, m), 6.72 (2H, d, J=8.8 Hz), 6.13 (1H, d, J=3.2 Hz), 6.00 (1H, s), 3.92 (2H, narrow m), 3.13-3.07 (2H, m), 2.73 (6H, s), 2.24 (3H, s), 2.06-1.97 (2H, m). MS (ES+): m/e 537.5 (30), 536.4 (100). MS (ES−): m/e 535.2 (30), 534.1 (100).

Compound 481: m.p. 186-188° C. ¹H NMR (300 MHz, DMSO-d₆): δ 8.13 (1H, d, J=2.0 Hz), 7.56 (1H, d, J=8.5 Hz), 7.38 (1H, dd, J=8.8, 2.0 Hz), 7.30-6.78 (8H, m), 6.26 (1H, s), 4.10 (1H, d, J=17.5 Hz), 3.99 (1H, d, J=17.5 Hz), 3.51 (3H, s). MS (ES+): m/e 509.3 (100). MS (ES−): m/e 509.0 (40), 507.0 (100).

Compound 482: m.p. 207-208° C. ¹H NMR (300 MHz, DMSO-d₆): δ 7.90 (1H, dd, J=8.8, 2.8 Hz), 7.58 (1H, dd, J=8.8, 4.7 Hz), 7.30-6.75 (9H, m), 6.26 (1H, s), 4.10 (1H, d, J=17.2 Hz), 3.98 (1H, d, J=17.2 Hz), 3.51 (3H, s). MS (ES+): m/e 494.5 (25), 493.4 (100). MS (ES−): m/e 492.1 (25), 491.1 (100).

Compound 483: m.p. 224-226° C. ¹H NMR (300 MHz, DMSO-d₆): δ 7.96 (1H, dd, J=7.9, 0.9 Hz), 7.46 (1H, dd, J=7.9, 0.9 Hz), 7.30-6.80 (9H, m), 6.31 (1H, s), 4.11 (1H, d, J=17.3 Hz), 3.99 (1H, d, J=17.3 Hz), 3.52 (3H, s). MS (ES+): m/e 511.2 (45), 509.3 (100). MS (ES−): m/e 509.0 (35), 507.0 (100).

Compound 484: m.p. 253-255° C. ¹H NMR (300 MHz, DMSO-d₆): δ 8.14 (1H, d, J=2.0 Hz), 7.62 (1H, d, J=8.5 Hz), 7.45-7.37 (3H, m), 7.16 (1H, t, J=7.9 Hz), 7.06 (2H, t, J=8.8 Hz), 6.96 (1H, d, J=7.3 Hz), 6.85 (1H, d, J=8.2 Hz), 6.79 (1H, t, J=7.3 Hz), 6.06 (1H, s), 4.09 (1H, d, J=17.2 Hz), 3.97 (1H, d, J=17.2 Hz), 3.51 (3H, s). MS (ES+): m/e 509.3 (100). MS (ES−): m/e 509.0 (35), 507.0 (100).

Compound 485: m.p. 223-224° C. ¹H NMR (300 MHz, DMSO-d₆): δ 7.90 (1H, dd, J=8.8, 2.7 Hz), 7.64 (1H, dd, J=8.8, 4.7 Hz), 7.41 (2H, dd, J=8.8, 5.6 Hz), 7.23 (1H, td, J=9.0, 2.7 Hz), 7.17 (1H, t, J=7.9 Hz), 7.06 (2H, t, J=8.7 Hz), 6.96 (1H, d, J=7.3 Hz), 6.85 (1H, d, J=8.2 Hz), 6.79 (1H, t, J=7.5 Hz), 6.05 (1H, s), 4.09 (1H, d, J=17.1 Hz), 3.97 (1H, d, J=17.1 Hz), 3.51 (3H, s). MS (ES+): m/e 494.5 (20), 493.3 (100). MS (ES−): m/e 492.2 (25), 491.0 (100).

Compound 486: m.p. 243-245° C. ¹H NMR (300 MHz, DMSO-d₆): δ 7.96 (1H, dd, J=8.0, 1.0 Hz), 7.52-7.42 (2H, m), 7.29 (1H, t, J=7.9 Hz), 7.17 (1H, td, J=8.2, 1.6 Hz), 7.07 (2H, t, J=8.9 Hz), 6.98 (1H, dd, J=7.4, 1.5 Hz), 6.86 (1H, d, J=8.2 Hz), 6.80 (1H, t, J=7.2 Hz), 6.07 (1H, s), 4.12 (1H, d, J=17.5 Hz), 3.98 (1H, d, J=17.5 Hz), 3.54 (3H, s). MS (ES+): m/e 509.2 (100). MS (ES−): m/e 508.9 (45), 507.0 (100).

Compound 487: m.p. 247-248° C. ¹H NMR (300 MHz, DMSO-d₆): δ 8.15 (1H, s), 7.62 (1H, d, J=8.5 Hz), 7.40 (2H, d, J=8.5 Hz), 7.39-7.36 (1H, m), 7.29 (2H, d, J=8.5 Hz), 7.17 (1H, t, J=7.6 Hz), 6.96 (1H, d, J=7.3 Hz), 6.85 (1H, d, J=8.2 Hz), 6.79 (1H, t, J=7.3 Hz), 6.04 (1H, s), 4.08 (1H, d, J=17.2 Hz), 3.98 (1H, d, J=17.2 Hz), 3.51 (3H, s). MS (ES+): m/e 529.1 (20), 527.1 (70), 525.2 (100). MS (ES−): m/e 526.0 (15), 524.9 (75), 523.0 (100).

Compound 488: m.p. 249-250° C. ¹H NMR (300 MHz, DMSO-d₆): δ 7.90 (1H, dd, J=8.8, 2.6 Hz), 7.64 (1H, dd, J=9.0, 4.7 Hz), 7.40 (2H, d, J=8.5 Hz), 7.29 (2H, d, J=8.5 Hz), 7.27-7.13 (2H, m), 6.96 (1H, dd, J=7.3, 1.4 Hz), 6.85 (1H, d, J=8.2 Hz), 6.79 (1H, t, J=7.3 Hz), 6.04 (1H, s), 4.08 (1H, d, J=17.2 Hz), 3.98 (1H, d, J=17.2 Hz), 3.51 (3H, s). MS (ES−): m/e 509.0 (45), 507.0 (100).

Compound 489: m.p. 176-178° C. ¹H NMR (300 MHz, DMSO-d₆): δ 7.97 (1H, dd, J=7.6, 0.6 Hz), 7.55-7.49 (1H, m), 7.49 (2H, d, J=9.0 Hz), 7.30 (2H, d, J=9.0 Hz), 7.29-7.25 (1H, m), 7.20-7.14 (1H, m), 7.00-6.80 (3H, m), 6.06 (1H, s), 4.11 (1H, d, J=17.2 Hz), 3.99 (1H, d, J=17.2 Hz), 3.54 (3H, s). MS (ES−): m/e 526.9 (15), 525.0 (70), 523.0 (100).

Compound 490: m.p. 146-148° C. ¹H NMR (300 MHz, DMSO-d₆): δ 7.50 (1H, d, J=3.5 Hz), 7.29 (2H, dd, J=8.6, 5.4 Hz), 7.11 (2H, t, J=8.7 Hz), 6.73 (1H, br), 6.31 (1H, d, J=3.5 Hz), 5.51 (1H, s), 3.31 (4H, m), 3.00 (2H, m), 2.84 (2H, m), 2.29 (3H, s), 1.61 (2H, m), 1.48 (2H, m), 1.35 (9H, s), 1.30 (9H, s). MS (ES+): m/e 616.5 (60), 516.4 (100). MS (ES−): m/e 615.3 (35), 614.3 (100).

Compound 491: m.p. 126-128° C. ¹H NMR (300 MHz, DMSO-d₆): δ 9.09 (2H, br), 8.11 (3H, br), 7.51 (1H, d, J=3.5 Hz), 7.37-7.30 (2H, m), 7.13 (2H, t, J=8.7 Hz), 6.32 (1H, d, J=3.5 Hz), 5.58 (1H, s), 3.50-2.50 (8H, br), 2.30 (3H, s), 2.00-1.78 (4H, br). MS (ES+): m/e 416.5 (100). MS (ES−): m/e 415.2 (25), 414.2 (100).

Compound 493: m.p. 206-207° C. ¹H NMR (300 MHz, DMSO-d₆): δ 8.13 (1H, d, J=2.0 Hz), 7.56 (1H, d, J=9.0 Hz), 7.38 (1H, dd, J=9.0, 2.0 Hz), 7.30-6.98 (4H, m), 6.24 (1H, s), 3.94 (2H, q, J=7.0 Hz), 3.20-3.06 (1H, m), 3.03-2.89 (1H, m), 2.50 (2H, obscured), 1.07 (3H, t, J=7.0 Hz). MS (ES+): m/e 491.1 (40), 489.2 (100). MS (ES−): m/e 489.1 (40), 487.2 (100).

Compound 494: m.p. 224-225° C. ¹H NMR (300 MHz, DMSO-d₆): δ 8.13 (1H, d, J=2.0 Hz), 7.63 (1H, d, J=8.8 Hz), 7.47-7.38 (3H, m), 7.05 (2H, t, J=8.6 Hz), 6.02 (1H, s), 3.94 (2H, q, J=7.0 Hz), 3.20-3.05 (1H, m), 3.01-2.86 (1H, m), 2.50 (2H, obscured), 1.07 (3H, t, J=7.0 Hz). MS (ES+): m/e 491.1 (40), 489.2 (100). MS (ES−): m/e 489.2 (35), 487.2 (100).

Compound 495: m.p. 244-245° C. ¹H NMR (300 MHz, DMSO-d₆): δ 8.13 (1H, s), 7.62 (1H, d, J=8.5 Hz), 7.41 (1H, d, J=8.5 Hz), 7.40 (2H, d, J=8.2 Hz), 7.28 (2H, d, J=8.2 Hz), 6.01 (1H, s), 3.94 (2H, q, J=7.0 Hz), 3.20-3.05 (1H, m), 3.02-2.87 (1H, m), 2.50 (2H, obscured), 1.06 (3H, t, J=7.0 Hz). MS (ES+): m/e 509.1 (20), 507.1 (80), 505.2 (100). MS (ES−): m/e 506.3 (20), 504.2 (75), 503.2 (100).

Compound 496: m.p. 184-185° C. ¹H NMR (300 MHz, DMSO-d₆): δ 7.89 (1H, dd, J=8.8, 2.6 Hz), 7.58 (1H, dd, J=8.8, 4.7 Hz), 7.30-7.00 (5H, m), 6.23 (1H, s), 3.94 (2H, q, J=7.0 Hz), 3.20-3.05 (1H, m), 3.02-2.87 (1H, m), 2.44 (2H, t, J=6.7 Hz), 1.07 (3H, t, J=7.0 Hz). MS (ES+): m/e 474.4 (25), 473.3 (100). MS (ES−): m/e 472.4 (20), 471.2 (100).

Compound 497: m.p. 201-202° C. ¹H NMR (300 MHz, DMSO-d₆): δ 7.89 (1H, dd, J=8.8, 2.6 Hz), 7.64 (1H, dd, J=8.8, 4.7 Hz), 7.42 (2H, dd, J=8.5, 5.6 Hz), 7.23 (1H, td, J=8.8, 2.6 Hz), 7.05 (2H, t, J=8.8 Hz), 6.02 (1H, s), 3.94 (2H, q, J=7.0 Hz), 3.11 (1H, dt, J=18.7, 6.6 Hz), 2.94 (1H, dt, J=18.7, 6.6 Hz), 2.43 (2H, t, J=6.6 Hz), 1.07 (3H, t, J=8.8 Hz). MS (ES+): m/e 474.4 (20), 473.2 (100). MS (ES−): m/e 472.4 (20), 471.2 (100).

Compound 498: m.p. 219-220° C. ¹H NMR (300 MHz, DMSO-d₆): δ 7.89 (1H, dd, J=8.8, 2.6 Hz), 7.64 (1H, dd, J=8.8, 4.7 Hz), 7.41 (2H, d, J=8.5 Hz), 7.28 (2H, d, J=8.5 Hz), 7.23 (1H, td, J=9.0, 2.6 Hz), 6.01 (1H, s), 3.94 (2H, q, J=7.0 Hz), 3.11 (1H, dt, J=18.7, 6.6 Hz), 2.94 (1H, dt, J=18.7, 6.6 Hz), 2.43 (2H, t, J=6.6 Hz), 1.07 (3H, t, J=7.0 Hz). MS (ES+): m/e 491.1 (40), 489.2 (100). MS (ES−): m/e 489.2 (40), 487.2 (100).

Compound 499: m.p. 180-182° C. ¹H NMR (300 MHz, DMSO-d₆): δ 7.95 (1H, dd, J=8.9, 0.9 Hz), 7.46 (1H, dd, J=7.9, 0.8 Hz), 7.30-6.98 (5H, m), 6.27 (1H, s), 3.95 (2H, q, J=7.0 Hz), 3.13 (1H, dt, J=19.0, 6.7 Hz), 3.00 (1H, dt, J=19.0, 6.7 Hz), 2.45 (2H, t, J=6.5 Hz), 1.07 (3H, t, J=7.0 Hz). MS (ES+): m/e 491.1 (45), 489.2 (100). MS (ES−): m/e 489.2 (35), 487.1 (100).

Compound 500: m.p. 178-179° C. ¹H NMR (300 MHz, DMSO-d₆): δ 7.95 (1H, dd, J=7.9, 0.8 Hz), 7.54-7.44 (3H, m), 7.28 (1H, t, J=7.9 Hz), 7.06 (2H, t, J=8.8 Hz), 6.04 (1H, s), 3.95 (2H, q, J=7.0 Hz), 3.12 (1H, dt, J=19.0, 6.7 Hz), 2.98 (1H, dt, J=19.0, 6.7 Hz), 2.44 (2H, t, J=6.7 Hz), 1.08 (3H, t, J=7.0 Hz). MS (ES+): m/e 491.1 (45), 489.2 (100). MS (ES−): m/e 489.2 (35), 487.2 (100).

Compound 501: m.p. 215-216° C. ¹H NMR (300 MHz, DMSO-d₆): δ 7.96 (1H, dd, J=7.9, 0.9 Hz), 7.50-7.43 (3H, m), 7.30-7.23 (3H, m), 6.02 (1H, s), 3.95 (2H, q, J=7.0 Hz), 3.12 (1H, dt, J=18.7, 6.7 Hz), 2.97 (1H, dt, J=18.7, 6.7 Hz), 2.44 (2H, t, J=6.7 Hz), 1.07 (3H, t, J=7.0 Hz). MS (ES+): m/e 506.2 (75), 505.2 (100). MS (ES−): m/e 507.2 (20), 505.2 (80), 503.2 (100).

Compound 502: m.p. 177-178° C. ¹H NMR (300 MHz, DMSO-d₆): δ 7.53-7.49 (2H, m), 7.33-7.25 (1H, m), 7.18-7.06 (3H, m), 6.34-6.30 (2H, m), 6.13 (1H, d, J=3.2 Hz), 5.55 (1H, s), 4.76 (1H, d, J=16.1 Hz), 3.86 (1H, d, J=16.1 Hz), 2.28 (3H, s). MS (ES+): m/e 382.2 (100). MS (ES−): m/e 381.3 (20), 380.2 (100).

Compound 503: m.p. 213-214° C. ¹H NMR (300 MHz, DMSO-d₆): δ 7.54 (1H, t, J=1.0 Hz), 7.48 (1H, d, J=3.5 Hz), 7.22 (2H, dd, J=8.8, 5.4 Hz), 7.09 (2H, t, J=8.8 Hz), 6.34 (1H, dd, J=3.1, 1.9 Hz), 6.30 (1H, dd, J=3.5, 1.8 Hz), 6.15 (1H, d, J=3.2 Hz), 5.26 (1H, s), 4.76 (1H, d, J=15.8 Hz), 3.83 (1H, d, J=15.8 Hz), 2.28 (3H, s). MS (ES+): m/e 382.2 (100). MS (ES−): m/e 381.4 (20), 380.2 (100).

Compound 504: m.p. 193-194° C. ¹H NMR (300 MHz, DMSO-d₆): δ 7.51 (1H, d, J=3.5 Hz), 7.42 (1H, dd, J=5.0, 0.9 Hz), 7.36-7.29 (1H, m), 7.21-7.04 (3H, m), 6.91 (1H, dd, J=4.9, 3.5 Hz), 6.80 (1H, d, J=3.2 Hz), 6.32 (1H, d, J=3.2 Hz), 5.58 (1H, s), 4.90 (1H, d, J=15.8 Hz), 4.04 (1H, d, J=15.8 Hz), 2.28 (3H, s). MS (ES+): m/e 398.2 (100). MS (ES−): m/e 397.3 (20), 396.2 (100).

Compound 505: m.p. 225-226° C. ¹H NMR (300 MHz, DMSO-d₆): δ 7.49 (1H, d, J=3.5 Hz), 7.42 (1H, dd, J=5.0, 1.0 Hz), 7.25 (2H, dd, J=8.8, 5.4 Hz), 7.10 (2H, t, J=8.8 Hz), 6.91 (1H, dd, J=5.0, 3.5 Hz), 6.81 (1H, d, J=3.5 Hz), 5.29 (1H, s), 4.89 (1H, d, J=15.8 Hz), 4.02 (1H, d, J=15.8 Hz), 2.28 (3H, s). MS (ES+): m/e 398.2 (100). MS (ES−): m/e 397.3 (15), 396.2 (100).

Compound 564: m.p. 231-232° C. ¹H NMR (300 MHz, DMSO-d₆): δ 9.32 (1H, s), 8.35 (1H, d, J=2.0 Hz), 8.02 (1H, d, J=8.8 Hz), 7.69 (1H, dd, J=8.8, 2.0 Hz), 7.57 (1H, d, J=3.2 Hz), 7.39 (1H, br t, J=6.9 Hz), 7.17-7.09 (1H, m), 7.03-6.94 (2H, m), 6.57 (1H, s), 6.36 (1H, dd, J=3.8, 1.1 Hz), 2.32 (3H, s). MS (ES+): m/e 436.2 (20), 435.1 (100). MS (ES−): m/e 433.8 (25), 432.8 (100).

Compound 565: m.p. 221-222° C. ¹H NMR (300 MHz, DMSO-d₆): δ 9.32 (1H, s), 8.40 (1H, d, J=2.0 Hz), 7.99 (1H, d, J=9.0 Hz), 7.78 (1H, dd, J=9.0, 2.0 Hz), 7.54 (1H, d, J=3.5 Hz), 7.39 (2H, dd, J=8.8, 5.5 Hz), 6.97 (2H, t, J=8.8 Hz), 6.41 (1H, s), 6.35 (1H, d, J=3.5 Hz), 2.32 (3H, s). MS (ES+): m/e 436.1 (25), 435.2 (100). MS (ES−): m/e 433.9 (25), 432.9 (100).

Compound 566: m.p. 91-93° C. ¹H NMR (300 MHz, DMSO-d₆): δ 7.80-7.70 (2H, m), 7.57-7.47 (3H, m), 7.40-7.00 (4H, m), 6.37 (1H, d, J=3.5 Hz), 5.96 (1H, s), 5.13 (1H, d, J=14 Hz), 4.64 (1H, d, J=14 Hz), 2.32 (3H, s). MS (ES+): m/e 433.2 (25), 432.2 (100). MS (ES−): m/e 430.9 (25), 429.9 (100).

Compound 567: m.p. 96-100° C. ¹H NMR (300 MHz, DMSO-d₆): δ 8.98 (1H, br), 7.80-7.70 (2H, m), 7.56-7.46 (2H, m), 7.42 (1H, d, J=3.5 Hz), 7.39-7.00 (4H, m), 6.30 (1H, d, J=3.5 Hz), 5.80 (1H, s), 5.14 (1H, d, J=14 Hz), 4.61 (1H, d, J=14 Hz), 2.32 (3H, s). MS (ES+): m/e 433.2 (25), 432.2 (100). MS (ES−): m/e 430.8 (25), 429.9 (100).

Compound 568: m.p. 258-260° C. ¹H NMR (300 MHz, DMSO-d₆): δ 8.12 (1H, d, J=2.0 Hz), 7.56 (1H, d, J=8.8 Hz), 7.38 (1H, dd, J=8.8, 2.0 Hz), 7.27-7.18 (2H, m), 7.11 (1H, br t, J=9.4 Hz), 7.02 (1H, t, J=7.4 Hz), 6.24 (1H, s), 3.23-3.08 (1H, m), 3.00-2.92 (1H, m), 2.37 (2H, t, J=6.5 Hz). MS (ES+): m/e 463.0 (40), 461.0 (100). MS (ES−): m/e 460.8 (35), 458.8 (100).

Compound 569: m.p. 262-263° C. ¹H NMR (300 MHz, DMSO-d₆): δ 8.13 (1H, d, J=2.0 Hz), 7.63 (1H, d, J=8.5 Hz), 7.45-7.37 (3H, m), 7.05 (2H, t, J=8.9 Hz), 6.02 (1H, s), 3.09 (1H, dt, J=19.0, 6.6 Hz), 2.90 (1H, dt, J=19.0, 6.6 Hz), 2.37 (2H, t, J=6.6 Hz). MS (ES+): m/e 463.0 (35), 461.0 (100). MS (ES−): m/e 460.8 (40), 458.8 (100).

Compound 570: m.p. 216-218° C. ¹H NMR (300 MHz, DMSO-d₆): δ 7.96 (1H, d, J=7.9 Hz), 7.54-7.44 (3H, m), 7.28 (1H, t, J=7.9 Hz), 7.11-7.00 (2H, m), 6.19-6.09 (2H, m), 6.06 (0.7H, s), 6.01 (0.3H, s), 3.80-1.00 (7H, m). MS (ES+): m/e 483.1 (35), 481.1 (100). MS (ES−): m/e 480.8 (35), 478.8 (100).

Compound 571: m.p. 262-263° C. ¹H NMR (300 MHz, DMSO-d₆): δ 8.12 (1H, d, J=2.0 Hz), 7.56 (1H, d, J=8.5 Hz), 7.38 (1H, dd, J=8.5, 2.0 Hz), 7.27-7.01 (4H, m), 6.25 (1H, s), 3.88-3.76 (1H, m), 2.18-2.00 (3H, m), 1.94-1.80 (2H, m), 1.69-1.58 (1H, m). MS (ES+): m/e 445.1 (35), 443.1 (100). MS (ES−): m/e 442.9 (40), 440.9 (100).

Compound 572: m.p. 271-273° C. ¹H NMR (300 MHz, DMSO-d₆): δ 8.12 (1H, d, J=2.0 Hz), 7.62 (1H, d, J=8.5 Hz), 7.46-7.36 (3H, m), 7.06 (2H, t, J=8.8 Hz), 6.03 (1H, s), 3.85-3.75 (1H, m), 2.14-1.99 (3H, m), 1.95-1 (78, 2H, m), 1.69-1.54 (1H, m). MS (ES+): m/e 445.0 (40), 443.1 (100). MS (ES−): m/e 442.9 (40), 440.9 (100).

Compound 573: m.p. 220-221° C. ¹H NMR (300 MHz, DMSO-d₆): δ 7.94 (1H, dd, J=7.9, 0.9 Hz), 7.46 (1H, dd, J=7.9, 0.9 Hz), 7.27 (1H, t, J=7.9 Hz), 7.24-7.10 (3H, m), 7.01 (1H, t, J=7.5 Hz), 6.29 (1H, s), 3.87-3.78 (1H, m), 2.15-2.03 (3H, m), 1.95-1.81 (2H, m), 1.68-1.59 (1H, m). MS (ES+): m/e 445.1 (35), 443.1 (100). MS (ES−): m/e 442.9 (35), 440.8 (100).

Compound 574: m.p. 232-233° C. ¹H NMR (300 MHz, DMSO-d₆): δ 7.94 (1H, dd, J=7.9, 1.1 Hz), 7.50-7.43 (3H, m), 7.27 (1H, t, J=7.9 Hz), 7.07 (2H, t, J=8.8 Hz), 6.05 (1H, s), 3.89-3.77 (1H, m), 2.20-2.10 (1H, m), 2.09-2.00 (2H, m), 1.97-1.83 (2H, m), 1.70-1.59 (1H, m). MS (ES+): m/e 445.0 (40), 443.1 (100). MS (ES−): m/e 442.9 (35), 440.8 (100).

Compound 575: m.p. 219-220° C. ¹H NMR (300 MHz, DMSO-d₆): δ 8.12 (1H, d, J=2.3 Hz), 7.56 (1H, d, J=8.5 Hz), 7.38 (1H, dd, J=8.5, 2.3 Hz), 7.28-7.04 (4H, m), 7.00 (2H, d, J=8.5 Hz), 6.76 (2H, d, J=8.5 Hz), 6.24 (1H, s), 4.00 (2H, t, J=4.5 Hz), 3.60 (2H, t, J=4.5 Hz), 3.27 (3H, s), 3.00 (2H, t, J=7.4 Hz), 2.66 (2H, t, J=7.4 Hz). MS (ES+): m/e 569.1 (35), 567.1 (100). MS (ES−): m/e 567.0 (45), 564.9 (100).

Compound 576: m.p. 226-227° C. ¹H NMR (300 MHz, DMSO-d₆): δ 8.13 (1H, d, J=2.0 Hz), 7.62 (1H, d, J=8.8 Hz), 7.45-7.37 (3H, m), 7.06 (2H, t, J=8.9 Hz), 6.98 (2H, d, J=8.4 Hz), 6.75 (2H, d, J=8.4 Hz), 6.03 (1H, s), 4.00 (2H, t, J=4.7 Hz), 3.60 (2H, t, J=4.7 Hz), 3.27 (3H, s), 2.99 (2H, t, J=7.3 Hz), 2.66 (2H, t, J=7.3 Hz). MS (ES+): m/e 569.1 (45), 567.2 (100). MS (ES−): m/e 566.9 (45), 564.9 (100).

Compound 577: m.p. 179-180° C. ¹H NMR (300 MHz, DMSO-d₆): δ 7.94 (1H, dd, J=7.9, 1.1 Hz), 7.45 (1H, dd, J=7.9, 1.1 Hz), 7.27 (1H, t, J=7.9 Hz), 7.25-7.00 (4H, m), 7.01 (2H, d, J=8.5 Hz), 6.76 (2H, d, J=8.5 Hz), 6.29 (1H, s), 4.02-3.98 (2H, m), 3.62-3.58 (2H, m), 3.27 (3H, s), 3.02 (2H, t, J=7.6 Hz), 2.67 (2H, t, J=7.6 Hz). MS (ES+): m/e 569.1 (40), 567.2 (100). MS (ES−): m/e 566.9 (40), 564.9 (100).

Compound 578: m.p. 182-183° C. ¹H NMR (300 MHz, DMSO-d₆): δ 7.95 (1H, dd, J=8.0, 1.2 Hz), 7.50-7.44 (3H, m), 7.28 (1H, t, J=7.9 Hz), 7.06 (2H, t, J=8.9 Hz), 7.01 (2H, d, J=8.5 Hz), 6.76 (2H, d, J=8.5 Hz), 6.05 (1H, s), 4.02-3.98 (2H, m), 3.62-3.59 (2H, m), 3.27 (3H, s), 3.05-2.99 (2H, m), 2.68 (2H, t, J=7.6 Hz). MS (ES+): m/e 569.1 (45), 567.1 (100). MS (ES−): m/e 566.8 (45), 564.9 (100).

Compound 579: m.p. 205-206° C. ¹H NMR (300 MHz, DMSO-d₆): δ 8.12 (0.7H, d, J=2.3 Hz), 7.75 (0.3H, d, J=2.0 Hz), 7.62-7.00 (6H, m), 6.26-5.56 (3H, m), 3.80-0.85 (7H, m). MS (ES+): m/e 483.1 (40), 481.1 (100). MS (ES−): m/e 480.8 (35), 478.9 (100).

Compound 593: m.p. 197-198° C. ¹H NMR (300 MHz, DMSO-d₆): δ 10.81 (1H, d, J=0.6 Hz), 7.48 (1H, d, J=3.5 Hz), 7.35-7.07 (7H, m), 7.03 (1H, td, J=7.3, 0.9 Hz), 6.91 (1H, td, J=7.9, 1.1 Hz), 6.32 (1H, dd, J=3.5, 0.9 Hz), 5.69 (1H, br s), 3.85-3.75 (1H, m), 3.00-2.90 (2H, m), 2.75-2.67 (1H, m), 2.28 (3H, s). MS (ES+): m/e 446.3 (25), 445.3 (100). MS (ES−): m/e 444.3 (20), 443.3 (100).

Compound 594: m.p. 157-158° C. ¹H NMR (300 MHz, DMSO-d₆): δ 10.82 (1H, br), 7.45 (1H, d, J=3.5 Hz), 7.31 (2H, d, J=8.8 Hz), 7.24-7.02 (6H, m), 6.92 (1H, t, J=7.5 Hz), 6.29 (1H, dd, J=3.5, 2.6 Hz), 5.39 (1H, s), 3.88-3.78 (1H, m), 3.00-2.84 (2H, m), 2.77-2.67 (1H, m), 2.28 (3H, s). MS (ES+): m/e 445.3 (100). MS (ES−): m/e 444.4 (20), 443.3 (100).

Compound 595: m.p. 165-166° C. ¹H NMR (300 MHz, DMSO-d₆): δ 10.82 (1H, s), 7.47 (1H, d, J=3.5 Hz), 7.35-7.28 (4H, m), 7.17 (2H, d, J=8.5 Hz), 7.10 (1H, d, J=2.0 Hz), 7.04 (1H, td, J=8.2, 1.2 Hz), 6.95-6.89 (1H, m), 6.30 (1H, dd, J=3.5, 0.9 Hz), 5.38 (1H, s), 3.87-3.77 (1H, m), 3.00-2.84 (2H, m), 2.78-2.68 (1H, m), 2.28 (3H, s). MS (ES+): m/e 463.1 (40), 461.2 (100). MS (ES−): m/e 461.2 (40), 459.2 (100).

Compound 596: m.p. 236-237° C. ¹H NMR (300 MHz, DMSO-d₆): δ 8.14 (1H, d, J=2.0 Hz), 7.67 (1H, d, J=8.8 Hz), 7.57 (1H, d, J=3.5 Hz), 7.41 (1H, dd, J=8.8, 2.0 Hz), 6.88-6.84 (2H, m), 6.68 (1H, d, J=8.5 Hz), 6.37 (1H, d, J=3.5 Hz), 6.17 (1H, s), 4.12 (4H, s), 2.33 (3H, s). MS (ES+): m/e 511.0 (45), 509.1 (100). MS (ES−): m/e 509.1 (45), 507.1 (100).

Compound 597: m.p. 236-237° C. ¹H NMR (300 MHz, DMSO-d₆): δ 7.90 (1H, dd, J=8.8, 2.6 Hz), 7.69 (1H, dd, J=8.8, 4.9 Hz), 7.56 (1H, d, J=3.5 Hz), 7.24 (1H, td, J=8.8, 2.6 Hz), 6.88-6.84 (2H, m), 6.69 (1H, d, J=8.8 Hz), 6.37 (1H, d, J=3.5 Hz), 6.17 (1H, s), 4.12 (4H, s), 2.34 (3H, s). MS (ES+): m/e 494.3 (20), 493.1 (100). MS (ES−): m/e 492.4 (20), 491.2 (100).

Compound 598: m.p. 251-252° C. ¹H NMR (300 MHz, DMSO-d₆): δ 7.96 (1H, dd, J=8.0, 1.0 Hz), 7.62 (1H, d, J=3.5 Hz), 7.50 (1H, dd, J=7.9, 1.2 Hz), 7.29 (1H, t, J=7.9 Hz), 7.00-6.94 (2H, m), 6.68 (1H, d, J=8.5 Hz), 6.39 (1H, dd, J=3.5, 1.0 Hz), 6.20 (1H, s), 4.12 (4H, s), 2.34 (3H, s). MS (ES+): m/e 511.0 (45), 509.1 (100). MS (ES−): m/e 509.1 (40), 507.2 (100).

Compound 599: m.p. 255-257° C. ¹H NMR (300 MHz, DMSO-d₆): δ 8.11 (1H, d, J=2.0 Hz), 8.02 (1H, s), 7.92-7.87 (1H, m), 7.80-7.73 (2H, m), 7.58-7.42 (5H, m), 7.33 (1H, dd, J=8.6, 2.1 Hz), 6.43 (1H, s), 6.32 (1H, d, J=3.5 Hz), 2.28 (3H, s). MS (ES+): m/e 503.0 (45), 501.1 (100). MS (ES−): m/e 501.1 (45), 499.1 (100).

Compound 600: m.p. 254-256° C. ¹H NMR (300 MHz, DMSO-d₆): δ 8.02 (1H, s), 7.91-7.86 (2H, m), 7.79-7.73 (2H, m), 7.61-7.40 (5H, m), 7.17 (1H, td, J=9.0, 2.6 Hz), 6.43 (1H, s), 6.32 (1H, dd, J=3.5, 0.9 Hz), 2.28 (3H, s). MS (ES+): m/e 485.1 (100). MS (ES−): m/e 484.3 (25), 483.2 (100).

Compound 601: m.p. 261-262° C. ¹H NMR (300 MHz, DMSO-d₆): δ 8.14 (1H, s), 7.93 (1H, dd, J=7.9, 0.9 Hz), 7.90-7.86 (1H, m), 7.80-7.74 (2H, m), 7.66 (1H, d, J=3.5 Hz), 7.58 (1H, dd, J=8.8, 1.8 Hz), 7.46-7.40 (3H, m), 7.24 (1H, t, J=7.9 Hz), 6.46 (1H, s), 6.35 (1H, dd, J=3.5, 0.9 Hz), 2.29 (3H, s). MS (ES+): m/e 503.1 (40), 501.1 (100). MS (ES−): m/e 501.0 (50), 499.1 (100).

Compound 602: m.p. 229-230° C. ¹H NMR (300 MHz, DMSO-d₆): δ 8.13 (1H, d, J=2.0 Hz), 7.64 (1H, d, J=8.5 Hz), 7.54 (1H, d, J=3.5 Hz), 7.39 (1H, dd, J=8.5, 2.1 Hz), 7.13 (1H, t, J=8.0 Hz), 6.96-6.92 (2H, m), 6.75-6.71 (1H, m), 6.36 (1H, dd, J=3.5, 0.9 Hz), 6.24 (1H, s), 3.66 (3H, s), 2.33 (3H, s). MS (ES+): m/e 483.0 (45), 481.1 (100). MS (ES−): m/e 480.9 (50), 479.2 (100).

Compound 603: m.p. 201-202° C. ¹H NMR (300 MHz, DMSO-d₆): δ 7.90 (1H, dd, J=8.8, 2.8 Hz), 7.66 (1H, dd, J=8.8, 4.7 Hz), 7.54 (1H, d, J=3.5 Hz), 7.23 (1H, td, J=9.1, 2.8 Hz), 7.14 (1H, t, J=7.9 Hz), 6.96-6.92 (2H, m), 6.75-6.71 (1H, m), 6.36 (1H, d, J=3.5 Hz), 6.24 (1H, s), 3.66 (3H, s), 2.33 (3H, s). MS (ES+): m/e 466.4 (20), 465.1 (100). MS (ES−): m/e 464.4 (20), 463.2 (100).

Compound 604: m.p. 255-256° C. ¹H NMR (300 MHz, DMSO-d₆): δ 7.96 (1H, dd, J=8.0, 1.0 Hz), 7.61 (1H, d, J=3.5 Hz), 7.49 (1H, dd, J=7.9, 1.1 Hz), 7.28 (1H, t, J=7.9 Hz), 7.13 (1H, t, J=7.8 Hz), 7.08-7.01 (2H, m), 6.73 (1H, dd, J=7.9, 1.2 Hz), 6.38 (1H, dd, J=3.5, 0.9 Hz), 6.27 (1H, s), 3.68 (3H, s), 2.33 (3H, s). MS (ES+): m/e 483.1 (40), 481.1 (100). MS (ES−): m/e 481.1 (40), 479.2 (100).

Compound 605: m.p. 229-230° C. ¹H NMR (300 MHz, DMSO-d₆): δ 8.13 (1H, d, J=2.0 Hz), 7.71 (1H, d, J=8.8 Hz), 7.64 (1H, d, J=3.5 Hz), 7.42 (1H, dd, J=8.8, 2.0 Hz), 6.79 (2H, s), 6.38 (1H, d, J=3.5 Hz), 6.21 (1H, s), 3.69 (6H, s), 3.53 (3H, s), 2.34 (3H, s). MS (ES+): m/e 543.0 (45), 541.2 (100). MS (ES−): m/e 541.2 (45), 539.2 (100).

Compound 606: m.p. 244-245° C. ¹H NMR (300 MHz, DMSO-d₆): δ 7.90 (1H, dd, J=8.8, 2.6 Hz), 7.73 (1H, dd, J=8.9, 4.8 Hz), 7.64 (1H, d, J=3.5 Hz), 7.26 (1H, td, J=9.2, 2.8 Hz), 6.79 (2H, s), 6.39 (1H, dd, J=3.5, 0.9 Hz), 6.21 (1H, s), 3.69 (6H, s), 3.53 (3H, s), 2.34 (3H, s). MS (ES+): m/e 526.4 (25), 525.2 (100). MS (ES−): m/e 523.3 (100).

Compound 607: m.p. 251-252° C. ¹H NMR (300 MHz, DMSO-d₆): δ 7.97 (1H, dd, J=8.0, 1.0 Hz), 7.69 (1H, d, J=3.5 Hz), 7.51 (1H, dd, J=7.9, 0.8 Hz), 7.29 (1H, t, J=7.9 Hz), 6.85 (2H, s), 6.40 (1H, dd, J=3.5, 0.7 Hz), 6.23 (1H, s), 3.71 (6H, s), 3.54 (3H, s), 2.34 (3H, s). MS (ES+): m/e 543.0 (50), 541.2 (100). MS (ES−): m/e 541.2 (50), 539.2 (100).

Compound 608: m.p. 235-236° C. ¹H NMR (300 MHz, DMSO-d₆): δ 8.16 (1H, d, J=2.3 Hz), 7.75 (1H, d, J=8.8 Hz), 7.55 (1H, d, J=3.5 Hz), 7.45 (1H, dd, J=8.8, 2.3 Hz), 6.46 (1H, d, J=2.9 Hz), 6.396 (1H, dd, J=2.9, 0.8 Hz), 6.393 (1H, s), 5.91 (1H, dd, J=3.5, 0.9 Hz), 2.36 (3H, s), 2.06 (3H, s). MS (ES−): m/e 454.8 (30), 452.8 (100).

Compound 610: m.p. 224-225° C. ¹H NMR (300 MHz, DMSO-d₆): δ 7.92 (1H, dd, J=8.8, 2.7 Hz), 7.77 (1H, dd, J=9.0, 4.7 Hz), 7.56 (1H, d, J=3.5 Hz), 7.28 (1H, td, J=9.0, 2.6 Hz), 6.45 (1H, d, J=2.9 Hz), 6.39 (1H, dd, J=2.9, 0.9 Hz), 6.38 (1H, s), 5.91 (1H, dd, J=3.5, 0.9 Hz), 2.36 (3H, s), 2.06 (3H, s). MS (ES−): m/e 437.2 (100).

Compound 611: m.p. 237-238° C. ¹H NMR (300 MHz, DMSO-d₆): δ 8.13 (1H, d, J=2.0 Hz), 7.60 (1H, d, J=3.5 Hz), 7.58-7.54 (2H, m), 7.45-7.31 (3H, m), 7.17 (1H, br d, J=8.5 Hz), 6.36 (1H, d, J=3.5 Hz), 6.29 (1H, s), 2.32 (3H, s). MS (ES+): m/e 536.9 (50), 535.1 (100). MS (ES−): m/e 535.2 (40), 533.2 (100).

Compound 612: m.p. 232-233° C. ¹H NMR (300 MHz, DMSO-d₆): δ 7.90 (1H, dd, J=8.6, 2.8 Hz), 7.61-7.53 (3H, m), 7.43 (1H, dt, J=7.9, 0.5 Hz), 7.34 (1H, t, J=7.9 Hz), 7.24 (1H, td, J=9.0, 2.6 Hz), 7.17 (1H, dt, J=8.2, 1.2 Hz), 6.36 (1H, dd, J=3.5, 0.9 Hz), 6.28 (1H, s), 2.32 (3H, s). MS (ES+): m/e 520.3 (25), 519.2 (100). MS (ES−): m/e 518.3 (20), 517.2 (100).

Compound 613: m.p. 248-249° C. ¹H NMR (300 MHz, DMSO-d₆): δ 7.96 (1H, dd, J=7.9, 1.2 Hz), 7.65 (1H, d, J=3.5 Hz), 7.58 (1H, br s), 7.50-7.46 (2H, m), 7.34 (1H, t, J=8.0 Hz), 7.28 (1H, t, J=7.9 Hz), 7.18 (1H, dm, J=8.4 Hz), 6.37 (1H, dd, J=3.5, 0.9 Hz), 6.30 (1H, s), 2.32 (3H, s). MS (ES+): m/e 537.0 (45), 535.1 (100). MS (ES−): m/e 535.1 (45), 533.2 (100).

Compound 614: m.p. 207-210° C. ¹H NMR (300 MHz, DMSO-d₆): δ 8.61 (1H, d, J=3.2 Hz), 8.06 (1J, d, J=2.3 Hz), 7.65 (1H, d, J=8.8 Hz), 7.36 (1H, dd, J=8.8, 2.3 Hz), 7.11 (2H, d, J=8.8 Hz), 6.41 (2H, d, J=8.8 Hz), 6.12 (1H, d, J=3.2, 1.1 Hz), 5.96 (1H, s), 3.18 (4H, q, J=7.0 Hz), 2.24 (3H, s), 0.98 (6H, t, J=7.0 Hz). MS (ES+): m/e 524.2 (60), 522.3 (100). MS (ES−): m/e 522.3 (40), 520.2 (100).

Compound 615: m.p. 209-212° C. ¹H NMR (300 MHz, DMSO-d₆): δ 8.59 (1H, d, J=3.2 Hz), 7.82 (1H, dd, J=8.8, 2.7 Hz), 7.66 (1H, dd, J=8.8, 4.1 Hz), 7.19 (1H, td, J=9.1, 2.7 Hz), 7.11 (2H, d, J=8.8 Hz), 6.41 (2H, d, J=8.8 Hz), 6.12 (1H, dd, J=3.2, 0.8 Hz), 5.97 (1H, s), 3.18 (4H, q, J=7.3 Hz), 2.25 (3H, s), 0.98 (6H, t, J=7.3 Hz). MS (ES−): m/e 505.5 (20), 504.3 (100).

Compound 616: m.p. 223-226° C. ¹H NMR (300 MHz, DMSO-d₆): δ 8.51 (1H, br), 7.90 (1H, d, J=7.3 Hz), 7.46 (1H, d, J=7.9 Hz), 7.26-7.19 (3H, m), 6.41 (2H, d, J=8.8 Hz), 6.16 (1H, d, J=3.2 Hz), 6.03 (1H, s), 3.19 (4H, q, J=7.0 Hz), 2.26 (3H, s), 0.98 (6H, t, J=7.0 Hz). MS (ES−): m/e 522.2 (40), 520.2 (100).

Compound 617: m.p. 260-261° C. ¹H NMR (300 MHz, DMSO-d₆): δ 8.70 (1H, dd, J=9.3, 0.4 Hz), 8.08 (1H, d, J=2.3 Hz), 7.86 (1H, d, J=8.2 Hz), 7.72 (1H, t, J=7.9 Hz), 7.59-7.23 (7H, m), 7.09 (1H, d, J=8.8 Hz), 6.31 (1H, d, J=8.8 Hz), 2.24 (3H, s). MS (ES+): m/e 503.1 (40), 501.1 (100). MS (ES−): m/e 501.1 (40), 499.2 (100).

Compound 618: m.p. 251-252° C. ¹H NMR (300 MHz, DMSO-d₆): δ 8.71 (1H, d, J=9.0 Hz), 7.90-7.10 (11H, m), 6.31 (1H, d, J=3.8 Hz), 2.24 (3H, s). MS (ES+): m/e 485.2 (100). MS (ES−): m/e 484.3 (25), 483.2 (100).

Compound 619: m.p. 249-250° C. ¹H NMR (300 MHz, DMSO-d₆): δ 8.75 (1H, d, J=8.8 Hz), 7.95-7.15 (11H, m), 6.33 (1H, dd, J=3.5, 0.9 Hz), 2.25 (3H, s). MS (ES+): m/e 503.0 (40), 501.1 (100). MS (ES−): m/e 501.2 (45), 499.1 100.

Compound 620: m.p. 259-260° C. ¹H NMR (300 MHz, DMSO-d₆): δ 8.14 (1H, d, J=2.0 Hz), 7.71 (2H, d, J=8.5 Hz), 7.64 (2H, d, J=8.5 Hz), 7.60-7.57 (2H, m), 7.38 (1H, dd, J=8.8, 2.0 Hz), 6.35 (1H, d, J=2.9 Hz), 6.30 (1H, s), 2.32 (3H, s). MS (ES+): m/e 478.0 (45), 476.1 (100). MS (ES−): m/e 476.1 (45), 474.1 (100).

Compound 621: m.p. 255-256° C. ¹H NMR (300 MHz, DMSO-d₆): δ 7.90 (1H, dd, J=8.8, 2.6 Hz), 7.72-7.58 (6H, m), 7.22 (1H, td, J=9.0, 2.6 Hz), 6.35 (1H, d, J=3.5 Hz), 6.30 (1H, s), 2.32 (3H, s). MS (ES+): m/e 461.4 (20), 460.1 (100). MS (ES−): m/e 459.3 (25), 458.2 (100).

Compound 622: m.p. 273-274° C. ¹H NMR (300 MHz, DMSO-d₆): δ 7.96 (1H, dd, J=7.9, 1.2 Hz), 7.71 (4H, s), 7.65 (1H, d, J=3.5 Hz), 7.47 (1H, dd, J=7.9, 1.2 Hz), 7.28 (1H, t, J=7.9 Hz), 6.37 (1H, dd, J=3.5, 0.9 Hz), 6.31 (1H, s), 2.32 (3H, s). MS (ES+): m/e 478.0 (45), 476.1 (100). MS (ES−): m/e 476.1 (40), 474.1 (100).

Compound 623: m.p. 253-254° C. ¹H NMR (300 MHz, DMSO-d₆): δ 8.41 (1H, d, J=2.3 Hz), 7.65 (1H, d, J=3.2 Hz), 7.59 (1H, d, J=8.5 Hz), 7.39 (1H, d, J=8.5, 2.3 Hz), 7.30-7.12 (2H, m), 7.05-6.95 (1H, m), 6.51 (1H, s), 6.37 (1H, d, J=3.2 Hz), 2.32 (3H, s). MS (ES+): m/e 488.9 (45), 487.0 (100). MS (ES−): m/e 487.0 (45), 485.1 (100).

Compound 624: m.p. 239-240° C. ¹H NMR (300 MHz, DMSO-d₆): δ 7.90 (1H, dd, J=8.8, 2.7 Hz), 7.65 (1H, d, J=3.5 Hz), 7.60 (1H, dd, J=8.9, 4.8 Hz), 7.29-7.12 (3H, m), 7.04-6.96 (1H, m), 6.51 (1H, s), 6.37 (1H, dd, J=3.5, 1.0 Hz), 2.32 (3H, s). MS (ES+): m/e 472.3 (25), 471.1 (100). MS (ES−): m/e 470.3 (20), 469.2 (100).

Compound 625: m.p. 274-275° C. ¹H NMR (300 MHz, DMSO-d₆): δ 7.96 (1H, dd, J=8.0, 1.0 Hz), 7.68 (1H, d, J=3.5 Hz), 7.47 (1H, dd, J=7.9, 1.1 Hz), 7.28 (1H, t, J=7.9 Hz), 7.26-6.93 (3H, m), 6.56 (1H, s), 6.38 (1H, dd, J=3.5, 0.9 Hz), 2.33 (3H, s). MS (ES+): m/e 488.9 (45), 487.0 (100). MS (ES−): m/e 487.1 (35), 485.1 (100).

Compound 626: m.p. 254-256° C. ¹H NMR (300 MHz, DMSO-d₆): δ 8.12 (1H, d, J=2.0 Hz), 7.59-7.56 (2H, m), 7.38 (1H, dd, J=8.8, 2.3 Hz), 7.16-6.92 (4H, m), 6.43 (1H, s), 6.35 (1H, dd, J=3.5, 0.9 Hz), 2.80 (3H, s), 2.30 (3H, s). MS (ES+): m/e 467.0 (45), 465.1 (100). MS (ES−): m/e 465.0 (45), 463.1 (100).

Compound 627: m.p. 214-215° C. ¹H NMR (300 MHz, DMSO-d₆): δ 7.88 (1H, dd, J=8.8, 2.6 Hz), 7.63-7.56 (2H, m), 7.26-6.92 (5H, m), 6.43 (1H, s), 6.35 (1H, dd, J=3.5, 0.9 Hz), 2.80 (3H, s), 2.30 (3H, s). MS (ES+): m/e 450.1 (25), 449.1 (100). MS (ES−): m/e 447.8 (25), 446.8 (100).

Compound 628: m.p. 244-245° C. ¹H NMR (300 MHz, DMSO-d₆): δ 7.95 (1H, dd, J=8.0, 0.9 Hz), 7.61 (1H, d, J=3.5 Hz), 7.46 (1H, dd, J=7.9, 1.2 Hz), 7.27 (1H, t, J=7.9 Hz), 7.13 (1H, d, J=7.3 Hz), 7.05-6.91 (3H, m), 6.47 (1H, s), 6.36 (1H, dd, J=3.5, 0.8 Hz), 2.87 (3H, s), 2.30 (3H, s). MS (ES+): m/e 465.1 (100). MS (ES−): m/e 465.0 (40), 463.2 (100).

| | | |
|---|---|---|
| Compound 164 | Compound 165 | Compound 166 |
| MS (ES+): m/e 433.0. | MS (ES+): m/e 447.0. | MS (ES+): m/e 451.0. |
| Compound 167 | Compound 168 | Compound 169 |
| MS (ES+): m/e 435.0. | MS (ES+): m/e 529.0. | MS (ES−): m/e 511.0. |
| Compound 170 | Compound 171 | Compound 172 |
| MS (ES−): m/e 509.0. | MS (ES−): m/e 523.0. | MS (ES−): m/e 529.0. |
| Compound 173 | Compound 174 | Compound 175 |
| MS (ES−): m/e 493.0. | MS (ES−): m/e 551.0. | MS (ES−): m/e 549.0. |
| Compound 176 | Compound 177 | Compound 178 |
| MS (ES−): m/e 538.0. | MS (ES−): m/e 499.0. | MS (ES−): m/e 483.0. |
| Compound 179 | Compound 180 | Compound 194 |
| MS (ES−): m/e 465.0. | MS (ES+): m/e 485.0. | MS (ES−): m/e 467.0. |
| Compound 195 | Compound 196 | Compound 197 |
| MS (ES+): m/e 507.5. | MS (ES+): m/e 487.0. | MS (ES−): m/e 479.0. |
| Compound 198 | | |
| MS (ES−): m/e 511.0. | | |
| Compound 199 | Compound 200 | Compound 201 |
| MS (ES−): m/e 449.0. | MS (ES−): m/e 455.5. | MS (ES+): m/e 441.5. |
| Compound 203 | Compound 204 | Compound 205 |
| MS (ES+): m/e 449.5. | MS (ES−): m/e 476.5. | MS (ES+): m/e 476.5. |
| Compound 206 | Compound 207 | Compound 208 |
| MS (ES+): m/e 487.5. | MS (ES−): m/e 494.0. | MS (ES−): m/e 505.5. |
| Compound 209 | Compound 210 | Compound 211 |
| MS (ES+): m/e 498.0. | MS (ES+): m/e 465.0. | MS (ES+): m/e 467.0. |
| Compound 212 | Compound 213 | Compound 214 |
| MS (ES+): m/e 412.5. | MS (ES+): m/e 431.5. | MS (ES+): m/e 469.5. |
| Compound 215 | Compound 216 | Compound 226 |
| MS (ES−): m/e 494.5. | MS (ES+): m/e 469.5. | MS (ES−): m/e 460.5. |
| Compound 227 | Compound 228 | Compound 229 |
| MS (ES+): m/e 461.5. | MS (ES+): m/e 451.5. | MS (ES−): m/e 485.5. |
| Compound 230 | Compound 231 | Compound 232 |
| MS (ES+): m/e 437.5. | MS (ES+): m/e 485.5. | MS (ES+): m/e 481.0. |
| Compound 233 | Compound 234 | Compound 235 |
| MS (ES+): m/e 449.5. | MS (ES+): m/e 431.0. | MS (ES+): m/e 492.5. |
| Compound 236 | Compound 237 | Compound 238 |
| MS (ES+): m/e 481.5. | MS (ES+): m/e 492.5. | MS (ES+): m/e 447.5. |
| Compound 239 | Compound 241 | Compound 242 |
| MS (ES+): m/e 496.5. | MS (ES+): m/e 518.5. | MS (ES+): m/e 467.5. |
| Compound 243 | Compound 258 | Compound 259 |
| MS (ES+): m/e 509.5. | MS (ES+): m/e 443.5. | MS (ES+): m/e 421.5. |
| Compound 260 | Compound 261 | Compound 262 |
| MS (ES+): m/e 432.5. | MS (ES+): m/e 368.0. | MS (ES+): m/e 477.5. |
| Compound 263 | Compound 264 | Compound 265 |
| MS (ES+): m/e 507.5. | MS (ES+): m/e 457.5. | MS (ES+): m/e 441.5. |
| Compound 266 | Compound 267 | Compound 268 |
| MS (ES+): m/e 452.0. | MS (ES+): m/e 491.5. | MS (ES+): m/e 453.5. |
| Compound 269 | Compound 270 | Compound 271 |
| MS (ES+): m/e 451.0. | MS (ES+): m/e 480.0. | MS (ES+): m/e 489.5. |
| Compound 272 | Compound 273 | Compound 274 |
| MS (ES+): m/e 483.0. | MS (ES+): m/e 386.0. | MS (ES+): m/e 469.5. |
| Compound 275 | Compound 276 | Compound 277 |
| MS (ES+): m/e 419.0. | MS (ES+): m/e 386.0. | MS (ES+): m/e 384.0. |
| Compound 278 | Compound 279 | Compound 280 |
| MS (ES+): m/e 441.5. | MS (ES+): m/e 469.0. | MS (ES+): m/e 453.5. |
| Compound 281 | Compound 282 | Compound 286 |
| MS (ES+): m/e 402.0. | MS (ES+): m/e 503.5. | MS (ES+): m/e 453.5. |
| Compound 287 | Compound 288 | Compound 289 |
| MS (ES+): m/e 437.0. | MS (ES+): m/e 465.0. | MS (ES+): m/e 513.0. |
| Compound 290 | Compound 291 | Compound 292 |
| MS (ES+): m/e 463.0. | MS (ES+): m/e 449.0. | MS (ES+): m/e 509.5. |
| Compound 293 | Compound 294 | Compound 295 |
| MS (ES+): m/e 465.0. | MS (ES+): m/e 467.0. | MS (ES+): m/e 465.0. |
| Compound 296 | Compound 297 | Compound 298 |
| MS (ES+): m/e 435.0. | MS (ES+): m/e 491.0. | MS (ES+): m/e 509.5. |
| Compound 299 | Compound 300 | Compound 301 |
| MS (ES+): m/e 525.5. | MS (ES+): m/e 547.0. | MS (ES+): m/e 527.0. |
| Compound 302 | Compound 303 | Compound 305 |
| MS (ES+): m/e 436.5. | MS (ES+): m/e 398.0. | MS (ES+): m/e 447.0. |
| Compound 306 | Compound 307 | Compound 308 |
| MS (ES+): m/e 513.0. | MS (ES+): m/e 425.0. | MS (ES+): m/e 471.0. |
| Compound 309 | Compound 310 | Compound 311 |
| MS (ES+): m/e 447.0. | MS (ES+): m/e 495.5. | MS (ES+): m/e 461.5. |
| Compound 312 | Compound 313 | Compound 314 |
| MS (ES+): m/e 483.0. | MS (ES+): m/e 479.0. | MS (ES+): m/e 495.0. |
| Compound 315 | Compound 316 | Compound 317 |
| MS (ES+): m/e 364.2. | MS (ES+): m/e 398.0. | MS (ES+): m/e 522.0. |
| Compound 318 | Compound 319 | Compound 320 |
| MS (ES+): m/e 485.0. | MS (ES+): m/e 459.5. | MS (ES+): m/e 461.5. |
| Compound 321 | Compound 322 | Compound 323 |
| MS (ES+): m/e 444.0. | MS (ES+): m/e 461.0. | MS (ES+): m/e 443.5. |
| Compound 324 | Compound 325 | Compound 326 |
| MS (ES+): m/e 382.0. | MS (ES+): m/e 409.0. | MS (ES+): m/e 467.0. |
| Compound 327 | Compound 328 | Compound 329 |
| MS (ES+): m/e 495.1. | MS (ES+): m/e 488.5. | MS (ES+): m/e 477.5. |
| Compound 330 | Compound 331 | Compound 332 |
| MS (ES+): m/e 485.0. | MS (ES+): m/e 506.5. | MS (ES+): m/e 479.0. |
| Compound 333 | Compound 334 | Compound 335 |
| MS (ES+): m/e 501.0. | MS (ES+): m/e 382.5. | MS (ES+): m/e 380.0. |
| Compound 336 | Compound 337 | Compound 338 |
| MS (ES+): m/e 512.0. | MS (ES+): m/e 471.0. | MS (ES+): m/e 476.5. |
| Compound 339 | Compound 340 | Compound 341 |
| MS (ES+): m/e 467.0. | MS (ES+): m/e 431.0. | MS (ES+): m/e 497.5. |
| Compound 342 | Compound 343 | Compound 344 |
| MS (ES+): m/e 462.5. | MS (ES+): m/e 418.0. | MS (ES+): m/e 463.5. |
| Compound 345 | Compound 346 | Compound 347 |
| MS (ES+): m/e 475.5. | MS (ES+): m/e 482.0. | MS (ES+): m/e 494.5. |
| Compound 348 | Compound 349 | Compound 350 |
| MS (ES+): m/e 455.5. | MS (ES+): m/e 448.5. | MS (ES+): m/e 466.0. |
| Compound 351 | Compound 352 | Compound 353 |
| MS (ES+): m/e 481.0. | MS (ES+): m/e 457.5. | MS (ES+): m/e 469.0. |
| Compound 354 | Compound 355 | Compound 356 |
| MS (ES+): m/e 483.0. | MS (ES+): m/e 467.0. | MS (ES+): m/e 475.5. |
| Compound 357 | Compound 358 | Compound 359 |
| MS (ES+): m/e 466.0. | MS (ES+): m/e 485.0. | MS (ES+): m/e 469.5. |
| Compound 360 | Compound 361 | Compound 362 |
| MS (ES+): m/e 496.5. | MS (ES+): m/e 481.5. | MS (ES+): m/e 462.5. |
| Compound 363 | Compound 364 | Compound 365 |
| MS (ES+): m/e 491.5. | MS (ES+): m/e 493.5. | MS (ES+): m/e 479.0. |
| Compound 366 | Compound 367 | Compound 368 |
| MS (ES+): m/e 467.5. | MS (ES+): m/e 502.5. | MS (ES+): m/e 479.5. |
| Compound 369 | Compound 370 | Compound 371 |
| MS (ES+): m/e 469.0. | MS (ES+): m/e 467.0. | MS (ES+): m/e 464.0. |
| Compound 372 | Compound 373 | Compound 374 |
| MS (ES+): m/e 444.5. | MS (ES+): m/e 444.5. | MS (ES+): m/e 478.0. |
| Compound 375 | Compound 376 | Compound 377 |
| MS (ES+): m/e 462.5. | S (ES+): m/e 412.5. | MS (ES+): m/e 450.0. |
| Compound 378 | Compound 379 | Compound 380 |
| MS (ES+): m/e 466.0. | MS (ES+): m/e 427.5. | MS (ES+): m/e 416.0. |
| Compound 381 | Compound 382 | Compound 383 |
| MS (ES+): m/e 450.0. | MS (ES+): m/e 390.0. | MS (ES+): m/e 460.5. |
| Compound 384 | Compound 385 | Compound 386 |
| MS (ES+): m/e 388.5. | MS (ES+): m/e 400.0. | MS (ES+): m/e 489.0. |
| Compound 387 | Compound 388 | Compound 389 |
| MS (ES+): m/e 471.0. | MS (ES+): m/e 450.0. | MS (ES+): m/e 483.0. |
| Compound 390 | Compound 391 | Compound 392 |
| MS (ES+): m/e 499.0. | MS (ES+): m/e 400.0. | MS (ES+): m/e 543.0. |
| Compound 393 | Compound 394 | Compound 395 |
| MS (ES+): m/e 506.5. | MS (ES+): m/e 491.5. | MS (ES+): m/e 515.0. |
| Compound 396 | Compound 397 | Compound 398 |
| MS (ES+): m/e 539.0. | MS (ES+): m/e 489.0. | MS (ES+): m/e 521.5. |
| Compound 399 | Compound 400 | Compound 401 |
| MS (ES+): m/e 495.0. | MS (ES+): m/e 473.0. | MS (ES+): m/e 438.0. |
| Compound 402 | Compound 403 | Compound 404 |
| MS (ES+): m/e 510.0. | MS (ES+): m/e 448.0. | MS (ES+): m/e 462.5. |
| Compound 405 | Compound 406 | Compound 407 |
| MS (ES+): m/e 507.5. | MS (ES+): m/e 527.0. | MS (ES+): m/e 494.0. |
| Compound 408 | Compound 409 | Compound 410 |
| MS (ES+): m/e 432.0. | MS (ES+): m/e 527.0. | MS (ES+): m/e 518.5. |
| Compound 411 | Compound 412 | Compound 413 |
| MS (ES+): m/e 510.5. | MS (ES+): m/e 554.0. | MS (ES+): m/e 479.0. |
| Compound 414 | Compound 415 | Compound 416 |
| MS (ES+): m/e 483.0. | MS (ES+): m/e 471.0. | MS (ES+): m/e 478.0. |
| Compound 417 | Compound 418 | Compound 419 |
| MS (ES+): m/e 482.0. | MS (ES+): m/e 494.0. | MS (ES+): m/e 491.5. |
| Compound 506 | Compound 507 | Compound 508 |
| MS (ES+): m/e 418.1. | MS (ES+): m/e 418.1. | MS (ES−): m/e 495.0. |
| Compound 509 | Compound 510 | Compound 511 |
| MS (ES−): m/e 511.0. | MS (ES+): m/e 434.2. | MS (ES+): m/e 418.1. |
| Compound 512 | Compound 513 | Compound 514 |
| MS (ES+): m/e 438.0. | MS (ES−): m/e 435.9. | MS (ES−): m/e 435.8. |
| Compound 515 | Compound 516 | Compound 517 |
| MS (ES−): m/e 435.8. | MS (ES+): m/e 438.0. | MS (ES−): m/e 418.4. |
| Compound 518 | Compound 519 | Compound 520 |
| MS (ES−): m/e 434.1. | MS (ES−): m/e 445.5. | MS (ES−): m/e 444.6. |
| Compound 521 | Compound 522 | Compound 523 |
| MS (ES−): m/e 433.8. | MS (ES−): m/e 446.8. | MS (ES+): m/e 452.0. |
| Compound 524 | Compound 525 | Compound 526 |
| MS (ES+): m/e 452.0. | MS (ES−): m/e 431.9. | MS (ES+): m/e 452.0. |
| Compound 527 | Compound 528 | Compound 529 |
| MS (ES+): m/e 452.0. | MS (ES−): m/e 510.6. | MS (ES+): m/e 436.0. |

### 6.2.2 Example 1a

### 4-(1-Acetylpyrrolidine-2-carbonyl)-1-(6-chlorobenzo[d]thiazol-2-yl)-5-(4-fluorophenyl)-3-hydroxy-1H-pyrrol-2(5H)-one (Compound 646)

Part A.

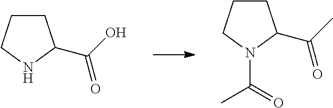

A mixture of proline (10.0 g, 86.9 mmol), acetic anhydride (50.0 mL, 530 mmol) and pyridine (35.0 mL, 433 mmol) was heated to 100° C. for 12 hours, then cooled and poured into water (500 mL). Solid sodium bicarbonate was added until neutral pH was achieved, and the mixture was extracted with ethyl acetate (2×400 mL). The extracts were washed with brine, combined, dried over magnesium sulfate, filtered and evaporated. The residual oil was eluted on a silica gel column with ethyl acetate, and the pure product, 1,2-diacetylpyrrolidine, isolated as an oil, TLC R_{F} 0.15 (ethyl acetate). ¹H NMR (300 MHz, CDCl₃): δ 4.55-4.48 (1H, m), 3.60-3.50 (1H, m), 3.49-3.39 (1H, m), 2.47-2.39 (1H, m), 2.11 (3H, s), 2.04 (3H, s), 2.02-1.80 (3H, m). MS (ES+): m/e no M+H.

Part B.

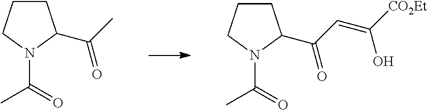

Sodium metal (2.67 g, 116 mmol) was dissolved in absolute ethanol (120 mL), and treated with a solution of 1,2-diacetylpyrrolidine (9.0 g, 58 mmol) and diethyl oxalate (32.0 mL, 236 mmol) in ethanol (30 mL). The mixture was stirred overnight, then poured into dilute HCl and extracted twice with ethyl acetate. The extracts were washed with brine, combined, dried over magnesium sulfate, filtered and evaporated. The crude product (ethyl 4-(1-acetylpyrrolidin-2-yl)-2-hydroxy-4-oxobut-2-enoate, 2.93 g, 11.5 mmol, 20%) was sufficiently pure for the next step. ¹H NMR (300 MHz, CDCl₃): δ 6.41 (1H, s), 4.60 (1H, dd, J=8.8, 3.8 Hz), 4.33 (2H, q, J=7.3 Hz), 3.71-3.61 (1H, m), 3.60-3.51 (1H, m), 2.26-2.16 (1H, m), 2.12 (3H, s), 2.11-1.93 (3H, m), 1.35 (3H, t, J=7.3 Hz). MS (ES+): m/e 256.2 (100). MS (ES−): m/e 255.0 (10), 253.9 (100).

Part C.

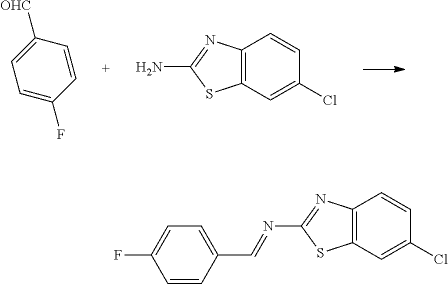

A solution of 4-fluorobenzaldehyde (3.50 g, 32.0 mmol) and 2-amino-6-chlorobenzothiazole (5.00 g, 27.1 mmol) in benzene (50 mL) was treated with a small amount of toluenesulfonic acid, and the resulting mixture was heated to reflux with azetropic removal of water. After 24 hours, 1 mL excess of the aldehyde reagent was added, and the refluxing was continued for an additional 12 hours. The mixture was cooled, and the resulting precipitate was collected by filtration, recrystallized from ethanol and dried under high vacuum to give the pure product, 6-chloro-N-(4-fluorobenzylidene)benzo[d]thiazol-2-amine, as a yellow crystalline solid (7.08 g, 24.3 mmol, 90%), m.p. 168-169° C. (ethanol). ¹H NMR (300 MHz, CDCl₃): δ 9.03 (1H, s), 8.07-8.00 (2H, m), 7.94-7.80 (2H, m), 7.43 (1H, dd, J=8.8, 2.0 Hz), 7.25-7.18 (2H, m). ¹⁹F NMR (282 MHz, CDCl₃): δ −104.14 (1F, s). MS (ES+): m/e 293.0 (40), 291.0 (100).

Part D.

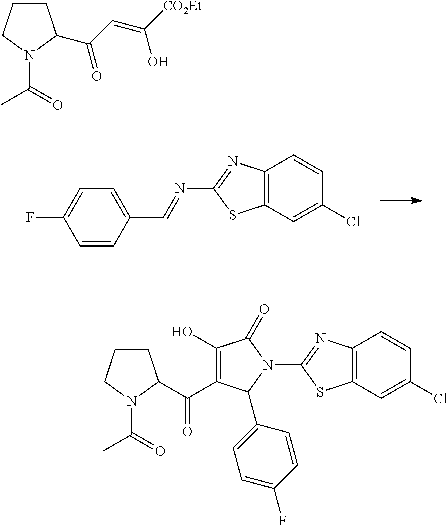

A solution of ethyl 4-(1-acetylpyrrolidin-2-yl)-2-hydroxy-4-oxobut-2-enoate (311 mg, 1.22 mmol) and 6-chloro-N-(4-fluorobenzylidene)benzo[d]thiazol-2-amine (354 mg, 1.22 mmol) in 3 mL ethanol and 1 mL acetic acid was heated to 80° C. for 12 hours, then cooled and partially evaporated. The residual material was partitioned between diethyl ether and dilute aq. sodium bicarbonate, and the resulting solid was collected by filtration, washed with ether and dried under vacuum to give the title compound (195 mg, 0.390 mmol, 32%), m.p. 268-272° C. ¹H NMR (300 MHz, CDCl₃): δ 7.75-6.85 (7H, m), 6.06 (1H, br), 5.04 (1H, br), 3.51 (2H, br), 2.43-2.10 (4H, br), 2.10 (3H, s). MS (ES+): m/e 502.1 (40), 500.2 (100). MS (ES−): m/e 499.9 (35), 497.9 (100).

The following compounds were prepared using modifications of the procedures given in Example 1a:

Compound 647: m.p. 148-152° C. TLC R_{F} 0.10 (ethyl acetate). MS (ES+): m/e 517.9 (35), 516.0 (100). MS (ES−): m/e 515.6 (40), 513.7 (100).

Compound 648: m.p. 293-297° C. ¹H NMR (300 MHz, CDCl₃): δ 7.40-6.90 (7H, br), 6-1.8 (11H, v br). MS (ES+): m/e 502.1 (40), 500.2 (100). MS (ES−): m/e 499.9 (40), 497.9 (100).

Compound 649: m.p. 266-269° C. ¹H NMR (300 MHz, CDCl₃): δ 7.60-7.00 (8H, v br), 6.4-0.6 (14H, v br). MS (ES+): m/e 518.0 (40), 516.2 (100). MS (ES−): m/e 515.9 (35), 513.8 (100)

### 7. EQUIVALENTS

Those skilled in the art will recognize or be able to ascertain using no more than routine experimentation, many equivalents to the specific embodiments described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present disclosure not to be limited in scope by the specific embodiments described herein. Indeed, various modifications of the embodiments in addition to those described herein will become apparent to those skilled in the art from the foregoing description and accompanying figures. Such modifications are intended to fall within the scope of the appended claims.

1. A compound having the following formula I: 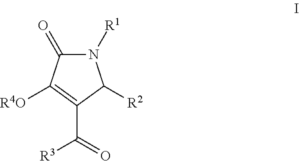
or a pharmaceutically acceptable salt thereof, wherein:
R¹ is H, substituted or unsubstituted aryl, substituted or unsubstituted heteroaryl, substituted or unsubstituted aryl(C₁₋₈)alkyl, substituted or unsubstituted heteroaryl(C₁₋₈)alkyl, or substituted or unsubstituted cycloalkyl;
R² is substituted or unsubstituted (C₁₋₈)alkyl, substituted or unsubstituted aryl, substituted or unsubstituted heteroaryl, substituted or unsubstituted aryl(C₁₋₈)alkyl, substituted or unsubstituted cycloalkyl, or substituted or unsubstituted heterocycloalkyl;
R³ is substituted or unsubstituted (C₁₋₈)alkyl, (C₁₋₈)alkoxy, substituted or unsubstituted aryl, substituted or unsubstituted heteroaryl, substituted or unsubstituted cycloalkyl, substituted or unsubstituted heterocycloalkyl, a 7-membered saturated or partially unsaturated bridged cyclic hydrocarbon (C₁₋₈)alkyl-O—(C₁₋₈)alkyl, aryl-O—(C₁₋₈)alkyl wherein the aryl ring can be substituted or unsubstituted, or R⁵OOC—(C₁₋₈)alkyl;
R⁴ is H, C₁₋₈alkyl, (C₁₋₈)alkyl(CO), substituted or unsubstituted aryl(C₁₋₈)alkyl, substituted or unsubstituted heteroaryl(C₁₋₈)alkyl, substituted or unsubstituted cycloalkyl; and
R⁵ is H or substituted or unsubstituted C₁₋₈alkyl,
wherein:
when R⁴ is H, R³ is not ethoxy;
when R⁴ is methyl, R³ is not ethoxy;
when R⁴ is H, R¹ is not substituted or unsubstituted phenyl;
R¹ is not pyridine;
and the compound is not:
4-(benzofuran-2-carbonyl)-5-(3-ethoxy-4-hydroxyphenyl)-3-hydroxy-1-(5(naphthalen-1-ylmethylthio)-1,3,4-thiadiazol-2-yl)-1H-pyrrol-2(5H)-one;
4-(benzofuran-2-carbonyl)-1-(5-(benzylthio)-1,3,4-thiadiazol-2-yl)-5-(3-ethoxy-4-hydroxyphenyl)-3-hydroxy-1H-pyrrol-2(5H)-one;
4-(benzofuran-2-carbonyl)-3-hydroxy-5-(3-hydroxyphenyl)-1-(6-methylbenzo[d]thiazol-2-yl)-1H-pyrrol-2(5H)-one;
4-(benzofuran-2-carbonyl)-5-(4-(benzyloxy)-3-methoxyphenyl)-3-hydroxy-1-(5-methyl-1,3,4-thiadiazol-2-yl)-1H-pyrrol-2 (5H)-one;
1-(5-acetyl-4-methylthiazol-2-yl)-4-(benzofuran-2-carbonyl)-5-(4-(benzyloxy)phenyl)-3-hydroxy-1H-pyrrol-2(5H)-one;
1-(5-acetyl-4-methylthiazol-2-yl)-4-(benzofuran-2-carbonyl)-5-(4-(benzyloxy)-3-methoxyphenyl)-3-hydroxy-1H-pyrrol-2(5H)-one;
4-(benzofuran-2-carbonyl)-1-(5-(4-chlorobenzylthio)-1,3,4-thiadiazol-2-yl)-3-hydroxy-5-(3-hydroxyphenyl)-1H-pyrrol-2(5H)-one;
4-benzoyl-3-hydroxy-5-phenyl-1-(pyridin-2-yl)-1H-pyrrol-2(5H)-one;
4-acetyl-3-hydroxy-5-phenyl-1-(pyridin-2-yl)-1H-pyrrol-2(5H)-one; or
4-benzoyl-3-hydroxy-5-phenyl-1-(thiazol-2-yl)-1H-pyrrol-2(5H)-one.

2. The compound of claim 1, wherein R¹ is substituted or unsubstituted heteroaryl.

3. The compound of claim 1, wherein R² is substituted or unsubstituted aryl.

4. The compound of claim 3, wherein R² is substituted phenyl.

5. The compound of claim 1, wherein R³ is substituted or unsubstituted aryl.

6. The compound of claim 5, wherein R³ is substituted phenyl.

7. The compound of claim 1, wherein R⁴ is H.

8. A pharmaceutical composition comprising a compound having the following formula I: 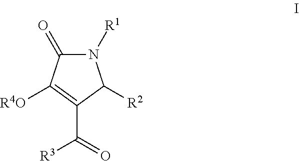
or a pharmaceutically acceptable salt thereof, wherein:
R¹ is H, substituted or unsubstituted aryl, substituted or unsubstituted heteroaryl, substituted or unsubstituted aryl(C₁₋₈)alkyl, substituted or unsubstituted heteroaryl(C₁₋₈)alkyl, or substituted or unsubstituted cycloalkyl;
R² is substituted or unsubstituted (C₁₋₈)alkyl, substituted or unsubstituted aryl, substituted or unsubstituted heteroaryl, substituted or unsubstituted aryl(C₁₋₈)alkyl, substituted or unsubstituted cycloalkyl, or substituted or unsubstituted heterocycloalkyl;
R³ is substituted or unsubstituted (C₁₋₈)alkyl, (C₁₋₈)alkoxy, substituted or unsubstituted aryl, substituted or unsubstituted heteroaryl, substituted or unsubstituted cycloalkyl, substituted or unsubstituted heterocycloalkyl, a 7-membered saturated or partially unsaturated bridged cyclic hydrocarbon (C₁₋₈)alkyl-O—(C₁₋₈)alkyl, aryl-O—(C₁₋₈)alkyl wherein the aryl ring can be substituted or unsubstituted, or R⁵OOC—(C₁₋₈)alkyl;
R⁴ is H, C₁₋₈alkyl, (C₁₋₈)alkyl(CO), substituted or unsubstituted aryl(C₁₋₈)alkyl, substituted or unsubstituted heteroaryl(C₁₋₈)alkyl, substituted or unsubstituted cycloalkyl; and
R⁵ is H or substituted or unsubstituted C₁₋₈alkyl,
wherein:
when R⁴ is H, R³ is not ethoxy;
when R⁴ is H, R¹ is not substituted or unsubstituted phenyl;
R¹ is not pyridine.

9. The pharmaceutical composition of claim 8, suitable for oral, parenteral, mucosal, transdermal or topical administration.

10. The pharmaceutical composition of claim 9, suitable for oral administration.

11. A method of preventing or inhibiting replication of a bacterial organism, comprising contacting the bacterial organism with an effective amount of a compound having the following formula I: 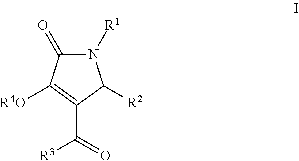
or a pharmaceutically acceptable salt thereof, wherein:
R¹ is H, substituted or unsubstituted aryl, substituted or unsubstituted heteroaryl, substituted or unsubstituted aryl(C₁₋₈)alkyl, substituted or unsubstituted heteroaryl(C₁₋₈)alkyl, or substituted or unsubstituted cycloalkyl;
R² is H, substituted or unsubstituted (C₁₋₈)alkyl, substituted or unsubstituted aryl, substituted or unsubstituted heteroaryl, substituted or unsubstituted aryl(C₁₋₈)alkyl, substituted or unsubstituted cycloalkyl, or substituted or unsubstituted heterocycloalkyl;
R³ is substituted or unsubstituted (C₁₋₈)alkyl, (C₁₋₈)alkoxy, substituted or unsubstituted aryl, substituted or unsubstituted heteroaryl, substituted or unsubstituted cycloalkyl, substituted or unsubstituted heterocycloalkyl, a 7-membered saturated or partially unsaturated bridged cyclic hydrocarbon (C₁₋₈)alkyl-O—(C₁₋₈)alkyl, aryl-O—(C₁₋₈)alkyl wherein the aryl ring can be substituted or unsubstituted, or R⁵OOC—(C₁₋₈)alkyl;
R⁴ is H, C₁₋₈alkyl, (C₁₋₈)alkyl(CO), substituted or unsubstituted aryl(C₁₋₈)alkyl, substituted or unsubstituted heteroaryl(C₁₋₈)alkyl, substituted or unsubstituted cycloalkyl; and
R⁵ is H or substituted or unsubstituted C₁₋₈alkyl,
wherein:
when R⁴ is H, R³ is not ethoxy;
when R⁴ is H, R¹ is not substituted or unsubstituted phenyl;
R¹ is not pyridine.

12. A method of preventing, treating or managing a bacterial infection in a subject, comprising administering to a subject in need thereof an effective amount of a compound having the following formula I: 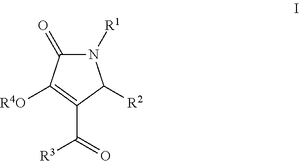
or a pharmaceutically acceptable salt thereof, wherein:
R¹ is H, substituted or unsubstituted aryl, substituted or unsubstituted heteroaryl, substituted or unsubstituted aryl(C₁₋₈)alkyl, substituted or unsubstituted heteroaryl(C₁₋₈)alkyl, or substituted or unsubstituted cycloalkyl;
R² is H, substituted or unsubstituted (C₁₋₈)alkyl, substituted or unsubstituted aryl, substituted or unsubstituted heteroaryl, substituted or unsubstituted aryl(C₁₋₈)alkyl, substituted or unsubstituted cycloalkyl, or substituted or unsubstituted heterocycloalkyl;
R³ is substituted or unsubstituted (C₁₋₈)alkyl, (C₁₋₈)alkoxy, substituted or unsubstituted aryl, substituted or unsubstituted heteroaryl, substituted or unsubstituted cycloalkyl, substituted or unsubstituted heterocycloalkyl, a 7-membered saturated or partially unsaturated bridged cyclic hydrocarbon (C₁₋₈)alkyl-O—(C₁₋₈)alkyl, aryl-O—(C₁₋₈)alkyl wherein the aryl ring can be substituted or unsubstituted, or R⁵OOC—(C₁₋₈)alkyl;
R⁴ is H, C₁₋₈alkyl, (C₁₋₈)alkyl(CO), substituted or unsubstituted aryl(C₁₋₈)alkyl, substituted or unsubstituted heteroaryl(C₁₋₈)alkyl, substituted or unsubstituted cycloalkyl; and
R⁵ is H or substituted or unsubstituted C₁₋₈alkyl,
wherein:
when R⁴ is H, R³ is not ethoxy;
when R⁴ is H, R¹ is not substituted or unsubstituted phenyl;
R¹ is not pyridine.

13. A compound, or a pharmaceutically acceptable salt thereof, wherein the compound is:

| | |
|---|---|
| 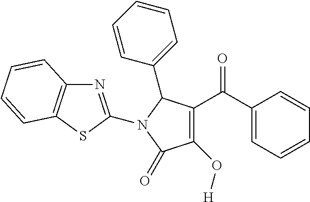 | 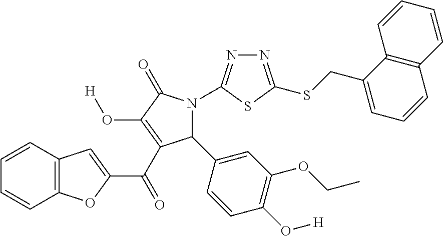 |
| 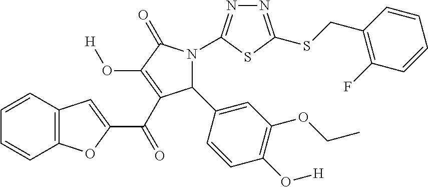 | 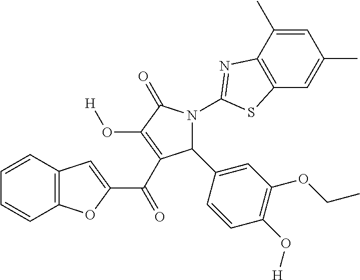 |
| 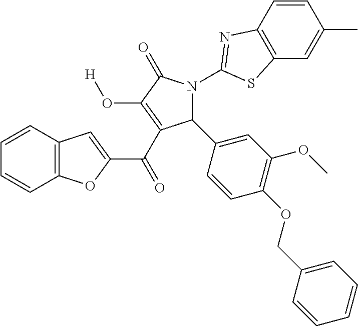 | 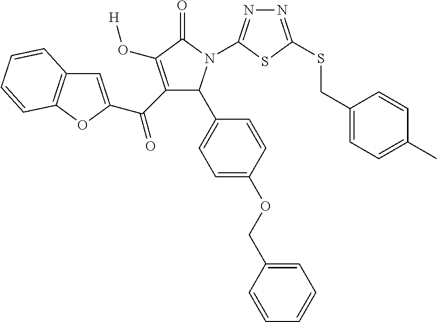 |
| 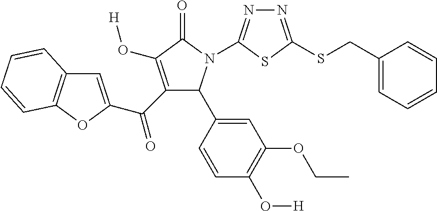 | 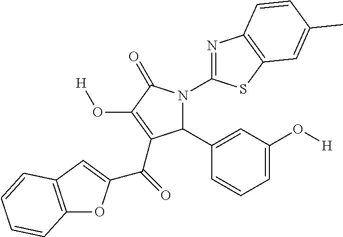 |
| 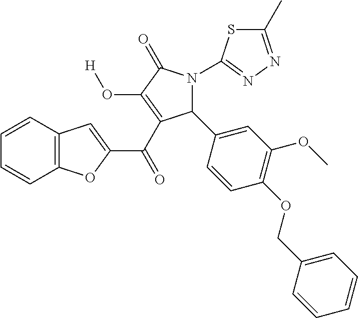 | 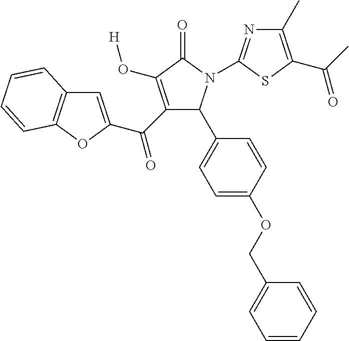 |
| 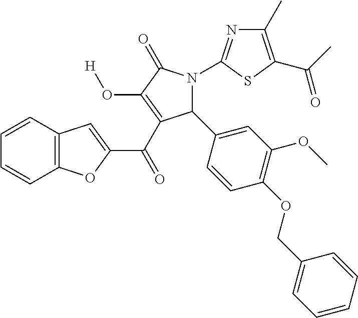 | 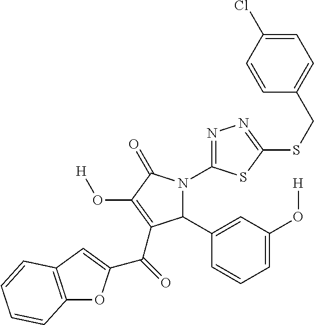 |
| 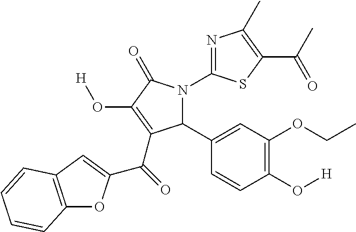 | 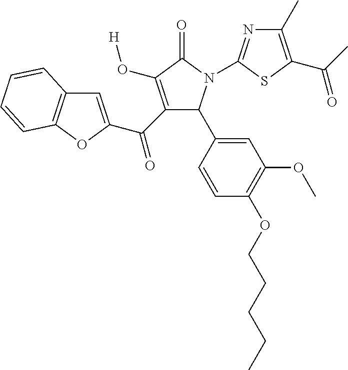 |
| 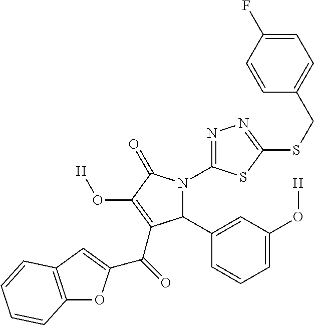 | 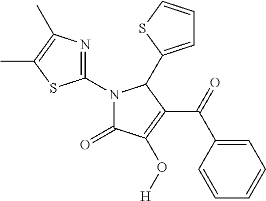 |
| 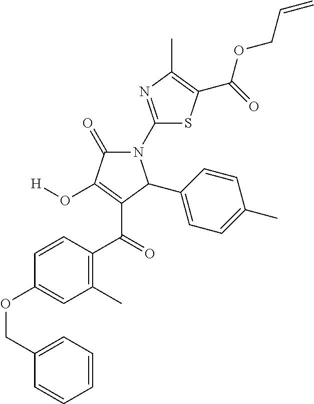 | 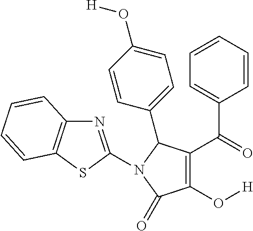 |
| 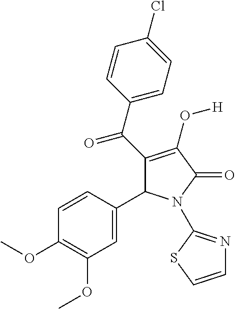 | 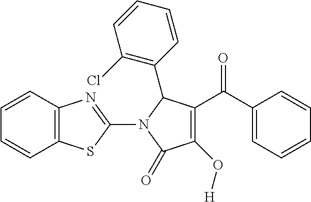 |
| 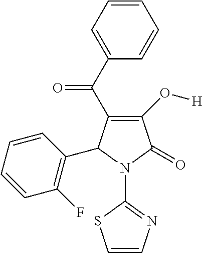 | 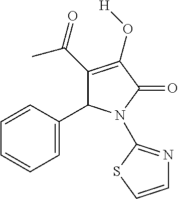 |
| 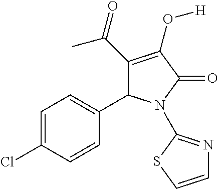 | 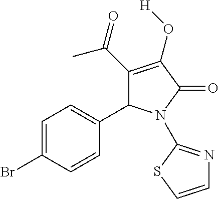 |
| 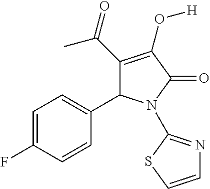 | 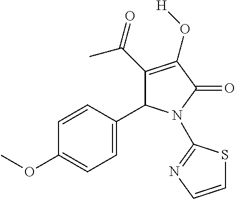 |
| 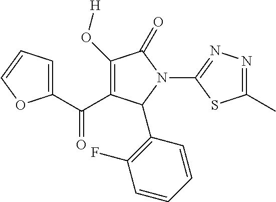 | 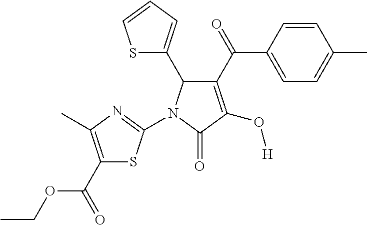 |
| 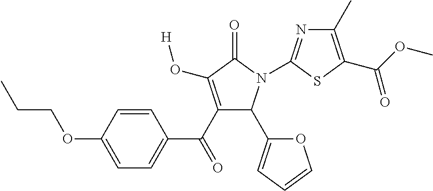 | 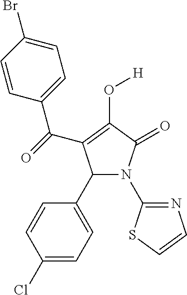 |
| 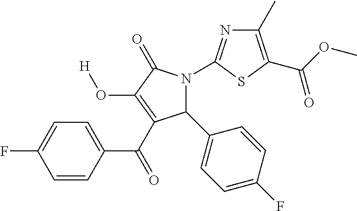 | 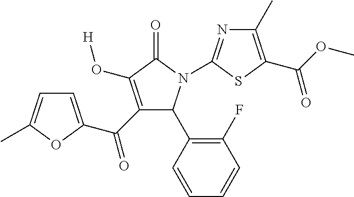 |
| 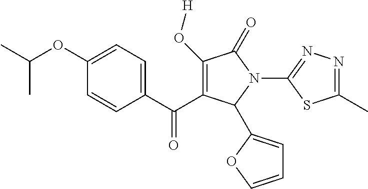 | 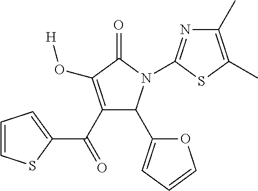 |
| 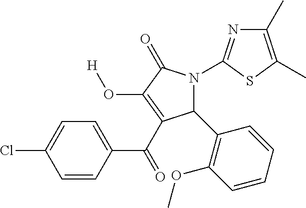 | 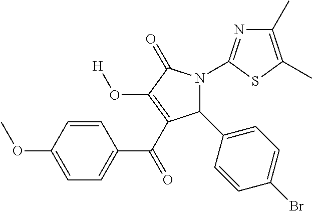 |
| 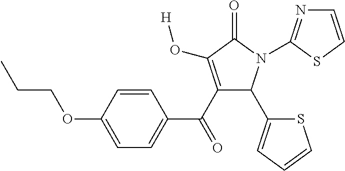 | 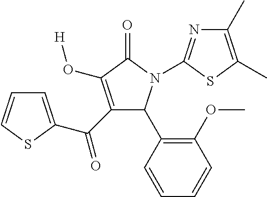 |
| 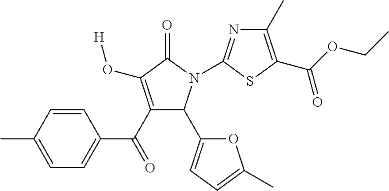 | 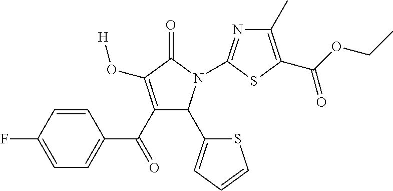 |
| 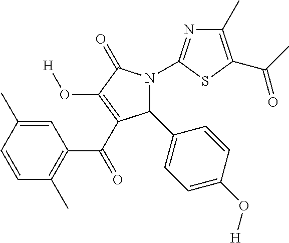 | 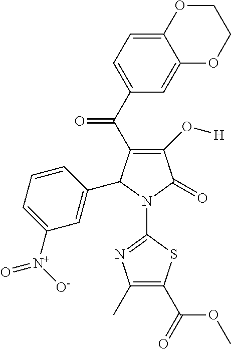 |
| 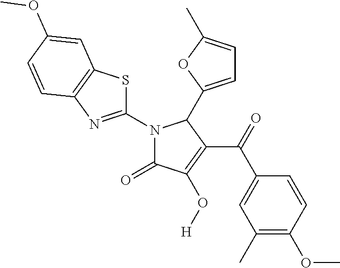 | 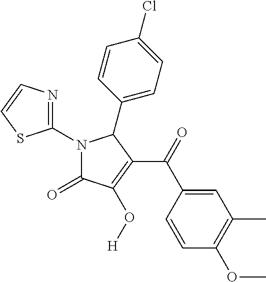 |
| 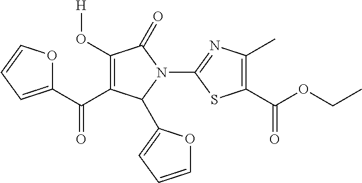 | 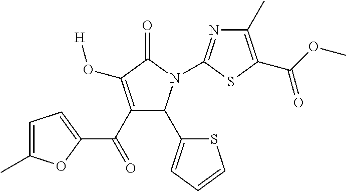 |
| 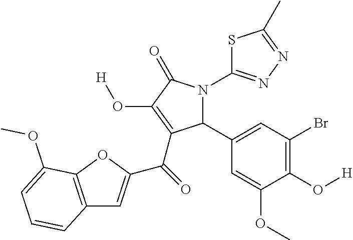 | 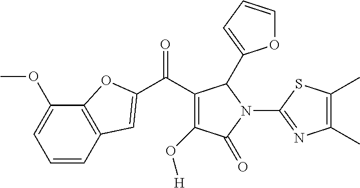 |
| 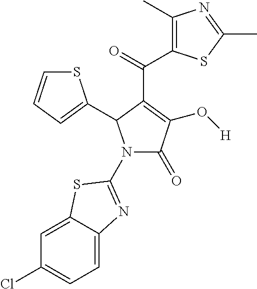 | 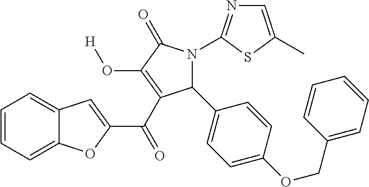 |
| 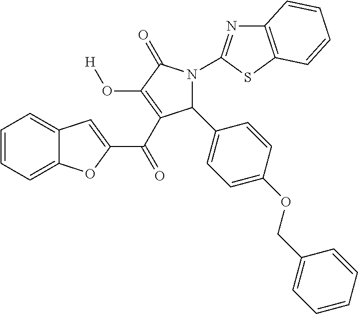 | 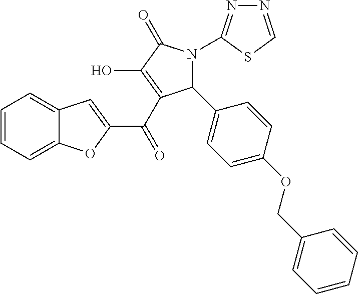 |
| 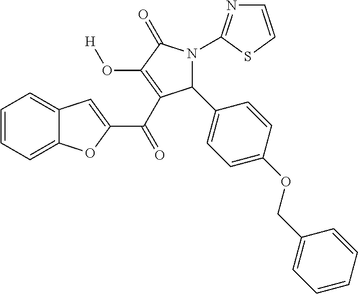 | 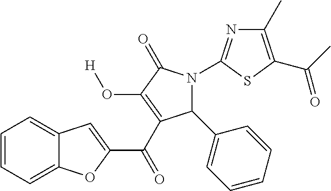 |
| 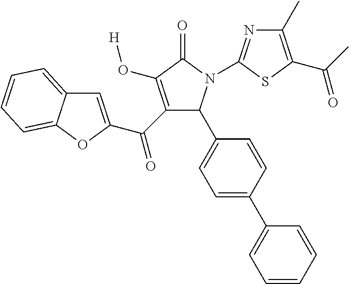 | 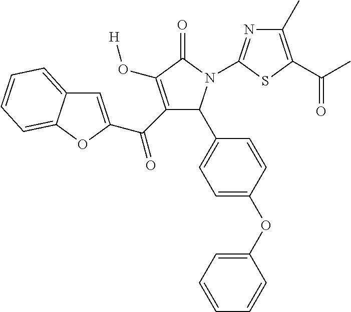 |
| 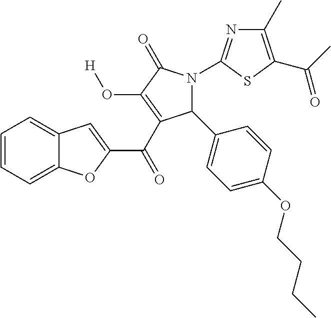 | 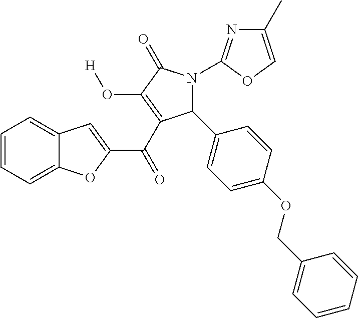 |
| 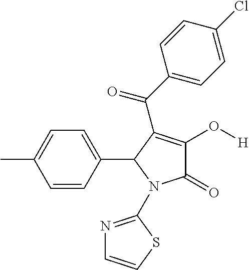 | 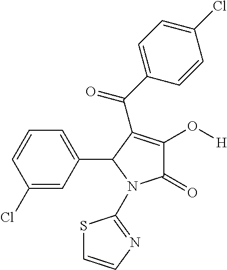 |
| 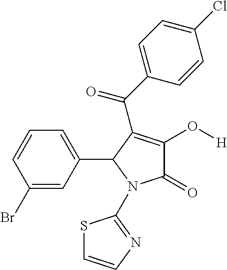 | 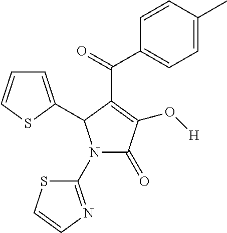 |
| 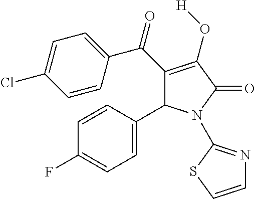 | 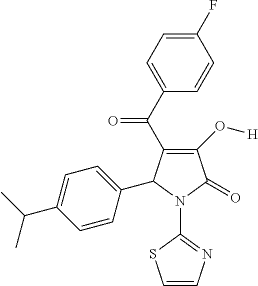 |
| 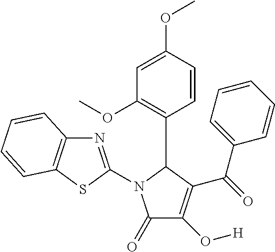 | 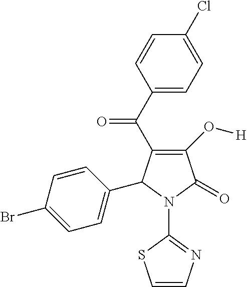 |
| 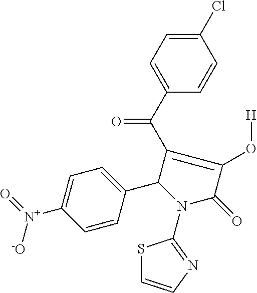 | 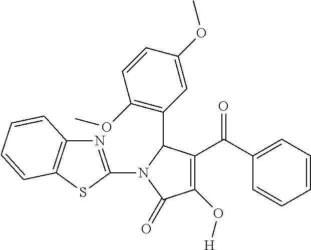 |
| 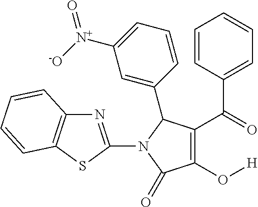 | 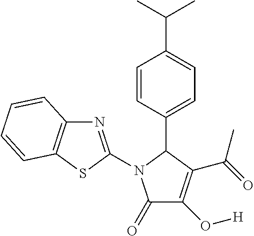 |
| 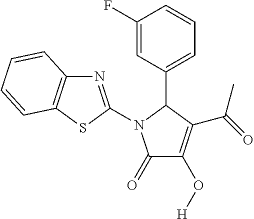 | 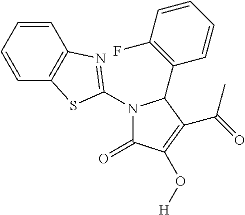 |
| 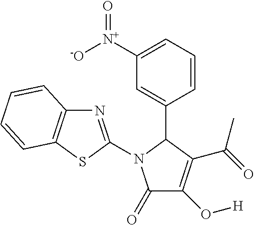 | 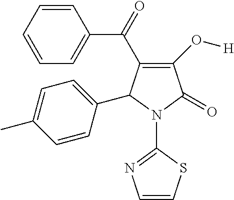 |
| 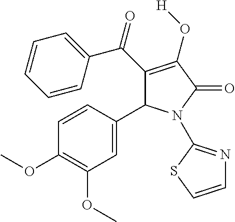 | 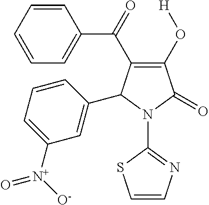 |
| 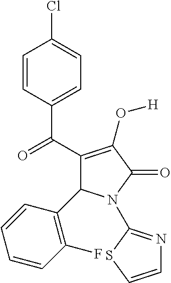 | 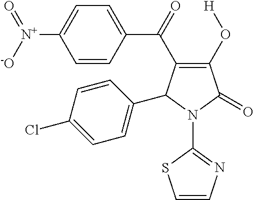 |
| 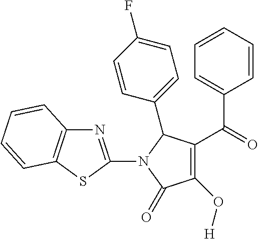 | 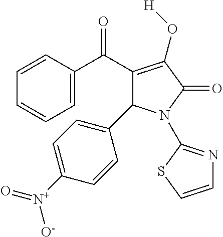 |
| 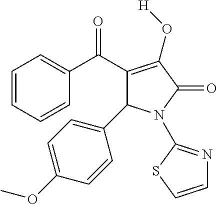 | 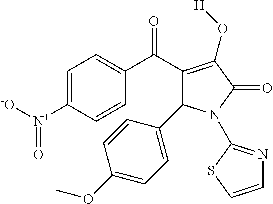 |
| 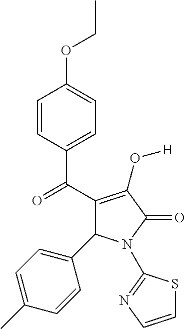 | 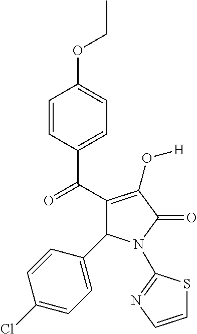 |
| 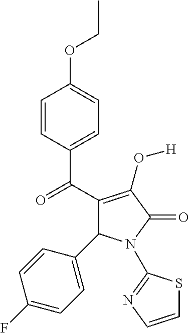 | 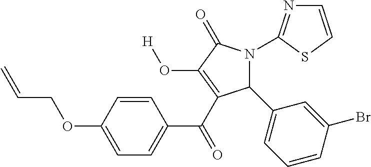 |
| 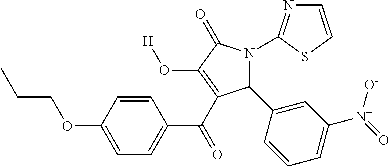 | 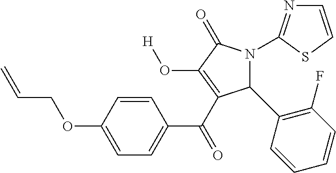 |
| 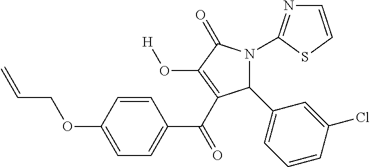 | 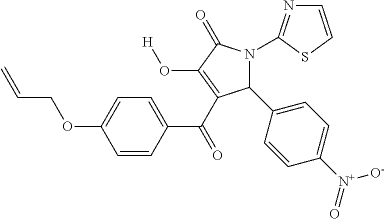 |
| 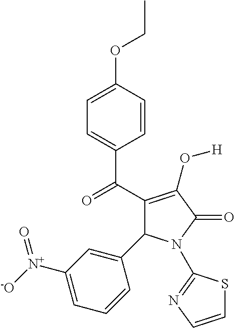 | 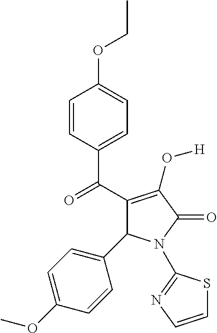 |
| 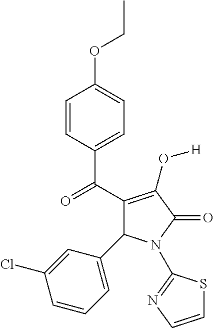 | 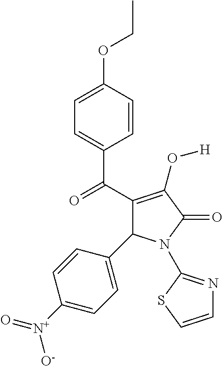 |
| 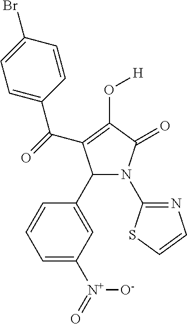 | 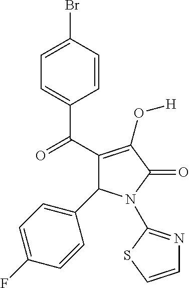 |
| 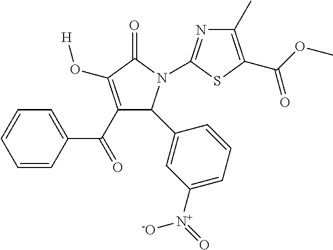 | 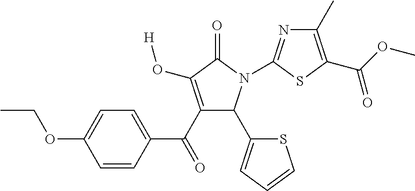 |
| 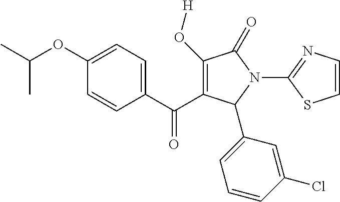 | 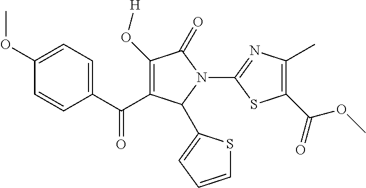 |
| 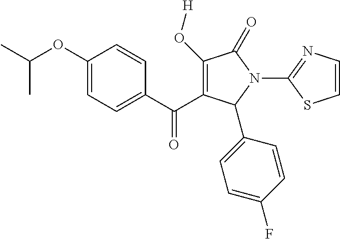 | 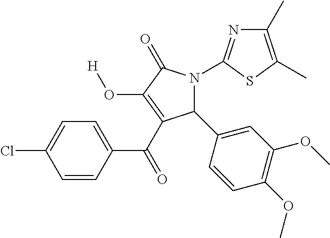 |
| 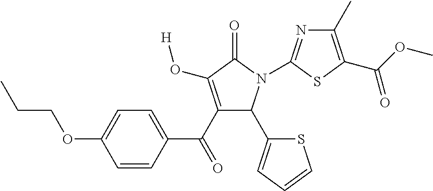 | 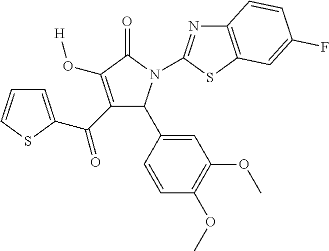 |
| 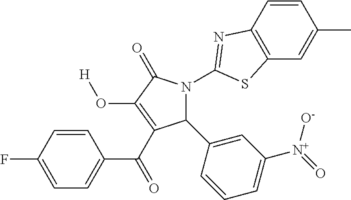 | 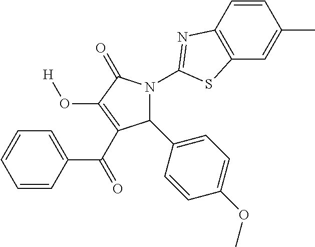 |
| 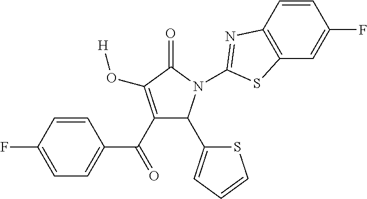 | 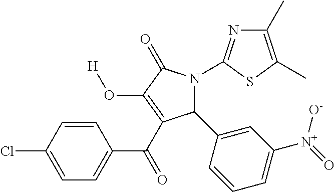 |
| 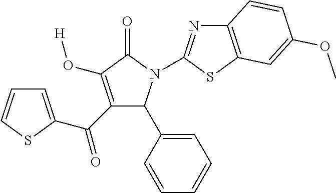 | 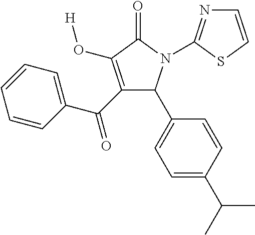 |
| 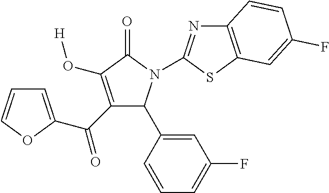 | 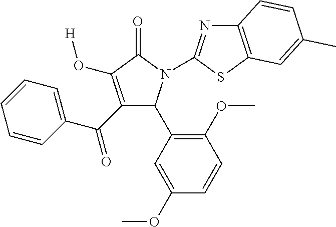 |
| 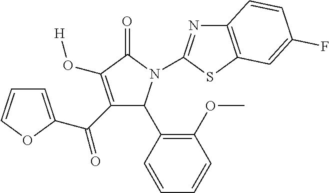 | 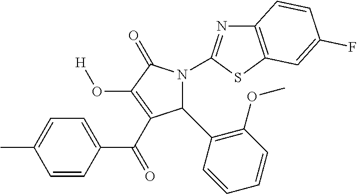 |
| 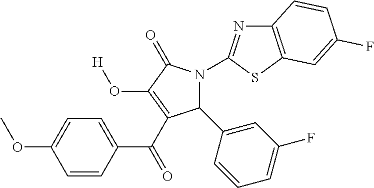 | 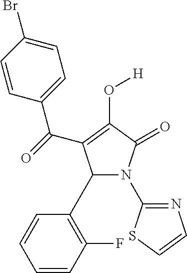 |
| 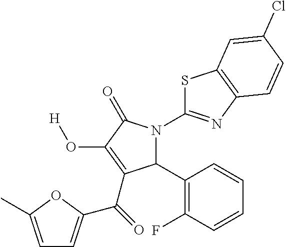 | 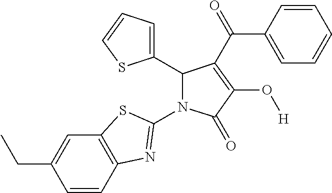 |
| 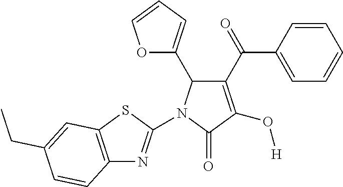 | 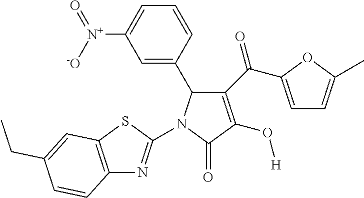 |
| 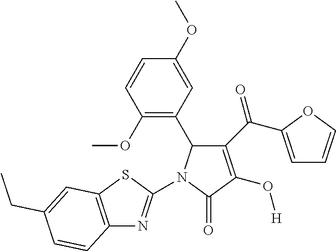 | 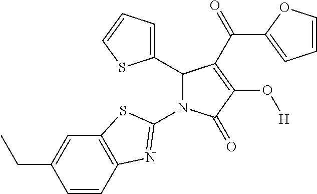 |
| 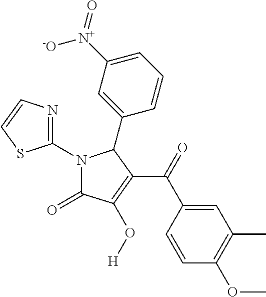 | 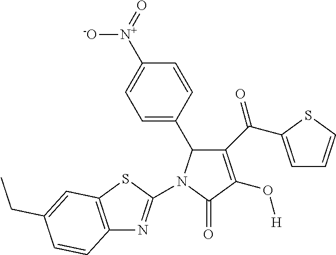 |
| 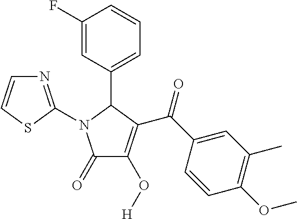 | 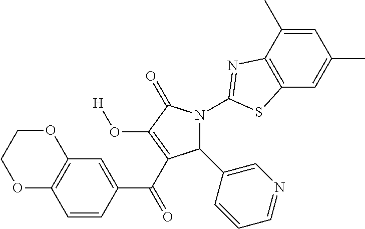 |
| 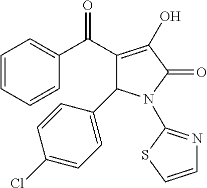 | 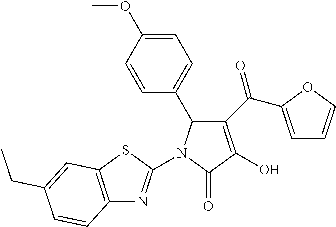 |
| 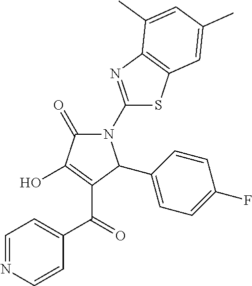 | 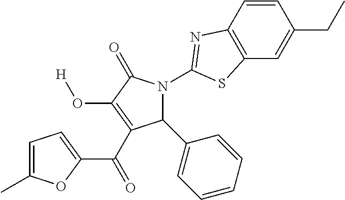 |
| 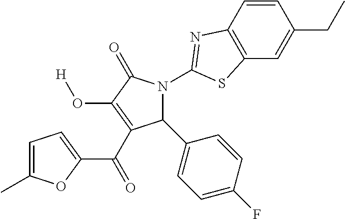 | 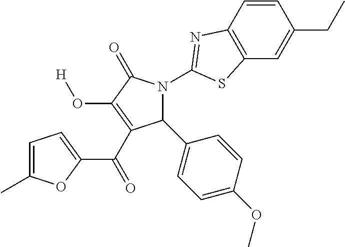 |
| 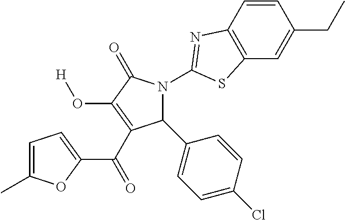 | 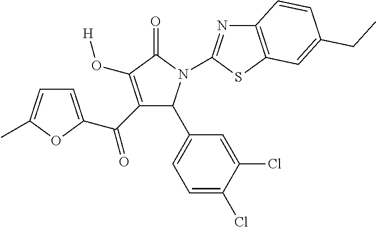 |
| 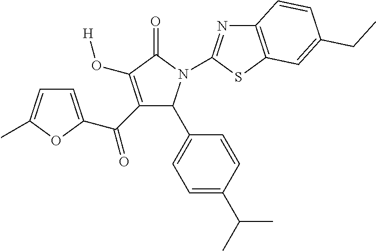 | 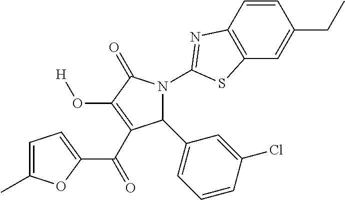 |
| 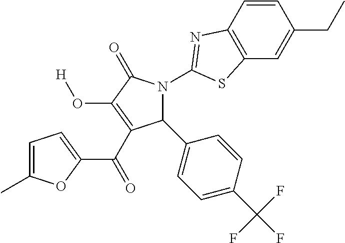 | 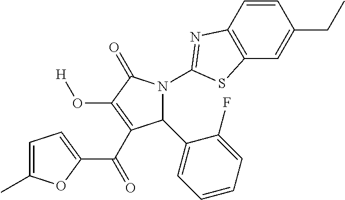 |
| 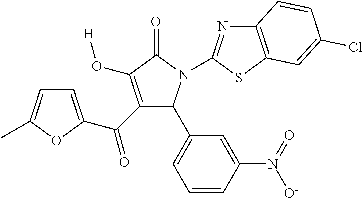 | 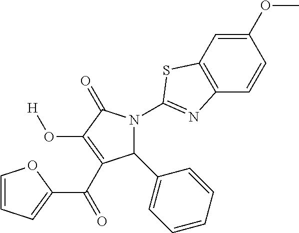 |
| 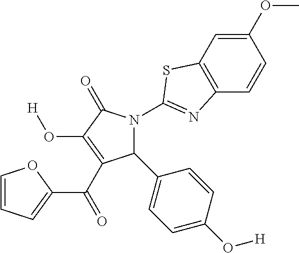 | 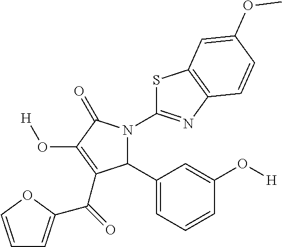 |
| 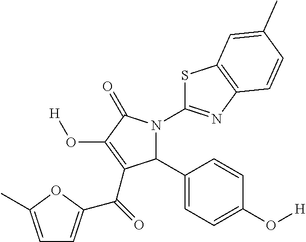 | 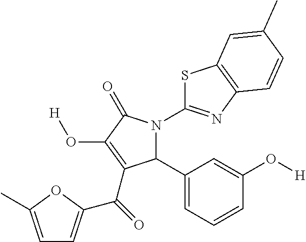 |
| 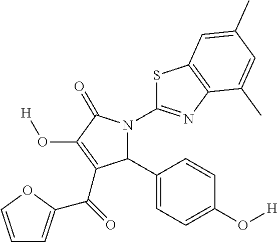 | 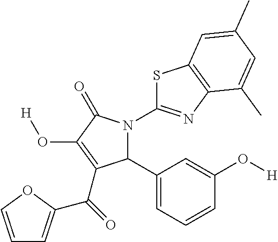 |
| 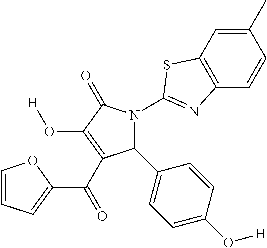 | 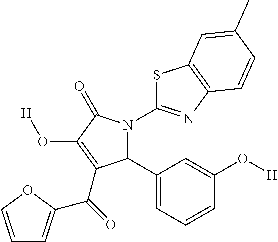 |
| 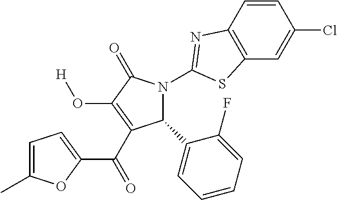 | 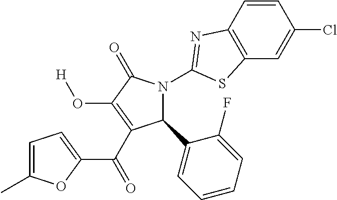 |
| 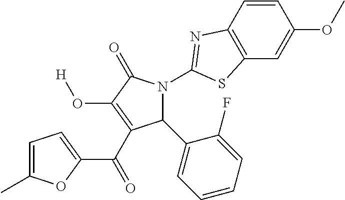 | 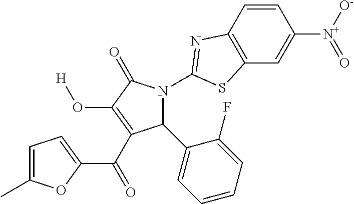 |
| 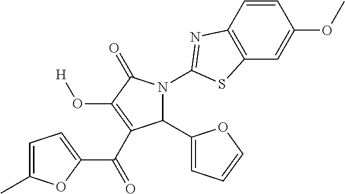 | 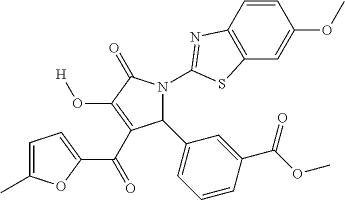 |
| 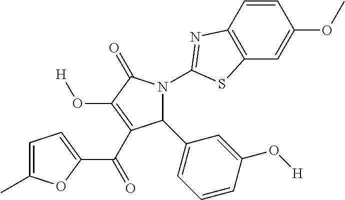 | 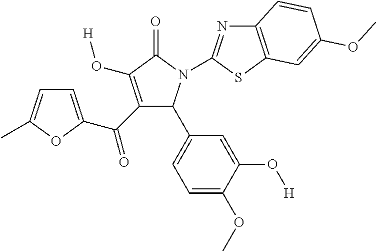 |
| 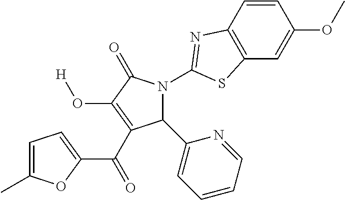 | 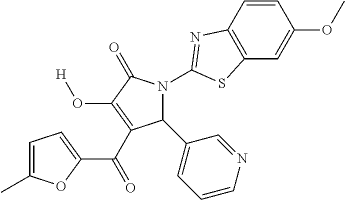 |
| 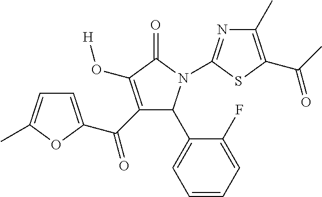 | 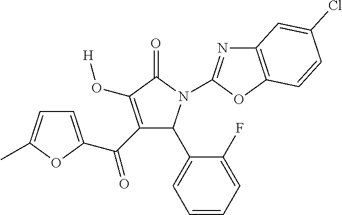 |
| 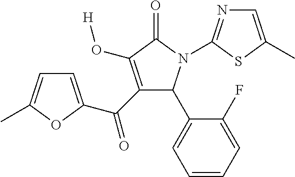 | 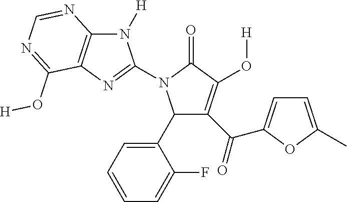 |
| 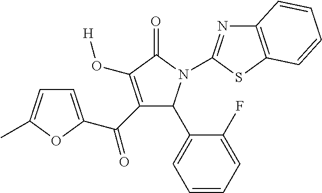 | 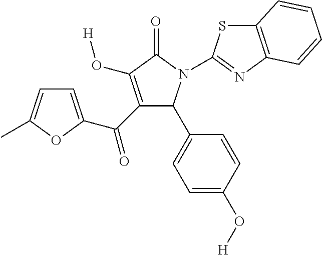 |
| 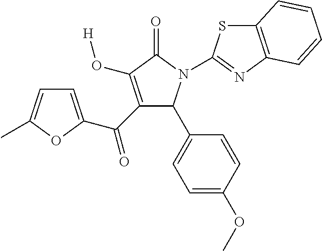 | 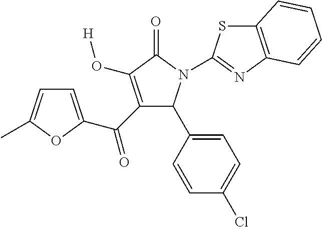 |
| 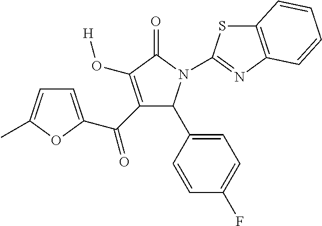 | 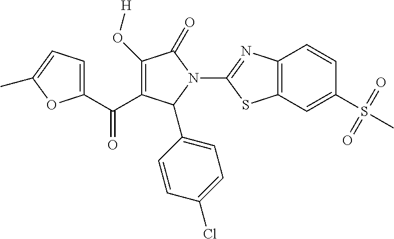 |
| 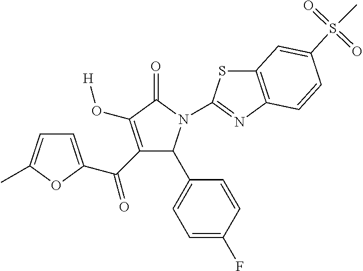 | 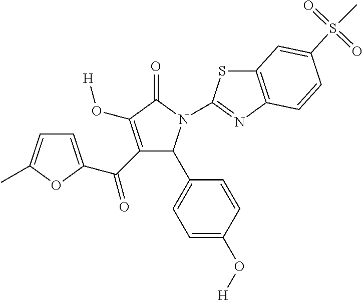 |
| 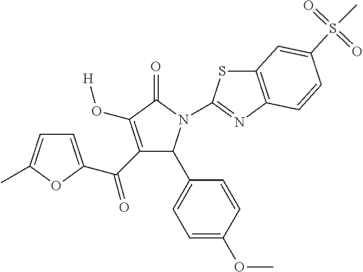 | 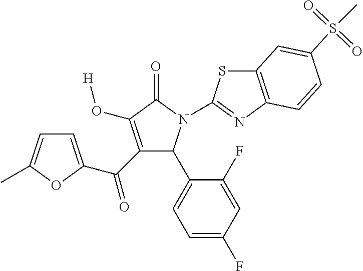 |
| 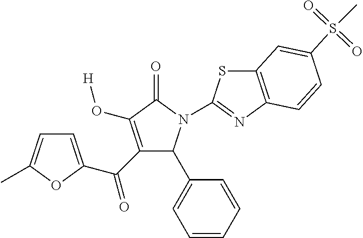 | 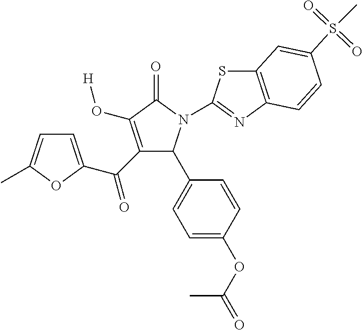 |
| 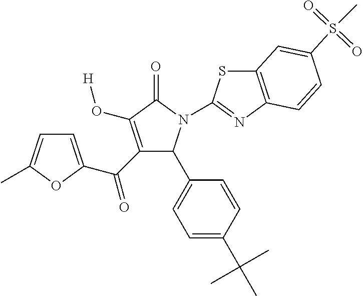 | 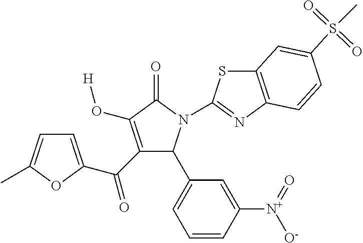 |
| 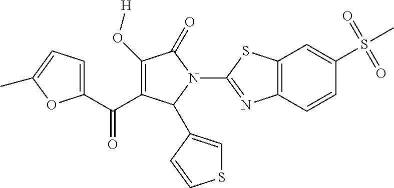 | 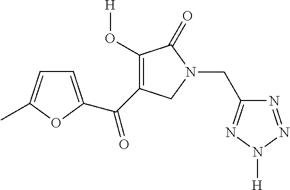 |
| 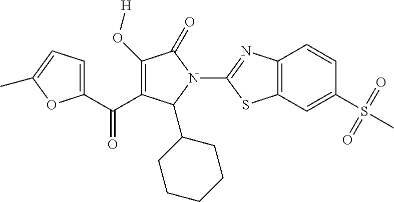 | 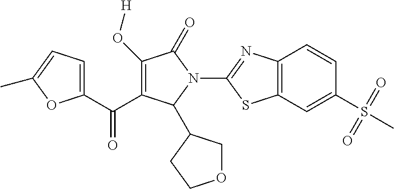 |
| 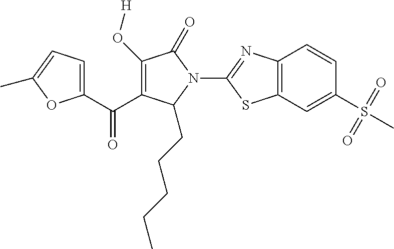 | 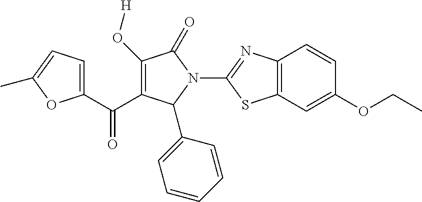 |
| 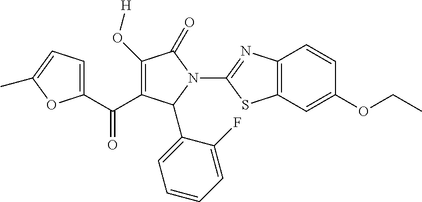 | 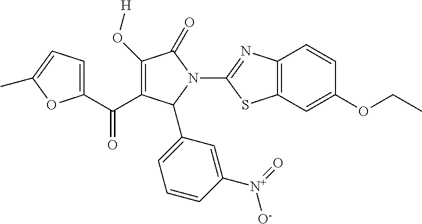 |
| 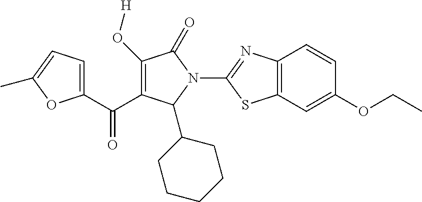 | 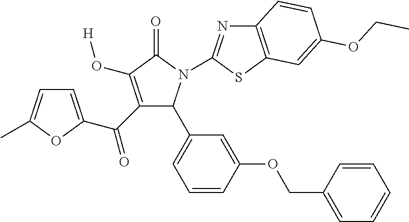 |
| 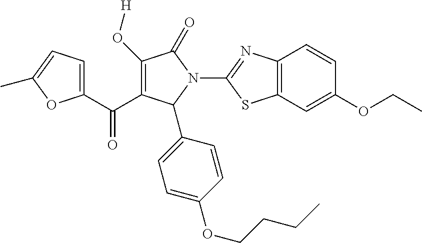 | 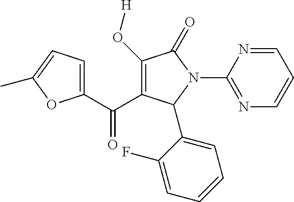 |
| 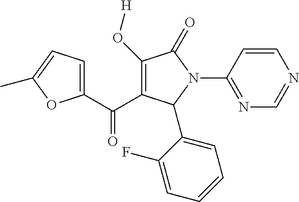 | 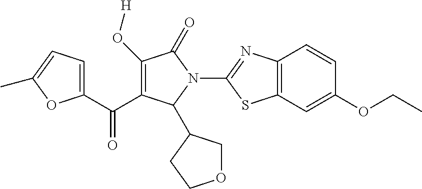 |
| 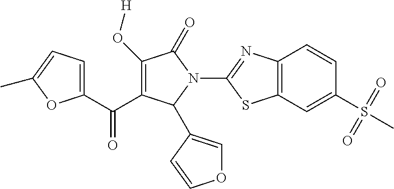 | 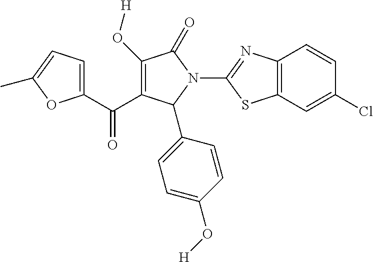 |
| 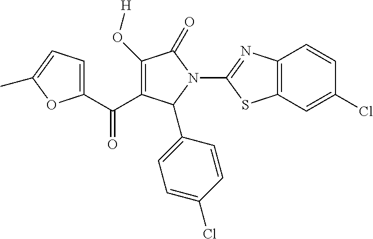 | 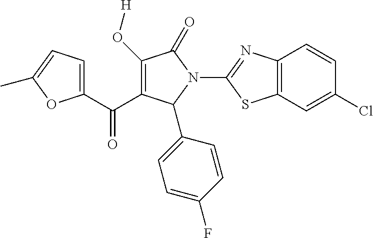 |
| 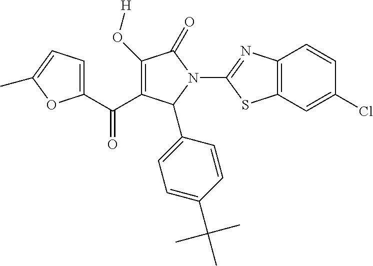 | 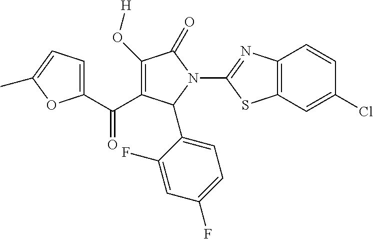 |
| 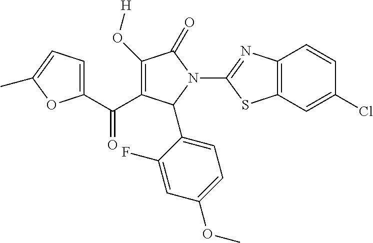 | 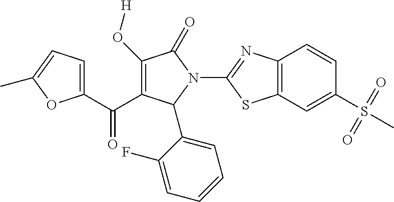 |
| 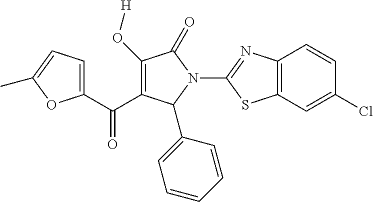 | 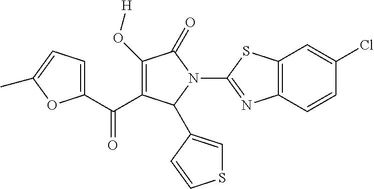 |
| 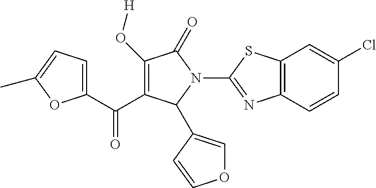 | 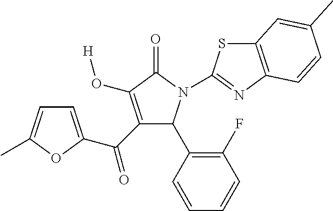 |
| 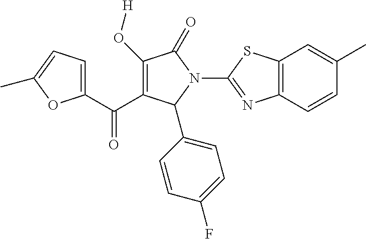 | 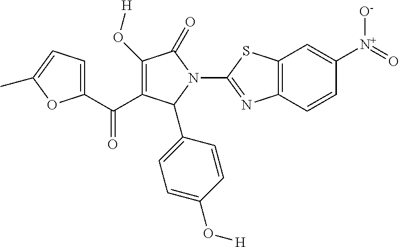 |
| 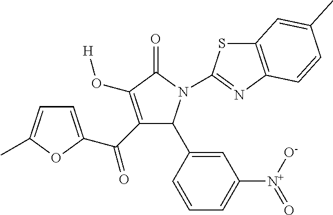 | 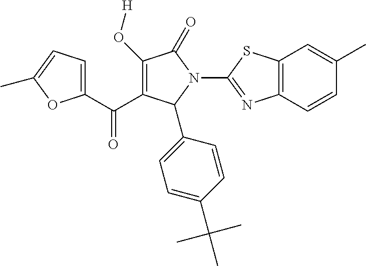 |
| 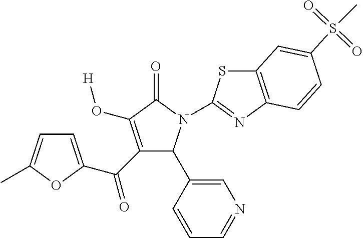 | 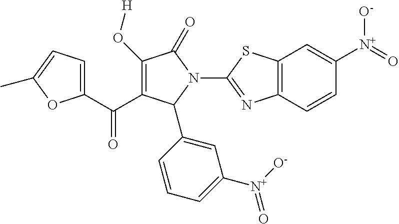 |
| 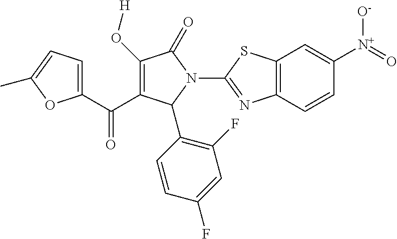 | 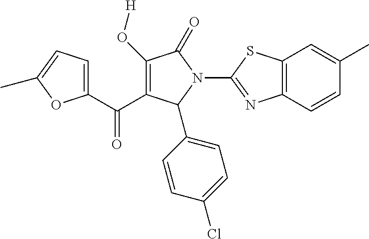 |
| 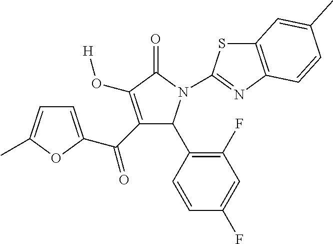 | 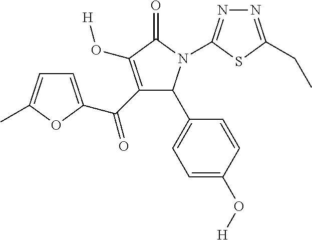 |
| 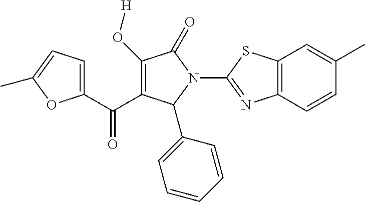 | 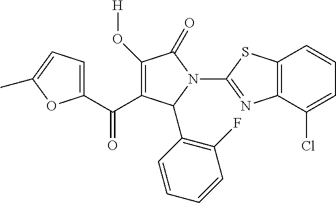 |
| 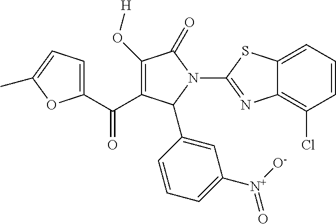 | 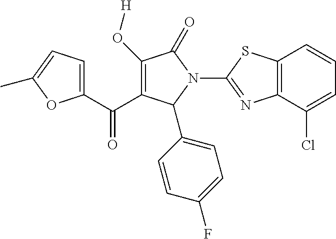 |
| 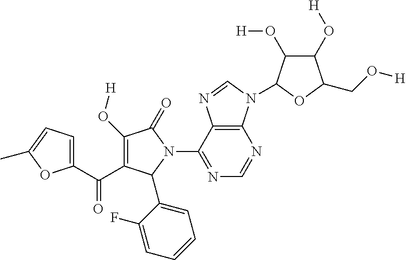 | 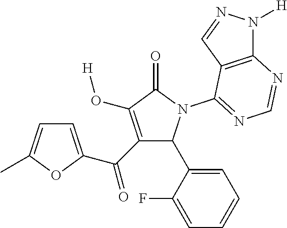 |
| 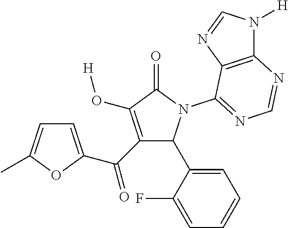 | 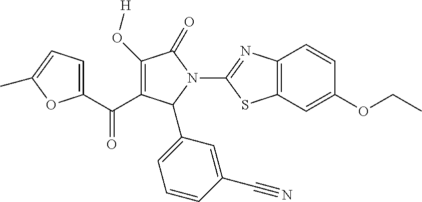 |
| 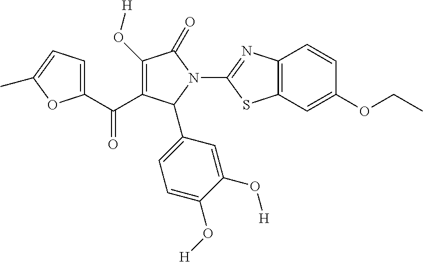 | 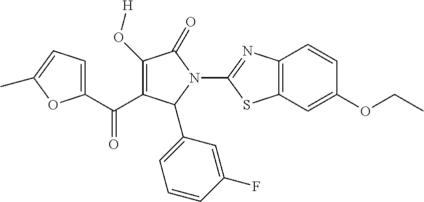 |
| 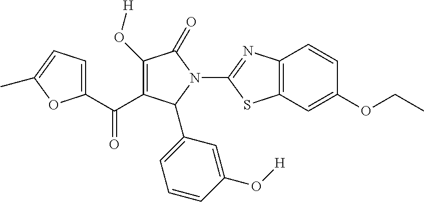 | 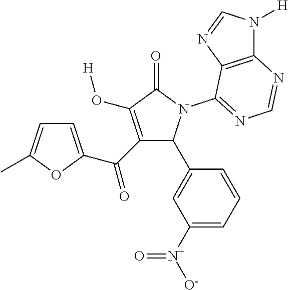 |
| 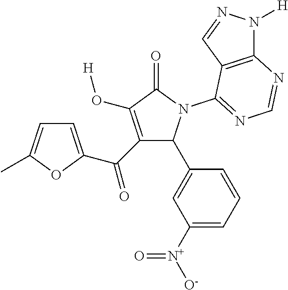 | 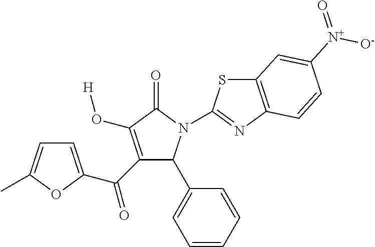 |
| 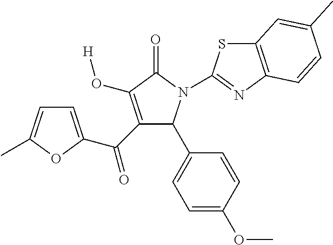 | 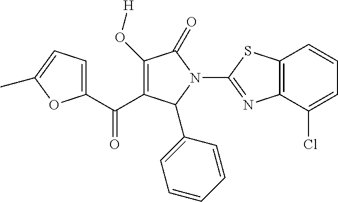 |
| 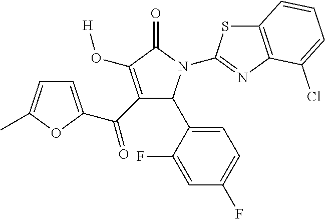 | 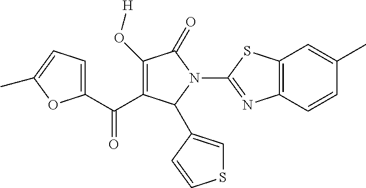 |
| 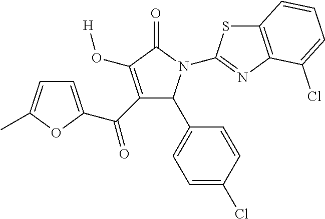 | 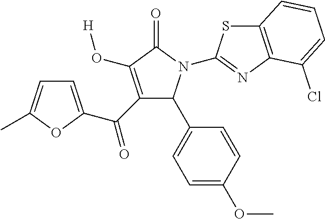 |
| 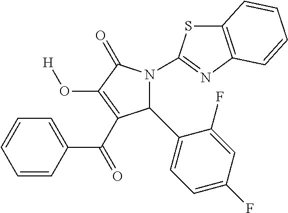 | 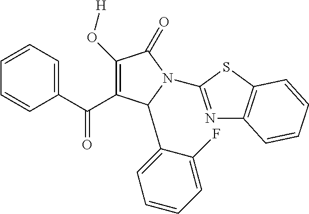 |
| 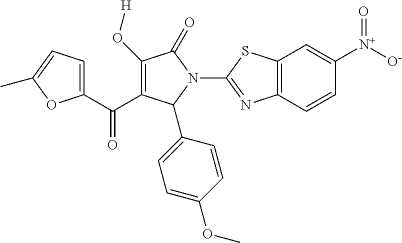 | 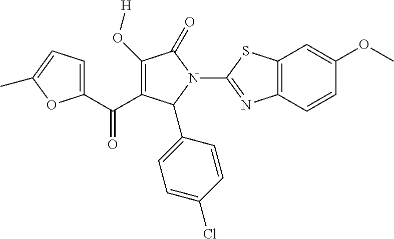 |
| 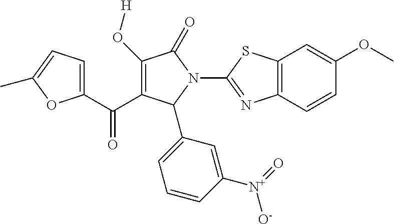 | 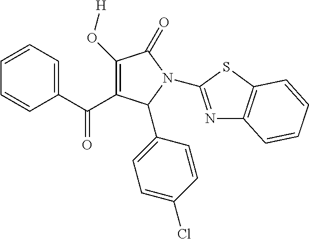 |
| 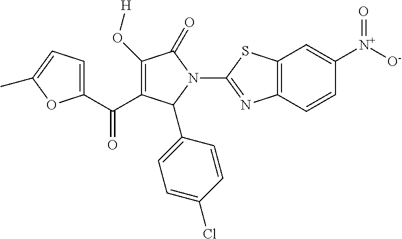 | 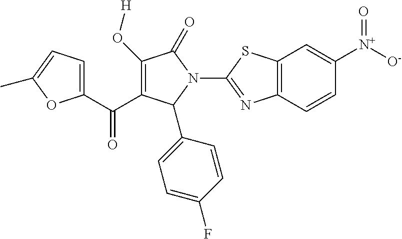 |
| 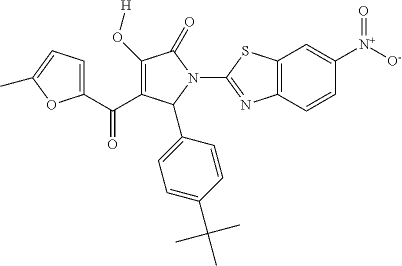 | 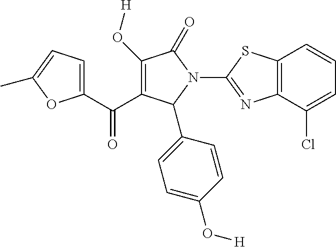 |
| 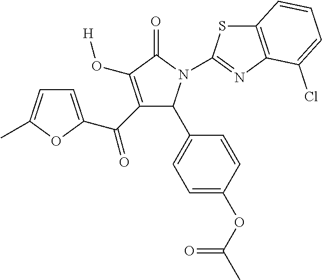 | 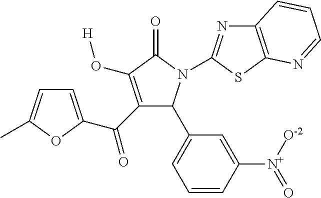 |
| 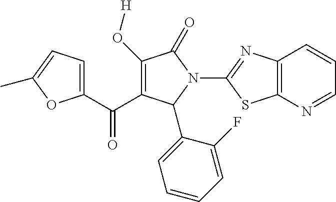 | 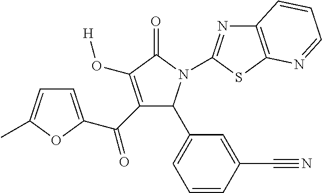 |
| 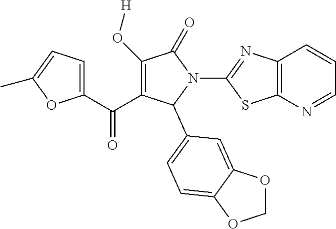 | 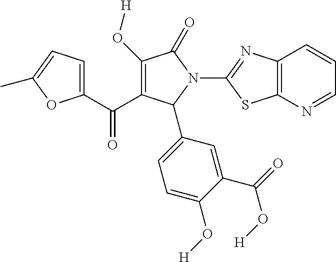 |
| 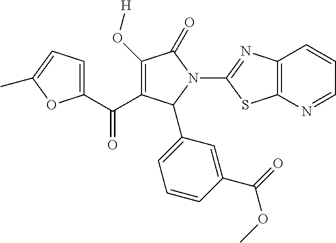 | 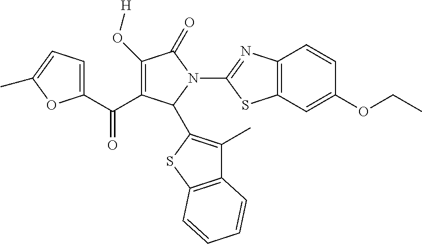 |
| 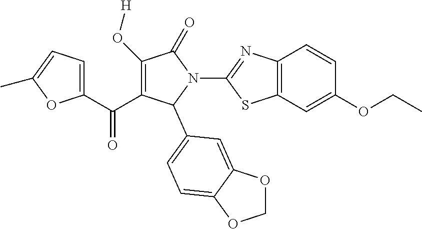 | 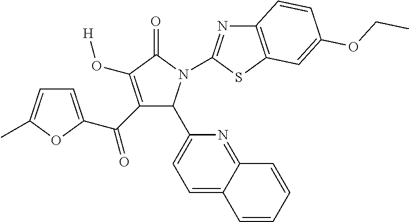 |
| 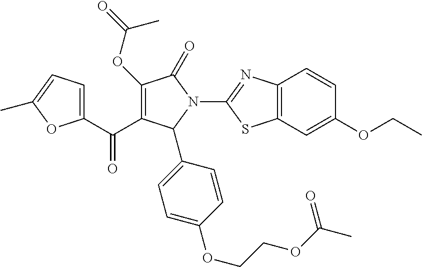 | 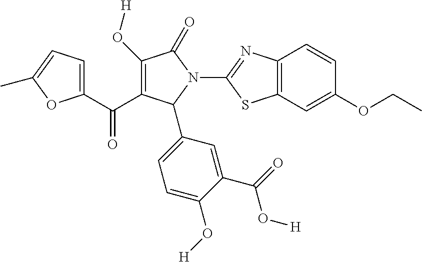 |
| 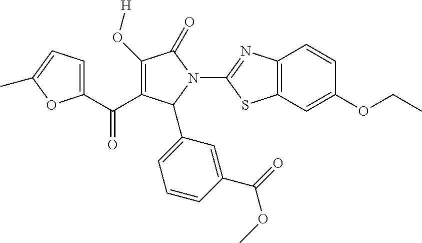 | 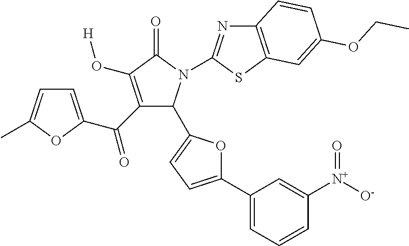 |
| 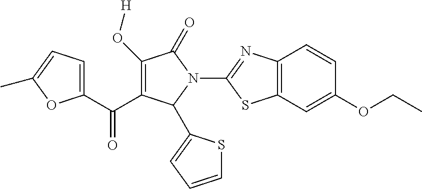 | 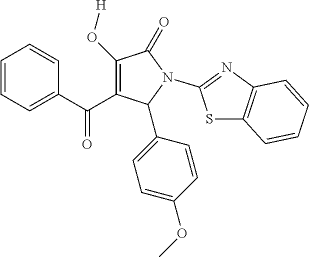 |
| 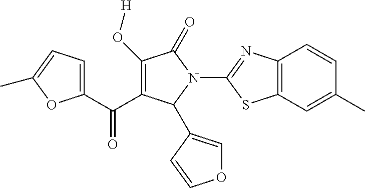 | 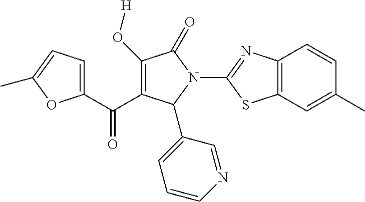 |
| 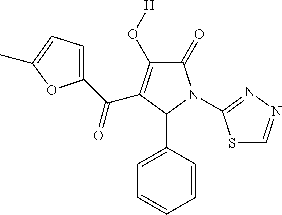 | 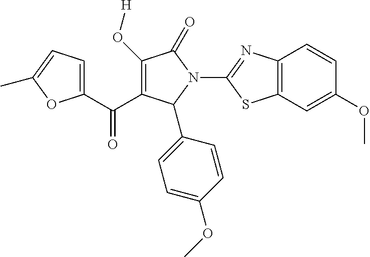 |
| 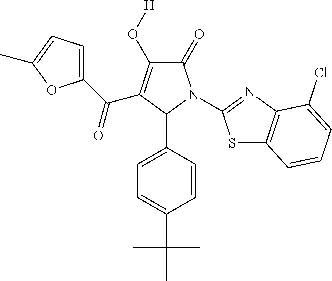 | 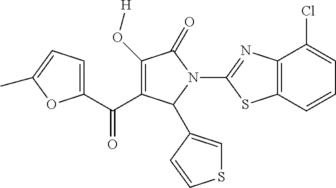 |
| 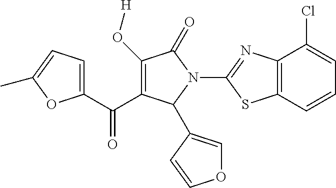 | 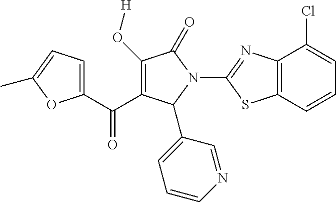 |
| 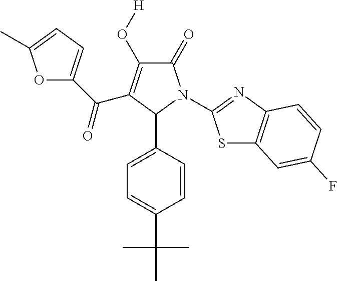 | 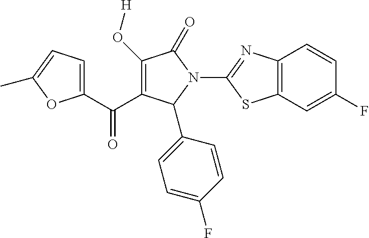 |
| 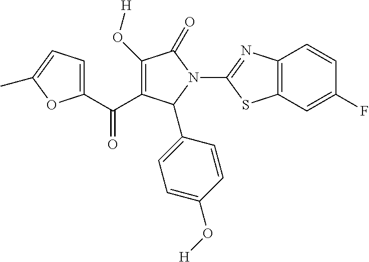 | 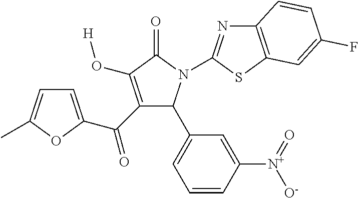 |
| 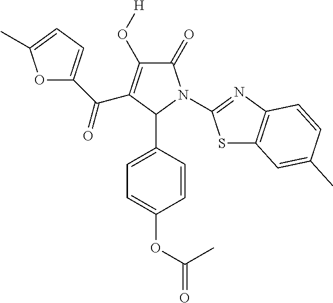 | 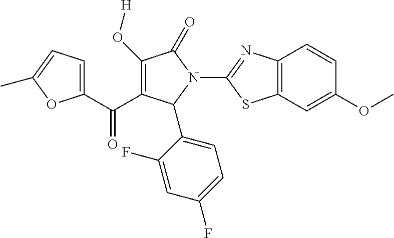 |
| 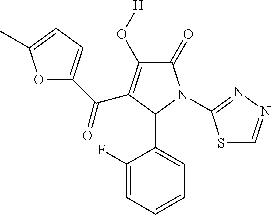 | 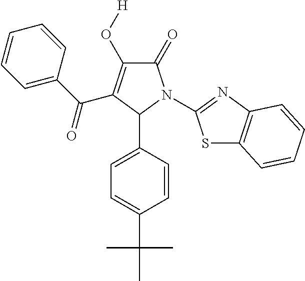 |
| 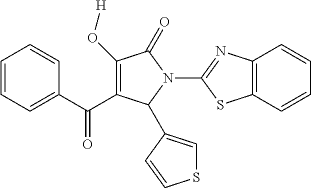 | 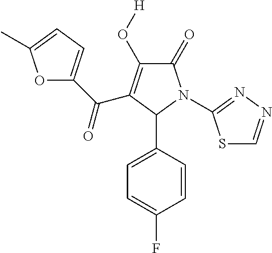 |
| 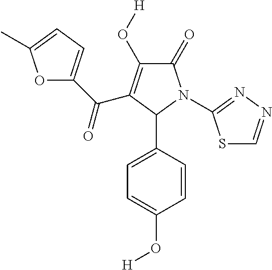 | 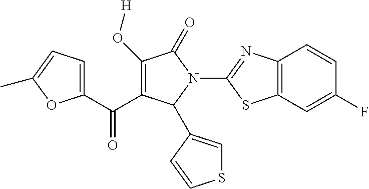 |
| 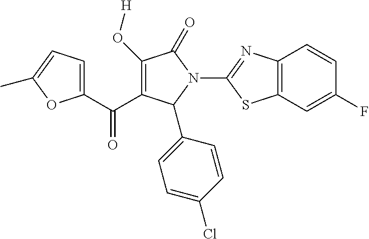 | 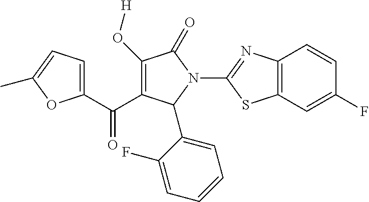 |
| 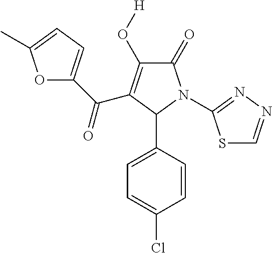 | 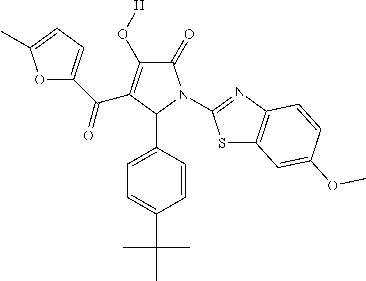 |
| 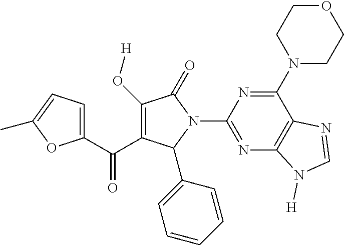 | 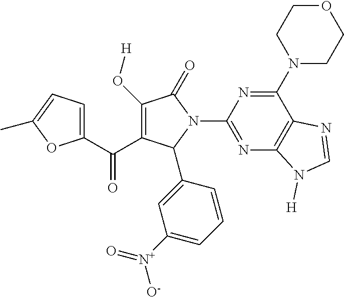 |
| 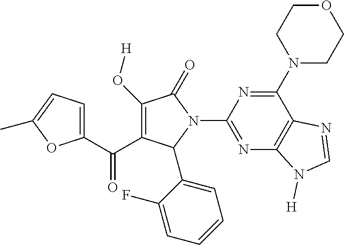 | 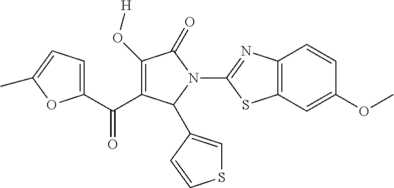 |
| 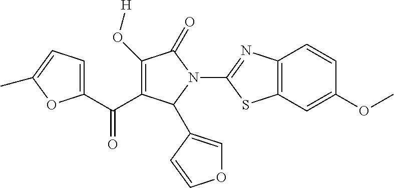 | 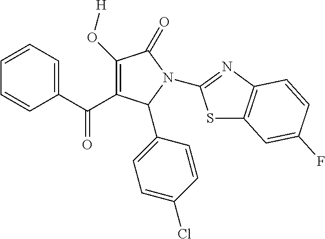 |
| 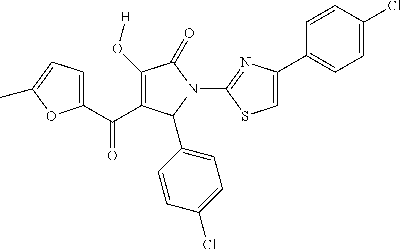 | 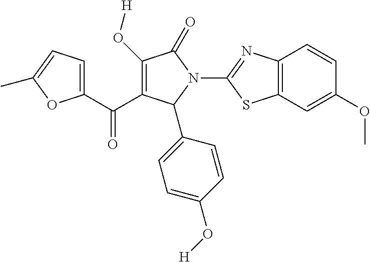 |
| 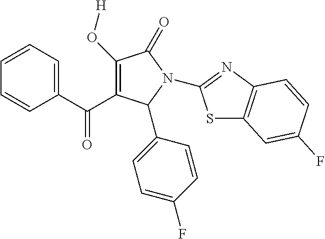 | 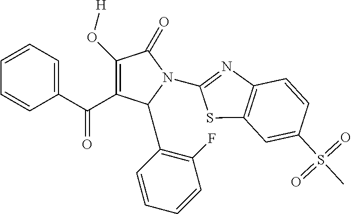 |
| 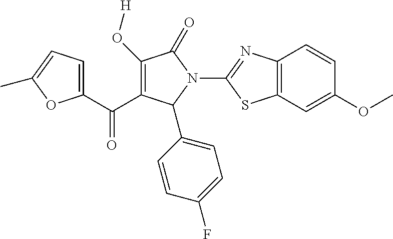 | 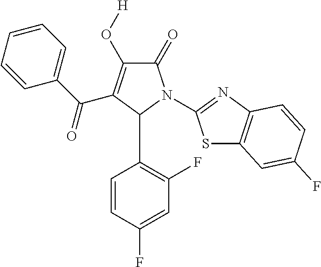 |
| 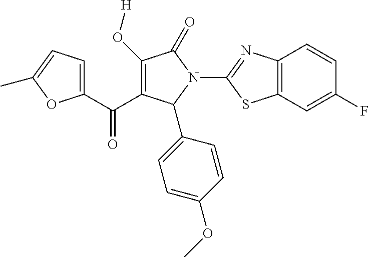 | 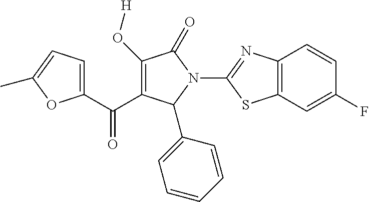 |
| 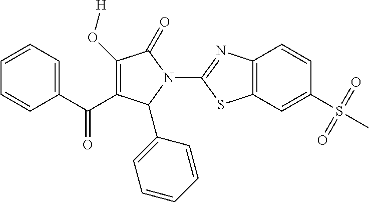 | 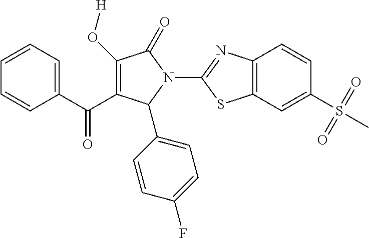 |
| 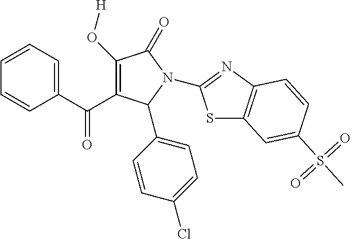 | 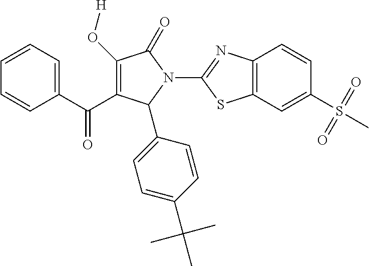 |
| 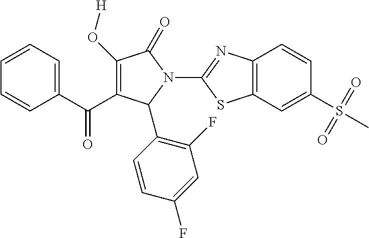 | 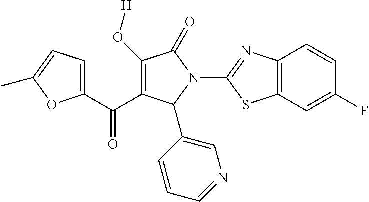 |
| 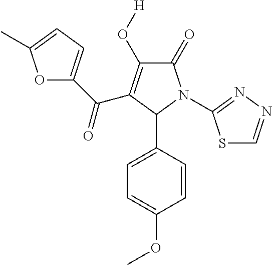 | 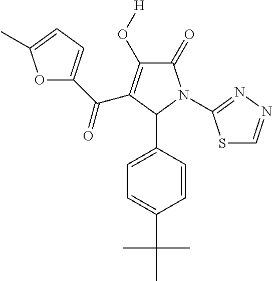 |
| 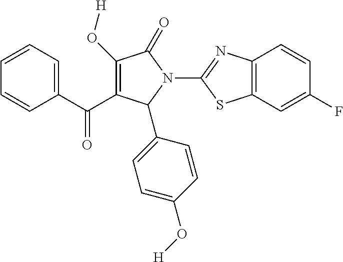 | 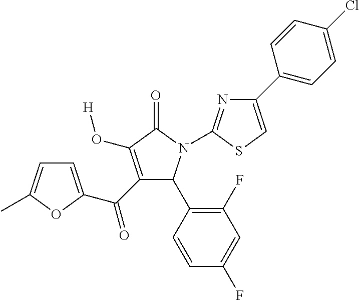 |
| 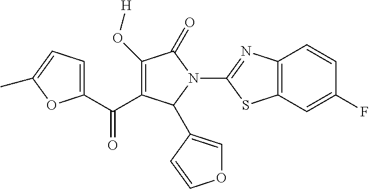 | 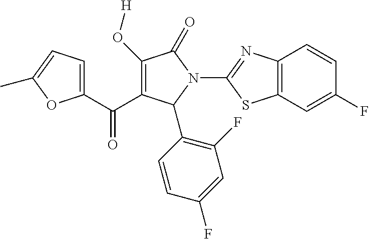 |
| 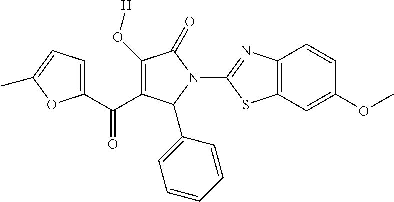 | 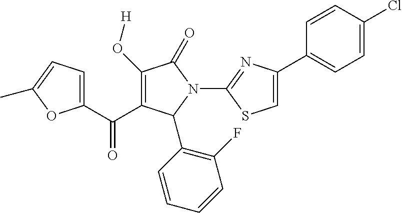 |
| 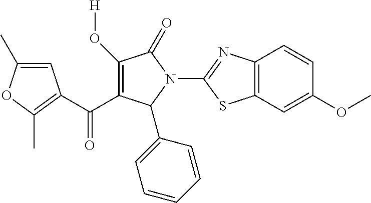 | 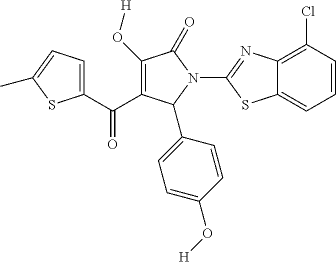 |
| 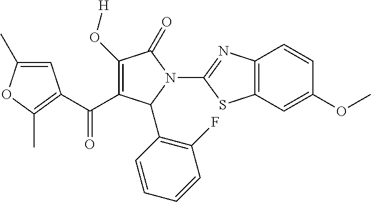 | 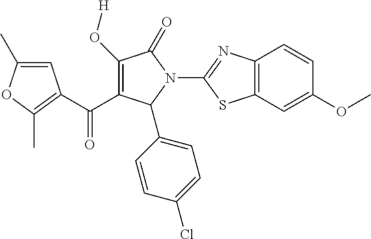 |
| 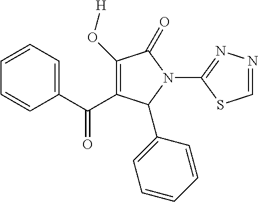 | 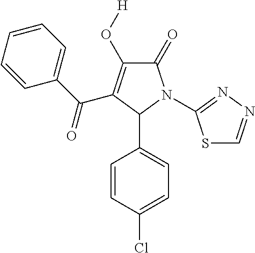 |
| 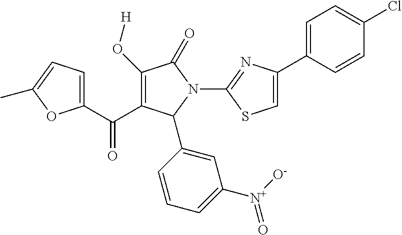 | 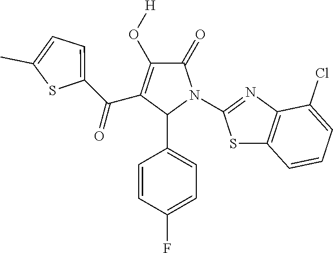 |
| 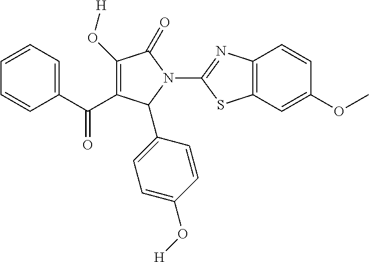 | 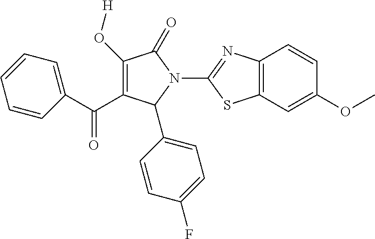 |
| 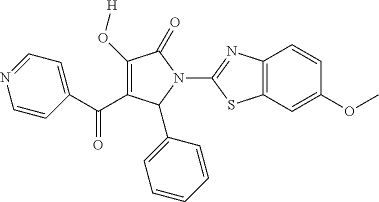 | 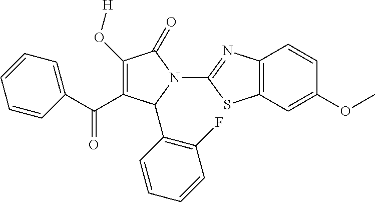 |
| 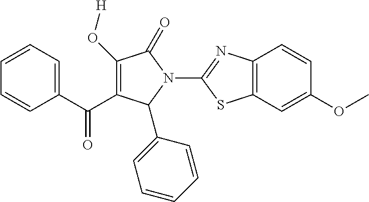 | 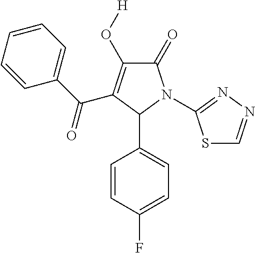 |
| 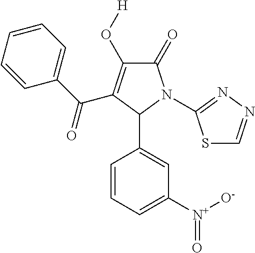 | 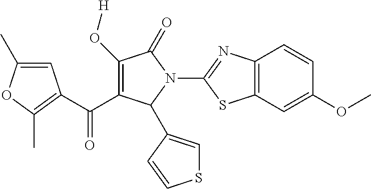 |
| 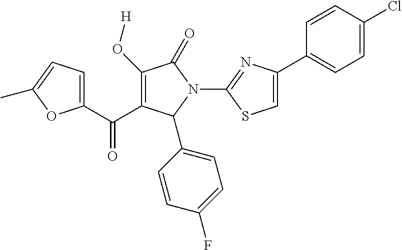 | 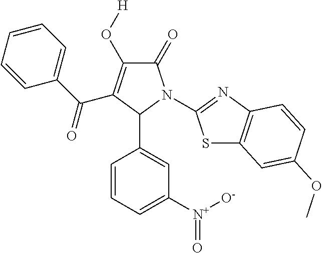 |
| 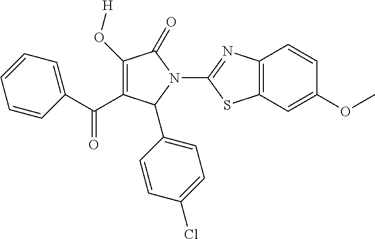 | 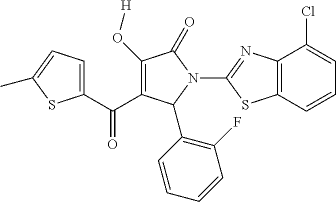 |
| 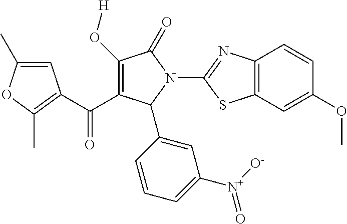 | 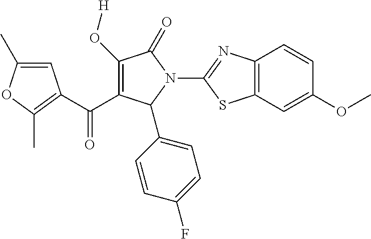 |
| 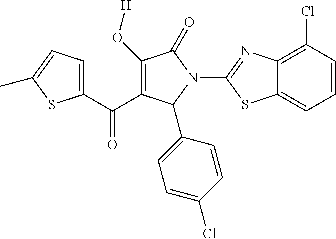 | 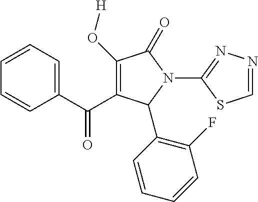 |
| 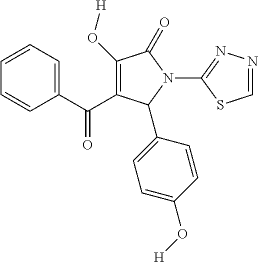 | 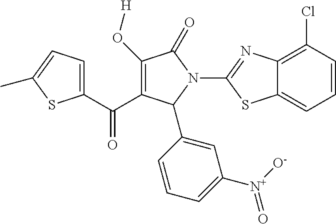 |
| 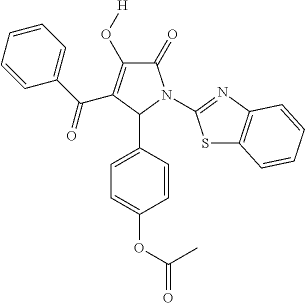 | 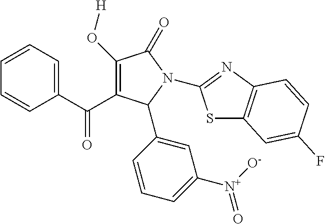 |
| 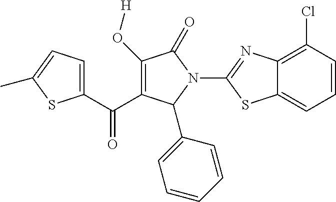 | 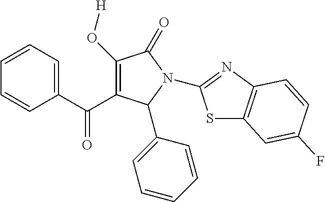 |
| 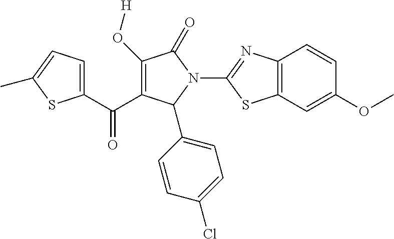 | 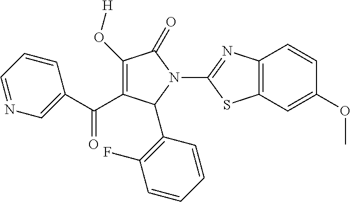 |
| 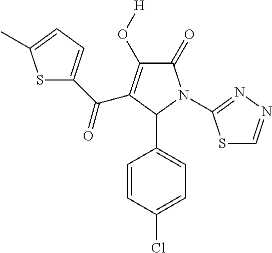 | 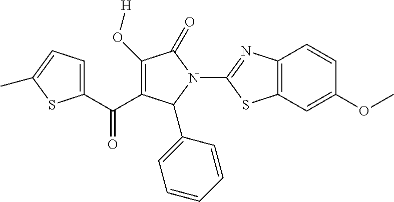 |
| 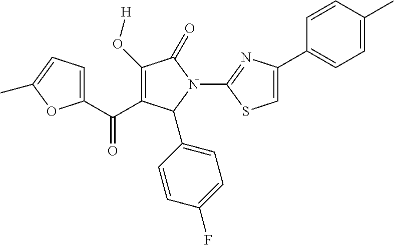 | 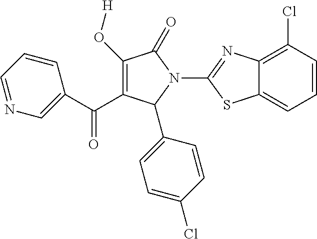 |
| 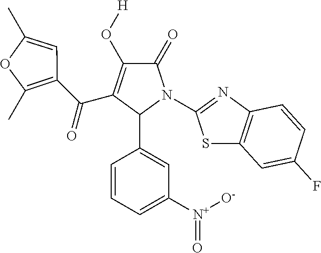 | 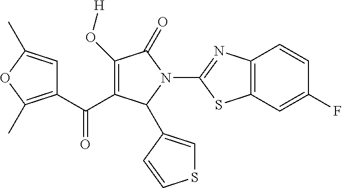 |
| 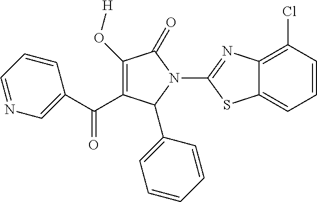 | 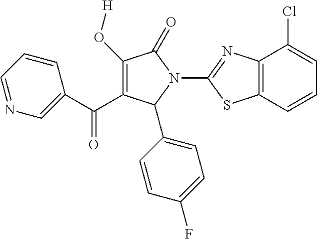 |
| 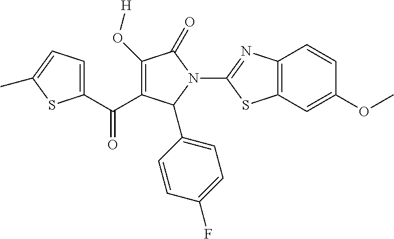 | 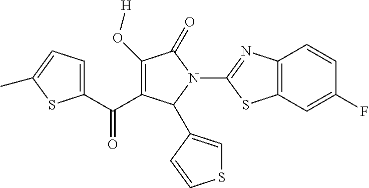 |
| 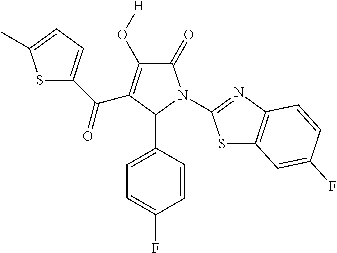 | 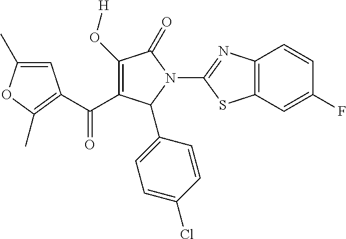 |
| 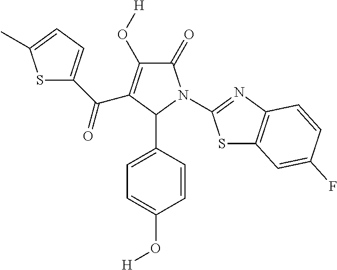 | 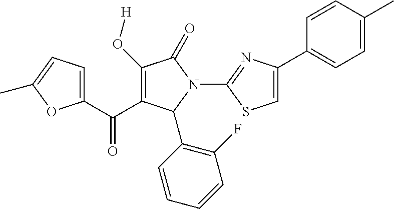 |
| 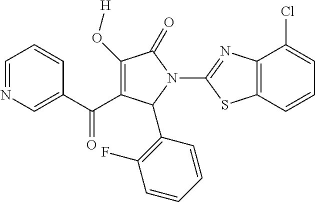 | 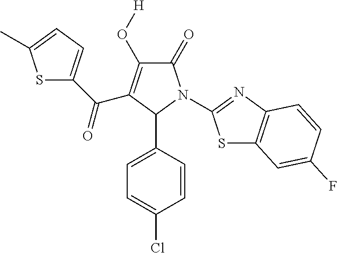 |
| 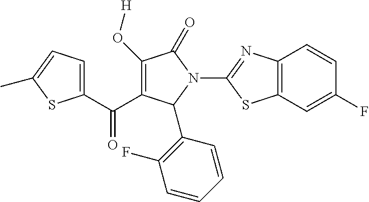 | 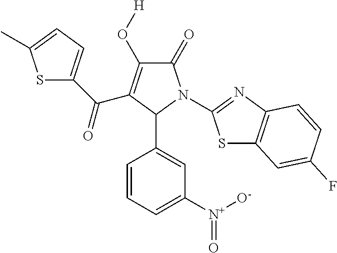 |
| 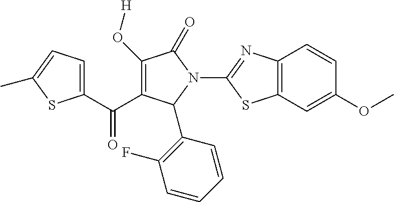 | 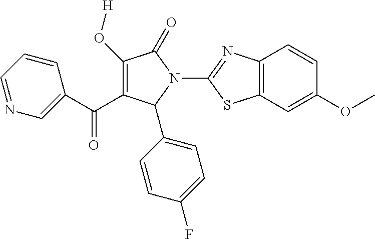 |
| 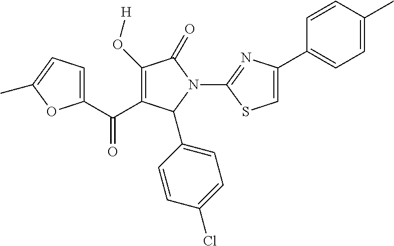 | 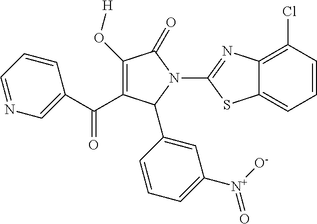 |
| 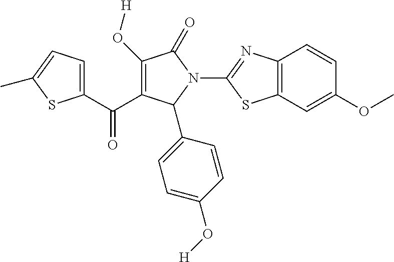 | 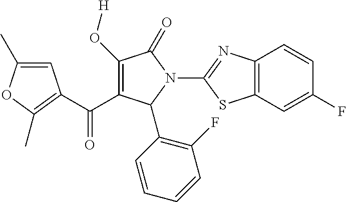 |
| 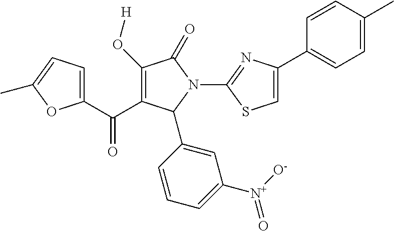 | 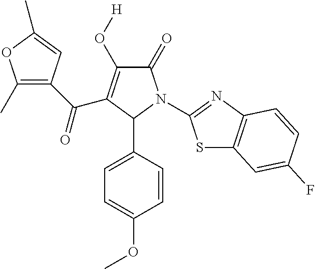 |
| 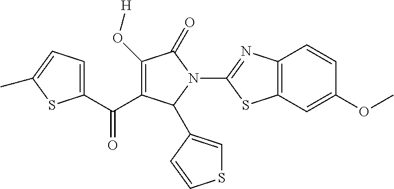 | 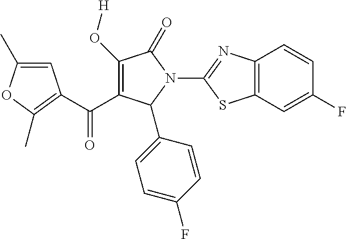 |
| 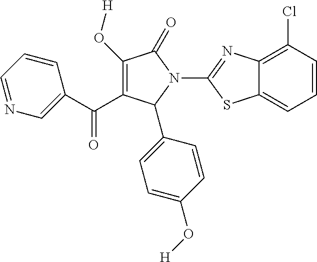 | 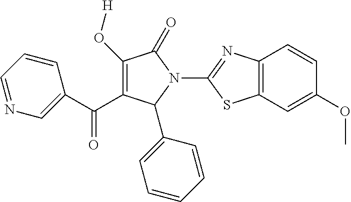 |
| 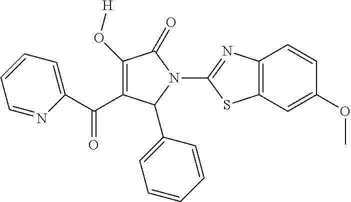 | 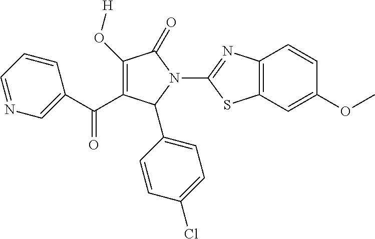 |
| 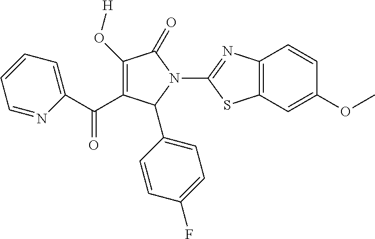 | 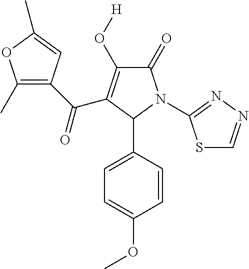 |
| 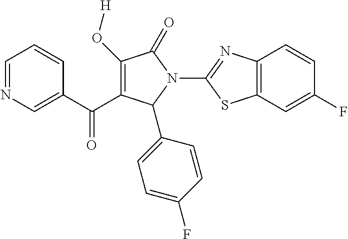 | 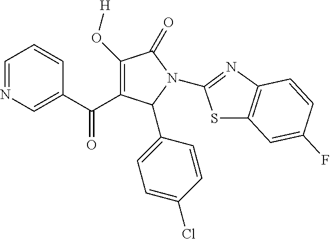 |
| 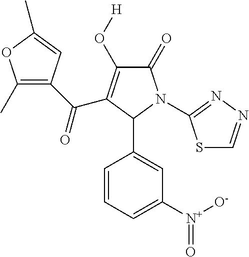 | 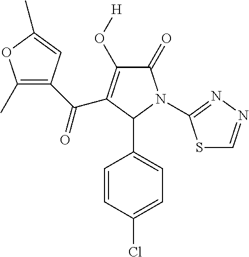 |
| 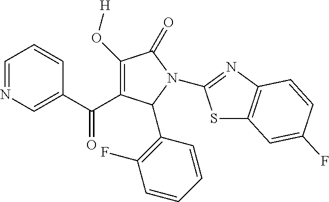 | 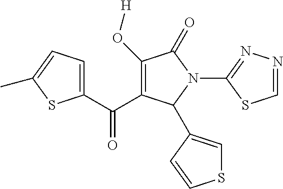 |
| 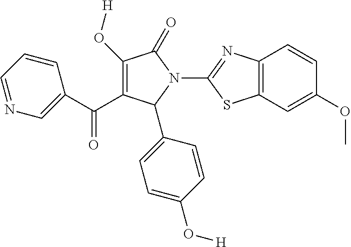 | 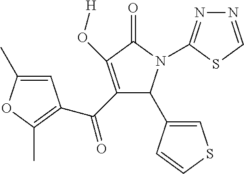 |
| 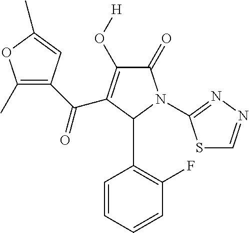 | 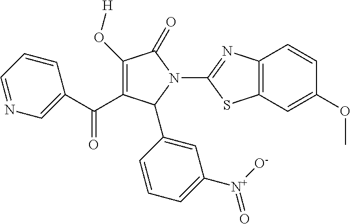 |
| 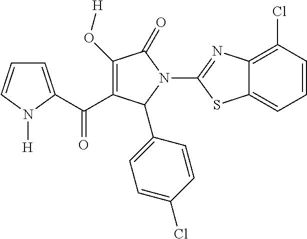 | 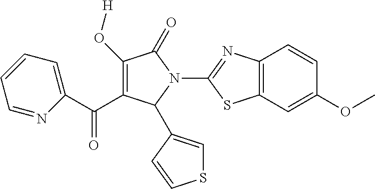 |
| 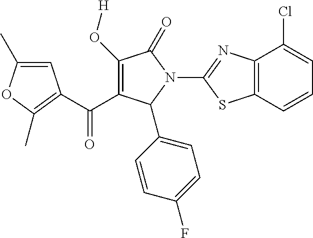 | 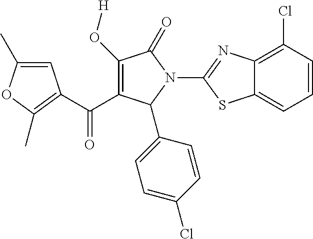 |
| 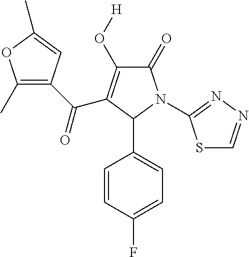 | 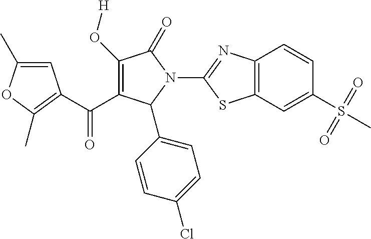 |
| 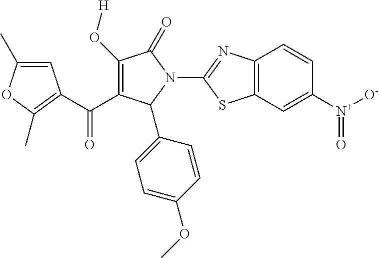 | 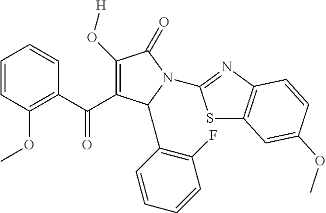 |
| 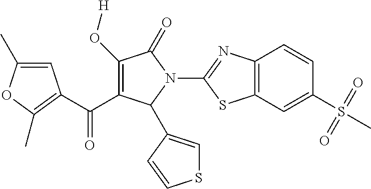 | 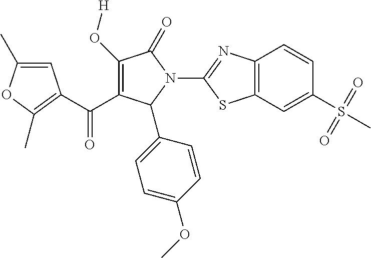 |
| 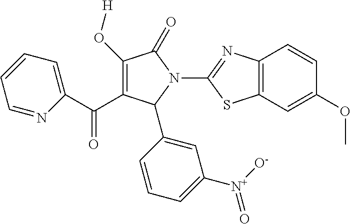 | 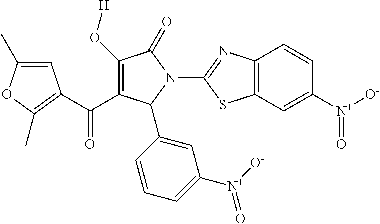 |
| 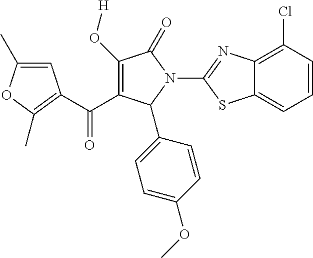 | 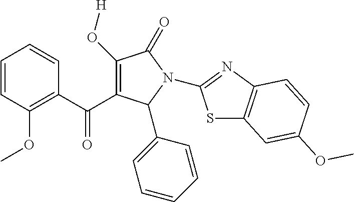 |
| 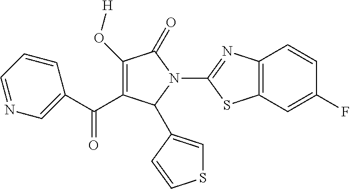 | 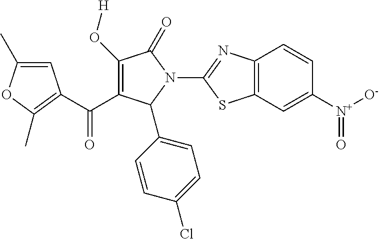 |
| 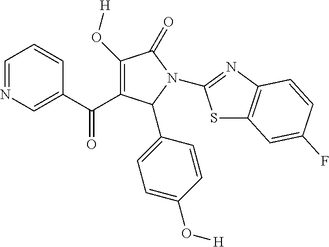 | 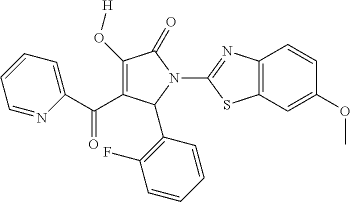 |
| 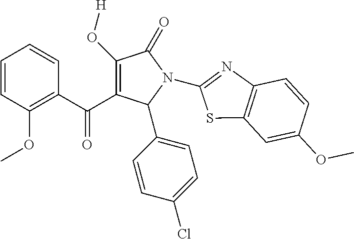 | 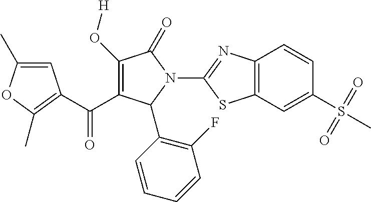 |
| 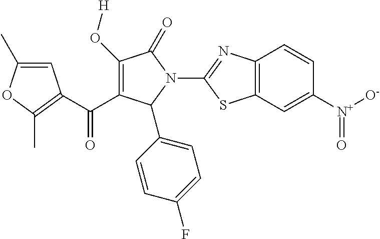 | 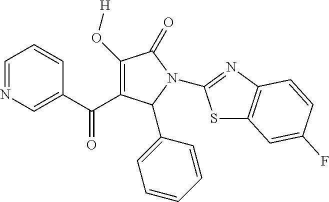 |
| 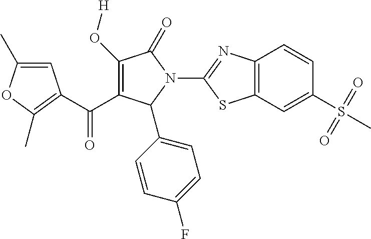 | 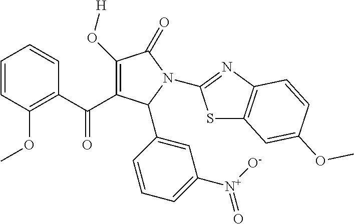 |
| 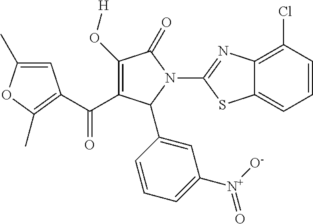 | 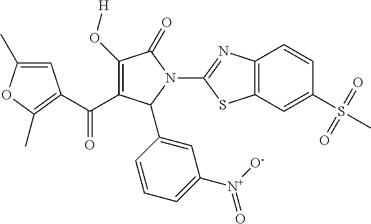 |
| 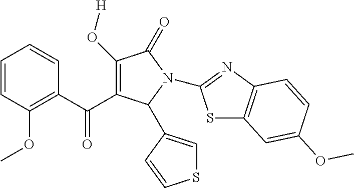 | 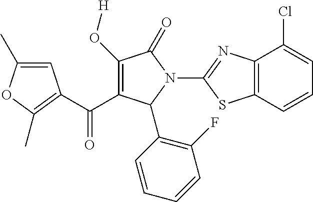 |
| 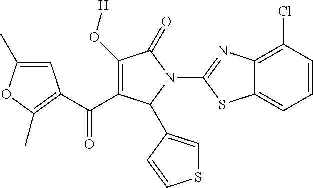 | 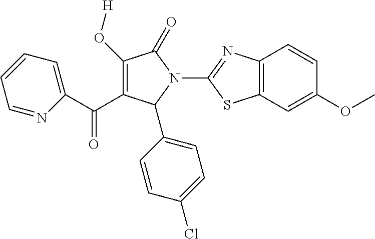 |
| 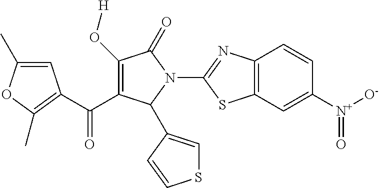 | 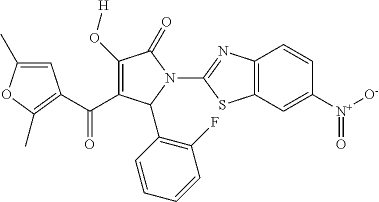 |
| 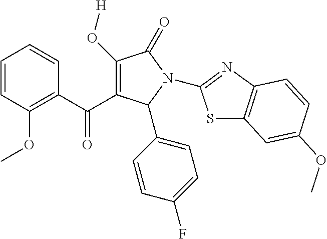 | 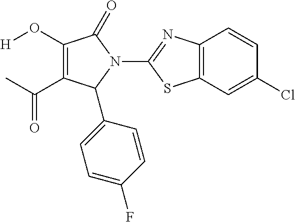 |
| 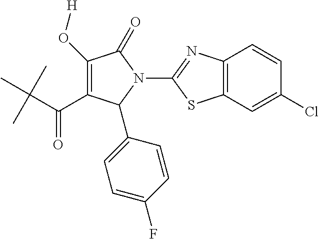 | 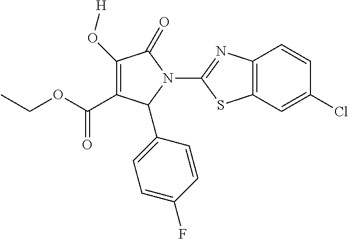 |
| 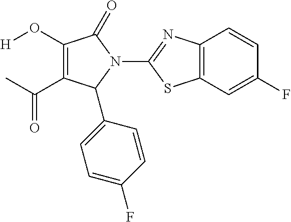 | 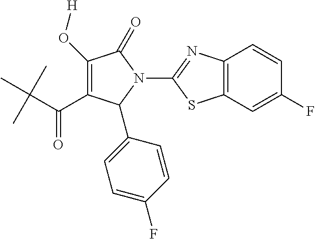 |
| 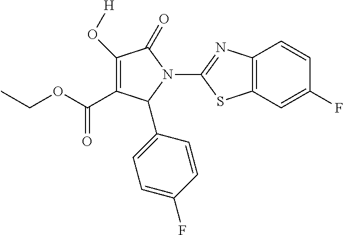 | 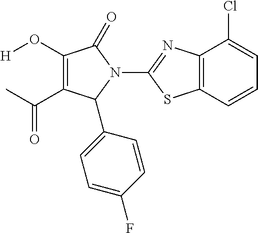 |
| 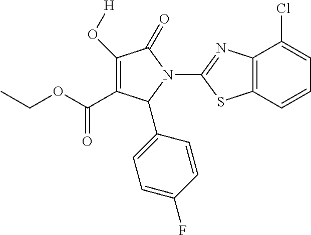 | 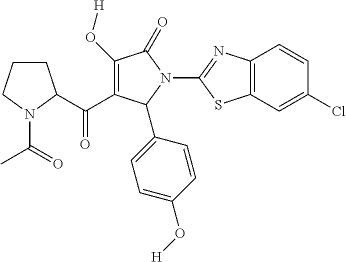 |
| 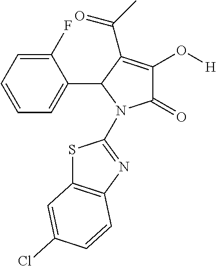 | 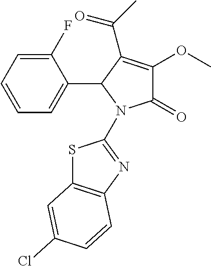 |
| 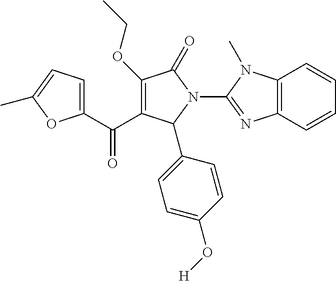 | 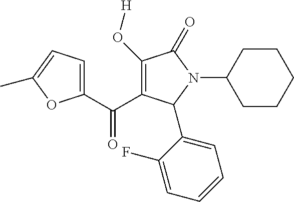 |
| 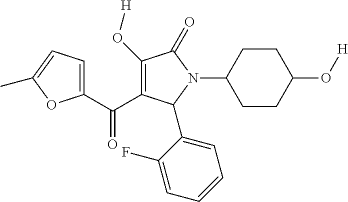 | 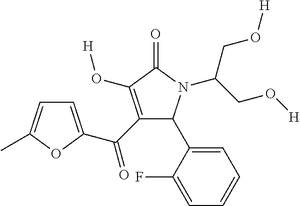 |
| 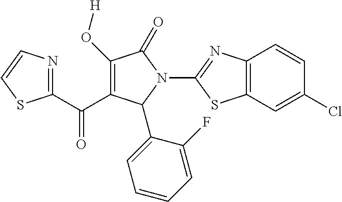 | 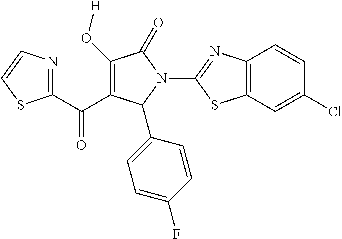 |
| 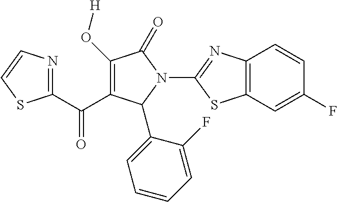 | 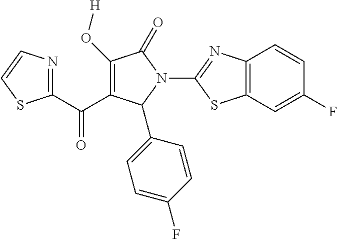 |
| 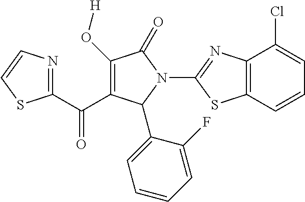 | 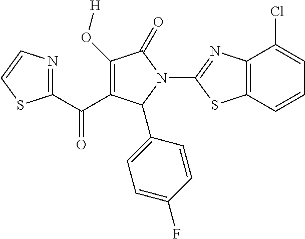 |
| 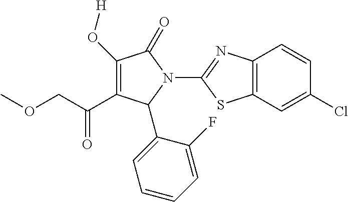 | 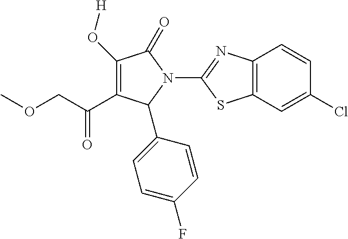 |
| 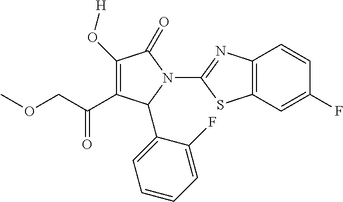 | 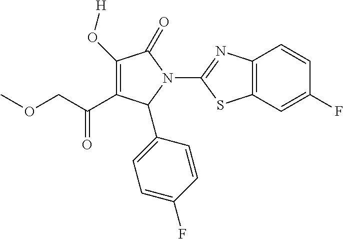 |
| 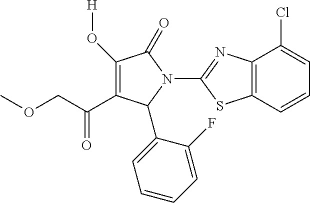 | 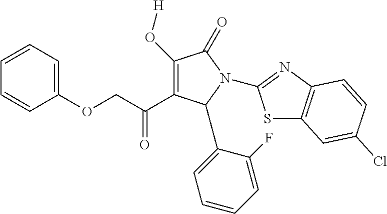 |
| 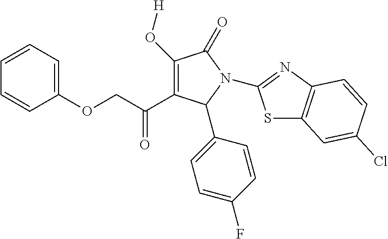 | 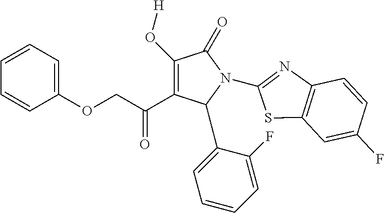 |
| 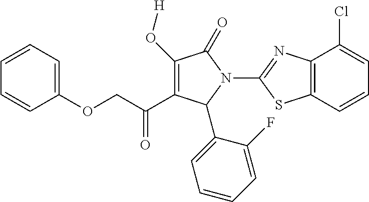 | 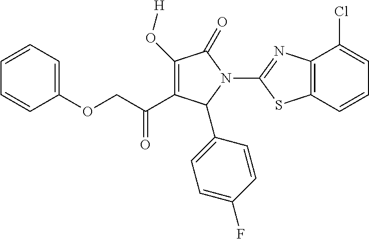 |
| 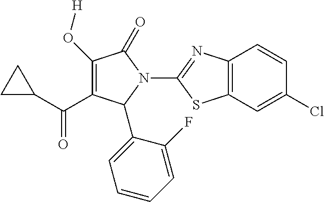 | 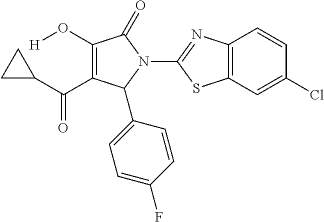 |
| 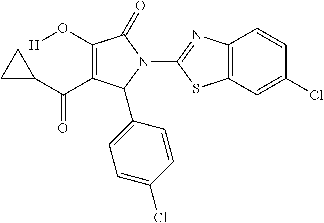 | 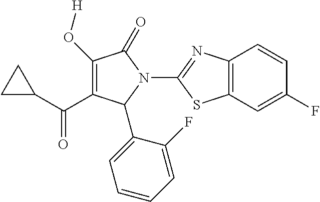 |
| 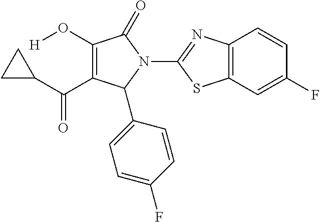 | 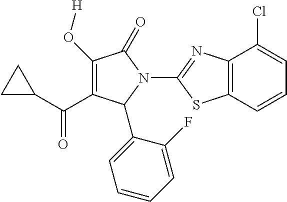 |
| 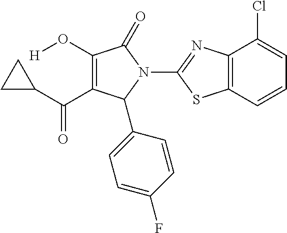 | 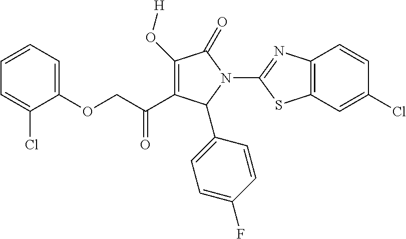 |
| 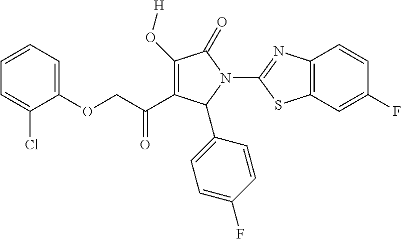 | 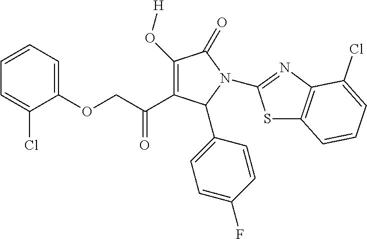 |
| 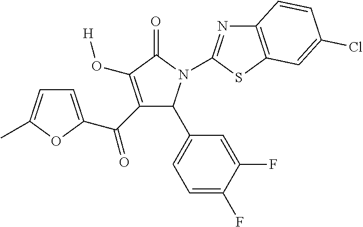 | 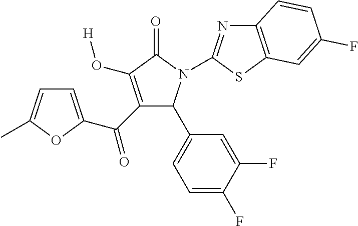 |
| 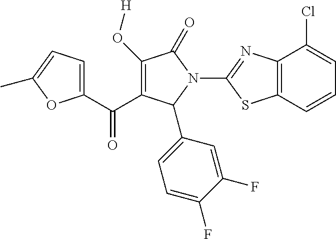 | 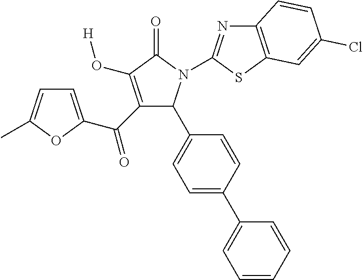 |
| 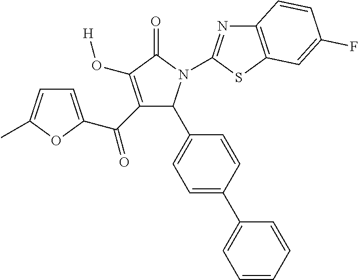 | 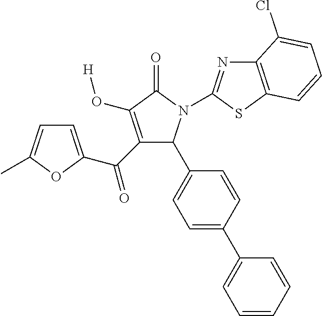 |
| 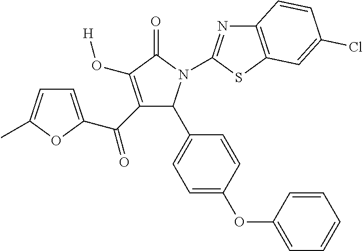 | 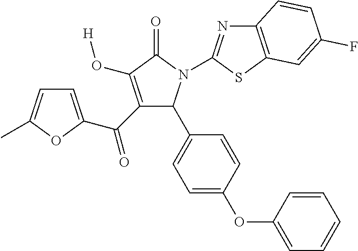 |
| 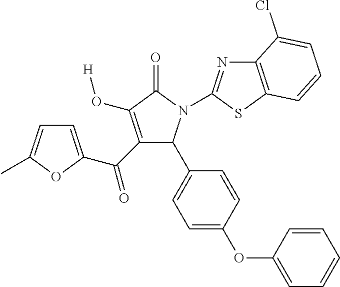 | 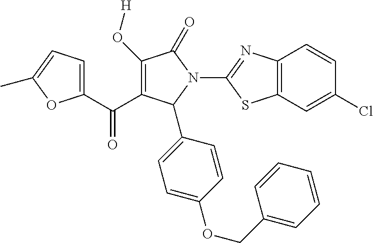 |
| 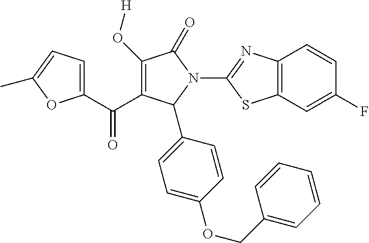 | 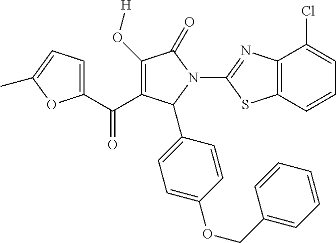 |
| 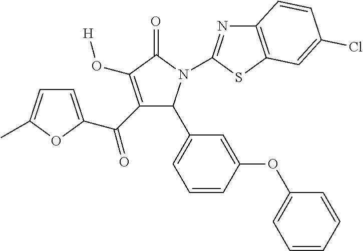 | 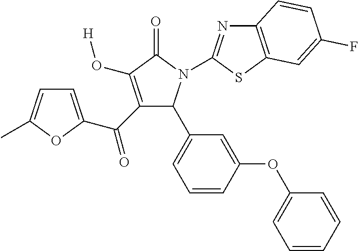 |
| 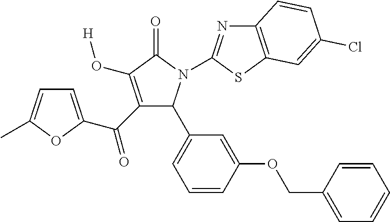 | 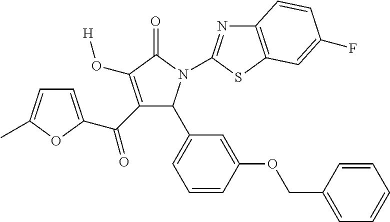 |
| 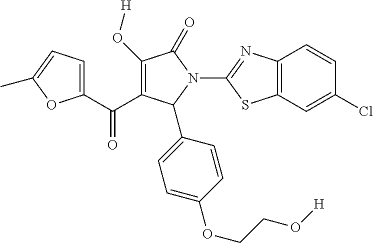 | 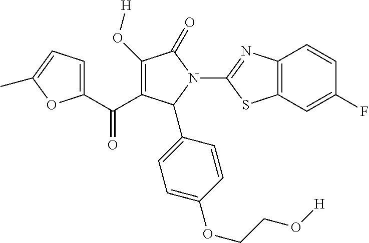 |
| 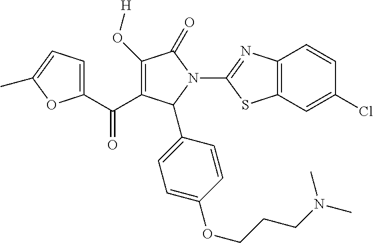 | 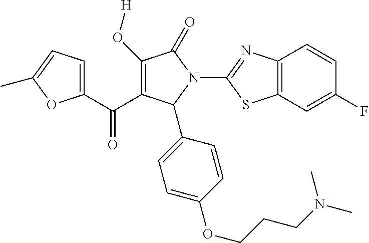 |
| 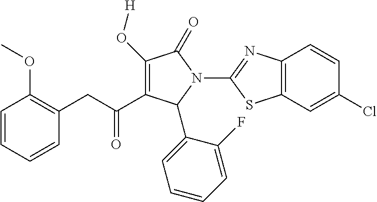 | 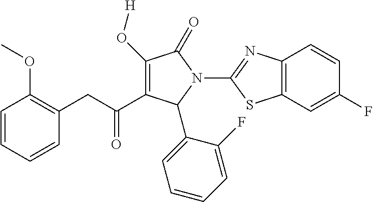 |
| 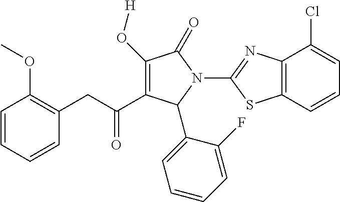 | 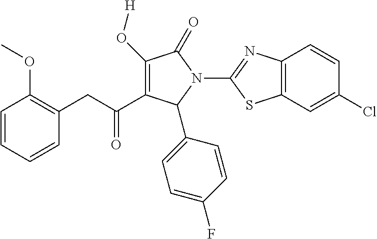 |
| 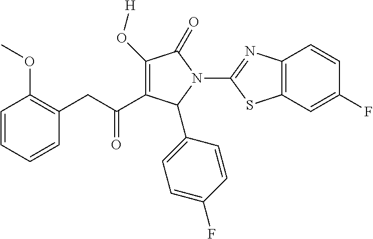 | 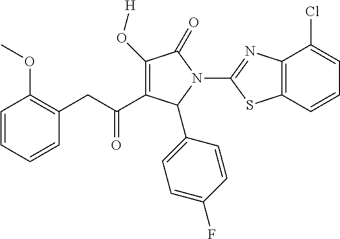 |
| 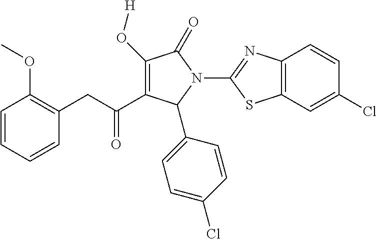 | 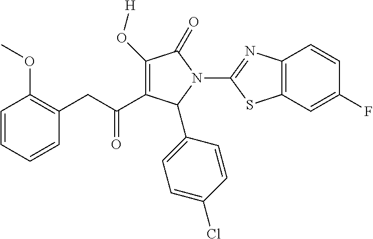 |
| 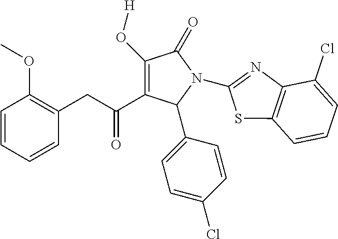 | 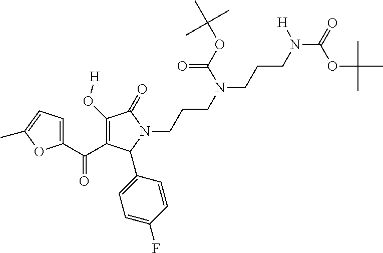 |
| 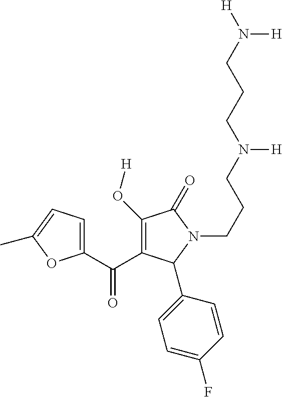 | 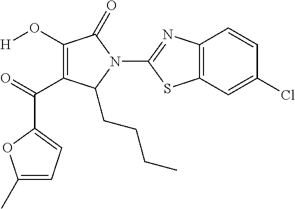 |
| 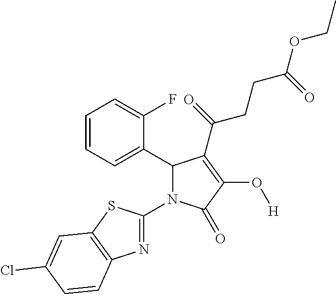 | 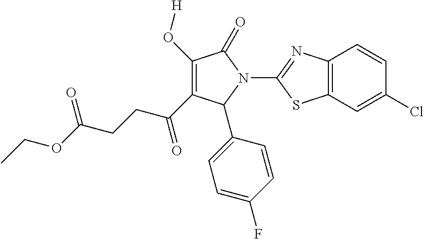 |
| 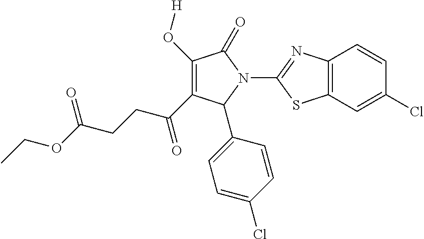 | 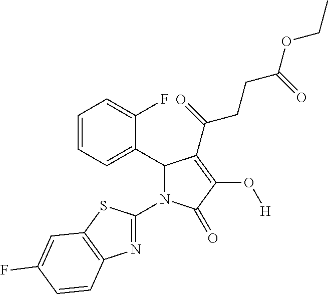 |
| 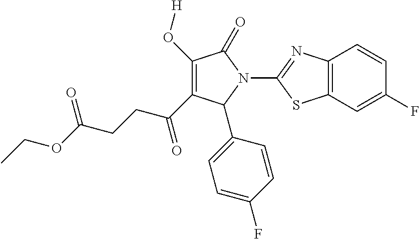 | 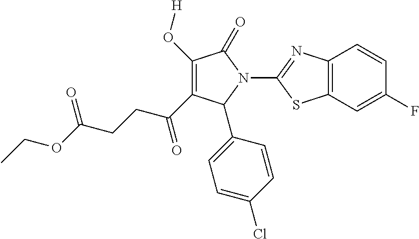 |
| 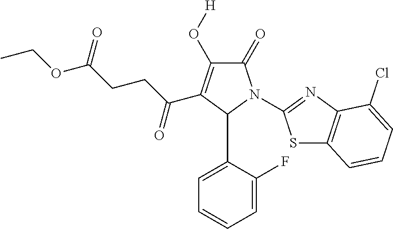 | 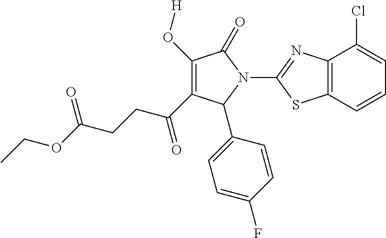 |
| 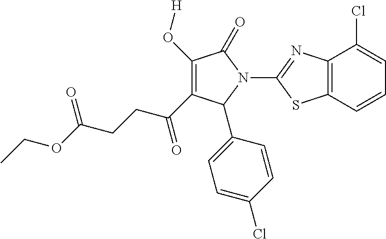 | 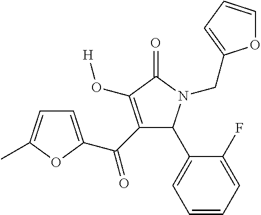 |
| 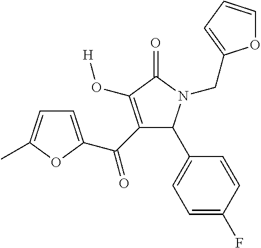 | 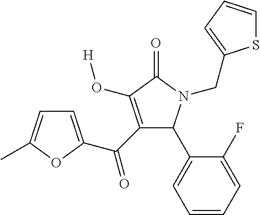 |
| 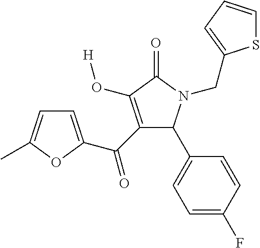 | 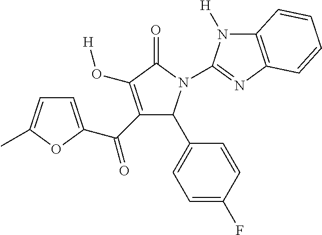 |
| 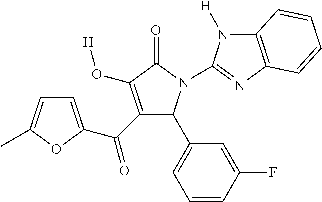 | 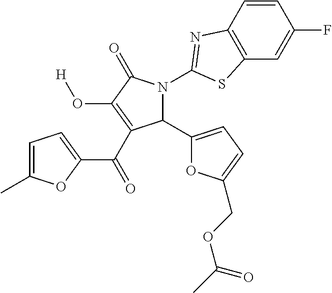 |
| 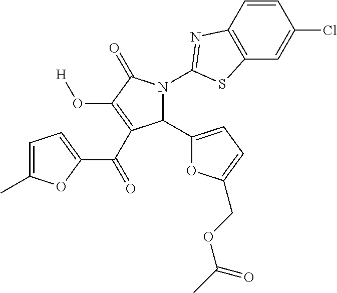 | 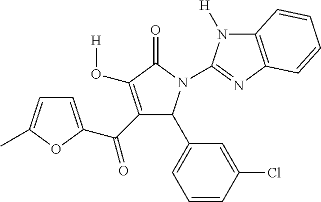 |
| 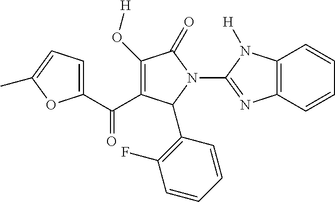 | 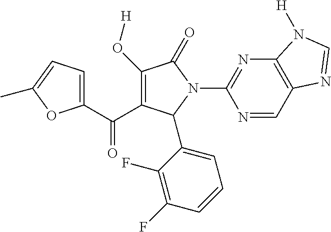 |
| 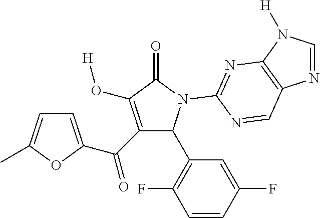 | 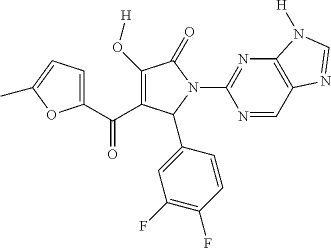 |
| 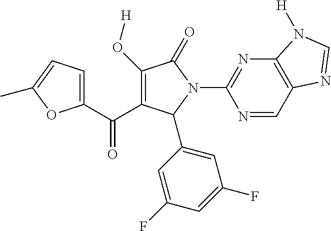 | 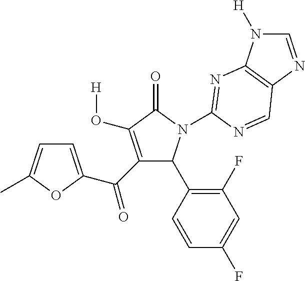 |
| 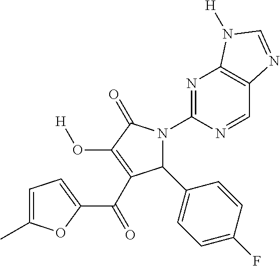 | 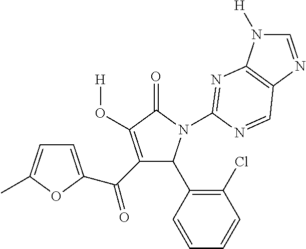 |
| 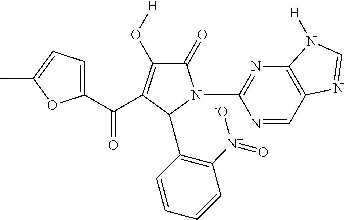 | 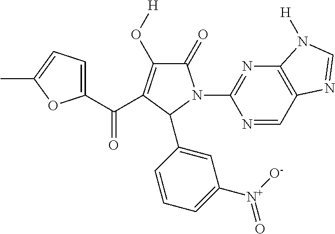 |
| 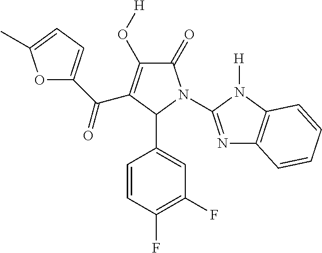 | 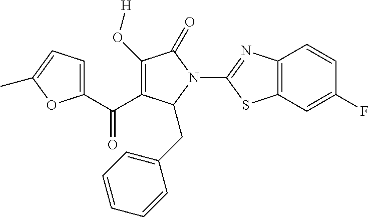 |
| 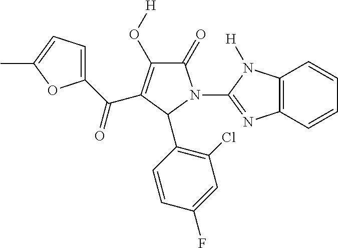 | 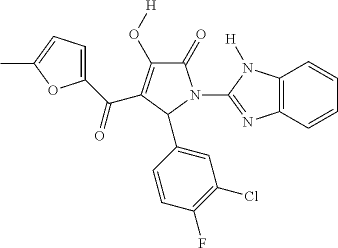 |
| 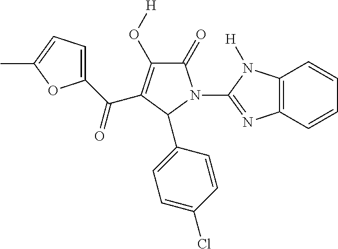 | 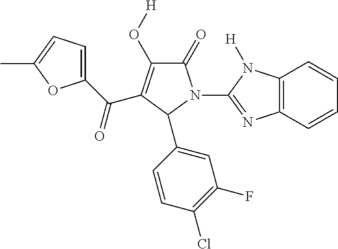 |
| 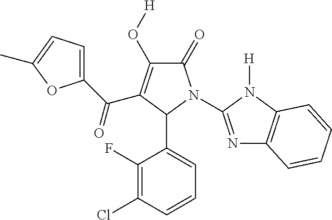 | 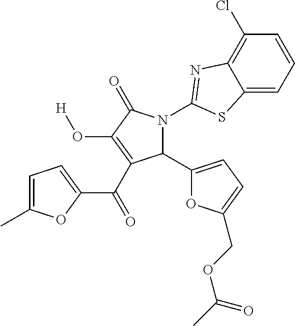 |
| 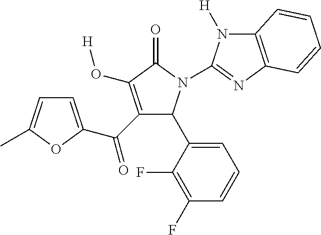 | 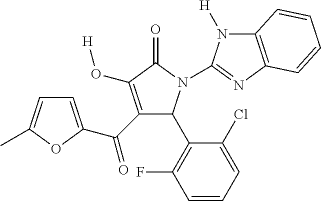 |
| 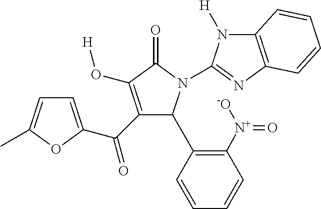 | 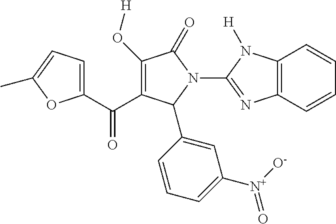 |
| 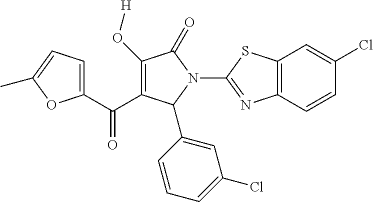 | 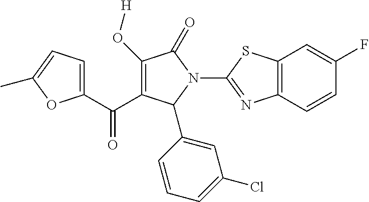 |
| 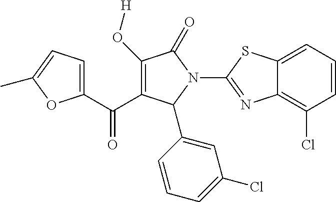 | 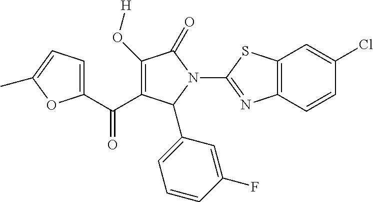 |
| 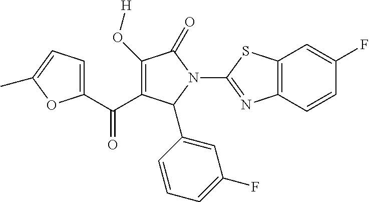 | 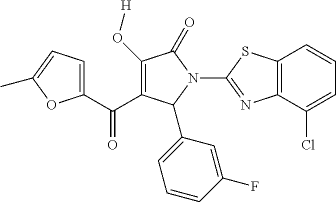 |
| 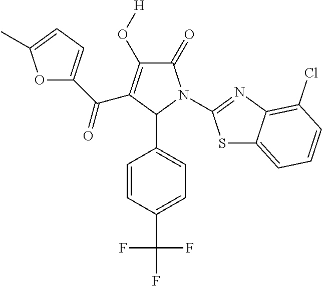 | 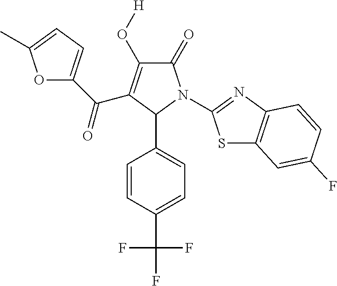 |
| 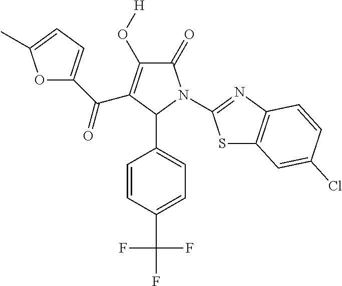 | 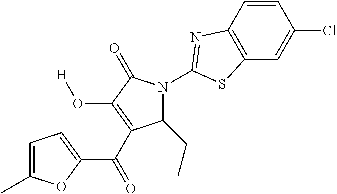 |
| 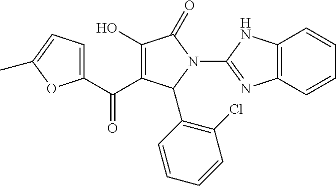 | 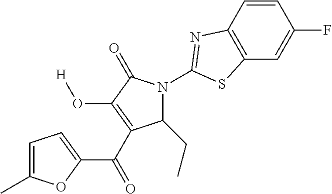 |
| 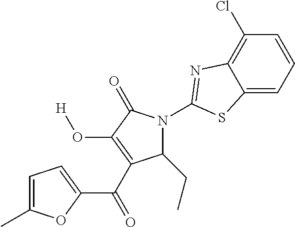 | 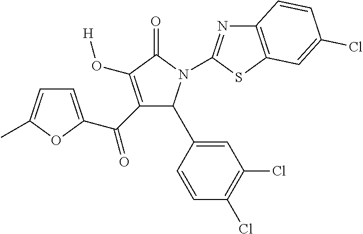 |
| 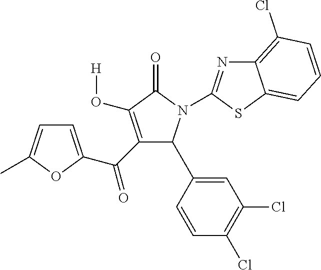 | 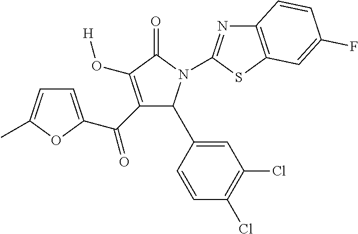 |
| 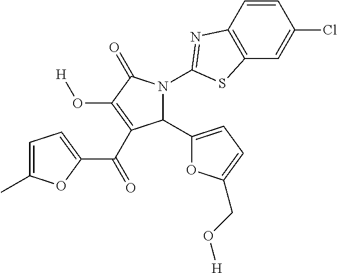 | 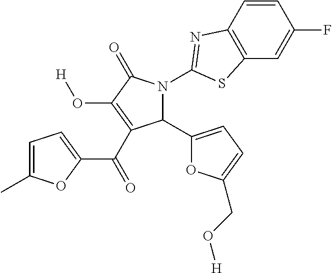 |
| 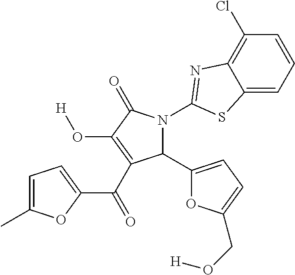 | 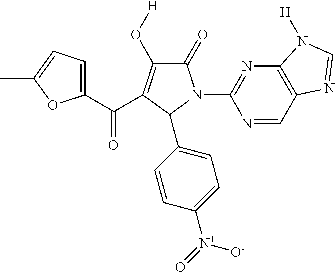 |
| 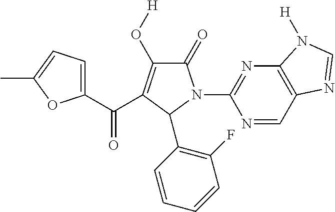 | 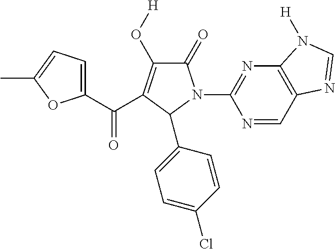 |
| 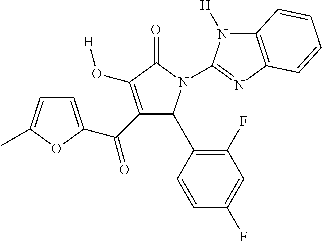 | 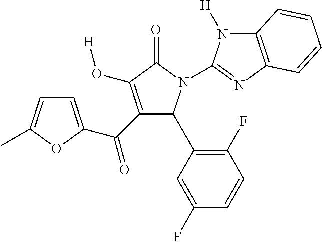 |
| 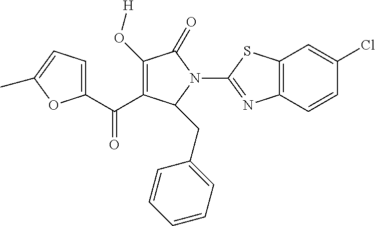 | 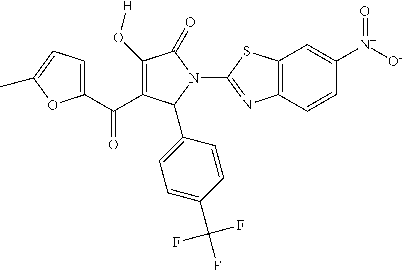 |
| 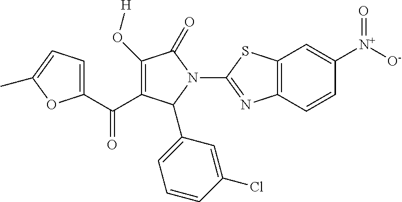 | 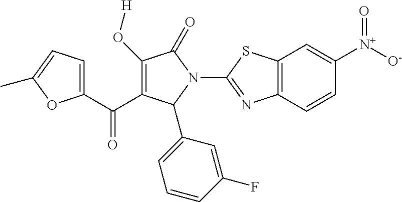 |
| 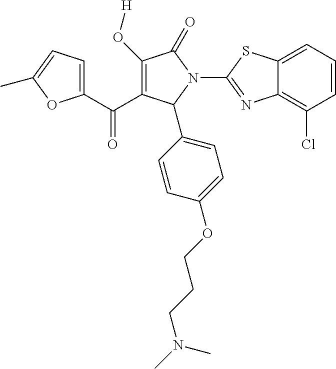 | 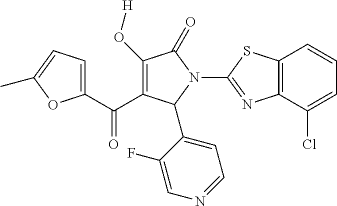 |
| 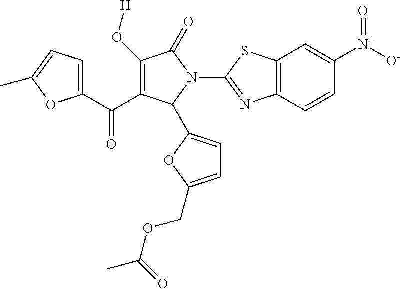 | 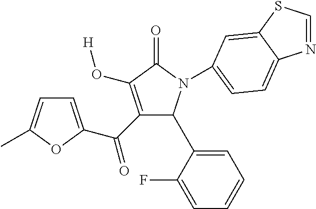 |
| 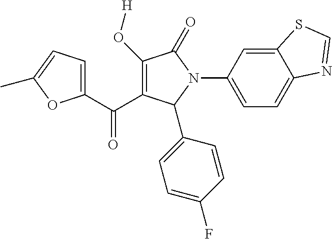 | 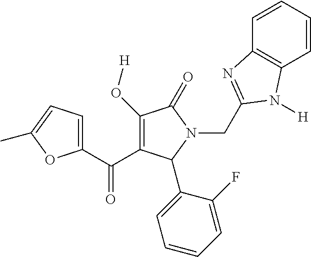 |
| 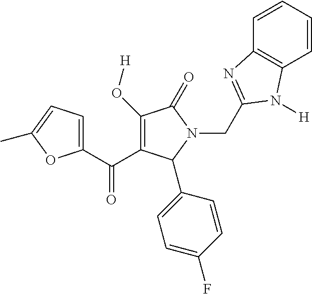 | 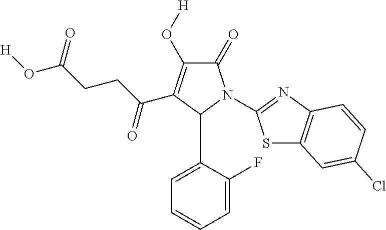 |
| 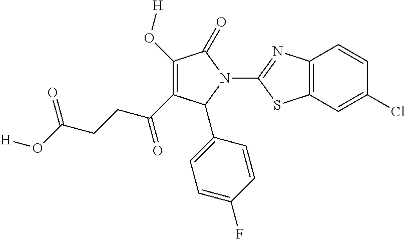 | 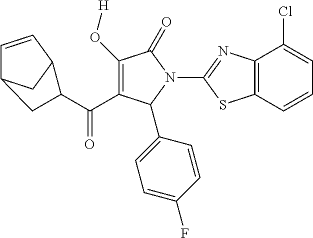 |
| 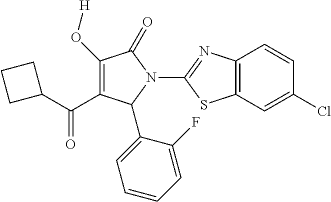 | 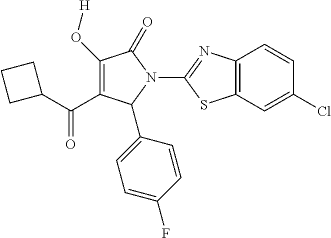 |
| 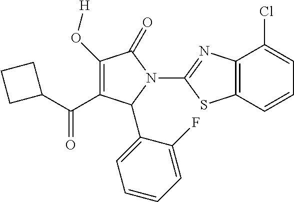 | 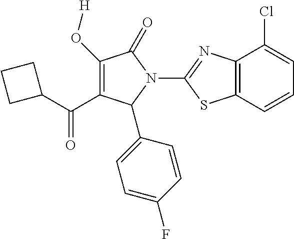 |
| 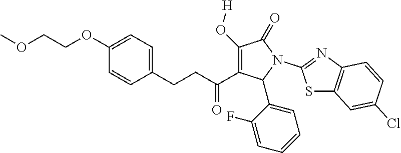 | 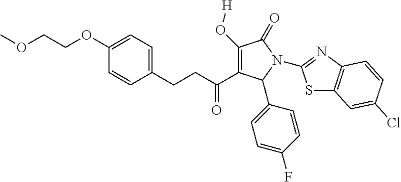 |
| 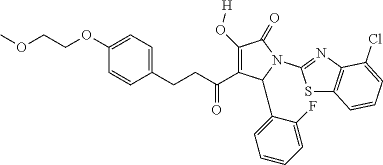 | 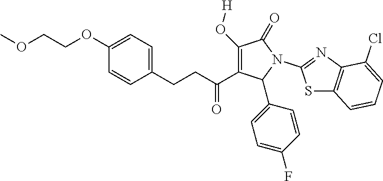 |
| 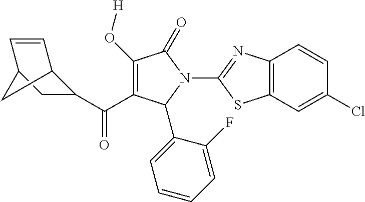 | 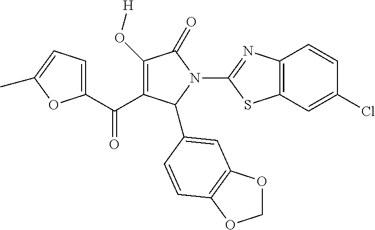 |
| 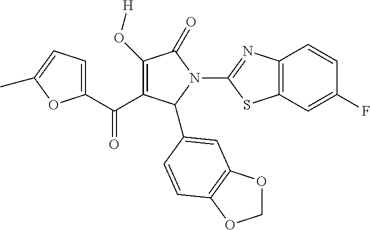 | 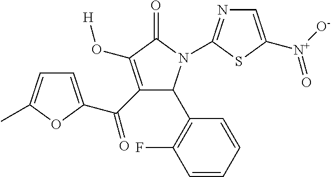 |
| 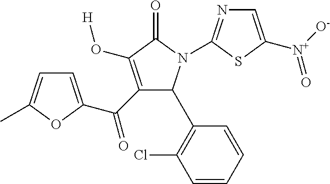 | 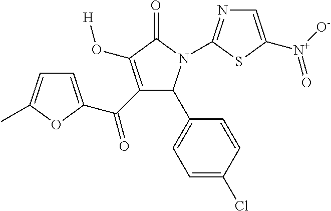 |
| 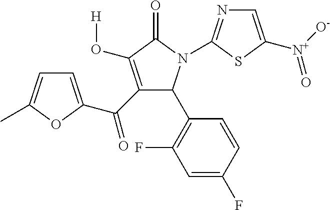 | 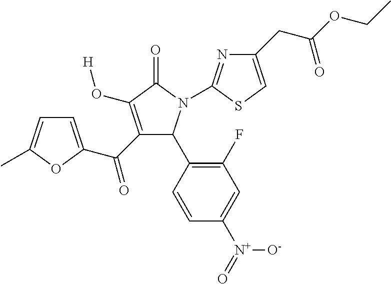 |
| 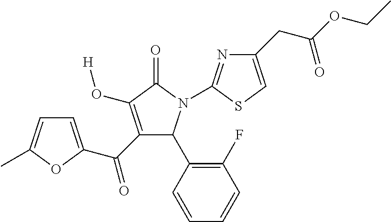 | 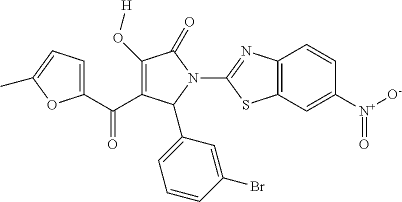 |
| 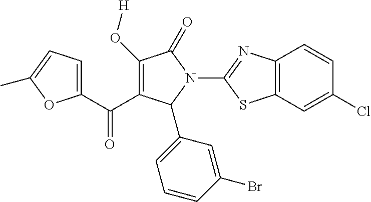 | 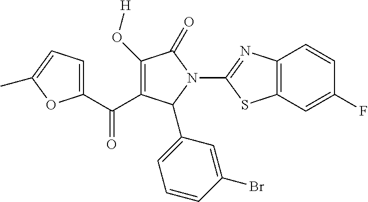 |
| 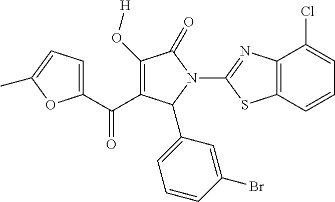 | 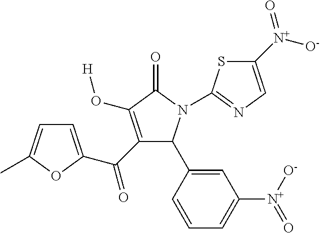 |
| 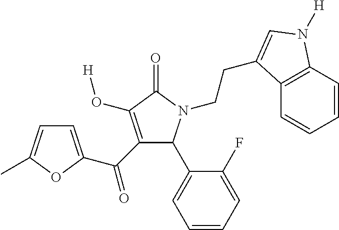 | 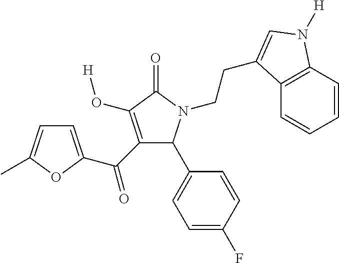 |
| 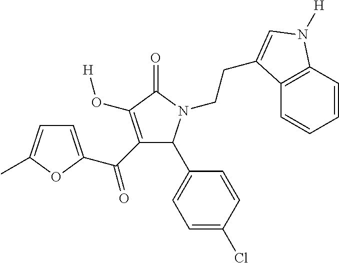 | 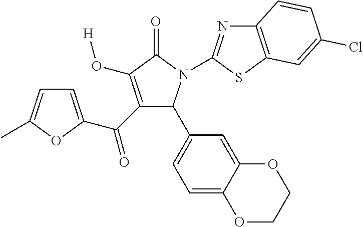 |
| 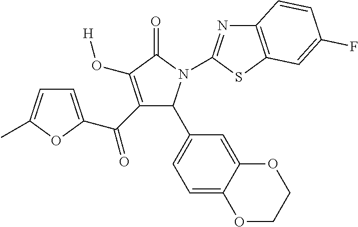 | 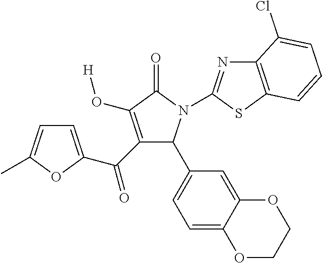 |
| 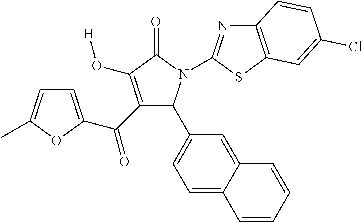 | 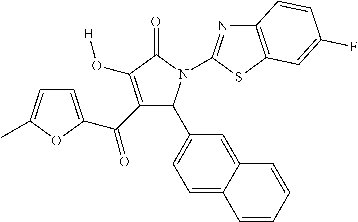 |
| 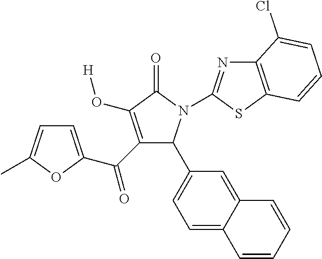 | 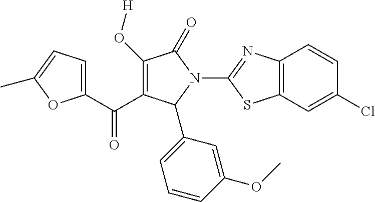 |
| 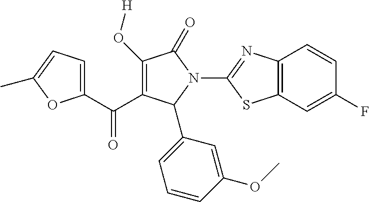 | 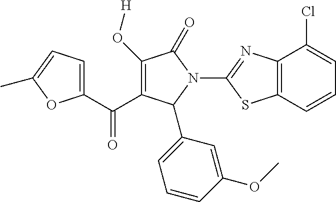 |
| 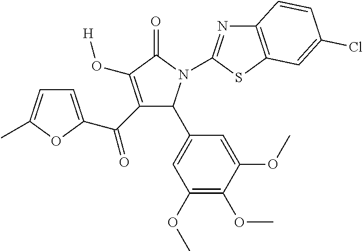 | 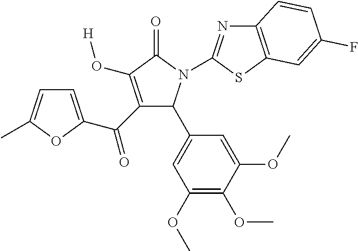 |
| 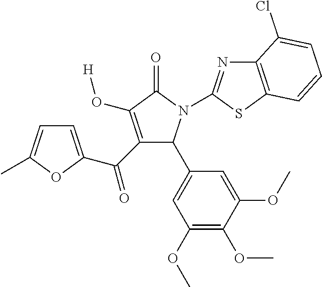 | 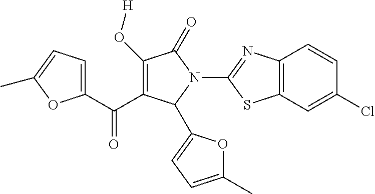 |
| 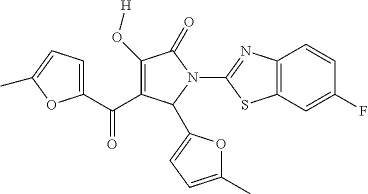 | 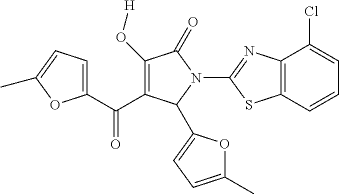 |
| 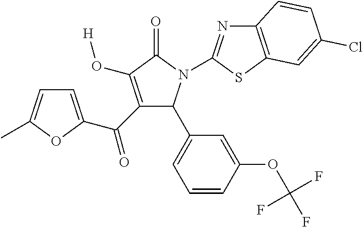 | 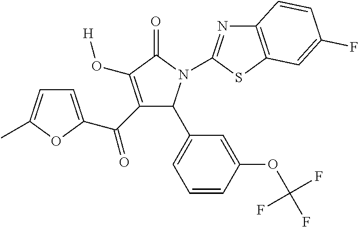 |
| 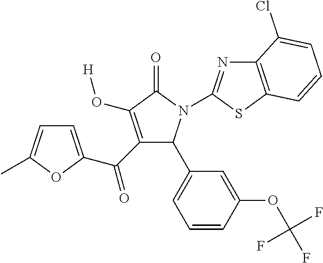 | 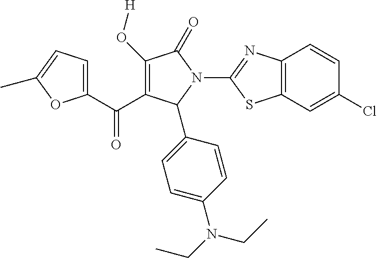 |
| 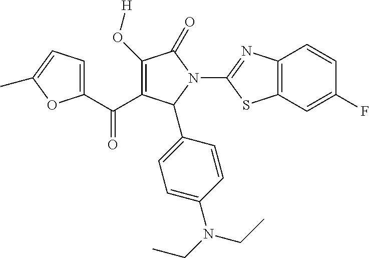 | 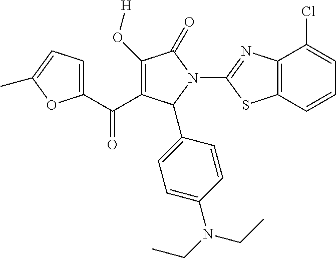 |
| 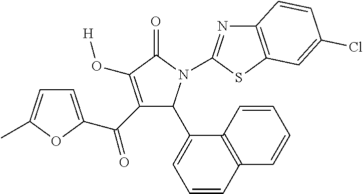 | 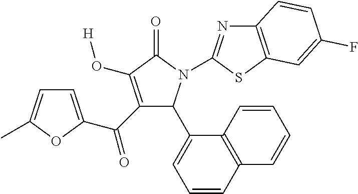 |
| 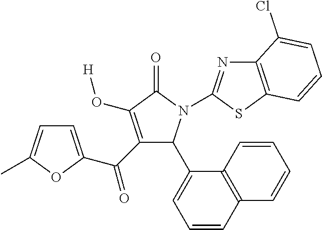 | 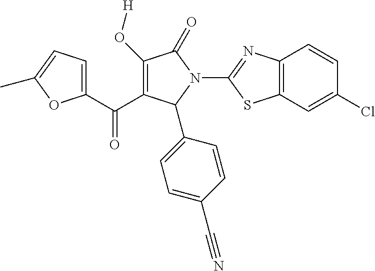 |
| 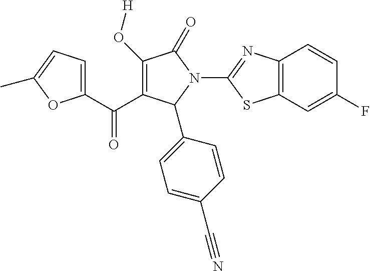 | 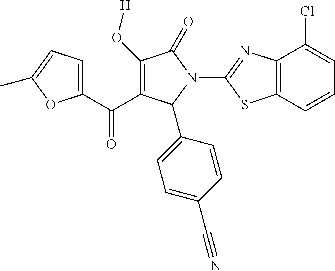 |
| 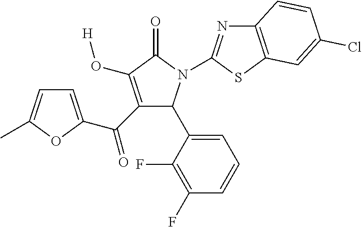 | 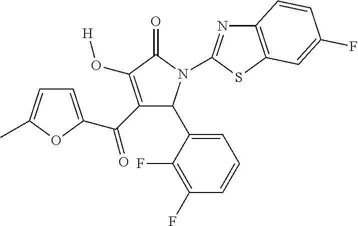 |
| 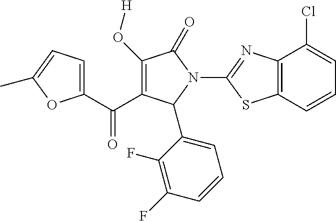 | 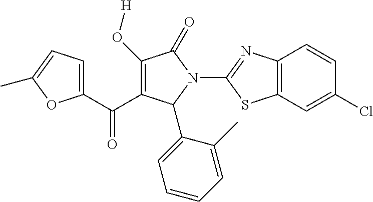 |
| 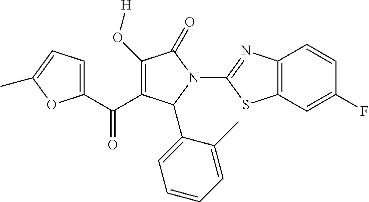 | 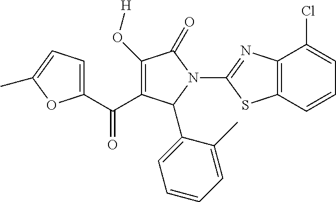 |
| 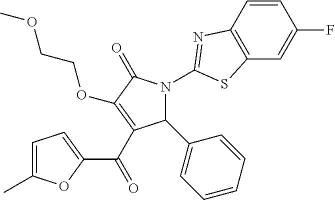 | 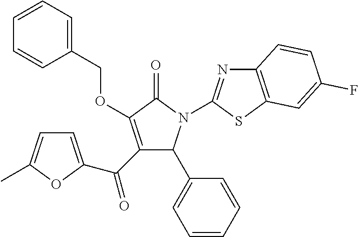 |
| 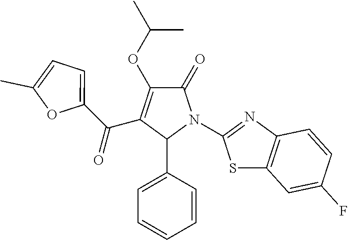 | 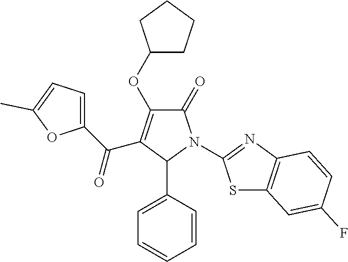 |
| 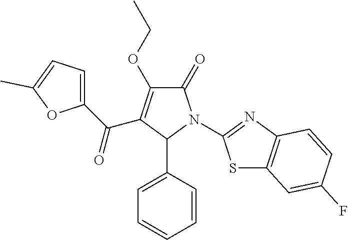 | 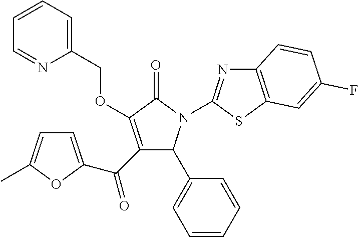 |
| 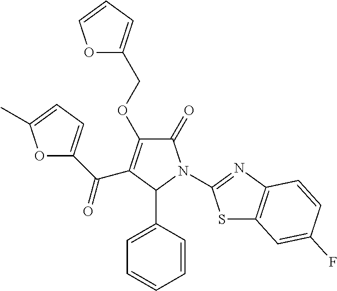 | 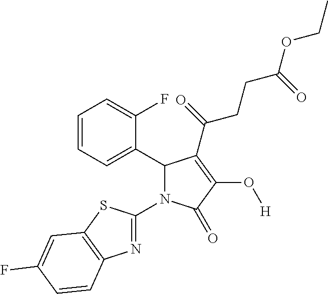 |
| 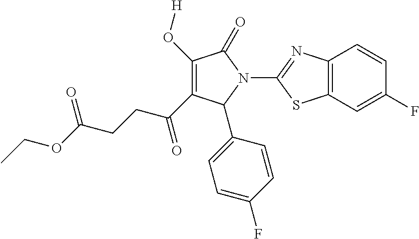 | 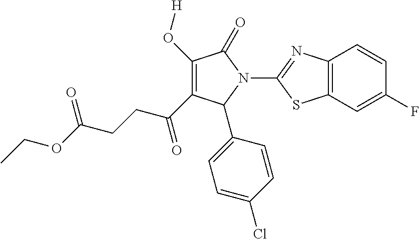 |
| 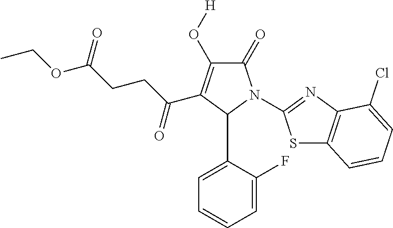 | 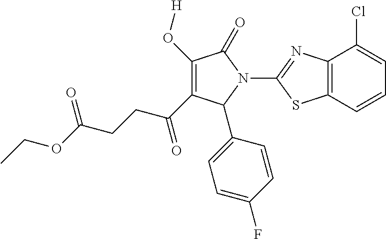 |
| 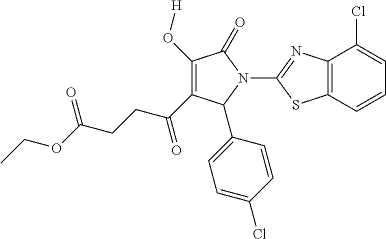 | 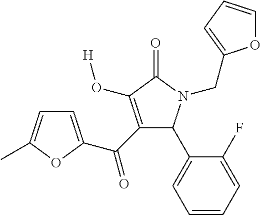 |
| 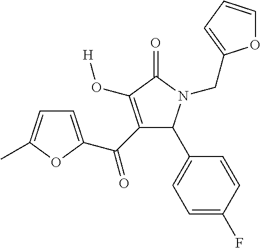 | 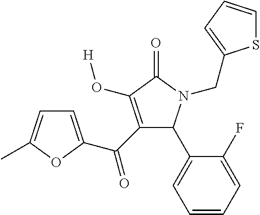 |
| 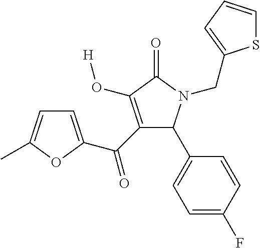 | 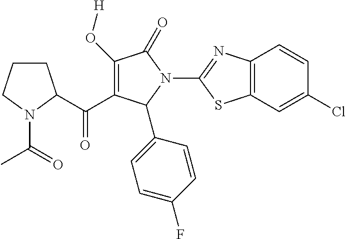 |
| 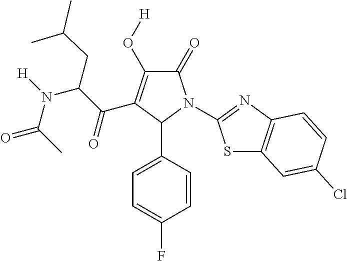 | 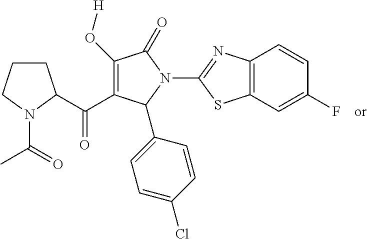 |
| 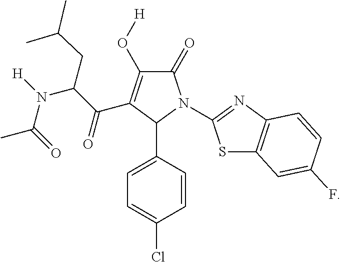 | |
